SEQUENCE LISTING (78 sequences)

SEQ ID NO: 1 - DNA, 3888 bp, Artificial Sequence
  Nucleotide Sequence of rBoNT/A(0)
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatcaactgatctacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 2 - protein, 1296 aa, Artificial Sequence
  Polypeptide Sequence of rBoNT/A(0)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 3 - DNA, 2634 bp, Artificial Sequence
  Nucleotide Sequence of rLHN/A
atggagttcgttaacaaacagttcaactataaagacccagttaacggtgttgacattgcttacatcaaaatcccgaacgctggccagatgcagccggtaaaggcattcaaaatccacaacaaaatctgggttatcccggaacgtgatacctttactaacccggaagaaggtgacctgaacccgccaccggaagcgaaacaggtgccggtatcttactatgactccacctacctgtctaccgataacgaaaaggacaactacctgaaaggtgttactaaactgttcgagcgtatttactccaccgacctgggccgtatgctgctgactagcatcgttcgcggtatcccgttctggggcggttctaccatcgataccgaactgaaagtaatcgacactaactgcatcaacgttattcagccggacggttcctatcgttccgaagaactgaacctggtgatcatcggcccgtctgctgatatcatccagttcgagtgtaagagctttggtcacgaagttctgaacctcacccgtaacggctacggttccactcagtacatccgtttctctccggacttcaccttcggttttgaagaatccctggaagtagacacgaacccactgctgggcgctggtaaattcgcaactgatcctgcggttaccctggctcacgaactgattcatgcaggccaccgcctgtacggtatcgccatcaatccgaaccgtgtcttcaaagttaacaccaacgcgtattacgagatgtccggtctggaagttagcttcgaagaactgcgtacttttggcggtcacgacgctaaattcatcgactctctgcaagaaaacgagttccgtctgtactactataacaagttcaaagatatcgcatccaccctgaacaaagcgaaatccatcgtgggtaccactgcttctctccagtacatgaagaacgtttttaaagaaaaatacctgctcagcgaagacacctccggcaaattctctgtagacaagttgaaattcgataaactttacaaaatgctgactgaaatttacaccgaagacaacttcgttaagttctttaaagttctgaaccgcaaaacctatctgaacttcgacaaggcagtattcaaaatcaacatcgtgccgaaagttaactacactatctacgatggtttcaacctgcgtaacaccaacctggctgctaattttaacggccagaacacggaaatcaacaacatgaacttcacaaaactgaaaaacttcactggtctgttcgagttttacaagctgctgtgcgtcgacggcatcattacctccaaaactaaatctgacgatgacgataaaaacaaagcgctgaacctgcagtgtatcaaggttaacaactgggatttattcttcagcccgagtgaagacaacttcaccaacgacctgaacaaaggtgaagaaatcacctcagatactaacatcgaagcagccgaagaaaacatctcgctggacctgatccagcagtactacctgacctttaatttcgacaacgagccggaaaacatttctatcgaaaacctgagctctgatatcatcggccagctggaactgatgccgaacatcgaacgtttcccaaacggtaaaaagtacgagctggacaaatataccatgttccactacctgcgcgcgcaggaatttgaacacggcaaatcccgtatcgcactgactaactccgttaacgaagctctgctcaacccgtcccgtgtatacaccttcttctctagcgactacgtgaaaaaggtcaacaaagcgactgaagctgcaatgttcttgggttgggttgaacagcttgtttatgattttaccgacgagacgtccgaagtatctactaccgacaaaattgcggatatcactatcatcatcccgtacatcggtccggctctgaacattggcaacatgctgtacaaagacgacttcgttggcgcactgatcttctccggtgcggtgatcctgctggagttcatcccggaaatcgccatcccggtactgggcacctttgctctggtttcttacattgcaaacaaggttctgactgtacaaaccatcgacaacgcgctgagcaaacgtaacgaaaaatgggatgaagtttacaaatatatcgtgaccaactggctggctaaggttaatactcagatcgacctcatccgcaaaaaaatgaaagaagcactggaaaaccaggcggaagctaccaaggcaatcattaactaccagtacaaccagtacaccgaggaagaaaaaaacaacatcaacttcaacatcgacgatctgtcctctaaactgaacgaatccatcaacaaagctatgatcaacatcaacaagttcctgaaccagtgctctgtaagctatctgatgaactccatgatcccgtacggtgttaaacgtctggaggacttcgatgcgtctctgaaagacgccctgctgaaatacatttacgacaaccgtggcactctgatcggtcaggttgatcgtctgaaggacaaagtgaacaataccttatcgaccgacatcccttttcagctcagtaaatatgtcgataaccaacgccttttgtccactctagaagcacaccatcatcaccaccatcaccatcaccat

SEQ ID NO: 4 - protein, 878 aa, Artificial Sequence
  Polypeptide Sequence of rLHN/A
MEFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVDGIITSKTKSDDDDKNKALNLQCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTLEAHHHHHHHHHH

SEQ ID NO: 5 - DNA, 1329 bp, Artificial Sequence
  Nucleotide Sequence of rL/A
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgggtctagaagcacaccatcatcaccaccatcaccatcaccat

SEQ ID NO: 6 - protein, 443 aa, Artificial Sequence
  Polypeptide Sequence of rL/A
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLGLEAHHHHHHHHHH

SEQ ID NO: 7 - DNA, 1299 bp, Artificial Sequence
  Nucleotide Sequence of rHC/A
atgcatcatcaccatcaccacaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 8 - protein, 433 aa, Artificial Sequence
  Polypeptide Sequence of rHC/A
MHHHHHHKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 9 - DNA, 3873 bp, Artificial Sequence
  Nucleotide Sequence of rBoNT/B(0)
atgccggtgacgattaacaacttcaactacaacgacccgattgacaacaacaacattatcatgatggaaccgccgtttgcacgcggcacgggccgttattacaaagcgtttaaaatcaccgatcgtatttggattatcccggaacgctacacgtttggttataaaccggaagacttcaacaaaagctctggcatcttcaaccgtgatgtttgcgaatactacgatccggactacctgaacaccaacgataagaaaaacatttttctgcaaacgatgatcaaactgttcaatcgcattaaaagcaaaccgctgggtgaaaaactgctggaaatgattatcaatggcattccgtatctgggtgatcgtcgcgtgccgctggaagaatttaacaccaatatcgcgagtgttacggtcaacaaactgatttccaatccgggtgaagtcgaacgtaaaaaaggcatcttcgccaacctgatcatcttcggcccgggtccggtgctgaacgaaaatgaaaccattgatatcggtattcagaaccattttgcctcacgcgaaggcttcggcggtattatgcaaatgaaattttgcccggaatatgtgtcggttttcaacaatgttcaggaaaacaaaggtgcaagcatctttaatcgtcgcggctatttctctgatccggctctgatcctgatgcaccaactgatttatgtgctgcacggcctgtatggtatcaaagtggatgacctgccgatcgttccgaacgagaaaaaatttttcatgcagagcaccgacgcaattcaagctgaagaactgtatacgtttggcggtcaggacccgtctattatcaccccgagcaccgacaaaagcatctacgataaagtgctgcaaaactttcgtggcattgttgaccgcctgaataaagtcctggtgtgtatctctgatccgaacatcaacatcaacatctacaaaaacaaattcaaagacaaatacaaattcgttgaagattctgaaggcaaatatagtattgacgtcgaatcctttgataaactgtacaaaagtctgatgttcggtttcaccgaaacgaacatcgcggaaaactacaaaatcaaaacccgcgcctcctatttcagcgactctctgccgccggttaaaatcaaaaatctgctggataacgaaatttatacgatcgaagaaggtttcaacatcagcgataaagacatggaaaaagaataccgtggccagaataaagcaatcaacaaacaggcgtatgaagaaattagtaaagaacatctggcggtctacaaaattcagatgtgcaaatccgtgaaagccccgggtatttgtatcgatgttgacaatgaagacctgtttttcatcgccgataaaaacagtttttccgatgacctgtcaaaaaatgaacgcatcgaatacaacacccaatcgaactacatcgaaaacgatttcccgatcaacgaactgattctggatacggacctgattagtaaaatcgaactgccgtcagaaaacaccgaatcgctgacggactttaatgttgatgtcccggtgtatgaaaaacagccggcaattaagaaaatttttaccgatgaaaacacgatcttccagtacctgtacagccaaacctttccgctggacattcgcgatatctctctgacgagttcctttgatgacgcactgctgttcagcaacaaagtgtactcctttttctcaatggattacatcaaaaccgctaacaaagtggttgaagcgggcctgtttgccggttgggtgaaacagatcgttaacgatttcgtcatcgaagccaacaaaagtaacacgatggataaaattgctgatatctccctgattgtcccgtatattggcctggcactgaatgtgggtaacgaaacggcgaaaggcaattttgaaaacgccttcgaaattgcaggcgcttcaatcctgctggaatttattccggaactgctgatcccggtcgtgggtgcgttcctgctggaatcttacatcgacaacaaaaacaaaatcatcaaaaccattgataacgcgctgacgaaacgtaacgaaaaatggtcagatatgtacggcctgattgttgcccagtggctgagcaccgtcaacacgcaattttacaccatcaaagaaggtatgtacaaagcgctgaattatcaggcgcaagccctggaagaaatcatcaaataccgctacaacatctacagcgaaaaagaaaaatctaacatcaacatcgactttaatgatatcaacagcaaactgaacgaaggtatcaaccaggcaatcgataacatcaacaacttcatcaacggctgctcagtgtcgtatctgatgaagaaaatgatcccgctggctgttgaaaaactgctggattttgacaacaccctgaagaaaaacctgctgaactacatcgatgaaaacaaactgtacctgatcggctcagccgaatacgaaaaatcgaaagtgaacaaatacctgaaaaccatcatgccgtttgacctgagtatttacaccaacgatacgatcctgatcgaaatgttcaacaaatacaactccgaaattctgaacaatattatcctgaacctgcgttacaaagacaacaatctgatcgatctgagcggctatggtgcaaaagttgaagtctacgacggtgtcgaactgaacgataaaaaccagttcaaactgacctcatcggctaactcaaaaattcgtgtgacgcagaaccaaaacatcatcttcaactcggtctttctggacttcagcgtgtctttctggattcgcatcccgaaatataaaaatgatggcatccagaactacatccataacgaatacaccatcatcaactgtatgaaaaacaacagtggttggaaaatttccatccgtggcaaccgcattatctggaccctgattgatatcaatggtaaaacgaaaagcgtgtttttcgaatacaacatccgtgaagatatctctgaatacatcaatcgctggtttttcgtgaccattacgaacaatctgaacaatgcgaaaatctatatcaacggcaaactggaaagtaataccgacatcaaagatattcgtgaagttatcgccaacggtgaaatcatcttcaaactggatggcgacatcgatcgcacccagttcatttggatgaaatacttctccatcttcaacacggaactgagtcagtccaatatcgaagaacgctacaaaatccaatcatactcggaatacctgaaagatttctggggtaacccgctgatgtacaacaaagaatactacatgttcaacgcgggcaacaaaaactcatacatcaaactgaaaaaagattcgccggtgggtgaaatcctgacccgtagcaaatacaaccagaactctaaatacatcaactatcgcgatctgtacattggcgaaaaatttattatccgtcgcaaaagcaactctcagagtattaatgatgacatcgtgcgtaaagaagactacatctatctggatttctttaatctgaaccaagaatggcgcgtttatacctacaaatacttcaaaaaagaagaagagaaactgttcctggccccgattagcgacagcgatgaattttacaacaccatccagatcaaagaatacgatgaacagccgacgtatagttgccaactgctgttcaaaaaagacgaagaatccaccgatgaaattggcctgattggtatccaccgtttctatgaaagcggtatcgttttcgaagaatacaaagattacttctgtatctctaaatggtatctgaaagaagtcaaacgcaaaccgtacaacctgaaactgggctgcaactggcaatttatcccgaaagacgaaggctggaccgaa

SEQ ID NO: 10 - protein, 1291 aa, Artificial Sequence
  Polypeptide Sequence of rBoNT/B(0)
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHQLIYVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 11 - DNA, 3873 bp, Artificial Sequence
  Nucleotide Sequence of rBoNT/C(0)
atgccgatcacgattaataatttcaactatagcgatccggtggacaataagaatattctgtatctggatactcatctgaatacgctggctaacgaaccggagaaagcgttccgcatcacaggcaacatctgggttattcccgatcgcttttcacgcaacagcaaccctaatctgaacaaacctcctcgtgtcaccagtcctaaatccggttattacgacccaaactatctgagtacggatagcgataaagatccctttctgaaagagatcattaagctgttcaaacgcattaactctcgcgaaattggggaagagctgatctatcggctttcgacagatatcccgttcccaggtaacaataataccccgattaatactttcgactttgatgttgatttcaattctgtggatgtgaaaacgcgtcaaggcaataattgggtgaaaactggtagcattaacccgagtgtaattatcacaggtccccgtgagaacatcatcgacccggaaacctctaccttcaagctgacgaacaacacgtttgctgcacaggaagggtttggtgccctgtcaatcatttccatctcaccgcgtttcatgttaacctactccaatgccacaaatgatgttggcgaaggacgttttagcaaatcagaattttgcatggacccaattctcattctgatgggcacgctgaacaatgcgatgcacaacttgtatggcattgctattccaaacgatcaaaccattagctccgttaccagtaatatcttctatagccagtataatgtcaaattggagtatgccgaaatttacgcctttggaggcccgaccattgacctgattccgaaatctgcacgcaaatacttcgaagaaaaggcgttagattactatcgcagcatcgcgaaacgcctgaactcgattaccacggccaatccgtcgtcgttcaacaaatacattggtgaatataaacagaaactgattcgcaaatatcggtttgtcgtagaaagctctggtgaagtgactgtaaaccgcaacaaatttgtcgaactctacaacgagttgacccaaatctttaccgagtttaactacgcaaagatctataacgtacagaaccgcaagatttatcttagcaatgtatacacaccggttactgcgaacatcttagacgacaatgtgtatgatattcagaatggctttaacatcccgaaatcaaatctgaacgttctgtttatgggccagaacctgagtcgtaatccagcactgcgtaaagtgaacccggaaaatatgctctacttgtttaccaaattttgccacaaagcgattgatggccgctctctctataacaaaacgctggattgtcgtgagttacttgtgaagaacactgatttaccgttcattggggatatctccgacgtgaaaaccgatatcttcctgcgcaaagacattaatgaagaaacggaagtcatctattaccccgacaatgtgagcgttgatcaggtcattttatcgaagaacacctccgaacatggtcagttggatttgctgtaccctagcattgactcggagagtgaaatccttccgggcgaaaatcaagtgttttacgacaaccgtacccaaaatgttgattatttgaattcttattactacctggaatctcagaaattgagcgacaatgtggaagatttcacgttcacacgctccattgaggaagcgctggataatagcgcgaaagtgtatacgtatttccctaccttggcgaataaagtaaatgctggtgtccagggaggcttatttctgatgtgggcgaatgatgtggtagaagattttacgaccaatattttgcgtaaggacaccttagataaaattagcgatgttagcgccatcatcccctatattggcccagcactgaatatctcgaactctgtgcgtcgcggaaacttcaccgaagcatttgcggtgaccggggttactattctgttggaagcctttccggagtttactattccggcgctgggtgcgtttgtgatttattcgaaagtacaagaacgcaatgaaattatcaaaaccatcgataattgcctggaacaacgcattaaacgctggaaggattcttatgaatggatgatgggcacctggttatcccgtattatcacacagtttaacaacatctcgtatcagatgtacgattcactgaactaccaagcaggggcgatcaaagccaagatcgacttagaatacaagaaatattcaggtagcgataaagagaatattaaaagccaggttgaaaacctgaagaactctctggatgtcaaaatttcagaggctatgaacaacattaacaaatttatccgcgaatgtagcgtcacgtatctgtttaaaaacatgctcccgaaagtgattgatgagctcaacgagtttgatcgcaacacaaaggccaaactgattaacctgattgatagtcacaatattattttagtcggtgaagttgacaagctgaaggctaaggtcaataacagctttcagaacactattccgtttaatattttctcctatacgaacaatagtctgctgaaagacattatcaacgaatacttcaacaatattaatgacagcaaaattctgagcctgcagaatcgtaagaatacgctggtagataccagtggatataatgcggaagtctcagaagagggtgatgtacagctgaacccgatctttccgttcgactttaaactggggtctagtggtgaagatcgcggtaaagtgatcgttacccaaaacgagaacattgtgtataacagcatgtacgagagtttctcaatttctttctggattcgcatcaataaatgggtttctaatttgcctggctataccatcattgatagcgtcaaaaacaactcgggctggtcgattggcattattagcaactttctggtgtttaccctgaaacagaatgaggattcggaacagagcattaacttctcctacgacatcagcaacaatgcaccagggtataacaaatggttcttcgtaacggtgacgaacaatatgatgggcaatatgaaaatctacattaacgggaaacttatcgacaccattaaagtgaaagagcttactgggatcaattttagtaaaaccattacctttgagatcaacaaaattccggacacgggtctgattacctccgattcggataatatcaatatgtggattcgcgacttttatatcttcgccaaagaacttgatggcaaagatatcaacattttgtttaattccctgcagtataccaatgtcgttaaggactattggggcaatgatctccgctacaataaagaatactacatggttaacatcgactatctcaatcgctacatgtatgctaactcgcgtcaaattgtgtttaacacacgtcgtaacaacaacgattttaacgaaggttataaaatcattatcaaacggatccgcggcaatacgaacgatactcgtgttcgtggcggtgacattctgtatttcgacatgacgattaataataaagcgtacaatctgttcatgaagaacgaaaccatgtacgccgataaccattccactgaagatatctacgcaatcggacttcgcgaacagaccaaagacattaacgacaacatcatctttcagattcaaccgatgaataatacctactactatgcctcccagatcttcaaaagtaatttcaacggcgaaaacatttcaggcatttgctcaatcggcacttatcggttccggttaggtggtgattggtatcgtcacaactaccttgttcccacagtgaaacaaggcaactatgcatcgctcttagaaagcacatctacgcattggggttttgtgccagtcagtgaa

SEQ ID NO: 12 - protein, 1291 aa, Artificial Sequence
  Polypeptide Sequence of rBoNT/C(0)
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMGTLNNAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 13 - DNA, 3756 bp, Artificial Sequence
  Nucleotide Sequence of rBoNT/E(0)
atgccgaaaatcaactctttcaactacaacgacccggttaacgaccgtaccatcctgtatatcaaaccgggtggttgccaggagttctacaaatctttcaacatcatgaaaaacatctggatcatcccggaacgtaacgttatcggtaccaccccgcaggacttccacccgccgacctctctgaaaaacggtgactcttcttactacgacccgaactacctccagtctgacgaagaaaaagaccgtttcctgaaaatcgttaccaaaatcttcaaccgtatcaacaacaacctgtctggtggtatcctgctggaagaactgtctaaagctaacccgtacctgggtaacgacaacaccccggacaaccagttccacatcggtgacgcttctgctgttgaaatcaaattctctaacggttctcaggacatcctgctgccgaacgttatcatcatgggtgctgaaccggacctgttcgaaaccaactcttctaacatctctctgcgtaacaactacatgccgtctaaccacggtttcggttctatcgctatcgttaccttctctccggaatactctttccgtttcaacgacaacagcatgaacgagttcatccaggacccggctctgaccctgatgcaccaactgatctactctctgcacggtctgtacggtgctaaaggtatcaccaccaaatacaccatcacccagaaacagaacccgctgatcaccaacatccgtggtaccaacatcgaagagttcctgaccttcggtggtaccgacctgaacatcatcacctctgctcagtctaacgacatctacaccaacctgctggctgactacaaaaaaatcgcttctaaactgtctaaagttcaggtttctaacccgctgctgaacccgtacaaagacgttttcgaagctaaatacggtctggacaaagacgcttctggtatctactctgttaacatcaacaaattcaacgacatcttcaaaaaactgtactctttcaccgagttcgacctggcgaccaaattccaggttaaatgccgtcagacctacatcggtcagtacaaatacttcaaactgtctaacctgctgaacgactctatctacaacatctctgaaggttacaacatcaacaacctgaaagttaacttccgtggtcagaacgctaacctgaacccgcgtatcatcaccccgatcaccggtcgtggtctggttaaaaaaatcatccgtttctgcaagaatattgtaagcgttaaaggaataagaaaaagtatctgcatcgaaatcaacaacggtgaactgttcttcgttgcttctgaaaactcttacaacgacgacaacatcaacaccccgaaagaaatcgacgacaccgttacctctaacaacaactacgaaaacgacctggaccaggttatcctgaacttcaactctgaatctgctccgggtctgtctgacgaaaaactgaacctgaccatccagaacgacgcttacatcccgaaatacgactctaacggtacctctgacatcgaacagcacgacgttaacgaactgaacgttttcttctacctggacgctcagaaagttccggaaggtgaaaacaacgttaacctgacctcttctatcgacaccgctctgctggaacagccgaaaatctacaccttcttctcttctgagttcatcaacaacgttaacaaaccggttcaggctgctctgttcgtttcttggattcagcaggttctggttgacttcaccaccgaagctaaccagaaatctaccgttgacaaaatcgctgacatctctatcgttgttccgtacatcggtctggctctgaacatcggtaacgaagctcagaaaggtaacttcaaagacgctctggaactgctgggtgctggtatcctgctggagttcgaaccggaactgctgatcccgaccatcctggttttcaccatcaaatctttcctgggttcttctgacaacaaaaacaaagttatcaaagctatcaacaacgctctgaaagaacgtgacgaaaaatggaaagaagtttactctttcatcgtttctaactggatgaccaaaatcaacacccagttcaacaaacgtaaagaacagatgtaccaggctctccagaaccaggttaacgctatcaaaaccatcatcgaatctaaatacaactcttacaccctggaagaaaaaaacgaactgaccaacaaatacgacatcaaacagatcgaaaacgaactgaaccagaaagtttctatcgctatgaacaacatcgaccgtttcctgaccgaatcttctatctcttacctgatgaaactcatcaacgaagttaaaatcaacaaactgcgtgaatacgacgaaaacgttaaaacctacctgctgaactacatcatccagcacggttctatcctgggtgaatctcagcaggaactgaactctatggttaccgacaccctgaacaactctatcccgttcaaactgtcttcttacaccgacgacaaaatcctgatctcttacttcaacaaattctttaaacgcattaagagttcatcggttctgaatatgcggtacaaaaatgataaatatgtcgatacttctggatatgatagcaatatcaacattaacggcgacgtgtataaatatccgacaaataaaaaccagtttgggatatataacgacaagctgtcggaggtcaatatttctcaaaacgactatatcatttacgataataaatataaaaactttagcattagtttttgggttcgtatacctaattatgacaataaaattgtaaatgtgaataacgagtataccattataaactgtatgcgcgacaataacagtggttggaaggtatcgctgaaccataatgagattatctggaccctgcaggataatgcaggtataaaccagaaactggcttttaactatggaaacgcaaatgggatctcagattacattaataaatggatttttgttaccattacgaacgatcgcttaggcgactcaaaactttatattaatggcaatctgatagatcagaaatcaatcttaaatttgggcaatattcatgtctctgataacatcttgttcaagatcgttaattgcagttacactcgttatattggcattcgttactttaatatcttcgataaagaactggacgagacggaaatccagactctgtattcaaacgagcccaatactaatatattgaaagatttttggggtaactatcttttatatgataaagaatactatctcctgaatgtattgaagccaaacaatttcatagatagacgcaaggatagcacattaagtatcaacaatatcagatctactatactgttagcaaatcgcctctactccggtattaaagtgaagattcagcgggttaataactccagtaccaatgataatctggtccgtaagaacgatcaggtatacatcaatttcgtcgcgagcaaaactcatctcttcccgctttacgccgatacagctacgacaaacaaggaaaaaaccataaaaatttccagctccggaaacagattcaatcaagtagttgtaatgaactctgtgggtaataattgtacgatgaactttaagaataacaatgggaacaatattggacttttgggcttcaaagccgacacagtggtggcgtccacctggtattacacgcacatgcgggaccatacgaattcgaacggttgcttctggaactttatctcggaagaacacgggtggcaagaaaaa

SEQ ID NO: 14 - protein, 1252 aa, Artificial Sequence
  Polypeptide Sequence of rBoNT/E(0)
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHQLIYSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 15 - DNA, 3834 bp, Artificial Sequence
  Nucleotide Sequence of rBoNT/F(0)
atgccggtggtcatcaacagcttcaactacaacgacccagtaaacgacgacacgatcctgtatatgcaaatcccgtatgaagagaagagcaagaagtactataaggcctttgaaatcatgcgcaatgtgtggattattccggagcgtaatacgattggtactgacccaagcgacttcgatccacctgcgtctttggaaaacggctcgtccgcatattacgacccgaattacctgaccaccgatgcggagaaagatcgttatttgaaaaccaccatcaagctgttcaaacgcattaacagcaatccggcaggtgaggtcctgctgcaagagattagctacgcaaagccttatctgggtaatgagcatacgcctattaacgagtttcacccggttacccgcactaccagcgttaacatcaagtcctcgaccaacgtgaagtctagcattatcctgaacctgctggttctgggtgccggtccggacatcttcgaaaactctagctacccggtgcgtaaactgatggatagcggcggtgtttatgacccgagcaatgacggttttggcagcatcaatatcgtgacgtttagcccggagtacgagtacaccttcaatgatatcagcggtggttacaattcttctaccgagagcttcatcgccgacccggcgatcagcctggcacaccaactgatctatgcattgcatggcttgtacggtgcccgtggtgtgacgtataaagagactatcaaggttaagcaggcacctctgatgattgcggaaaagccgattcgcctggaagagttcctgaccttcggcggtcaagatttgaacatcattacctcggccatgaaagagaaaatctataacaatttgctggccaactatgaaaagattgcaacgcgcttgtctcgtgttaactccgctccgccggaatacgacattaatgagtacaaagactactttcaatggaaatatggcctggacaaaaatgcggatggttcttataccgtgaatgaaaacaaattcaatgaaatctacaagaaactgtacagcttcaccgaaatcgatctggcgaacaagttcaaagtcaaatgtcgtaatacctacttcatcaaatatggcttcctgaaagtcccgaacctgctggacgatgacatctataccgtcagcgaaggcttcaacatcggcaatctggccgtgaataatcgtggtcagaacatcaaactgaatccgaaaatcattgactccatcccagacaagggcctggttgagaaaatcgtgaagttctgcaaaagcgttattccgcgtaaaggtacgaaagcaccgcctcgcctgtgcattcgcgttaacaaccgtgagttgttctttgtggcatctgaaagcagctacaacgagaacgacatcaacacccctaaagaaattgatgataccacgaacctgaataacaattatcgcaacaatctggacgaggtgatcctggattacaattcggaaaccattccgcaaattagcaatcagacgctgaacaccctggttcaggacgatagctacgttccgcgttacgactccaatggtactagcgagattgaagaacacaacgtagtggacttgaacgttttcttttatctgcacgcccagaaggttccggagggcgaaaccaatattagcctgaccagctcgatcgacaccgcgctgtctgaggagagccaagtctacacctttttcagcagcgagtttatcaacactattaacaagccagttcatgctgcattgtttatctcttggattaaccaggtgattcgcgactttacgacggaggcgacccagaagtctaccttcgacaaaattgcagacatctccctggtcgtcccatacgtcggcctggcgttgaatattggcaatgaagttcaaaaagagaacttcaaagaagcgttcgagctgctgggtgcaggcatcctgctggagttcgtgccggaactgttgatcccgaccatcctggtgttcaccattaagagcttcattggatcctccgagaataagaacaagatcatcaaggcgatcaataacagcctgatggagcgtgaaacgaagtggaaagaaatctatagctggattgttagcaattggctgactcgtattaacacgcaattcaacaagcgtaaagagcaaatgtaccaagccctgcaaaaccaagttgacgccatcaaaacggtaattgaatacaagtacaacaattacacgagcgatgagcgcaaccgcctggaaagcgaatacaacatcaacaacattcgcgaagaattgaacaagaaagtgagcctggcgatggagaacattgagcgttttatcaccgaaagcagcatcttttacctgatgaaattgattaatgaggcgaaagtctcgaaactgcgtgagtacgacgaaggtgtgaaagagtatctgctggattacattagcgagcaccgtagcatcttgggtaactcggttcaggagctgaacgatctggtgacctctaccctgaacaatagcatcccgttcgaactgagcagctataccaatgacaagattctgattctgtatttcaataaactgtataagaagatcaaggataacagcattctggatatgcgttacgaaaacaataagtttatcgacatttctggttacggcagcaacatttccatcaatggcgatgtctacatctacagcaccaatcgcaaccagttcggcatctactctagcaaaccgagcgaagttaacatcgcacagaacaatgatattatttataacggtcgttatcaaaacttctctatcagcttttgggtccgtatcccgaagtacttcaataaagtcaatctgaataatgaatacacgatcatcgactgcattcgcaataacaacagcggttggaaaatcagcctgaattacaacaaaattatttggaccctgcaagatacggcgggtaacaatcagaaactggtgtttaactacacgcaaatgatcagcatttctgactatatcaacaagtggatctttgttaccatcaccaataatcgtctgggcaatagccgtatttacatcaacggtaacctgattgatgagaaaagcatcagcaacctgggcgatattcacgtcagcgacaacattctgttcaaaattgttggttgtaacgatacccgttacgtcggcatccgttatttcaaggttttcgatacggagctgggtaaaacggaaatcgaaacgttgtactccgatgaaccagatccgagcattctgaaggacttttggggtaactacttgctgtacaataaacgttactatctgctgaatctgttgcgcaccgacaagagcattacccaaaacagcaatttcctgaacattaatcagcaacgcggcgtataccaaaaaccgaacatcttcagcaatacgcgcctgtatactggtgttgaagtgatcattcgtaagaacggtagcaccgacattagcaacacggacaatttcgtccgtaagaatgacctggcgtacattaacgtcgtggaccgtgatgtcgagtatcgtctgtacgcagacatcagcattgcgaaaccggaaaagattatcaagctgatccgtaccagcaacagcaacaacagcctgggtcagatcattgtgatggacagcattggtaataactgcacgatgaacttccagaacaacaatggtggtaatatcggtctgctgggttttcacagcaataatctggttgcttccagctggtactacaataacattcgtaaaaacacgtctagcaatggttgtttttggagctttatcagcaaagagcacggctggcaagaaaat

SEQ ID NO: 16 - protein, 1278 aa, Artificial Sequence
  Polypeptide Sequence of rBoNT/F(0)
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHQLIYALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 17 - DNA, 3942 bp, Artificial Sequence
  Nucleotide Sequence of rBoNT/A(0) (His-tagged)
atgccgtttgtgaacaagcagttcaactataaagatccggttaatggtgtggatatcgcctatatcaaaattccgaatgcaggtcagatgcagccggttaaagcctttaaaatccataacaaaatttgggtgattccggaacgtgatacctttaccaatccggaagaaggtgatctgaatccgcctccggaagcaaaacaggttccggttagctattatgatagcacctatctgagcaccgataacgagaaagataactatctgaaaggtgtgaccaaactgtttgaacgcatttatagtaccgatctgggtcgtatgctgctgaccagcattgttcgtggtattccgttttggggtggtagcaccattgataccgaactgaaagttattgacaccaactgcattaatgtgattcagccggatggtagctatcgtagcgaagaactgaatctggttattattggtccgagcgcagatatcattcagtttgaatgtaaaagctttggccacgaagttctgaatctgacccgtaatggttatggtagtacccagtatattcgtttcagtccggattttacctttggctttgaagaaagcctggaagttgatacaaatccgctgttaggtgcaggtaaatttgcaaccgatccggcagttaccctggcacaccagctgatttatgccggtcatcgtctgtatggtattgccattaatccgaatcgtgtgttcaaagtgaataccaacgcctattatgaaatgagcggtctggaagtgagttttgaagaactgcgtacctttggtggtcatgatgccaaatttatcgatagcctgcaagaaaatgaatttcgcctgtactactataacaaattcaaggatattgcgagcaccctgaataaagccaaaagcattgttggcaccaccgcaagcctgcagtatatgaaaaatgtgtttaaagaaaaatatctgctgagcgaagataccagcggtaaatttagcgttgacaaactgaaattcgataaactgtacaagatgctgaccgagatttataccgaagataacttcgtgaagtttttcaaagtgctgaaccgcaaaacctacctgaactttgataaagccgtgttcaaaatcaacatcgtgccgaaagtgaactataccatctatgatggttttaacctgcgcaataccaatctggcagcaaactttaatggtcagaacaccgaaatcaacaacatgaactttaccaaactgaagaacttcaccggtctgttcgaattttacaaactgctgtgtgttcgtggcattattaccagcaaaaccaaaagtctggataaaggctacaataaagccctgaatgatctgtgcattaaggtgaataattgggacctgttttttagcccgagcgaggataatttcaccaacgatctgaacaaaggcgaagaaattaccagcgataccaatattgaagcagccgaagaaaacattagcctggatctgattcagcagtattatctgaccttcaacttcgataatgagccggaaaatatcagcattgaaaacctgagcagcgatattattggccagctggaactgatgccgaatattgaacgttttccgaacggcaaaaaatacgagctggataaatacaccatgttccattatctgcgtgcccaagaatttgaacatggtaaaagccgtattgcactgaccaatagcgttaatgaagcactgctgaacccgagccgtgtttataccttttttagcagcgattacgtgaaaaaggttaacaaagcaaccgaagcagccatgtttttaggttgggttgaacagctggtttatgatttcaccgatgaaaccagcgaagttagcaccaccgataaaattgcagatattaccatcatcatcccgtatatcggtccggcactgaatattggcaatatgctgtataaagacgattttgtgggtgccctgatttttagcggtgcagttattctgctggaatttattccggaaattgccattccggttctgggcacctttgcactggtgagctatattgcaaataaagttctgaccgtgcagaccatcgataatgcactgagcaaacgtaacgaaaaatgggatgaagtgtacaagtatatcgtgaccaattggctggcaaaagttaacacccagattgacctgattcgcaagaagatgaaagaagcactggaaaatcaggcagaagcaaccaaagccattatcaactatcagtataaccagtacaccgaagaagagaaaaataacatcaacttcaacatcgacgatctgtccagcaaactgaacgaaagcatcaacaaagccatgattaacattaacaaatttctgaaccagtgcagcgtgagctatctgatgaatagcatgattccgtatggtgtgaaacgtctggaagattttgatgcaagcctgaaagatgccctgctgaaatatatctatgataatcgtggcaccctgattggtcaggttgatcgtctgaaagataaagtgaacaacaccctgagtaccgatattccttttcagctgagcaaatatgtggataatcagcgtctgctgtcaacctttaccgaatacattaagaacatcatcaacaccagcattctgaacctgcgttatgaaagcaatcatctgattgatctgagccgttatgccagcaaaatcaatataggcagcaaggttaacttcgacccgattgacaaaaatcagatacagctgtttaatctggaaagcagcaaaattgaggtgatcctgaaaaacgccattgtgtataatagcatgtacgagaatttctcgaccagcttttggattcgtatcccgaaatactttaatagcatcagcctgaacaacgagtacaccattattaactgcatggaaaacaatagcggctggaaagttagcctgaattatggcgaaattatctggaccctgcaggatacccaagaaatcaaacagcgtgtggttttcaaatacagccagatgattaatatcagcgactatatcaaccgctggatttttgtgaccattaccaataatcgcctgaataacagcaagatctatattaacggtcgtctgattgaccagaaaccgattagtaatctgggtaatattcatgcgagcaacaacatcatgtttaaactggatggttgtcgtgatacccatcgttatatttggatcaagtacttcaacctgttcgataaagagttgaacgaaaaagaaattaaagacctgtatgataaccagagcaacagcggtattctgaaggatttttggggagattatctgcagtatgacaaaccgtattatatgctgaatctgtacgacccgaataaatacgtggatgtgaataatgttggcatccgtggttatatgtacctgaaaggtccgcgtggtagcgttatgaccacaaacatttatctgaatagcagcctgtatcgcggaaccaaattcatcattaaaaagtatgccagcggcaacaaggataatattgtgcgtaataatgatcgcgtgtacattaacgttgtggtgaagaataaagaatatcgcctggcaaccaatgcaagccaggcaggcgttgaaaaaattctgagtgccctggaaattccggatgttggtaatctgagccaggttgttgtgatgaaaagcaaaaatgatcagggcatcaccaacaagtgcaaaatgaatctgcaggacaataacggcaacgatattggttttattggcttccaccagttcaacaatattgcgaaactggttgcaagcaattggtataatcgtcagattgaacgtagcagtcgtaccctgggttgtagctgggaatttatccctgtggatgatggttggggtgaacgtccgctggaaaacctgtattttcaaggtgcaagtcatcatcaccatcaccaccatcattaa

SEQ ID NO: 18 - protein, 1313 aa, Artificial Sequence
  Polypeptide Sequence of rBoNT/A(0) (His-tagged)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPLENLYFQGASHHHHHHHH

SEQ ID NO: 19 - DNA, 2649 bp, Artificial Sequence
  Nucleotide Sequence of rLHN/A (His-tagged)
atgccgtttgtgaacaagcagttcaactataaagatccggttaatggtgtggatatcgcctatatcaaaattccgaatgcaggtcagatgcagccggttaaagcctttaaaatccataacaaaatttgggtgattccggaacgtgatacctttaccaatccggaagaaggtgatctgaatccgcctccggaagcaaaacaggttccggttagctattatgatagcacctatctgagcaccgataacgagaaagataactatctgaaaggtgtgaccaaactgtttgaacgcatttatagtaccgatctgggtcgtatgctgctgaccagcattgttcgtggtattccgttttggggtggtagcaccattgataccgaactgaaagttattgacaccaactgcattaatgtgattcagccggatggtagctatcgtagcgaagaactgaatctggttattattggtccgagcgcagatatcattcagtttgaatgtaaatcctttggccacgaagttctgaatctgacccgtaatggttatggtagtacccagtatattcgtttcagtccggattttacctttggctttgaagaaagcctggaagttgatacaaatccgctgttaggtgcaggtaaatttgcaaccgatccggcagttaccctggcacatgaactgattcatgccggtcatcgtctgtatggtattgcaattaatccgaaccgtgtgttcaaagtgaataccaacgcatattatgaaatgagcggtctggaagtgtcatttgaagaactgcgtacctttggtggtcatgatgccaaatttatcgatagcctgcaagaaaatgaatttcgcctgtactactataacaaattcaaggatattgcgagcaccctgaataaagccaaaagcattgttggcaccaccgcaagcctgcagtatatgaaaaatgtgtttaaagaaaaatatctgctgagcgaagataccagcggtaaatttagcgttgacaaactgaaattcgataaactgtacaagatgctgaccgagatttataccgaagataacttcgtgaagtttttcaaagtgctgaaccgcaaaacctacctgaactttgataaagccgtgttcaaaatcaacatcgtgccgaaagtgaactataccatctatgatggttttaacctgcgcaataccaatctggcagcaaactttaatggtcagaacaccgaaatcaacaacatgaactttaccaaactgaagaacttcaccggtctgttcgaattttacaaactgctgtgtgttcgtggcattattaccagcaaaaccaaaagtctggataaaggctacaataaagccctgaatgatctgtgcattaaggtgaataattgggacctgttttttagcccgagcgaggataatttcaccaacgatctgaacaaaggcgaagaaattaccagcgataccaatattgaagcagccgaagaaaacattagcctggatctgattcagcagtattatctgaccttcaacttcgataatgagccggaaaatatcagcattgaaaacctgagcagcgatattattggccagctggaactgatgccgaatattgaacgttttccgaacggcaaaaaatacgagctggataaatacaccatgttccattatctgcgtgcccaagaatttgaacatggtaaaagccgtattgcactgaccaatagcgttaatgaagcactgctgaacccgagccgtgtttataccttttttagcagcgattacgtgaaaaaggttaacaaagcaaccgaagcagccatgtttttaggttgggttgaacagctggtttatgatttcaccgatgaaaccagcgaagttagcaccaccgataaaattgcagatattaccatcatcatcccgtatatcggtccggcactgaatattggcaatatgctgtataaagacgattttgtgggtgccctgatttttagcggtgcagttattctgctggaatttattccggaaattgccattccggttctgggcacctttgcactggtgagctatattgcaaataaagttctgaccgtgcagaccatcgataatgcactgagcaaacgtaacgaaaaatgggatgaagtgtacaagtatatcgtgaccaattggctggcaaaagttaacacccagattgacctgattcgcaagaagatgaaagaagcactggaaaatcaggcagaagcaaccaaagccattatcaactatcagtataaccagtacaccgaagaagagaaaaataacatcaacttcaacatcgacgatctgtccagcaaactgaacgaaagcatcaacaaagccatgattaacattaacaaatttctgaaccagtgcagcgtgagctatctgatgaatagcatgattccgtatggtgtgaaacgtctggaagattttgatgcaagcctgaaagatgccctgctgaaatatatctatgataatcgtggcaccctgattggtcaggttgatcgtctgaaagataaagtgaacaacaccctgagtaccgatattccttttcagctgagcaaatatgtggataatcagcgtctgctgtcaaccgaaaatctgtatttccagggtgcaagtcatcatcaccatcaccaccatcattaa

SEQ ID NO: 20 - protein, 882 aa, Artificial Sequence
  Polypeptide Sequence of rLHN/A (His-tagged)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTENLYFQGASHHHHHHHH

SEQ ID NO: 21 - DNA, 1323 bp, Artificial Sequence
  Nucleotide Sequence of rHC/A (His-tagged)
atgcatcatcaccatcaccacgaaaatctatacttccaaggaaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctgtaa

SEQ ID NO: 22 - protein, 440 aa, Artificial Sequence
  Polypeptide Sequence of rHC/A (His-tagged)
MHHHHHHENLYFQGKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 23 - DNA, 1326 bp, Artificial Sequence
  Nucleotide Sequence of rLC/A (His-tagged)
atgccgtttgtgaacaagcagttcaactataaagatccggttaatggtgtggatatcgcctatatcaaaattccgaatgcaggtcagatgcagccggttaaagcctttaaaatccataacaaaatttgggtgattccggaacgtgatacctttaccaatccggaagaaggtgatctgaatccgcctccggaagcaaaacaggttccggttagctattatgatagcacctatctgagcaccgataacgagaaagataactatctgaaaggtgtgaccaaactgtttgaacgcatttatagtaccgatctgggtcgtatgctgctgaccagcattgttcgtggtattccgttttggggtggtagcaccattgataccgaactgaaagttattgacaccaactgcattaatgtgattcagccggatggtagctatcgtagcgaagaactgaatctggttattattggtccgagcgcagatatcattcagtttgaatgtaaatcctttggccacgaagttctgaatctgacccgtaatggttatggtagtacccagtatattcgtttcagtccggattttacctttggctttgaagaaagcctggaagttgatacaaatccgctgttaggtgcaggtaaatttgcaaccgatccggcagttaccctggcacatgaactgattcatgccggtcatcgtctgtatggtattgcaattaatccgaaccgtgtgttcaaagtgaataccaacgcatattatgaaatgagcggtctggaagtgtcatttgaagaactgcgtacctttggtggtcatgatgccaaatttatcgatagcctgcaagaaaatgaatttcgcctgtactactataacaaattcaaggatattgcgagcaccctgaataaagccaaaagcattgttggcaccaccgcaagcctgcagtatatgaaaaatgtgtttaaagaaaaatatctgctgagcgaagataccagcggtaaatttagcgttgacaaactgaaattcgataaactgtacaagatgctgaccgagatttataccgaagataacttcgtgaagtttttcaaagtgctgaaccgcaaaacctacctgaactttgataaagccgtgttcaaaatcaacatcgtgccgaaagtgaactataccatctatgatggttttaacctgcgcaataccaatctggcagcaaactttaatggtcagaacaccgaaatcaacaacatgaactttaccaaactgaagaacttcaccggtctgtttgaagagaatctgtatttccagggtgcaagtcatcatcaccatcaccaccatcattaa

SEQ ID NO: 24 - protein, 441 aa, Artificial Sequence
  Polypeptide Sequence of rLC/A (His-tagged)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEENLYFQGASHHHHHHHH

SEQ ID NO: 25 - DNA, 3918 bp, Artificial Sequence
  Nucleotide Sequence of rBoNT/FA(0) (His-tagged)
atgccggttgtgattaacagcttcaattatgatgatccggtgaacgataacaccatcatttatatccgtccgccttattatgaaaccagcaacacctatttcaaagccttccagattatggataacgtgtggattattccggaacgttatcgtctgggtattgatccgagcctgtttaatccgcctgttagcctgaaagcaggtagtgatggttattttgatccgaattatctgagcaccaacaccgagaaaaacaaatacctgcagattatgatcaagctgttcaaacgcattaatagcaaaccggcaggtcagattctgctggaagaaatcaaaaatgcaattccgtatctgggcaacagctatacccaagaagaacagtttaccaccaataatcgtaccgtgagctttaatgttaaactggccaatggtaatatcgttcagcagatggcaaatctgattatttggggtccgggtcctgatctgaccacaaataaaaccggtggtatcatctatagcccgtatcagagcatggaagcaaccccgtataaagatggttttggtagcattatgaccgtggaatttagtccggaatatgcaaccgcctttaacgatatttcaattgcaagccatagtccgtcgctgtttatcaaagatccggcactgattctgatgcaccagctgatttatgttctgcatggtctgtatggcacctatatcaccgaatacaaaattaccccgaatgtggttcagagctatatgaaagttaccaaaccgattaccagcgcagaatttctgacctttggtggtcgtgatcgcaatattgttccgcagagcattcagagccagctgtataacaaagttctgagcgattataaacgtattgccagccgtctgaataaagttaataccgcaaccgcactgatcaacatcgatgaattcaaaaacctgtacgagtggaaataccagtttgccaaagatagcaatggtgtgtatagcgtggatctgaacaaatttgagcagctgtacaaaaaaatctatagcttcaccgaattcaacctggcctatgagtttaaaatcaaaacccgtctgggttatctggccgaaaattttggtccgttttatctgccgaatctgctggatgatagcatttataccgaagtggatggttttaacattggtgcactgagcattaactatcagggtcagaatattggcagcgatatcaacagcatcaaaaaactgcaaggtcagggtgttgttagccgtgttgttcgtctgtgtagcaatagcaataccaaaaacagcctgtgcattaccgttaataatcgcgacctgttttttatcgcaagccaagaaagctatggcgagaataccattaacacctataaagagattgacgataccaccacactggatccgagctttgaagatattctggataaagtgatcctgaacttcaacgaacaggttattccgcagatgccgaatcgtaatgttagcaccgatattcagaaagacaactacatcccgaaatacgattataaccgcaccgacattatcgatagctatgaagttggtcgcaactacaacacctttttctatctgaatgcccagaaatttagcccgaacgaaagcaatattaccctgaccagcagctttgatacaggtctgttagaaggtagcaaagtgtatacctttttcagcagcgatttcattaacaacatcaacaaaccggttcaggccctgctgtttattgaatgggttaaacaggtgattcgcgattttaccaccgaagcaaccaaaacctcaaccgttgataaactgaaagatattagcctggtggtgccgtatattggtctggcactgaatattggtgatgagatctacaaacagcattttgcagaagcagttgaactggttggtgcaggtctgctgctggaattttcaccggaatttcttattccgacgctgctgatttttaccatcaaaggttatctgaccggtagcattcgcgataaagacaaaatcattaaaaccctggataacgccctgaatgttcgtgatcagaaatggaaagaactgtatcgttgggttgttagcaaatggctgaccaccattaatacgcagttcaacaaacgcaaagaacaaatgtacaaagccctgaaaaatcaggccaccgccattaaaaagatcatcgagaacaaatataacaactataccaccgatgaaaaaagcaagatcgatagcagctataacatcaacgaaattgaacgcaccctgaacgaaaaaatcaatctggccatgaaaaacatcgagcagtttattaccgaaagcagcattgcctatctgatcaatatcatcaacaacgaaacgatccagaaactgaaaagctatgatgacctggttcgtcgttatctgctgggttatattcgtaatcatagcagcattctgggcaatagcgttgaagaactgaattccaaagtgaacaaccatctggataatggcattccgtttgaactgagcagttataccaatgatagcctgctgatccgctacttcaataaaaactatggcgaactgaagtacaactgcattctgaacatcaaatatgagatggatcgtgacaaactggttgatagcagcggttatcgtagccgtatcaatattggtacaggcgtcaaatttagcgagatcgataaaaatcaagtgcagctgagcaatctggaatccagcaaaattgaagtcattctgaataacggcgtcatctataacagcatgtatgaaaacttttcgaccagcttttggattcgcattccgaaatactttcgcaacatcaataacgagtacaagatcatcagctgtatgcagaataatagcggttgggaagtgagcctgaattttagcaatatgaactcgaaaatcatctggaccctgcaggataccgaaggtatcaaaaaaaccgttgtgtttcagtacacccagaacattaacattagcgactatatcaaccgctggatctttgtgaccattacaaataatcgtctgagcaacagcaaaatctacattaatggtcgcctgatcaacgaagaaagcattagcgatctgggtaatatccatgccagcaacaacattatgtttaaactggatggttgccgtgatccgcatcgttatatctggattaaatactttaacctgtttgacaaagagctgaacaagaaagaaattaaagatctgtacgacaaccagagcaatagcggtattctgaaagatttctggggtgattatctgcagtatgacaaaccgtattatatgctgaatctgtatgacccgaataagtatctggatgtgaataatgttggcatccgtggctatatgtatctgaaaggtccgcgtggtcgtattgtgaccaccaacatttatctgaatagcaccctgtatatgggcaccaaattcatcattaagaaatatgccagcggcaacaaagataacattgtgcgtaataatgatcgcgtgtatattaacgtggtggtgaagaataaagaatatcgcctggcaaccaatgcaagccaggcaggcgttgaaaaaattctgagcgcagttgaaatcccggatgttggtaatctgagccaggttgttgtgatgaaaagcgaaaatgatcagggcattcgcaacaagtgtaaaatgaatctgcaagacaataacggcaacgatattggctttatcggctttcaccagtttaataacattgcaaaactggtggccagcaactggtataaccgtcagattggtaaagcaagccgtacctttggttgtagctgggaatttatcccggttgatgatggttggggtgaaagcagcctggaaaatctgtatttccagggtgccagtcatcatcaccaccatcaccatcactga

SEQ ID NO: 26 - protein, 1305 aa, Artificial Sequence
  Polypeptide Sequence of rBoNT/FA(0) (His-tagged)
MPVVINSFNYDDPVNDNTIIYIRPPYYETSNTYFKAFQIMDNVWIIPERYRLGIDPSLFNPPVSLKAGSDGYFDPNYLSTNTEKNKYLQIMIKLFKRINSKPAGQILLEEIKNAIPYLGNSYTQEEQFTTNNRTVSFNVKLANGNIVQQMANLIIWGPGPDLTTNKTGGIIYSPYQSMEATPYKDGFGSIMTVEFSPEYATAFNDISIASHSPSLFIKDPALILMHQLIYVLHGLYGTYITEYKITPNVVQSYMKVTKPITSAEFLTFGGRDRNIVPQSIQSQLYNKVLSDYKRIASRLNKVNTATALINIDEFKNLYEWKYQFAKDSNGVYSVDLNKFEQLYKKIYSFTEFNLAYEFKIKTRLGYLAENFGPFYLPNLLDDSIYTEVDGFNIGALSINYQGQNIGSDINSIKKLQGQGVVSRVVRLCSNSNTKNSLCITVNNRDLFFIASQESYGENTINTYKEIDDTTTLDPSFEDILDKVILNFNEQVIPQMPNRNVSTDIQKDNYIPKYDYNRTDIIDSYEVGRNYNTFFYLNAQKFSPNESNITLTSSFDTGLLEGSKVYTFFSSDFINNINKPVQALLFIEWVKQVIRDFTTEATKTSTVDKLKDISLVVPYIGLALNIGDEIYKQHFAEAVELVGAGLLLEFSPEFLIPTLLIFTIKGYLTGSIRDKDKIIKTLDNALNVRDQKWKELYRWVVSKWLTTINTQFNKRKEQMYKALKNQATAIKKIIENKYNNYTTDEKSKIDSSYNINEIERTLNEKINLAMKNIEQFITESSIAYLINIINNETIQKLKSYDDLVRRYLLGYIRNHSSILGNSVEELNSKVNNHLDNGIPFELSSYTNDSLLIRYFNKNYGELKYNCILNIKYEMDRDKLVDSSGYRSRINIGTGVKFSEIDKNQVQLSNLESSKIEVILNNGVIYNSMYENFSTSFWIRIPKYFRNINNEYKIISCMQNNSGWEVSLNFSNMNSKIIWTLQDTEGIKKTVVFQYTQNINISDYINRWIFVTITNNRLSNSKIYINGRLINEESISDLGNIHASNNIMFKLDGCRDPHRYIWIKYFNLFDKELNKKEIKDLYDNQSNSGILKDFWGDYLQYDKPYYMLNLYDPNKYLDVNNVGIRGYMYLKGPRGRIVTTNIYLNSTLYMGTKFIIKKYASGNKDNIVRNNDRVYINVVVKNKEYRLATNASQAGVEKILSAVEIPDVGNLSQVVVMKSENDQGIRNKCKMNLQDNNGNDIGFIGFHQFNNIAKLVASNWYNRQIGKASRTFGCSWEFIPVDDGWGESSLENLYFQGASHHHHHHHH

SEQ ID NO: 27 - DNA, 2634 bp, Artificial Sequence
  Nucleotide Sequence of rLHN/FA (His-tagged)
atgccggttgtgattaacagcttcaattatgatgatccggtgaacgataacaccatcatttatatccgtccgccttattatgaaaccagcaacacctatttcaaagccttccagattatggataacgtgtggattattccggaacgttatcgtctgggtattgatccgagcctgtttaatccgcctgttagcctgaaagcaggtagtgatggttattttgatccgaattatctgagcaccaacaccgagaaaaacaaatacctgcagattatgatcaagctgttcaaacgcattaatagcaaaccggcaggtcagattctgctggaagaaatcaaaaatgcaattccgtatctgggcaacagctatacccaagaagaacagtttaccaccaataatcgtaccgtgagctttaatgttaaactggccaatggtaatatcgttcagcagatggcaaatctgattatttggggtccgggtcctgatctgaccacaaataaaaccggtggtatcatctatagcccgtatcagagcatggaagcaaccccgtataaagatggttttggtagcattatgaccgtggaatttagtccggaatatgcaaccgcctttaacgatatttcaattgcaagccatagtccgtcgctgtttatcaaagatccggcactgattctgatgcatgaactgattcatgttctgcatggtctgtatggcacctatattaccgaatacaaaattaccccgaatgtggtgcagagctatatgaaagttaccaaaccgattaccagcgcagaatttctgacctttggtggtcgtgatcgcaatattgttccgcagagcattcagagccagctgtataacaaagttctgagcgattataaacgtattgccagccgtctgaataaagttaataccgcaaccgcactgatcaacatcgatgaattcaaaaacctgtacgagtggaaataccagtttgccaaagatagcaatggtgtgtatagcgtggatctgaacaaatttgagcagctgtacaaaaaaatctatagcttcaccgaattcaacctggcctatgagtttaaaatcaaaacccgtctgggttatctggccgaaaattttggtccgttttatctgccgaatctgctggatgatagcatttataccgaagtggatggttttaacattggtgcactgagcattaactatcagggtcagaatattggcagcgatatcaacagcatcaaaaaactgcaaggtcagggtgttgttagccgtgttgttcgtctgtgtagcaatagcaataccaaaaacagcctgtgcattaccgttaataatcgcgacctgttttttatcgcaagccaagaaagctatggcgagaataccattaacacctataaagagattgacgataccaccacactggatccgagctttgaagatattctggataaagtgatcctgaacttcaacgaacaggttattccgcagatgccgaatcgtaatgttagcaccgatattcagaaagacaactacatcccgaaatacgattataaccgcaccgacattatcgatagctatgaagttggtcgcaactacaacacctttttctatctgaatgcccagaaatttagcccgaacgaaagcaatattaccctgaccagcagctttgatacaggtctgttagaaggtagcaaagtgtatacctttttcagcagcgatttcattaacaacatcaacaaaccggttcaggccctgctgtttattgaatgggttaaacaggtgattcgcgattttaccaccgaagcaaccaaaacctcaaccgttgataaactgaaagatattagcctggtggtgccgtatattggtctggcactgaatattggtgatgagatctacaaacagcattttgcagaagcagttgaactggttggtgcaggtctgctgctggaattttcaccggaatttcttattccgacgctgctgatttttaccatcaaaggttatctgaccggtagcattcgcgataaagacaaaatcattaaaaccctggataacgccctgaatgttcgtgatcagaaatggaaagaactgtatcgttgggttgttagcaaatggctgaccaccattaatacgcagttcaacaaacgcaaagaacaaatgtacaaagccctgaaaaatcaggccaccgccattaaaaagatcatcgagaacaaatataacaactataccaccgatgaaaaaagcaagatcgatagcagctataacatcaacgaaattgaacgcaccctgaacgaaaaaatcaatctggccatgaaaaacatcgagcagtttattacagaaagcagcattgcctacctgatcaatatcatcaacaacgaaaccattcagaaactgaaaagctatgatgacctggttcgtcgttatctgctgggttatattcgtaatcatagcagcattctgggcaatagcgttgaagaactgaattccaaagtgaacaaccatctggataatggcattccgtttgaactgagcagttataccaatgatagcctgctgatccgctacttcaataaaaactatggcgaagagaacctgtatttccagggtgccagtcatcatcaccaccatcaccatcactga

SEQ ID NO: 28 - protein, 877 aa, Artificial Sequence
  Polypeptide Sequence of rLHN/FA (His-tagged)
MPVVINSFNYDDPVNDNTIIYIRPPYYETSNTYFKAFQIMDNVWIIPERYRLGIDPSLFNPPVSLKAGSDGYFDPNYLSTNTEKNKYLQIMIKLFKRINSKPAGQILLEEIKNAIPYLGNSYTQEEQFTTNNRTVSFNVKLANGNIVQQMANLIIWGPGPDLTTNKTGGIIYSPYQSMEATPYKDGFGSIMTVEFSPEYATAFNDISIASHSPSLFIKDPALILMHELIHVLHGLYGTYITEYKITPNVVQSYMKVTKPITSAEFLTFGGRDRNIVPQSIQSQLYNKVLSDYKRIASRLNKVNTATALINIDEFKNLYEWKYQFAKDSNGVYSVDLNKFEQLYKKIYSFTEFNLAYEFKIKTRLGYLAENFGPFYLPNLLDDSIYTEVDGFNIGALSINYQGQNIGSDINSIKKLQGQGVVSRVVRLCSNSNTKNSLCITVNNRDLFFIASQESYGENTINTYKEIDDTTTLDPSFEDILDKVILNFNEQVIPQMPNRNVSTDIQKDNYIPKYDYNRTDIIDSYEVGRNYNTFFYLNAQKFSPNESNITLTSSFDTGLLEGSKVYTFFSSDFINNINKPVQALLFIEWVKQVIRDFTTEATKTSTVDKLKDISLVVPYIGLALNIGDEIYKQHFAEAVELVGAGLLLEFSPEFLIPTLLIFTIKGYLTGSIRDKDKIIKTLDNALNVRDQKWKELYRWVVSKWLTTINTQFNKRKEQMYKALKNQATAIKKIIENKYNNYTTDEKSKIDSSYNINEIERTLNEKINLAMKNIEQFITESSIAYLINIINNETIQKLKSYDDLVRRYLLGYIRNHSSILGNSVEELNSKVNNHLDNGIPFELSSYTNDSLLIRYFNKNYGEENLYFQGASHHHHHHHH

SEQ ID NO: 29 - DNA, 1341 bp, Artificial Sequence
  Nucleotide Sequence of rHC/FA (His-tagged)
atgctgaagtataactgcatcctgaacatcaaatatgagatggatcgtgataaactggttgatagcagcggttatcgtagccgtatcaatattggcaccggtgtgaaatttagcgagatcgataaaaatcaggtgcagctgagcaatctggaaagcagcaaaattgaagtgattctgaataacggcgtgatctacaatagcatgtatgaaaacttttcgaccagcttctggattcgcattccgaaatactttcgcaacatcaacaacgagtacaagattatcagctgtatgcagaataatagcggttgggaagttagcctgaatttcagcaatatgaacagcaaaatcatttggaccctgcaggataccgaaggtatcaaaaaaaccgttgtgtttcagtacacccagaacattaacatcagcgattacattaaccgctggatctttgtgaccattaccaataatcgtctgagcaacagcaagatctatattaacggtcgcctgattaacgaagagagcattagcgatctgggtaatattcatgccagcaacaacatcatgtttaaactggatggttgtcgtgatccgcatcgttatatttggatcaaatacttcaacctgtttgataaagaactgaacaaaaaagaaatcaaagacctgtatgataaccagagcaatagcggcattctgaaagatttttggggtgattatctgcagtatgacaaaccgtattacatgctgaatctgtacgatccgaacaaatatctggatgtgaataatgtgggtatccgtggctatatgtatctgaaaggtccgcgtggtcgtattgttaccaccaacatttatctgaatagcaccctgtatatgggcaccaaattcatcattaaaaagtatgccagcggcaacaaagataacattgtgcgtaataatgatcgcgtgtatatcaatgtggtggtgaagaataaagaatatcgtctggccaccaatgcaagccaggcaggcgttgaaaaaattctgagcgcagttgaaattccggatgttggtaatctgagccaggttgttgttatgaaaagcgaaaatgatcagggcattcgcaacaaatgcaaaatgaatctgcaggacaataacggcaacgatattggttttattggcttccaccagttcaacaacattgcaaaactggtggcgagcaattggtataatcgtcagattggtaaagcaagccgtacctttggttgtagctgggaatttattccggttgatgatggttggggtgaaagcagcctggaaaatctgtattttcagggtgcaagtcatcatcaccaccatcaccatcattaa

SEQ ID NO: 30 - protein, 446 aa, Artificial Sequence
  Polypeptide Sequence of rHC/FA (His-tagged)
MLKYNCILNIKYEMDRDKLVDSSGYRSRINIGTGVKFSEIDKNQVQLSNLESSKIEVILNNGVIYNSMYENFSTSFWIRIPKYFRNINNEYKIISCMQNNSGWEVSLNFSNMNSKIIWTLQDTEGIKKTVVFQYTQNINISDYINRWIFVTITNNRLSNSKIYINGRLINEESISDLGNIHASNNIMFKLDGCRDPHRYIWIKYFNLFDKELNKKEIKDLYDNQSNSGILKDFWGDYLQYDKPYYMLNLYDPNKYLDVNNVGIRGYMYLKGPRGRIVTTNIYLNSTLYMGTKFIIKKYASGNKDNIVRNNDRVYINVVVKNKEYRLATNASQAGVEKILSAVEIPDVGNLSQVVVMKSENDQGIRNKCKMNLQDNNGNDIGFIGFHQFNNIAKLVASNWYNRQIGKASRTFGCSWEFIPVDDGWGESSLENLYFQGASHHHHHHHH

SEQ ID NO: 31 - DNA, 1347 bp, Artificial Sequence
  Nucleotide Sequence of rLC/FA (His-tagged)
atgccggttgtgattaacagcttcaattatgatgatccggtgaacgataacaccatcatttatatccgtccgccttattatgaaaccagcaacacctatttcaaagccttccagattatggataacgtgtggattattccggaacgttatcgtctgggtattgatccgagcctgtttaatccgcctgttagcctgaaagcaggtagtgatggttattttgatccgaattatctgagcaccaacaccgagaaaaacaaatacctgcagattatgatcaagctgttcaaacgcattaatagcaaaccggcaggtcagattctgctggaagaaatcaaaaatgcaattccgtatctgggcaacagctatacccaagaagaacagtttaccaccaataatcgtaccgtgagctttaatgttaaactggccaatggtaatatcgttcagcagatggcaaatctgattatttggggtccgggtcctgatctgaccacaaataaaaccggtggtatcatctatagcccgtatcagagcatggaagcaaccccgtataaagatggttttggtagcattatgaccgtggaatttagtccggaatatgcaaccgcctttaacgatatttcaattgcaagccatagtccgtcgctgtttatcaaagatccggcactgattctgatgcatgaactgattcatgttctgcatggtctgtatggcacctatattaccgaatacaaaattaccccgaatgtggtgcagagctatatgaaagttaccaaaccgattaccagcgcagaatttctgacctttggtggtcgtgatcgcaatattgttccgcagagcattcagagccagctgtataacaaagttctgagcgattataaacgtattgccagccgtctgaataaagttaataccgcaaccgcactgatcaacatcgatgaattcaaaaacctgtacgagtggaaataccagtttgccaaagatagcaatggtgtgtatagcgtggatctgaacaaatttgagcagctgtacaaaaaaatctatagcttcaccgaattcaacctggcctatgagtttaaaatcaaaacccgtctgggttatctggccgaaaattttggtccgttttatctgccgaatctgctggatgatagcatttataccgaagtggatggttttaacattggtgcactgagcattaactatcagggtcagaatattggcagcgatatcaacagcatcaaaaaactgcaaggtcagggtgttgttagccgtgttgttcgtctgtgtagcaatagcgaaaatctgtattttcagggtgccagtcatcatcaccaccatcaccatcactga

SEQ ID NO: 32 - protein, 448 aa, Artificial Sequence
  Polypeptide Sequence of rLC/FA (His-tagged)
MPVVINSFNYDDPVNDNTIIYIRPPYYETSNTYFKAFQIMDNVWIIPERYRLGIDPSLFNPPVSLKAGSDGYFDPNYLSTNTEKNKYLQIMIKLFKRINSKPAGQILLEEIKNAIPYLGNSYTQEEQFTTNNRTVSFNVKLANGNIVQQMANLIIWGPGPDLTTNKTGGIIYSPYQSMEATPYKDGFGSIMTVEFSPEYATAFNDISIASHSPSLFIKDPALILMHELIHVLHGLYGTYITEYKITPNVVQSYMKVTKPITSAEFLTFGGRDRNIVPQSIQSQLYNKVLSDYKRIASRLNKVNTATALINIDEFKNLYEWKYQFAKDSNGVYSVDLNKFEQLYKKIYSFTEFNLAYEFKIKTRLGYLAENFGPFYLPNLLDDSIYTEVDGFNIGALSINYQGQNIGSDINSIKKLQGQGVVSRVVRLCSNSENLYFQGASHHHHHHHH

SEQ ID NO: 33 - DNA, 3888 bp, Artificial Sequence
  Nucleotide Sequence of rBoNT/F(0) (His-tagged)
atgccggttgtgattaacagcttcaattataacgatccggtgaacgatgataccatcctgtatatgcagattccgtatgaagagaaaagcaaaaagtactacaaagcctttgagatcatgcgcaacgtttggattattccggaacgtaataccattggcaccgatccgagcgattttgatccgcctgcaagcctggaaaatggtagcagcgcatattatgatccgaattatctgaccaccgatgccgaaaaagatcgttatctgaaaaccaccatcaaactgttcaaacgcattaatagcaatccggcaggcgaagttctgctgcaagaaattagctatgcaaaaccgtatctgggcaatgaacataccccgattaatgaatttcatccggttacacgtaccacgagcgttaacattaaaagcagcaccaatgtgaagtccagcattattctgaatctgctggttttaggtgcaggtccggatatttttgaaaattcaagctatccggtgcgcaaactgatggatagcggtggtgtgtatgatccgtcaaatgatggttttggcagcattaacattgtgacctttagtccggaatatgaatacaccttcaacgatattagcggtggctataatagcagcaccgaaagttttattgcagatccggcaattagcctggcacaccagctgatttatgcactgcatggtctgtatggtgcacgtggtgttacctataaagaaaccattaaagttaaacaggcaccgctgatgattgcggaaaaaccgattcgtctggaagaatttctgacctttggtggtcaggatctgaacattattaccagcgcaatgaaagagaaaatctataataacctgctggccaactatgagaaaattgcaacccgtctgagccgtgttaatagcgcacctcctgaatatgatatcaacgagtataaagactattttcagtggaaatacggcctggataaaaatgcagatggtagctataccgtgaacgagaacaaatttaacgagatctacaaaaaactgtatagcttcaccgaaatcgatctggccaacaaattcaaagtgaaatgccgcaacacctacttcatcaaatatggctttctgaaagttccgaacctgcttgatgatgatatctataccgttagcgaaggctttaacattggtaatctggccgttaataatcgcggtcagaacattaaactgaacccgaaaattatcgatagcatcccggataaaggcctggttgaaaaaattgtgaaattctgcaaaagcgtgattccgcgtaaaggcaccaaagcaccgcctcgtctgtgtattcgtgtgaataatcgtgaactgttttttgttgcaagcgagagcagctataacgagaatgatattaacaccccgaaagagattgacgataccaccaatctgaataacaactatcgcaacaatctggatgaagtgatcctggattataacagcgaaaccattccgcagattagcaatcagaccctgaataccctggttcaggatgatagctatgttccgcgttatgatagcaatggcaccagcgaaattgaagaacataatgtggttgatctgaacgtgttcttttatctgcatgcacagaaagtgccggaaggtgaaaccaatattagcctgaccagcagcattgataccgcactgagcgaagaaagccaggtttataccttttttagcagcgaattcatcaacaccattaacaaaccggttcatgcagcactgtttattagctggattaatcaggtgattcgcgattttaccaccgaagcaacccagaaaagcacctttgataaaattgccgatattagtctggtggtgccgtatgttggtctggcactgaatattggtaatgaagtgcagaaagagaactttaaagaagccttcgaactgttaggtgccggtattctgctggaatttgtgccggaactgctgattccgaccattctggtttttaccattaagagctttattggcagcagcgagaacaagaacaaaatcattaaagccatcaacaacagcctgatggaacgcgaaaccaaatggaaagaaatttacagctggattgtgagcaattggctgacccgtatcaatacccagtttaacaaacgcaaagaacaaatgtatcaggccctgcagaatcaggttgatgcaattaaaaccgtgatcgaatacaaatacaacaactataccagcgacgaacgtaatcgcctggaaagcgaatacaacattaataacattcgcgaagaactgaacaaaaaagtgagcctggcaatggaaaacatcgaacgttttattaccgaaagcagcatcttctacctgatgaaactgattaacgaagccaaagttagcaaactgcgcgaatatgatgaaggcgttaaagaatatctgctggactatattagcgaacatcgtagcattctgggtaatagcgttcaagagctgaatgatctggttaccagcacactgaataatagcattccgtttgaactgagcagctacaccaacgataaaatcctgatcctgtacttcaacaaactgtacaagaagatcaaggacaacagcatactggatatgcgctatgaaaacaacaagttcattgatatcagcggctatggtagcaacattagcattaatggtgatgtgtatatctacagcaccaaccgcaatcagtttggtatttatagcagcaaaccgagcgaagttaatattgcgcagaataacgatatcatctacaacggtcgctatcagaactttagcattagcttttgggttcgcattccgaaatactttaacaaggtgaacctgaacaacgagtacaccattattgattgcattcgcaataataacagcggctggaaaatcagcctgaactataacaaaattatctggaccctgcaggataccgcaggtaataatcagaaactggtgtttaactacacccagatgattagcatcagcgactatatcaacaaatggatctttgtgaccattaccaacaatcgtctgggtaacagccgcatttatatcaatggcaatctgatcgacgaaaaaagcatttcaaatctgggcgatattcacgtgagcgataacattctgttcaaaattgttggctgcaacgatacccgttatgttggtattcgttacttcaaagtgtttgatacggaactgggcaaaacggaaattgaaaccctgtatagtgatgaaccggatccgagcattctgaaagatttttggggtaattatctgctgtacaacaaacgctactatctgctgaacctgctgcgtaccgataaaagcattacacagaatagcaactttctgaacatcaatcagcagcgtggtgtttatcagaaaccgaacatttttagcaacacccgtctgtataccggtgtggaagttattattcgtaaaaacggtagcaccgatatcagcaacaccgataactttgtgcgtaaaaatgacctggcctatattaacgttgttgatcgtgatgttgagtatcgtctgtatgcggatattagcattgccaaaccggaaaagattatcaaactgatccgtaccagcaacagcaataattcactgggtcagattatcgtgatggacagcattggtaacaattgcaccatgaatttccagaacaataacggtggtaatattggcctgctgggctttcatagcaataatctggttgcaagcagctggtattacaacaacatccgtaaaaataccagcagtaatggttgcttttggagctttatcagtaaagaacatggctggcaagaaaacgagaacctgtattttcagggtgcaagtcatcatcaccatcaccaccatcattaa

SEQ ID NO: 34 - protein, 1295 aa, Artificial Sequence
  Polypeptide Sequence of rBoNT/F(0) (His-tagged)
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHQLIYALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQENENLYFQGASHHHHHHHH

SEQ ID NO: 35 - DNA, 2649 bp, Artificial Sequence
  Nucleotide Sequence of rLHN/F (His-tagged)
atgccggttgtgattaacagcttcaattataacgatccggtgaacgatgataccatcctgtatatgcagattccgtatgaagagaaaagcaaaaagtactacaaagcctttgagatcatgcgcaacgtttggattattccggaacgtaataccattggcaccgatccgagcgattttgatccgcctgcaagcctggaaaatggtagcagcgcatattatgatccgaattatctgaccaccgatgccgaaaaagatcgttatctgaaaaccaccatcaaactgttcaaacgcattaatagcaatccggcaggcgaagttctgctgcaagaaattagctatgcaaaaccgtatctgggcaatgaacataccccgattaatgaatttcatccggttacacgtaccacgagcgttaacattaaaagcagcaccaatgtgaagtccagcattattctgaatctgctggttttaggtgcaggtccggatatttttgaaaattcaagctatccggtgcgcaaactgatggatagcggtggtgtgtatgatccgtcaaatgatggttttggcagcattaacattgtgacctttagtccggaatatgaatacaccttcaacgatattagcggtggctataatagcagcaccgaaagttttattgcagatccggcaattagcctggcacatgaactgattcatgcactgcatggtctgtatggtgcacgtggtgttacctataaagaaaccattaaagttaaacaggcaccgctgatgattgcggaaaaaccgattcgtctggaagaatttctgacctttggtggtcaggatctgaacattattaccagcgcaatgaaagagaaaatctataataacctgctggccaactatgagaaaattgcaacccgtctgagccgtgttaatagcgcacctcctgaatatgatatcaacgagtataaagactattttcagtggaaatacggcctggataaaaatgcagatggtagctataccgtgaacgagaacaaatttaacgagatctacaaaaaactgtatagcttcaccgaaatcgatctggccaacaaattcaaagtgaaatgccgcaacacctacttcatcaaatatggctttctgaaagttccgaacctgcttgatgatgatatctataccgttagcgaaggctttaacattggtaatctggccgttaataatcgcggtcagaacattaaactgaacccgaaaattatcgatagcatcccggataaaggcctggttgaaaaaattgtgaaattctgcaaaagcgtgattccgcgtaaaggcaccaaagcaccgcctcgtctgtgtattcgtgtgaataatcgtgaactgttttttgttgcaagcgagagcagctataacgagaatgatattaacaccccgaaagagattgacgataccaccaatctgaataacaactatcgcaacaatctggatgaagtgatcctggattataacagcgaaaccattccgcagattagcaatcagaccctgaataccctggttcaggatgatagctatgttccgcgttatgatagcaatggcaccagcgaaattgaagaacataatgtggttgatctgaacgtgttcttttatctgcatgcacagaaagtgccggaaggtgaaaccaatattagcctgaccagcagcattgataccgcactgagcgaagaaagccaggtttataccttttttagcagcgaattcatcaacaccattaacaaaccggttcatgcagcactgtttattagctggattaatcaggtgattcgcgattttaccaccgaagcaacccagaaaagcacctttgataaaattgccgatattagtctggtggtgccgtatgttggtctggcactgaatattggtaatgaagtgcagaaagagaactttaaagaagccttcgaactgttaggtgccggtattctgctggaatttgtgccggaactgctgattccgaccattctggtttttaccattaagagctttattggcagcagcgagaacaagaacaaaatcattaaagccatcaacaacagcctgatggaacgcgaaaccaaatggaaagaaatttacagctggattgtgagcaattggctgacccgtatcaatacccagtttaacaaacgcaaagaacaaatgtatcaggccctgcagaatcaggttgatgcaattaaaaccgtgatcgaatacaaatacaacaactataccagcgacgaacgtaatcgcctggaaagcgaatacaacattaataacattcgcgaagaactgaacaaaaaagtgagcctggcaatggaaaacatcgaacgttttattaccgaaagcagcatcttctacctgatgaaactgattaacgaagccaaagttagcaaactgcgcgaatatgatgaaggcgttaaagaatatctgctggactatattagcgaacatcgtagcattctgggtaatagcgttcaagagctgaatgatctggttaccagcacactgaataatagcattccgtttgaactgagcagctacaccaacgataaaatcctgatcctgtacttcaacaaactgtacaagaaagaaaacctgtattttcagggtgcaagccatcatcaccaccatcaccatcattaa

SEQ ID NO: 36 - protein, 882 aa, Artificial Sequence
  Polypeptide Sequence of rLHN/F (His-tagged)
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKENLYFQGASHHHHHHHH

SEQ ID NO: 37 - DNA, 1296 bp, Artificial Sequence
  Nucleotide Sequence of rHC/F (His-tagged)
atgatcaaggataacagcattctggatatgcgctatgagaacaacaaattcattgatattagcggctatggcagcaacattagcattaatggtgatgtgtatatctacagcaccaaccgtaatcagtttggcatttatagcagcaaaccgagcgaagttaatattgcccagaacaacgatatcatctataacggtcgctatcagaacttcagcattagcttttgggttcgcattccgaaatacttcaataaggtgaacctgaacaacgagtataccatcattgattgcattcgcaataataacagcggctggaaaattagcctgaactacaacaaaattatctggaccctgcaggataccgcaggtaataatcagaaactggtgtttaactacacccagatgattagcatcagcgactatatcaacaaatggatctttgtgaccattaccaataatcgcctgggtaatagccgcatttatatcaatggtaacctgatcgatgagaaaagcattagcaatctgggtgatattcatgtgagcgataacatcctgtttaaaatcgtgggttgtaacgatacccgttatgttggtattcgctacttcaaagtgtttgataccgaactgggtaaaaccgaaattgaaaccctgtatagtgatgaaccggatccgagcattctgaaagatttttggggtaattatctgctgtacaacaaacgctactatctgctgaatctgctgcgtaccgataaatcaattacccagaatagcaacttcctgaacattaatcagcagcgtggtgtttatcagaaaccgaacatttttagcaacacccgtctgtataccggtgtggaagttattattcgtaaaaatggcagcaccgatatcagcaacaccgataactttgttcgcaaaaatgatctggcgtatatcaacgttgttgatcgtgatgttgaatatcgtctgtatgccgatattagcattgccaaaccggaaaaaatcatcaaactgatccgtaccagcaacagcaataattcactgggtcagattattgtgatggatagcattggtaataactgcaccatgaactttcagaacaataacggtggtaatattggtctgctgggctttcatagtaataatctggttgcaagcagctggtattataacaacatccgtaaaaataccagcagcaatggttgcttttggagctttattagcaaagaacatggctggcaagaaaacgagaatctgtattttcagggtgcaagtcatcatcaccaccatcaccatcattaa

SEQ ID NO: 38 - protein, 431 aa, Artificial Sequence
  Polypeptide Sequence of rHC/F (His-tagged)
MIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQENENLYFQGASHHHHHHHH

SEQ ID NO: 39 - DNA, 1347 bp, Artificial Sequence
  Nucleotide Sequence of rLC/F (His-tagged)
atgccggttgtgattaacagcttcaattataacgatccggtgaacgatgataccatcctgtatatgcagattccgtatgaagagaaaagcaaaaagtactacaaagcctttgagatcatgcgcaacgtttggattattccggaacgtaataccattggcaccgatccgagcgattttgatccgcctgcaagcctggaaaatggtagcagcgcatattatgatccgaattatctgaccaccgatgccgaaaaagatcgttatctgaaaaccaccatcaaactgttcaaacgcattaatagcaatccggcaggcgaagttctgctgcaagaaattagctatgcaaaaccgtatctgggcaatgaacataccccgattaatgaatttcatccggttacacgtaccacgagcgttaacattaaaagcagcaccaatgtgaagtccagcattattctgaatctgctggttttaggtgcaggtccggatatttttgaaaattcaagctatccggtgcgcaaactgatggatagcggtggtgtgtatgatccgtcaaatgatggttttggcagcattaacattgtgacctttagtccggaatatgaatacaccttcaacgatattagcggtggctataatagcagcaccgaaagttttattgcagatccggcaattagcctggcacatgaactgattcatgcactgcatggtctgtatggtgcacgtggtgttacctataaagaaaccattaaagttaaacaggcaccgctgatgattgcggaaaaaccgattcgtctggaagaatttctgacctttggtggtcaggatctgaacattattaccagcgcaatgaaagagaaaatctataataacctgctggccaactatgagaaaattgcaacccgtctgagccgtgttaatagcgcacctcctgaatatgatatcaacgagtataaagactattttcagtggaaatacggcctggataaaaatgcagatggtagctataccgtgaacgagaacaaatttaacgagatctacaaaaaactgtatagcttcaccgaaatcgatctggccaacaaattcaaagtgaaatgccgcaacacctacttcatcaaatatggctttctgaaagttccgaacctgcttgatgatgatatctataccgttagcgaaggctttaacattggtaatctggccgttaataatcgcggtcagaacattaaactgaacccgaaaattatcgatagcatcccggataaaggcctggttgaaaaaattgtgaaattctgcaaaagcgagaacctgtattttcagggtgcaagtcatcatcaccatcaccaccatcattaa

SEQ ID NO: 40 - protein, 448 aa, Artificial Sequence
  Polypeptide Sequence of rLC/F (His-tagged)
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSENLYFQGASHHHHHHHH

SEQ ID NO: 41 - DNA, 1317 bp, Artificial Sequence
  Nucleotide Sequence of Cationic rHC/A (His-tagged)
atgatcatcaacaccagcattctgaacctgcgttatgaaagcaaacatctgattgatctgagccgttatgccagcaaaatcaatataggcagcaaggttaacttcgacccgattgacaaaaatcagatacagctgtttaatctggaaagcagcaaaattgaggtgatcctgaaaaaagcgatcgtgtataatagcatgtacgagaatttttcgaccagcttttggattcgcatcccgaaatactttaacaagattagcctgaacaacgagtataccatcattaactgcatggaaaacaatagcggttggaaagtcagcctgaattatggcgaaattatctggaccctgcaggataccaaagaaatcaaacagcgtgtggtgttcaaatacagccagatgattaatatcagcgactatatcaaccgctggatttttgtgaccattaccaataatcggctgaacaagagcaagatctatattaacggtcgtctgattgaccagaaaccgattagtaatctgggtaatattcatgcgagcaacaaaatcatgtttaaactggatggttgccgtgatacccatcgttatatttggatcaaatacttcaacctgttcgataaagagttgaacgaaaaagaaattaaagacctgtacgataaccagagcaatagcggcatactgaaagatttttggggagattatctgcagtatgacaaaccgtattatatgctgaatctgtacgacccgaataaatacgtggatgttaataatgtgggcatccgtggttatatgtacctgaaaggtccgcgtggtagcgttatgaccacaaacatttatctgaatagcagcctgtatcgcggaaccaaattcatcattaaaaagtatgccagcggcaacaaggataatattgtgcgtaataatgatcgcgtgtacattaacgttgtggtgaagaataaagaatatcgcctggcaaccaatgcaagccaggcaggcgttgaaaaaattctgagtgccctggaaattccggatgttggtaatctgagccaggttgttgtgatgaaaagcaaaaacgataaaggcatcaccaacaaatgcaagatgaatctgcaggacaataacggcaatgatattggcttcattggctttcaccagtttaacaacattgcaaaactggttgcgagcaattggtataatcgtcagattgaacgtagcagtcgtaccctgggttgtagctgggaatttatccctgtggatgatggttggggtgaacgtccgctgaagcttgcggccgcactcgagcaccaccaccaccaccactga

SEQ ID NO: 42 - protein, 438 aa, Artificial Sequence
  Polypeptide Sequence of Cationic rHC/A (His-tagged)
MIINTSILNLRYESKHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPLKLAAALEHHHHHH

SEQ ID NO: 43 - DNA, 1341 bp, Artificial Sequence
  Nucleotide Sequence of rHC/AB (His-tagged)
atgattctgaacaatattatcctgaacctgcgttacaaagacaacaatctgatcgatctgagcggctatggtgcaaaagttgaagtctacgacggtgtcgaactgaacgataaaaaccagttcaaactgacctcatcggctaactcaaaaattcgtgtgacgcagaaccaaaacatcatcttcaactcggtctttctggacttcagcgtgtctttctggattcgcatcccgaaatataaaaatgatggcatccagaactacatccataacgaatacaccatcatcaactgtatgaaaaacaacagtggttggaaaatttccatccgtggcaaccgcattatctggaccctgattgatatcaatggtaaaacgaaaagcgtgtttttcgaatacaacatccgtgaagatatctctgaatacatcaatcgctggtttttcgtgaccattacgaacaatctgaacaatgcgaaaatctatatcaacggcaaactggaaagtaataccgacatcaaagatattcgtgaagttatcgccaacggtgaaatcatcttcaaactggatggcgacatcgatcgcacccagttcatttggatgaaatacttctccatcttcaacacggaactgagtcagtccaatatcgaagaacgctacaaaatccaatcatactcggaatacctgaaagatttctggggtaacccgctgatgtacaacaaagaatactacatgttcaacgcgggcaacaaaaactcatacatcaaactgaaaaaagattcgccggtgggtgaaatcctgacccgtagcaaatacaaccagaactctaaatacatcaactatcgcgatctgtacattggcgaaaaatttattatccgtcgcaaaagcaactctcagagtattaatgatgacatcgtgcgtaaagaagactacatctatctggatttctttaatctgaaccaagaatggcgcgtttatacctacaaatacttcaaaaaagaagaaatgaaactgttcctggccccgatttacgacagcgatgaattttacaacaccatccagatcaaagaatacgatgaacagccgacgtatagttgccaactgctgttcaaaaaagacgaagaatccaccgatgaaattggcctgattggtatccaccgtttctatgaaagcggtatcgttttcgaagaatacaaagattacttctgtatctctaaatggtatctgaaagaagtcaaacgcaaaccgtacaacctgaaactgggctgcaactggcaatttatcccgaaagacgaaggctggaccgaaaagcttgcggccgcactcgagcaccaccaccaccaccactga

SEQ ID NO: 44 - protein, 446 aa, Artificial Sequence
  Polypeptide Sequence of rHC/AB (His-tagged)
MILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEKLAAALEHHHHHH

SEQ ID NO: 45 - DNA, 1306 bp, Artificial Sequence
  Nucleotide Sequence of rHC/A Variant Y1117V H1253K (His-tagged)
atgatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctggttgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttcaaacagttcaacaatattgctaaactggtagcgagcaattggtacaatcgtcagattgagcgcagcagccgtactttgggctgtagctgggagtttatcccggtcgatgatggttggggcgaacgtccgctgcaccatcaccatcaccatcaccatcaccatt

SEQ ID NO: 46 - protein, 435 aa, Artificial Sequence
  Polypeptide Sequence of rHC/A Variant Y1117V H1253K (His-tagged)
MIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLVDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFKQFNNIAKLVASNWYNRQIERSSRTLGCSWEFIPVDDGWGERPLHHHHHHHHHH

SEQ ID NO: 47 - DNA, 1308 bp, Artificial Sequence
  Nucleotide Sequence of rHC/A Variant Y1117V F1252Y H1253K L1278F (His-tagged)
atgatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctggttgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggttacaaacagttcaacaatattgctaaactggtagcgagcaattggtacaatcgtcagattgagcgcagcagccgtacttttggctgtagctgggagtttatcccggtcgatgatggttggggcgaacgtccgctgcaccatcaccatcaccatcaccatcaccattaa

SEQ ID NO: 48 - protein, 435 aa, Artificial Sequence
  Polypeptide Sequence of rHC/A Variant Y1117V F1252Y H1253K L1278F (His-tagged)
MIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLVDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGYKQFNNIAKLVASNWYNRQIERSSRTFGCSWEFIPVDDGWGERPLHHHHHHHHHH

SEQ ID NO: 49 - DNA, 1293 bp, Artificial Sequence
  Nucleotide Sequence of rHC/A Variant Y1117V F1252Y H1253K L1278H (His-tagged)
atgatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctggttgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggttacaaacagttcaacaatattgctaaactggtagcgagcaattggtacaatcgtcagattgagcgcagcagccgtactcatggctgtagctgggagtttatcccggtcgatgatggttggggcgaacgtccgctgcaccatcaccatcaccat

SEQ ID NO: 50 - protein, 431 aa, Artificial Sequence
  Polypeptide Sequence of rHC/A Variant Y1117V F1252Y H1253K L1278H (His-tagged)
MIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLVDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGYKQFNNIAKLVASNWYNRQIERSSRTHGCSWEFIPVDDGWGERPLHHHHHH

SEQ ID NO: 51 - protein, 1296 aa, Clostridium botulinum [sequence omitted]

SEQ ID NO: 52 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 53 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 54 - protein, 1276 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 55 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 56 - protein, 1278 aa, Clostridium botulinum
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 57 - protein, 1297 aa, Clostridium botulinum
  misc_feature (7)..(7): Xaa can be any naturally occurring amino acid
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 58 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 59 - protein, 1306 aa, Clostridium botulinum
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 60 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 61 - protein, 1304 aa, Artificial Sequence
  Polypeptide Sequence of mrBoNT/AB(0)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 62 - protein, 1296 aa, Artificial Sequence
  Polypeptide Sequence of mrBoNT/A(0)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESKHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 63 - protein, 1341 aa, Artificial Sequence
  Polypeptide Sequence of BoNT/XB(0) (His-tagged)
MGSMKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHQLVYVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICHKAIDGRSLYNKTLDCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 64 - protein, 1331 aa, Artificial Sequence
  Polypeptide Sequence of BoNT/XB(0)
MGSMKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHQLVYVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICHKAIDGRSLYNKTLDCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 65 - protein, 1308 aa, Artificial Sequence
  Polypeptide Sequence of BoNT/XB(0) Variant (His-tagged)
MGSMKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHQLVYVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICHKAIDGRSLYNKTLDCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 66 - protein, 1298 aa, Artificial Sequence
  Polypeptide Sequence of BoNT/XB(0) Variant
MGSMKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHQLVYVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICHKAIDGRSLYNKTLDCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 67 - protein, 1331 aa, Artificial Sequence
  Polypeptide Sequence of BoNT/XA(0)
MGSMKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHQLVYVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICHKAIDGRSLEVLFQGPLYNKTLDCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIEIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 68 - protein, 1298 aa, Artificial Sequence
  Polypeptide Sequence of BoNT/XA(0) Variant
MGSMKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHQLVYVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICHKAIDGRSLEVLFQGPLYNKTLDCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 69 - protein, 1311 aa, Artificial Sequence
  Polypeptide Sequence of BoNT/XD(0)
MGSMKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHQLVYVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICHKAIDGRSLYNKTLDCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIENDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 70 - protein, 1312 aa, Artificial Sequence
  Polypeptide Sequence of BoNT/XF(0)
MGSMKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHQLVYVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICHKAIDGRSLYNKTLDCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIEIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFKSNNLVASSWYYNNIRKNTSSNGCFWSFISKEHGWQEN

SEQ ID NO: 71 - protein, 8 aa, Artificial Sequence
  C1 Activation Loop Consensus Sequence
  SITE (2)..(2): Xaa is 1-10 residues of any amino acid
  VARIANT (4)..(4): Xaa is Asp or Glu
  SITE (7)..(7): Xaa is 4-15 residues of any amino acid
CXIXGRXC

SEQ ID NO: 72 - protein, 17 aa, Artificial Sequence
  C1 Activation Loop
CHKAIDGRSLYNKTLDC

SEQ ID NO: 73 - protein, 17 aa, Artificial Sequence
  C1 Activation Loop Variant
CHKAIEGRSLYNKTLDC

SEQ ID NO: 74 - protein, 441 aa, Artificial Sequence
  Polypeptide Sequence of rLC/A(0) (His-tagged)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEENLYFQGASHHHHHHHH

SEQ ID NO: 75 - protein, 882 aa, Artificial Sequence
  Polypeptide Sequence of rLHN/A(0) (His-tagged)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTENLYFQGASHHHHHHHH

SEQ ID NO: 76 - protein, 438 aa, Artificial Sequence
  Polypeptide Sequence of rLC/X(0)
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHQLVYVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKIAPRNGLLYNAIYRNSK

SEQ ID NO: 77 - protein, 8 aa, Artificial Sequence
  PreScission Protease Site
LEVLFQGP

SEQ ID NO: 78 - protein, 25 aa, Artificial Sequence
  C1 Activation Loop Variant 2
CHKAIDGRSLEVLFQGPLYNKTLDC

(19) United States
(12) Patent Application Publication
(10) Pub. No.: US 2025/0195626 A1
(43) Pub. Date: Jun. 19, 2025

(54) TREATMENT OF PAIN & INFLAMMATORY DISORDERS

(71) Applicant: IPSEN BIOPHARM LIMITED, Wrexham (GB)

(72) Inventors: Mikhail KALINICHEV, Wrexham (GB); Cindy PERIER, Wrexham (GB); Christine FAVRE, Wrexham (GB); Sylvie CORNET, Wrexham (GB); Johannes KRUPP, Wrexham (GB); Mark ELLIOTT, Wrexham (GB); Jacqueline Caroline MAIGNEL, Wrexham (GB); Hodan Ahmed IBRAHIM, Wrexham (GB)

(21) Appl. No.: 18/552,788

(22) PCT Filed: Mar. 30, 2022
(86) PCT No.: PCT/GB2022/050807

§ 371 Date: Sep. 27, 2023

(30) Foreign Application Priority Data
Mar. 30, 2021 (TW) 110111559
Mar. 30, 2021 (WO) PCT/GB2021/050783

(51) Int. Cl. A61K 38/48; A61P 13/02; A61P 13/10; A61P 25/04; A61P 29/00

(52) U.S. Cl. CPC A61K 38/4893; A61P 13/02; A61P 13/10; A61P 25/04; A61P 29/00; C12Y 304/24069

(57) ABSTRACT

The present invention is directed to a polypeptide for use in treating pain or an inflammatory disorder, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive. Also provided are corresponding methods of treatment and uses.

### FIELD OF THE INVENTION

The present invention relates to polypeptides for use in therapy, for example, the use of polypeptides for the treatment of pain or an inflammatory disorder.

### BACKGROUND

Bacteria in the genus Clostridia produce highly potent and specific protein toxins, which can poison neurons and other cells to which they are delivered. Examples of such clostridial toxins include the neurotoxins produced by C. tetani (TeNT) and by C. botulinum (BoNT) serotypes A-G, and X (see WO 2018/009903 A2), as well as those produced by C. baratii and C. butyricum. Both tetanus and botulinum toxins act by inhibiting the function of affected neurons, specifically the release of neurotransmitters. While botulinum toxin acts at the neuromuscular junction and inhibits cholinergic transmission in the peripheral nervous system, tetanus toxin acts in the central nervous system.

In nature, clostridial neurotoxins are synthesised as a single-chain polypeptide that is modified post-translationally by a proteolytic cleavage event to form two polypeptide chains joined together by a disulphide bond. Cleavage occurs at a specific cleavage site, often referred to as the activation site that is located between the cysteine residues that provide the inter-chain disulphide bond. It is this di-chain form that is the active form of the toxin. The two chains are termed the heavy chain (H-chain), which has a molecular mass of approximately 100 kDa, and the light chain (L-chain), which has a molecular mass of approximately 50 kDa. The H-chain comprises an N-terminal translocation component (H_{N} domain) and a C-terminal targeting component (H_{C} domain). The cleavage site is located between the L-chain and the translocation domain components. Following binding of the H_{C} domain to its target neuron and internalisation of the bound toxin into the cell via an endosome, the H_{N} domain translocates the L-chain across the endosomal membrane and into the cytosol, and the L-chain provides a protease function (also known as a non-cytotoxic protease).

Non-cytotoxic proteases act by proteolytically cleaving intracellular transport proteins known as SNARE proteins (e.g. SNAP-25, VAMP, or Syntaxin). The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins. Accordingly, once delivered to a desired target cell, the non-cytotoxic protease is capable of inhibiting cellular secretion from the target cell. The L-chain proteases of clostridial neurotoxins are non-cytotoxic proteases that cleave SNARE proteins.

In view of the ubiquitous nature of SNARE proteins, clostridial neurotoxins such as botulinum toxin have been successfully employed in a wide range of therapies.

The clostridial neurotoxins are some of the most potent toxins known. By way of example, botulinum neurotoxins have median lethal dose (LD₅₀) values for mice ranging from 0.5 to 5 ng/kg, depending on the serotype. Thus, use of said toxins is not without risk. Spread of toxin away from an administration site and into surrounding tissue or systemic circulation is believed to be responsible for undesirable side effects of clostridial neurotoxin treatment that in extreme cases may be life threatening. This can be a particular concern when using clostridial neurotoxins at high doses, concentrations, and/or injection volumes. Adverse effects that have been reported for commercial BoNT/A therapeutics include asthenia, generalised muscle weakness, diplopia, ptosis, dysphagia, dysphonia, dysarthria, urinary incontinence, and breathing difficulties. Swallowing and breathing difficulties can be life threatening and there have been reported deaths related to the spread of toxin effects.

The present invention overcomes one or more of the above-mentioned problems.

### SUMMARY OF THE INVENTION

The present inventors have found that catalytically inactive clostridial neurotoxins are effective at treating pain. This finding is particularly surprising, as catalytic activity resulting in SNARE protein cleavage was believed to be an essential mechanism of action underlying clostridial neurotoxin therapy. The polypeptides of the invention thus avoid the toxic side-effects associated with conventional catalytically active clostridial neurotoxin therapy and constitute a safer (substantially non-toxic) therapeutic. Advantageously, the polypeptides of the present invention may be dosed in greater amounts when compared to a conventional catalytically active clostridial neurotoxin therapeutic. Furthermore, the reduced toxicity of the polypeptides of the invention allows for ease of manufacture and handling throughout the product lifecycle and removes the need for physicians to perform complex (e.g. personalised) dosing regimen calculations aimed at avoiding toxicity in a subject.

Equally surprisingly, the present inventors have found that catalytically inactive clostridial neurotoxins are effective at treating inflammatory disorders.

### DETAILED DESCRIPTION

In one aspect, the invention provides a polypeptide (e.g. an analgesic polypeptide) for use in treating pain, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.

In one aspect, the invention provides a polypeptide (e.g. an analgesic polypeptide) for use in treating bladder pain, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.

In one aspect, the invention provides a polypeptide (e.g. an analgesic polypeptide) for use in treating bladder pain syndrome, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.

In a related aspect, there is provided a method for treating pain, the method comprising administering a polypeptide (e.g. an analgesic polypeptide) to a subject, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.

In a related aspect, there is provided a method for treating bladder pain, the method comprising administering a polypeptide (e.g. an analgesic polypeptide) to a subject, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.

In a related aspect, there is provided a method for treating bladder pain syndrome, the method comprising administering a polypeptide (e.g. an analgesic polypeptide) to a subject, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.

In another related aspect, the invention provides the use of a polypeptide (e.g. an analgesic polypeptide) in the manufacture of a medicament for treating pain, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.

In another related aspect, the invention provides the use of a polypeptide (e.g. an analgesic polypeptide) in the manufacture of a medicament for treating bladder pain, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.

In another related aspect, the invention provides the use of a polypeptide (e.g. an analgesic polypeptide) in the manufacture of a medicament for treating bladder pain syndrome, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.

The bladder pain is preferably caused by or associated with interstitial cystitis. The bladder pain syndrome is preferably interstitial cystitis.

The polypeptide of the invention may treat bladder pain by decreasing or inhibiting activity of a sensory neuron of the bladder (e.g. when compared to the activity, such as average activity for a given period, of the sensory neuron in the same subject prior to treatment). For example, the polypeptide may decrease or inhibit distension-induced activity of a sensory neuron of the bladder and/or chemical-induced activity of a sensory neuron of the bladder. The sensory neuron of the bladder is preferably an afferent nerve. The afferent nerve may comprise one or more sensory fibers (e.g. a bundle of nerve fibers). The term “fiber” preferably refers to an axon of a neuron. Such an afferent nerve may be an afferent nerve that senses chemical and/or mechanical stimulation in the bladder wall (e.g. relating to bladder filing). Preferably, the afferent nerve comprises one or more fibers of the pelvic and/or hypogastric nerves. For example, the afferent nerve may be the pelvic nerve and/or the hypogastric nerve.

When treating pain (e.g. bladder pain), it is preferred that the polypeptide comprises at least a catalytically inactive clostridial neurotoxin L-chain. More preferably, when treating pain (e.g. bladder pain), the polypeptide comprises a catalytically inactive clostridial neurotoxin L-chain, a clostridial neurotoxin translocation domain (H_{N} domain), and a clostridial neurotoxin receptor binding domain (H_{C} domain). Advantageously, such a polypeptide was more effective at treating pain when compared to polypeptides in which one of said components was absent.

The pain (e.g. bladder pain) treated by a polypeptide of the invention may be one or more of: hyperalgesia, allodynia, and/or visceral pain. For example, all of hyperalgesia, allodynia, and visceral pain may be treated.

The polypeptide of the invention preferably has analgesic properties. In other words, a polypeptide of the invention is preferably an analgesic polypeptide.

Preferably, a polypeptide of the invention neither promotes neuronal growth nor neuronal repair to treat pain. In other words, preferably, the polypeptide does not treat pain by any of the following means: by promoting neuronal growth, by promoting neuronal repair, or by promoting neuronal growth and repair.

In one aspect, the invention provides a polypeptide for use in treating an inflammatory disorder, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.

In a related aspect, there is provided a method for treating an inflammatory disorder, the method comprising administering a polypeptide to a subject, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.

In another related aspect, the invention provides the use of a polypeptide in the manufacture of a medicament for treating an inflammatory disorder, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.

The polypeptide of the invention may have anti-inflammatory properties. In other words, a polypeptide of the invention may be an anti-inflammatory polypeptide.

Where a polypeptide is used in treating an inflammatory disorder as described herein, the polypeptide may comprise a botulinum neurotoxin serotype X (BoNT/X) L-chain, a BoNT/X H_{N} domain, and/or a BoNT/X H_{C} domain, wherein when the polypeptide comprises a clostridial neurotoxin L-chain, the L-chain is catalytically inactive. For example, the polypeptide may be a chimeric botulinum neurotoxin (BoNT) comprising a catalytically inactive BoNT/X light-chain and translocation domain, and a receptor binding domain (H_{C} domain) from a different (i.e. non-BoNT/X) clostridial neurotoxin. Thus, in one aspect, the invention provides a polypeptide for use in treating an inflammatory disorder, wherein the polypeptide comprises a catalytically inactive BoNT/X light-chain and translocation domain, and a receptor binding domain (H_{C} domain) from a different (i.e. non-BoNT/X) clostridial neurotoxin (preferably a BoNT/B H_{C} domain). Corresponding methods of treatment and uses are also provided.

Preferably, a polypeptide of the invention neither promotes neuronal growth nor neuronal repair to treat an inflammatory condition. In other words, preferably, the polypeptide does not treat an inflammatory condition by any of the following means: by promoting neuronal growth, by promoting neuronal repair, or by promoting neuronal growth and repair

In one aspect, the invention provides a polypeptide for use in treating a lower urinary tract disorder, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.

In a related aspect, the invention provides a method for treating a lower urinary tract disorder, the method comprising administering a polypeptide to a subject, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.

In a related aspect, the invention provides the use of a polypeptide in the manufacture of a medicament for treating a lower urinary tract disorder, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.

A lower urinary tract disorder is preferably a bladder disorder.

The polypeptide of the invention may treat a lower urinary tract disorder (e.g. a bladder disorder) by decreasing or inhibiting activity of a sensory neuron of the bladder (e.g. when compared to the activity of the sensory neuron in the same subject prior to treatment). For example, the polypeptide may decrease or inhibit distension-induced activity of a sensory neuron of the bladder and/or chemical-induced activity of a sensory neuron of the bladder. The sensory neuron of the bladder is preferably an afferent nerve. The afferent nerve may comprise one or more sensory fibers (e.g. a bundle of nerve fibers). Such an afferent nerve may be an afferent nerve that senses chemical and/or mechanical stimulation in the bladder wall (e.g. relating to bladder filing). Preferably, the afferent nerve comprises one or more fibers of the pelvic and/or hypogastric nerves. For example, the afferent nerve may be the pelvic nerve and/or the hypogastric nerve.

By inhibiting activity of a sensory neuron (e.g. afferent nerve) of the bladder, the polypeptides may reduce or inhibit a sensory symptom of urgency associated with a lower urinary tract disorder. A sensory symptom of urgency is typically associated with bladder pain syndrome (e.g. interstitial cystitis), overactive bladder, and/or detrusor overactivity.

The lower urinary tract disorder may thus be a sensory neuron-associated disorder, e.g. an afferent nerve-associated disorder, in particular an afferent nerve-associated bladder disorder. An afferent nerve-associated disorder may be a disorder in which the afferent nerve has a role, whether said role is causative or otherwise. In some embodiments an afferent nerve-associated disorder is a disorder that is caused by or associated with afferent nerve hyperactivity and/or an altered central threshold for afferent impulses. The latter may comprise damage to central inhibitory pathways or the sensitization of afferent nerves. In such embodiments, neurotrophic factors such as nerve growth factor (NGF) or brain-derived neurotrophic factor (BDNF) levels may be increased and affect sensory neurons. Associated symptoms may include activation of the voiding reflex. Signalling in the insula, an area linked to the PAG (periacqueductal gray, one of the central structures involved in the control of micturition), may be altered, with incoherent firing thresholds, resulting in altered central threshold for afferent impulses and sensations of urgency.

Examples of afferent nerve-associated disorders may include bladder pain syndrome (preferably interstitial cystitis), overactive bladder, and detrusor overactivity.

Bladder pain syndrome is a chronic bladder health issue affecting around 6-14M patients in the US (ie. 5-11% of the adult US population). The International Continence Society (ICS) defines bladder pain syndrome as a condition characterised by chronic (>6 months) pelvic pain, pressure or discomfort perceived to be related to the urinary bladder, and that is accompanied by at least one other urinary symptom such as persistent urge to void or frequency. Frequency is the need to pass urine more often than normal. The average person typically urinates no more than 7 times per day and does not have to get up at night more than once to use the bathroom. A bladder pain syndrome sufferer may have to urinate frequently day and night, and as frequency becomes more severe, this leads to urgency. For some patients, this urge to urinate never goes away, even immediately after voiding. Interstitial cystitis may be defined as a more severe or advanced form of bladder pain syndrome, and may be further characterised by the presence of “typical cytoscopic and histological features”. For example, when compared with non-interstitial cystitis bladder pain syndrome patients, interstitial cystitis patients may have a higher incidence and degree of denuded epithelium, ulceration, pyuria and/or submucosal inflammation. Interstitial cystitis may be described as a chronic submucosal inflammatory disease.

By way of background, urine is formed by the nephrons of the kidney and is transported to the urinary bladder for storage before being expelled via the urethra. This process of cyclical filling-and-emptying is known as micturition. As the bladder fills, it stretches, simulating afferent signals (conveyed by afferent nerves). Conversely, efferent signals result in contraction of the bladder musculature and relaxation of the urethral sphincter, respectively. In addition to mechanoreceptors, various psychological factors (e.g. stress, sense of acceptable surroundings, and emotional status) play a crucial role in the timing and setting of micturition. Overactive bladder is described by the ICS as urinary urgency, usually accompanied by increased daytime frequency and/or nocturia, with urinary incontinence (OAB-wet) or without (OAB-dry), in the absence of urinary tract infection or other detectable disease.

The detrusor overactivity may be idiopathic detrusor overactivity or neurogenic detrusor overactivity. Said overactivity may be: (1) phasic, which may or may not lead to urinary incontinence; or (2) terminal, which is a single involuntary detrusor contraction that often results in complete bladder emptying.

Neurogenic detrusor overactivity may be associated with involuntary detrusor muscle contractions which occur near or at the maximum cystometric capacity, in the setting of a clinically relevant neurologic disease. These contractions generally cannot be suppressed resulting in urinary incontinence or even reflex bladder emptying (reflex voiding). Said disorder is frequently observed in patients with conditions such as multiple sclerosis (MS) and spinal cord injury (SCI). Thus, a subject treated in accordance with the present invention may have an SCI and/or MS.

When treating a lower urinary tract disorder, the polypeptide may be administered by any suitable method known in the art. For example, the polypeptide may be administered by way of injection to the bladder wall, e.g. intradetrusal injection.

When treating a lower urinary tract disorder (e.g. a bladder disorder), it is preferred that the polypeptide comprises at least a catalytically inactive clostridial neurotoxin L-chain. More preferably, when treating a lower urinary tract disorder (e.g. a bladder disorder), the polypeptide comprises a catalytically inactive clostridial neurotoxin L-chain, a clostridial neurotoxin translocation domain (H_{N} domain), and a clostridial neurotoxin receptor binding domain (H_{C} domain). Advantageously, such a polypeptide was more effective at treating a lower urinary tract disorder when compared to polypeptides in which one of said components was absent.

Preferably, a polypeptide of the invention neither promotes neuronal growth nor neuronal repair to treat a lower urinary tract disorder. In other words, preferably, the polypeptide does not treat a lower urinary tract disorder by any of the following means: by promoting neuronal growth, by promoting neuronal repair, or by promoting neuronal growth and repair The term “promotes neuronal growth and/or neuronal repair” encompasses an increase in the rate of neuronal growth and/or neuronal repair. The term “neuronal growth and/or neuronal repair” encompasses the rebuilding of damaged neuronal circuits, thereby restoring activity and/or neuronal communication in a network or population of neurons. Thus, the term “neuronal repair” as used herein encompasses repair of a specific neuron as well as repair of a neuronal circuit. The term also encompasses neuronal plasticity. The term “neuronal plasticity” as used herein encompasses axonal sprouting, dendritic sprouting, neurogenesis (e.g. the production of new neurons), maturation, differentiation, and/or synaptic plasticity (e.g. including changes to synaptic strength, activity, anatomy, and/or connectivity). The term “promotes neuronal growth and/or neuronal repair” also encompasses promoting the establishment of functional synapses (e.g. at or near to a site of injury). The term “neuronal growth” as used herein encompasses growth of any part of a neuron, including growth of axons and/or dendrites. Said term encompasses an increase neurite length, neurite number (e.g. number of neurites per cell), and/or an increase the length and/or numbers of projections from a cell body or cell membrane of a neuron, e.g. axonal growth of a neuron and/or axonal sprouting, e.g. a neuron in a subject. Said axonal growth may promote connections and/or chemical communication between neurons.

Preferably, a polypeptide of the invention does not promote a neuroimmune response to treat pain or an inflammatory disorder. Preferably, a polypeptide of the invention does not promote a neuroimmune response to treat a lower urinary tract disorder. A neuroimmune response in this context encompasses a microglial response. Thus, in one embodiment a polypeptide of the invention does not promote a microglial response to treat pain or an inflammatory condition. Thus, in one embodiment a polypeptide of the invention does not promote a microglial response to treat a lower urinary tract disorder.

In a preferred embodiment, the pain is not pain associated with, or caused by, a brain disorder. In another preferred embodiment, the inflammatory disorder is not an inflammatory brain disorder. The term “brain disorder” used in this context is interchangeable with “brain disease”. A “brain disorder” as used in this context encompasses a disorder that originates from within or outside the brain, and includes disorders associated with bodily insults that cause brain tissue damage. Examples of brain disorders encompassed in this context include any one (or more) of traumatic brain injury, cancer (e.g. a brain tumour), infectious disease (e.g. encephalitis, meningitis, a brain abscess, and encephalitis), stroke, a neurodegenerative disorder (e.g. Alzheimer's disease, Parkinson's disease, Parkinson's disease related disorders, motor neuron disease (e.g. amyotrophic lateral sclerosis), prion disease, Huntington's disease, spinocerebellar ataxia, ataxia, Hallervorden-Spatz disease, and frontotemporal lobar degeneration), brain aneurysm, multiple sclerosis, anoxic injury, toxic injury and metabolic injury. A brain disorder may be caused by traumatic brain injury, cancer, infectious disease (e.g. encephalitis, meningitis, a brain abscess, and encephalitis), stroke, a neurodegenerative disorder (e.g. Alzheimer's disease, Parkinson's disease, Parkinson's disease related disorders, motor neuron disease (e.g. amyotrophic lateral sclerosis), prion disease, Huntington's disease, spinocerebellar ataxia, ataxia, Hallervorden-Spatz disease, and frontotemporal lobar degeneration), brain aneurysm, multiple sclerosis, anoxic injury, toxic injury and/or metabolic injury.

Active clostridial neurotoxin L-chain has non-cytotoxic protease activity. Specifically, active clostridial neurotoxin L-chain has endopeptidase activity and is capable of cleaving a protein of the exocytic fusion apparatus in a target cell. A protein of the exocytic fusion apparatus is preferably a SNARE protein, such as SNAP25, synaptobrevin/VAMP, or syntaxin.

The term “catalytically inactive” as used herein in respect of a clostridial neurotoxin L-chain means that said L-chain exhibits substantially no non-cytotoxic protease activity, preferably the term “catalytically inactive” as used herein in respect of a clostridial neurotoxin L-chain means that said L-chain exhibits no non-cytotoxic protease activity. In one embodiment, a catalytically inactive clostridial neurotoxin L-chain is one that does not cleave a protein of the exocytic fusion apparatus in a target cell. The term “substantially no non-cytotoxic protease activity” means that the clostridial neurotoxin L-chain has less than 5% of the non-cytotoxic protease activity of a catalytically active clostridial neurotoxin L-chain, for example less than 2%, 1% or preferably less than 0.1% of the non-cytotoxic protease activity of a catalytically active clostridial neurotoxin L-chain. Non-cytotoxic protease activity can be determined in vitro by incubating a test clostridial neurotoxin L-chain with a SNARE protein and comparing the amount of SNARE protein cleaved by the test clostridial neurotoxin L-chain when compared to the amount of SNARE protein cleaved by a catalytically active clostridial neurotoxin L-chain under the same conditions. Routine techniques, such as SDS-PAGE and Western blotting can be used to quantify the amount of SNARE protein cleaved. Suitable in vitro assays are described in WO 2019/145577 A1, which is incorporated herein by reference.

Cell-based and in vivo assays may also be used to determine if a clostridial neurotoxin comprising an L-chain and a functional cell binding and translocation domain has non-cytotoxic protease activity. Assays such as the Digit Abduction Score (DAS) assay, the dorsal root ganglia (DRG) assay, spinal cord neuron (SCN) assay, and mouse phrenic nerve hemidiaphragm (PNHD) assay are routine in the art. A suitable assay for determining non-cytotoxic protease activity may be one described in Aoki K R, Toxicon 39: 1815-1820; 2001 or Donald et al (2018), Pharmacol Res Perspect, e00446, 1-14, which are incorporated herein by reference.

A catalytically inactive L-chain may have one or more mutations that inactivate said catalytic activity. Thus, a catalytically active L-chain (e.g. as described herein) may be modified to introduce one or more mutations that inactivate the catalytic activity of the L-chain. For example, a catalytically inactive L-chain may comprise a mutation of an active site residue. A mutation may be a substitution or a deletion, however a substitution is preferred, in particular substitution with a chemically-similar amino acid. Glutamic acid may be substituted with glutamine, histidine may be substituted with tyrosine, arginine may be substituted with glutamine, and/or tyrosine may be substituted with phenylalanine. Alternatively, any residue may be substituted with alanine.

A catalytically inactive BoNT/A L-chain may comprise a mutation at H223, E224, H227, E262, R363, and/or Y366, preferably at at least E224 and H227. Preferably, a catalytically inactive BoNT/A L-chain may comprise substitution at E224 with glutamine (E224Q) and substitution at H227 with tyrosine (H227Y). The position numbering corresponds to the amino acid positions of SEQ ID NO: 60 and can be determined by aligning a polypeptide with SEQ ID NO: 60. As the presence of a methionine residue at position 1 of SEQ ID NO: 60 is optional, the skilled person will take the presence/absence of the methionine residue into account when determining amino acid residue numbering. For example, where SEQ ID NO: 60 includes a methionine, the position numbering will be as defined above (e.g. His223 will be His223 of SEQ ID NO: 60). Alternatively, where the methionine is absent from SEQ ID NO: 60 the amino acid residue numbering should be modified by −1 (e.g. His223 will be His222 of SEQ ID NO: 60). Similar considerations apply when the methionine at position 1 of the other polypeptide sequences described herein is present/absent, and the skilled person will readily determine the correct amino acid residue numbering using techniques routine in the art.

A catalytically inactive BoNT/B L-chain may comprise a mutation at E231 and/or H234, preferably E231 and H234. Preferably, a catalytically inactive BoNT/B L-chain comprises substitution at E231 with glutamine (E231Q) and substation at H234 with tyrosine (H234Y). The position numbering corresponds to the amino acid positions of SEQ ID NO: 52 and can be determined by aligning a polypeptide with SEQ ID NO: 52. As the presence of a methionine residue at position 1 of SEQ ID NO: 52 is optional, the skilled person will take the presence/absence of the methionine residue into account when determining amino acid residue numbering.

A catalytically inactive BoNT/C L-chain may comprise a mutation at H229, E230 and/or H233, preferably H229, E230 and H233. Preferably, a catalytically inactive BoNT/C L-chain comprises substitution at H229 with glycine (H229G), substitution at E230 with threonine (E230T), and substitution at H233 with asparagine (H233N). The position numbering corresponds to the amino acid positions of SEQ ID NO: 53 and can be determined by aligning a polypeptide with SEQ ID NO: 53. As the presence of a methionine residue at position 1 of SEQ ID NO: 53 is optional, the skilled person will take the presence/absence of the methionine residue into account when determining amino acid residue numbering.

A catalytically inactive BoNT/D L-chain may comprise a mutation at H229, E230, H233 and/or H236, preferably at at least E230 and H236. Preferably, a catalytically inactive BoNT/D L-chain comprises at least substitution at E230 with glutamine (E230Q) and substitution at H236 with tyrosine (H236Y). The position numbering corresponds to the amino acid positions of SEQ ID NO: 54 and can be determined by aligning a polypeptide with SEQ ID NO: 54. As the presence of a methionine residue at position 1 of SEQ ID NO: 54 is optional, the skilled person will take the presence/absence of the methionine residue into account when determining amino acid residue numbering.

A catalytically inactive BoNT/E L-chain may comprise a mutation at E213 and/or H216, preferably at E213 and H216. Preferably, a catalytically inactive BoNT/E L-chain comprises substitution at E213 with glutamine (E213Q) and H216 with tyrosine (H216Y). The position numbering corresponds to the amino acid positions of SEQ ID NO: 55 and can be determined by aligning a polypeptide with SEQ ID NO: 55. As the presence of a methionine residue at position 1 of SEQ ID NO: 55 is optional, the skilled person will take the presence/absence of the methionine residue into account when determining amino acid residue numbering.

A catalytically inactive BoNT/F L-chain may comprise a mutation at E228 and/or H231, preferably at E228 and H231. Preferably, a catalytically inactive BoNT/F L-chain comprises substitution at E228 with glutamine (E228Q) and H231 with tyrosine (H231Y). The position numbering corresponds to the amino acid positions of SEQ ID NO: 56 and can be determined by aligning a polypeptide with SEQ ID NO: 56. As the presence of a methionine residue at position 1 of SEQ ID NO: 56 is optional, the skilled person will take the presence/absence of the methionine residue into account when determining amino acid residue numbering.

A catalytically inactive BoNT/G L-chain may comprise a mutation at E231 and/or H234, preferably at E231 and H234. Preferably, a catalytically inactive BoNT/G L-chain comprises substitution at E231 with glutamine (E231Q) and H234 with tyrosine (H234Y). The position numbering corresponds to the amino acid positions of SEQ ID NO: 57 and can be determined by aligning a polypeptide with SEQ ID NO: 57. As the presence of a methionine residue at position 1 of SEQ ID NO: 57 is optional, the skilled person will take the presence/absence of the methionine residue into account when determining amino acid residue numbering.

A catalytically inactive BoNT/X L-chain may comprise a mutation at E228 and/or H231, preferably at E228 and H231. Preferably, a catalytically inactive BoNT/X L-chain comprises substitution at E228 with glutamine (E228Q) and H231 with tyrosine (H231Y). The position numbering corresponds to the amino acid positions of SEQ ID NO: 59 and can be determined by aligning a polypeptide with SEQ ID NO: 59. As the presence of a methionine residue at position 1 of SEQ ID NO: 59 is optional, the skilled person will take the presence/absence of the methionine residue into account when determining amino acid residue numbering.

A catalytically inactive TeNT L-chain may comprise a mutation at E234, R372, and/or Y375, preferably at at least R372 and Y375 (e.g. at E234, R372, and Y375). Preferably, a catalytically inactive TeNT L-chain comprises substitution at R372 with glutamine or alanine (R372Q or R372A), more preferably with alanine, and substitution at Y375 with phenylalanine (Y375F). The position numbering corresponds to the amino acid positions of SEQ ID NO: 58 and can be determined by aligning a polypeptide with SEQ ID NO: 58. As the presence of a methionine residue at position 1 of SEQ ID NO: 58 is optional, the skilled person will take the presence/absence of the methionine residue into account when determining amino acid residue numbering.

The polypeptide of the invention may comprise a full-length clostridial neurotoxin (with the proviso that the L-chain is catalytically inactive) or fragments of clostridial neurotoxins that do not have non-cytotoxic protease activity (e.g. the H_{N} domain and/or H_{C} domain). In other words, the polypeptides of the invention do not have non-cytotoxic protease activity. See, however, the section herein entitled “Polypeptides comprising a catalytically active clostridial neurotoxin light-chain and lacking a functional H_{CC} domain or H_{C} domain”, which is directed to alternative polypeptides of the invention in which the L-chain does have catalytic activity (but wherein said polypeptides lack a functional H_{CC} domain or H_{C} domain and, thus, are associated with little/no toxicity [e.g. little/no non-cytotoxic activity]).

The term “clostridial neurotoxin” embraces toxins produced by C. botulinum (botulinum neurotoxin serotypes A, B, C₁, D, E, F, G, and X), C. tetani (tetanus neurotoxin), C. butyricum (botulinum neurotoxin serotype E), and C. baratii (botulinum neurotoxin serotype F). A reference BoNT/A sequence is shown as SEQ ID NO: 51. A reference BoNT/B sequence is shown as SEQ ID NO: 52. A reference BoNT/C sequence is shown as SEQ ID NO: 53. A reference BoNT/D sequence is shown as SEQ ID NO: 54. A reference BoNT/E sequence is shown as SEQ ID NO: 55. A reference BoNT/F sequence is shown as SEQ ID NO: 56. A reference BoNT/G sequence is shown as SEQ ID NO: 57. A reference TeNT sequence is shown as SEQ ID NO: 58. A reference BoNT/X sequence is shown as SEQ ID NO: 59. The term “clostridial neurotoxin” may also embrace newly discovered botulinum neurotoxin protein family members expressed by non-clostridial microorganisms, such as the Enterococcus encoded toxin which has closest sequence identity to BoNT/X, the Weissella oryzae encoded toxin called BoNT/Wo (NCBI Ref Seq: WP_027699549.1), which cleaves VAMP2 at W89-W90, the Enterococcus faecium encoded toxin (GenBank: OTO2244.1), which cleaves VAMP2 and SNAP25, and the Chryseobacteriumpipero encoded toxin (NCBI Ref.Seq: WP_034687872.1).

Thus, a clostridial neurotoxin may be selected from BoNT/A, BoNT/B, BoNT/C, BoNT/D, BoNT/E, BoNT/F, BoNT/G, BoNT/X, and TeNT (tetanus neurotoxin). Preferably, a clostridial neurotoxin is a botulinum neurotoxin, such as a botulinum neurotoxin selected from BoNT/A, BoNT/B, BoNT/C, BoNT/D, BoNT/E, BoNT/F, BoNT/G, and BoNT/X. For example, a clostridial neurotoxin H_{N} domain may be a H_{N} domain from BoNT A B, C₁, D, E, F, G, X or TeNT. Similarly, an L-chain may be an L-chain from BoNT A B, C₁, D, E, F, G, X or TeNT with the proviso that said L-chain is catalytically inactive (e.g. has been modified to render it catalytically inactive). More preferably, the clostridial neurotoxin is BoNT/A.

As discussed above, (full-length) clostridial neurotoxins are formed from two polypeptide chains, the heavy chain (H-chain), which has a molecular mass of approximately 100 kDa, and the light chain (L-chain), which has a molecular mass of approximately 50 kDa. The H-chain comprises a C-terminal targeting component (receptor binding domain or H_{C} domain) and an N-terminal translocation component (H_{N} domain). Botulinum neurotoxin (BoNT) is produced by C. botulinum in the form of a large protein complex, consisting of BoNT itself complexed to a number of accessory proteins. There are at present eight different classes of botulinum neurotoxin, namely: botulinum neurotoxin serotypes A, B, C1, D, E, F, G, and X all of which share similar structures and modes of action. Different BoNT serotypes can be distinguished based on inactivation by specific neutralising anti-sera, with such classification by serotype correlating with percentage sequence identity at the amino acid level. BoNT proteins of a given serotype are further divided into different subtypes on the basis of amino acid percentage sequence identity.

Conventional (catalytically active) BoNTs are absorbed in the gastrointestinal tract, and, after entering the general circulation, bind to the presynaptic membrane of cholinergic nerve terminals and prevent the release of their neurotransmitter acetylcholine. BoNT/B, BoNT/D, BoNT/F and BoNT/G cleave synaptobrevin/vesicle-associated membrane protein (VAMP); BoNT/C1, BoNT/A and BoNT/E cleave the synaptosomal-associated protein of 25 kDa (SNAP-25); and BoNT/C1 cleaves syntaxin. BoNT/X has been found to cleave SNAP-25, VAMP1, VAMP2, VAMP3, VAMP4, VAMP5, Ykt6, and syntaxin 1. Tetanus toxin is produced in a single serotype by C. tetani. C. butyricum produces BoNT/E, while C. baratii produces BoNT/F.

In one embodiment, a polypeptide of the invention may be encoded by a nucleotide sequence having at least 70% sequence identity to any one of SEQ ID NOs: 1, 3, 5, 7, 9, 11, 13, 15, 17, 19, 21, 23, 25, 27, 29, 31, 33, 35, 37, 39, 41, 43, 45, 47, or 49 with the proviso that when the polypeptide comprises a clostridial neurotoxin L-chain, the L-chain is catalytically inactive. In one embodiment, a polypeptide of the invention may be encoded by a nucleotide sequence having at least 80%, 90%, 95% or 98% sequence identity to any one of SEQ ID NOs: 1, 3, 5, 7, 9, 11, 13, 15, 17, 19, 21, 23, 25, 27, 29, 31, 33, 35, 37, 39, 41, 43, 45, 47, or 49 with the proviso that when the polypeptide comprises a clostridial neurotoxin L-chain, the L-chain is catalytically inactive. Preferably, a polypeptide of the invention may be encoded by a nucleotide sequence comprising any one of SEQ ID NOs: 1, 7, 9, 11, 13, 15, 17, 21, 25, 29, 33, 37, 41, 43, 45, 47, or 49.

In one embodiment a polypeptide of the invention may comprise a polypeptide sequence having at least 70% sequence identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 60, 61, 62, 63, 64, 65, 66, 67, 68, 69, 70, 74, 75 or 76 with the proviso that when the polypeptide comprises a clostridial neurotoxin L-chain, the L-chain is catalytically inactive. In one embodiment a polypeptide of the invention may comprise a polypeptide sequence having at least 80%, 90%, 95% or 98% sequence identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 60, 61, 62, 63, 64, 65, 66, 67, 68, 69, 70, 74, 75 or 76 with the proviso that when the polypeptide comprises a clostridial neurotoxin L-chain, the L-chain is catalytically inactive. Preferably, a polypeptide of the invention may comprise a polypeptide sequence of any one of SEQ ID NOs: 2, 8, 10, 12, 14, 16, 18, 22, 26, 30, 34, 38, 42, 44, 46, 48, 50, 61, 62, 63, 64, 65, 66, 67, 68, 69, 70, 74, 75 or 76.

In one embodiment a polypeptide of the invention may comprise a fragment of a polypeptide sequence having at least 70% sequence identity to any one of SEQ ID NOs: 2, 10, 12, 14, 16, 18, 26, 34, 51, 52, 53, 54, 55, 56, 57, 58, 59, 60, 61, 62, 63, 64, 65, 66, 67, 68, 69 or 70 with the proviso that when the polypeptide comprises a clostridial neurotoxin L-chain, the L-chain is catalytically inactive. In one embodiment a polypeptide of the invention may comprise a fragment of a polypeptide sequence having at least 80%, 90%, 95% or 98% sequence identity to any one of SEQ ID NOs: 2, 10, 12, 14, 16, 18, 26, 34, 51, 52, 53, 54, 55, 56, 57, 58, 59, 60, 61, 62, 63, 64, 65, 66, 67, 68, 69 or 70 with the proviso that when the polypeptide comprises a clostridial neurotoxin L-chain, the L-chain is catalytically inactive. Preferably, a polypeptide of the invention may comprise a fragment of a polypeptide sequence comprising any one of SEQ ID NOs: 2, 10, 12, 14, 16, 18, 26, 34, 51, 52, 53, 54, 55, 56, 57, 58, 59, 60, 61, 62, 63, 64, 65, 66, 67, 68, 69 or 70 with the proviso that when the polypeptide comprises a clostridial neurotoxin L-chain, the L-chain is catalytically inactive. The fragment may be a catalytically inactive L-chain, H_{N} domain or H_{C} domain of any of said SEQ ID NOs.

Preferably, a polypeptide of the invention comprises (or consists of) a catalytically inactive clostridial neurotoxin L-chain. Reference to a catalytically inactive clostridial neurotoxin in this context also encompasses a fragment of a clostridial neurotoxin L-chain. A fragment of a clostridial neurotoxin L-chain may have 5400, 5350, 5300, 5250, 5200, 5150, 5100 or 550 amino acid residues of a clostridial neurotoxin L-chain. In one embodiment, a fragment of a clostridial neurotoxin L-chain has at least 20, 30, 40, 50, 60, 70, 80, 90, 100, 120, 150 or 200 amino acid residues of a clostridial neurotoxin L-chain. For example, a fragment of a clostridial neurotoxin L-chain may have 20-400, 50-300 or 100-200 amino acid residues of a clostridial neurotoxin L-chain. It is preferred, however, that reference to a catalytically inactive clostridial neurotoxin is reference to a full-length catalytically inactive clostridial neurotoxin L-chain.

Examples of L-chain reference sequences include:
Botulinum type A neurotoxin: amino acid residues 1-448
Botulinum type B neurotoxin: amino acid residues 1-440
Botulinum type C1 neurotoxin: amino acid residues 1-441
Botulinum type D neurotoxin: amino acid residues 1-445
Botulinum type E neurotoxin: amino acid residues 1-422
Botulinum type F neurotoxin: amino acid residues 1-439
Botulinum type G neurotoxin: amino acid residues 1-441
Tetanus neurotoxin: amino acid residues 1-457

For recently-identified BoNT/X, the L-chain has been reported as corresponding to amino acids 1-439 thereof, with the L-chain boundary potentially varying by approximately 25 amino acids (e.g. 1-414 or 1-464).

The above-identified reference sequences should be considered a guide, as slight variations may occur according to sub-serotypes. By way of example, US 2007/0166332 (hereby incorporated by reference in its entirety) cites slightly different clostridial sequences:
Botulinum type A neurotoxin: amino acid residues M1-K448
Botulinum type B neurotoxin: amino acid residues M1-K441
Botulinum type C1 neurotoxin: amino acid residues M1-K449
Botulinum type D neurotoxin: amino acid residues M1-R445
Botulinum type E neurotoxin: amino acid residues M1-R422
Botulinum type F neurotoxin: amino acid residues M1-K439
Botulinum type G neurotoxin: amino acid residues M1-K446
Tetanus neurotoxin: amino acid residues M1-A457

Suitable clostridial neurotoxin L-chains are described herein.

A clostridial neurotoxin L-chain (or polypeptide) may comprise:
(i) a BoNT/A L-chain with the proviso that the L-chain is catalytically inactive (e.g. the L-chain has been inactivated by modification);
(ii) a BoNT/B L-chain with the proviso that the L-chain is catalytically inactive (e.g. the L-chain has been inactivated by modification);
(iii) a BoNT/C1 L-chain with the proviso that the L-chain is catalytically inactive (e.g. the L-chain has been inactivated by modification);
(iv) a BoNT/D L-chain with the proviso that the L-chain is catalytically inactive (e.g. the L-chain has been inactivated by modification);
(v) a BoNT/E L-chain with the proviso that the L-chain is catalytically inactive (e.g. the L-chain has been inactivated by modification);
(vi) a BoNT/F L-chain with the proviso that the L-chain is catalytically inactive (e.g. the L-chain has been inactivated by modification);
(vii) a BoNT/G L-chain with the proviso that the L-chain is catalytically inactive (e.g. the L-chain has been inactivated by modification);
(viii) a BoNT/X L-chain with the proviso that the L-chain is catalytically inactive (e.g. the L-chain has been inactivated by modification); or
(ix) a TeNT L-chain with the proviso that the L-chain is catalytically inactive (e.g. the L-chain has been inactivated by modification).

Said catalytically inactive clostridial neurotoxin L-chain (or polypeptide) may consist essentially of, preferably consist of, any one of the above with the proviso that the L-chain is catalytically inactive (e.g. the L-chain has been inactivated by modification).

In one embodiment a polypeptide comprises a polypeptide sequence having at least:
(i) 70% (e.g. at least 80%, 90%, 95%, or 98%) sequence identity to residues 1-448 of SEQ ID NO: 51 with the proviso that the L-chain is catalytically inactive (e.g. the L-chain has been inactivated by modification);
(ii) 70% (e.g. at least 80%, 90%, 95%, or 98%) sequence identity to residues 1-440 of SEQ ID NO: 52 with the proviso that the L-chain is catalytically inactive (e.g. the L-chain has been inactivated by modification);
(iii) 70% (e.g. at least 80%, 90%, 95%, or 98%) sequence identity to residues 1-441 of SEQ ID NO: 53 with the proviso that the L-chain is catalytically inactive (e.g. the L-chain has been inactivated by modification);
(iv) 70% (e.g. at least 80%, 90%, 95%, or 98%) sequence identity to residues 1-445 of SEQ ID NO: 54 with the proviso that the L-chain is catalytically inactive (e.g. the L-chain has been inactivated by modification);
(v) 70% (e.g. at least 80%, 90%, 95%, or 98%) sequence identity to residues 1-422 of SEQ ID NO: 55 with the proviso that the L-chain is catalytically inactive (e.g. the L-chain has been inactivated by modification);
(vi) 70% (e.g. at least 80%, 90%, 95%, or 98%) sequence identity to residues 1-439 of SEQ ID NO: 56 with the proviso that the L-chain is catalytically inactive (e.g. the L-chain has been inactivated by modification);
(vii) 70% (e.g. at least 80%, 90%, 95%, or 98%) sequence identity to residues 1-441 of SEQ ID NO: 57 with the proviso that the L-chain is catalytically inactive (e.g. the L-chain has been inactivated by modification);
(viii) 70% (e.g. at least 80%, 90%, 95%, or 98%) sequence identity to residues 1-414, 1-439, or 1-464 of SEQ ID NO: 59 with the proviso that the L-chain is catalytically inactive (e.g. the L-chain has been inactivated by modification); or
(ix) 70% (e.g. at least 80%, 90%, 95%, or 98%) sequence identity to residues 1-457 of SEQ ID NO: 58 with the proviso that the L-chain is catalytically inactive (e.g. the L-chain has been inactivated by modification).

In one embodiment a polypeptide comprises a polypeptide sequence having:
(i) residues 1-448 of SEQ ID NO: 51 that has been further modified to catalytically inactivate the L-chain;
(ii) residues 1-440 of SEQ ID NO: 52 that has been further modified to catalytically inactivate the L-chain;
(iii) residues 1-441 of SEQ ID NO: 53 that has been further modified to catalytically inactivate the L-chain;
(iv) residues 1-445 of SEQ ID NO: 54 that has been further modified to catalytically inactivate the L-chain;
(v) residues 1-422 of SEQ ID NO: 55 that has been further modified to catalytically inactivate the L-chain;
(vi) residues 1-439 of SEQ ID NO: 56 that has been further modified to catalytically inactivate the L-chain;
(vii) residues 1-441 of SEQ ID NO: 57 that has been further modified to catalytically inactivate the L-chain;
(viii) residues 1-414, 1-439, or 1-464 of SEQ ID NO: 59 that has been further modified to catalytically inactivate the L-chain; or
(ix) residues 1-457 of SEQ ID NO: 58 that has been further modified to catalytically inactivate the L-chain.

The polypeptide may consist essentially of a polypeptide sequence having at least:
(i) 70% (e.g. at least 80%, 90%, 95%, or 98%) sequence identity to residues 1-448 of SEQ ID NO: 51 with the proviso that the L-chain is catalytically inactive (e.g. the L-chain has been inactivated by modification);
(ii) 70% (e.g. at least 80%, 90%, 95%, or 98%) sequence identity to residues 1-440 of SEQ ID NO: 52 with the proviso that the L-chain is catalytically inactive (e.g. the L-chain has been inactivated by modification);
(iii) 70% (e.g. at least 80%, 90%, 95%, or 98%) sequence identity to residues 1-441 of SEQ ID NO: 53 with the proviso that the L-chain is catalytically inactive (e.g. the L-chain has been inactivated by modification);
(iv) 70% (e.g. at least 80%, 90%, 95%, or 98%) sequence identity to residues 1-445 of SEQ ID NO: 54 with the proviso that the L-chain is catalytically inactive (e.g. the L-chain has been inactivated by modification);
(v) 70% (e.g. at least 80%, 90%, 95%, 98%, or 100%) sequence identity to residues 1-422 of SEQ ID NO: 55 with the proviso that the L-chain is catalytically inactive (e.g. the L-chain has been inactivated by modification);
(vi) 70% (e.g. at least 80%, 90%, 95%, or 98%) sequence identity to residues 1-439 of SEQ ID NO: 56 with the proviso that the L-chain is catalytically inactive (e.g. the L-chain has been inactivated by modification);
(vii) 70% (e.g. at least 80%, 90%, 95%, or 98%) sequence identity to residues 1-441 of SEQ ID NO: 57 with the proviso that the L-chain is catalytically inactive (e.g. the L-chain has been inactivated by modification);
(viii) 70% (e.g. at least 80%, 90%, 95%, or 98%) sequence identity to residues 1-414, 1-439, or 1-464 of SEQ ID NO: 59 with the proviso that the L-chain is catalytically inactive (e.g. the L-chain has been inactivated by modification); or
(ix) 70% (e.g. at least 80%, 90%, 95%, or 98%) sequence identity to residues 1-457 of SEQ ID NO: 58 with the proviso that the L-chain is catalytically inactive (e.g. the L-chain has been inactivated by modification).

The polypeptide may consist essentially of a polypeptide sequence having:
(i) residues 1-448 of SEQ ID NO: 51 that has been further modified to catalytically inactivate the L-chain;
(ii) residues 1-440 of SEQ ID NO: 52 that has been further modified to catalytically inactivate the L-chain;
(iii) residues 1-441 of SEQ ID NO: 53 that has been further modified to catalytically inactivate the L-chain;
(iv) residues 1-445 of SEQ ID NO: 54 that has been further modified to catalytically inactivate the L-chain;
(v) residues 1-422 of SEQ ID NO: 55 that has been further modified to catalytically inactivate the L-chain;
(vi) residues 1-439 of SEQ ID NO: 56 that has been further modified to catalytically inactivate the L-chain;
(vii) residues 1-441 of SEQ ID NO: 57 that has been further modified to catalytically inactivate the L-chain;
(viii) residues 1-414, 1-439, or 1-464 of SEQ ID NO: 59 that has been further modified to catalytically inactivate the L-chain; or
(ix) residues 1-457 of SEQ ID NO: 58 that has been further modified to catalytically inactivate the L-chain.

The polypeptide may consist of a polypeptide sequence having at least:
(i) 70% (e.g. at least 80%, 90%, 95%, or 98%) sequence identity to residues 1-448 of SEQ ID NO: 51 with the proviso that the L-chain is catalytically inactive (e.g. the L-chain has been inactivated by modification);
(ii) 70% (e.g. at least 80%, 90%, 95%, or 98%) sequence identity to residues 1-440 of SEQ ID NO: 52 with the proviso that the L-chain is catalytically inactive (e.g. the L-chain has been inactivated by modification);
(iii) 70% (e.g. at least 80%, 90%, 95%, or 98%) sequence identity to residues 1-441 of SEQ ID NO: 53 with the proviso that the L-chain is catalytically inactive (e.g. the L-chain has been inactivated by modification);
(iv) 70% (e.g. at least 80%, 90%, 95%, or 98%) sequence identity to residues 1-445 of SEQ ID NO: 54 with the proviso that the L-chain is catalytically inactive (e.g. the L-chain has been inactivated by modification);
(v) 70% (e.g. at least 80%, 90%, 95%, or 98%) sequence identity to residues 1-422 of SEQ ID NO: 55 with the proviso that the L-chain is catalytically inactive (e.g. the L-chain has been inactivated by modification);
(vi) 70% (e.g. at least 80%, 90%, 95%, or 98%) sequence identity to residues 1-439 of SEQ ID NO: 56 with the proviso that the L-chain is catalytically inactive (e.g. the L-chain has been inactivated by modification);
(vii) 70% (e.g. at least 80%, 90%, 95%, or 98%) sequence identity to residues 1-441 of SEQ ID NO: 57 with the proviso that the L-chain is catalytically inactive (e.g. the L-chain has been inactivated by modification);
(viii) 70% (e.g. at least 80%, 90%, 95%, or 98%) sequence identity to residues 1-414, 1-439, or 1-464 of SEQ ID NO: 59 with the proviso that the L-chain is catalytically inactive (e.g. the L-chain has been inactivated by modification); or
(ix) 70% (e.g. at least 80%, 90%, 95%, or 98%) sequence identity to residues 1-457 of SEQ ID NO: 58 with the proviso that the L-chain is catalytically inactive (e.g. the L-chain has been inactivated by modification).

The polypeptide may consist of a polypeptide sequence having:
(i) residues 1-448 of SEQ ID NO: 51 that has been further modified to catalytically inactivate the L-chain;
(ii) residues 1-440 of SEQ ID NO: 52 that has been further modified to catalytically inactivate the L-chain;
(iii) residues 1-441 of SEQ ID NO: 53 that has been further modified to catalytically inactivate the L-chain;
(iv) residues 1-445 of SEQ ID NO: 54 that has been further modified to catalytically inactivate the L-chain;
(v) residues 1-422 of SEQ ID NO: 55 that has been further modified to catalytically inactivate the L-chain;
(vi) residues 1-439 of SEQ ID NO: 56 that has been further modified to catalytically inactivate the L-chain;
(vii) residues 1-441 of SEQ ID NO: 57 that has been further modified to catalytically inactivate the L-chain;
(viii) residues 1-414, 1-439, or 1-464 of SEQ ID NO: 59 that has been further modified to catalytically inactivate the L-chain; or
(ix) residues 1-457 of SEQ ID NO: 58 that has been further modified to catalytically inactivate the L-chain.

A clostridial neurotoxin L-chain may comprise a polypeptide sequence having at least 70% sequence identity to any one of SEQ ID NOs: 6, 24, 32, 40, 74 or 76 with the proviso that the L-chain is catalytically inactive (e.g. the L-chain has been inactivated by modification). In one embodiment a clostridial neurotoxin L-chain comprises a polypeptide sequence having at least 80%, 90%, 95% or 98% sequence identity to any one of SEQ ID NOs: 6, 24, 32, 40, 74 or 76 with the proviso that the L-chain is catalytically inactive (e.g. the L-chain has been inactivated by modification). Preferably, a clostridial neurotoxin L-chain comprises (more preferably consists of) a polypeptide sequence comprising any one of SEQ ID NOs: 6, 24, 32 or 40 that has been modified to catalytically inactivate the L-chain, for example SEQ ID NO: 74 or 76.

A clostridial neurotoxin L-chain may be one encoded by a nucleotide sequence having at least 70% sequence identity to any one of SEQ ID NOs: 5, 23, 31 or 39 with the proviso that the L-chain is catalytically inactive (e.g. the L-chain has been inactivated by modification). In one embodiment a clostridial neurotoxin L-chain is one encoded by a nucleotide sequence having at least 80%, 90%, 95% or 98% sequence identity to any one of SEQ ID NOs: 5, 23, 31 or 39 with the proviso that the L-chain is catalytically inactive (e.g. the L-chain has been inactivated by modification). Preferably, a clostridial neurotoxin L-chain is one encoded by a nucleotide sequence comprising any one of SEQ ID NOs: 5, 23, 31 or 39 that has been modified to catalytically inactivate the encoded L-chain.

Given that catalytic activity of the light-chain was not required for efficacy in treating pain, it is credible that a polypeptide that does not comprise an L-chain (or only comprises a fragment of an L-chain) can treat pain. For similar reasons, it is credible that a polypeptide that does not comprise an L-chain (or only comprises a fragment of an L-chain) can treat an inflammatory condition. Thus, in one embodiment, the polypeptide may comprise a clostridial neurotoxin translocation domain (H_{N} domain) and/or a clostridial neurotoxin receptor binding domain (H_{C} domain). In one embodiment, a polypeptide of the invention does not comprise both a clostridial neurotoxin translocation domain (H_{N} domain) and a clostridial neurotoxin receptor binding domain (H_{C} domain).

In one embodiment, a polypeptide of the invention comprises (or consists of) a clostridial neurotoxin heavy chain (H-chain). Said H-chain comprises a clostridial neurotoxin translocation domain (H_{N} domain) and a receptor binding domain (H_{C} domain). Reference to a clostridial neurotoxin H-chain in this context also encompasses a fragment of a clostridial neurotoxin H-chain. A fragment of a clostridial neurotoxin H-chain may have ≤800, ≤700, ≤600, 5500, ≤400, ≤350, ≤300, ≤250, ≤200, ≤150, ≤100 or ≤50 amino acid residues of a clostridial neurotoxin H-chain. In one embodiment, a fragment of a clostridial neurotoxin H-chain has at least 20, 30, 40, 50, 60, 70, 80, 90, 100, 120, 150 or 200 amino acid residues of a clostridial neurotoxin H-chain. For example, a fragment of a clostridial neurotoxin H-chain may have 20-800, 30-600, 40-400, 50-300 or 100-200 amino acid residues of a clostridial neurotoxin H-chain. It is preferred, however, that reference to an H-chain is reference to a full-length H-chain.

In one embodiment, a polypeptide of the invention comprises (or consists of) a clostridial neurotoxin translocation domain (H_{N} domain). Reference to a clostridial neurotoxin translocation domain in this context also encompasses a fragment of a translocation domain. A fragment of a clostridial neurotoxin translocation domain may have ≤400, ≤350, ≤300, ≤250, ≤200, ≤150, ≤100 or ≤50 amino acid residues of a clostridial neurotoxin translocation domain. In one embodiment, a fragment of a clostridial neurotoxin translocation domain has at least 20, 30, 40, 50, 60, 70, 80, 90, 100, 120, 150 or 200 amino acid residues of a clostridial neurotoxin translocation domain. For example, a fragment of a clostridial neurotoxin translocation domain may have 20-400, 50-300 or 100-200 amino acid residues of a clostridial neurotoxin translocation domain. It is preferred, however, that reference to a translocation domain is reference to a full-length translocation domain.

The translocation domain is a fragment of the H-chain of a clostridial neurotoxin approximately equivalent to the amino-terminal half of the H-chain, or the domain corresponding to that fragment in the intact H-chain. In one embodiment the H_{C} function of the H-chain may be removed by deletion of the H_{C} amino acid sequence (either at the DNA synthesis level, or at the post-synthesis level by nuclease or protease treatment). Alternatively, the H_{C} function may be inactivated by chemical or biological treatment. Thus, in some embodiments the H-chain may be incapable of binding to the Binding Site on a target cell to which native clostridial neurotoxin (i.e. holotoxin) binds.

Examples of suitable (reference) Translocation Domains include:
Botulinum type A neurotoxin—amino acid residues (449-871)
Botulinum type B neurotoxin—amino acid residues (441-858)
Botulinum type C neurotoxin—amino acid residues (442-866)
Botulinum type D neurotoxin—amino acid residues (446-862)
Botulinum type E neurotoxin—amino acid residues (423-845)
Botulinum type F neurotoxin—amino acid residues (440-864)
Botulinum type G neurotoxin—amino acid residues (442-863)
Tetanus neurotoxin—amino acid residues (458-879)

The above-identified reference sequence should be considered a guide as slight variations may occur according to sub-serotypes. By way of example, US 2007/0166332 (hereby incorporated by reference thereto) cites slightly different clostridial sequences:
Botulinum type A neurotoxin—amino acid residues (A449-K871)
Botulinum type B neurotoxin—amino acid residues (A442-S858)
Botulinum type C neurotoxin—amino acid residues (T450-N866)
Botulinum type D neurotoxin—amino acid residues (D446-N862)
Botulinum type E neurotoxin—amino acid residues (K423-K845)
Botulinum type F neurotoxin—amino acid residues (A440-K864)
Botulinum type G neurotoxin—amino acid residues (S447-S863)
Tetanus neurotoxin—amino acid residues (S458-V879)

In the context of the present invention, a variety of clostridial neurotoxin H_{N} regions comprising a translocation domain can be useful in aspects of the present invention. The H_{N} regions from the heavy chains of clostridial neurotoxins are approximately 410-430 amino acids in length and comprise a translocation domain. Research has shown that the entire length of a H_{N} region from a clostridial neurotoxin heavy chain is not necessary for the translocating activity of the translocation domain. Thus, aspects of this embodiment can include clostridial neurotoxin H_{N} regions comprising a translocation domain having a length of, for example, at least 350 amino acids, at least 375 amino acids, at least 400 amino acids and at least 425 amino acids. Other aspects of this embodiment can include clostridial neurotoxin H_{N} regions comprising a translocation domain having a length of, for example, at most 350 amino acids, at most 375 amino acids, at most 400 amino acids and at most 425 amino acids.

For further details on the genetic basis of toxin production in Clostridium botulinum and C. tetani, see Henderson et al (1997) in The Clostridia: Molecular Biology and Pathogenesis, Academic press.

The term H_{N} embraces naturally-occurring neurotoxin H_{N} portions, and modified H_{N} portions having amino acid sequences that do not occur in nature and/or synthetic amino acid residues. In one embodiment said modified H_{N} portions still demonstrate the above-mentioned translocation function.

A clostridial neurotoxin translocation domain of the invention may comprise a polypeptide sequence having at least:
(i) 70% (e.g. at least 80%, 90%, 95%, 98%, or 100%) sequence identity to residues 449-871 of SEQ ID NO: 51;
(ii) 70% (e.g. at least 80%, 90%, 95%, 98%, or 100%) sequence identity to residues 441-858 of SEQ ID NO: 52;
(iii) 70% (e.g. at least 80%, 90%, 95%, 98%, or 100%) sequence identity to residues 442-866 of SEQ ID NO: 53;
(iv) 70% (e.g. at least 80%, 90%, 95%, 98%, or 100%) sequence identity to residues 446-862 of SEQ ID NO: 54;
(v) 70% (e.g. at least 80%, 90%, 95%, 98%, or 100%) sequence identity to residues 423-845 of SEQ ID NO: 55;
(vi) 70% (e.g. at least 80%, 90%, 95%, 98%, or 100%) sequence identity to residues 440-864 of SEQ ID NO: 56;
(vii) 70% (e.g. at least 80%, 90%, 95%, 98%, or 100%) sequence identity to residues 442-863 of SEQ ID NO: 57;
(viii) 70% (e.g. at least 80%, 90%, 95%, 98%, or 100%) sequence identity to residues 415-917, 440-892, or 465-867 of SEQ ID NO: 59; or
(ix) 70% (e.g. at least 80%, 90%, 95%, 98%, or 100%) sequence identity to residues 458-879 of SEQ ID NO: 58.

A clostridial neurotoxin translocation domain of the invention may consist essentially of a polypeptide sequence having at least:
(i) 70% (e.g. at least 80%, 90%, 95%, 98%, or 100%) sequence identity to residues 449-871 of SEQ ID NO: 51;
(ii) 70% (e.g. at least 80%, 90%, 95%, 98%, or 100%) sequence identity to residues 441-858 of SEQ ID NO: 52;
(iii) 70% (e.g. at least 80%, 90%, 95%, 98%, or 100%) sequence identity to residues 442-866 of SEQ ID NO: 53;
(iv) 70% (e.g. at least 80%, 90%, 95%, 98%, or 100%) sequence identity to residues 446-862 of SEQ ID NO: 54;
(v) 70% (e.g. at least 80%, 90%, 95%, 98%, or 100%) sequence identity to residues 423-845 of SEQ ID NO: 55;
(vi) 70% (e.g. at least 80%, 90%, 95%, 98%, or 100%) sequence identity to residues 440-864 of SEQ ID NO: 56;
(vii) 70% (e.g. at least 80%, 90%, 95%, 98%, or 100%) sequence identity to residues 442-863 of SEQ ID NO: 57;
(viii) 70% (e.g. at least 80%, 90%, 95%, 98%, or 100%) sequence identity to residues 415-917, 440-892, or 465-867 of SEQ ID NO: 59; or
(ix) 70% (e.g. at least 80%, 90%, 95%, 98%, or 100%) sequence identity to residues 458-879 of SEQ ID NO: 58.

A clostridial neurotoxin translocation domain of the invention may consist of a polypeptide sequence having at least:
(i) 70% (e.g. at least 80%, 90%, 95%, 98%, or 100%) sequence identity to residues 449-871 of SEQ ID NO: 51;
(ii) 70% (e.g. at least 80%, 90%, 95%, 98%, or 100%) sequence identity to residues 441-858 of SEQ ID NO: 52;
(iii) 70% (e.g. at least 80%, 90%, 95%, 98%, or 100%) sequence identity to residues 442-866 of SEQ ID NO: 53;
(iv) 70% (e.g. at least 80%, 90%, 95%, 98%, or 100%) sequence identity to residues 446-862 of SEQ ID NO: 54;
(v) 70% (e.g. at least 80%, 90%, 95%, 98%, or 100%) sequence identity to residues 423-845 of SEQ ID NO: 55;
(vi) 70% (e.g. at least 80%, 90%, 95%, 98%, or 100%) sequence identity to residues 440-864 of SEQ ID NO: 56;
(vii) 70% (e.g. at least 80%, 90%, 95%, 98%, or 100%) sequence identity to residues 442-863 of SEQ ID NO: 57;
(viii) 70% (e.g. at least 80%, 90%, 95%, 98%, or 100%) sequence identity to residues 415-917, 440-892, or 465-867 of SEQ ID NO: 59; or
(ix) 70% (e.g. at least 80%, 90%, 95%, 98%, or 100%) sequence identity to residues 458-879 of SEQ ID NO: 58.

In one embodiment, a polypeptide of the invention comprises (or consists of) a clostridial neurotoxin receptor binding domain (H_{C} domain). Reference to a clostridial neurotoxin receptor binding domain (H_{C}) in this context also encompasses a fragment of a clostridial neurotoxin receptor binding domain (H_{C}). A fragment of a clostridial neurotoxin receptor binding domain (H_{C}) may have ≤350, ≤300, ≤250, ≤200, ≤150, ≤100 or ≤50 amino acid residues of a clostridial neurotoxin receptor binding domain (H_{C}). In one embodiment, a fragment of a clostridial neurotoxin receptor binding domain (H_{C}) has at least 20, 30, 40, 50, 60, 70, 80, 90, 100, 120, 150 or 200 amino acid residues of a clostridial neurotoxin receptor binding domain (H_{C}). For example, a fragment of a clostridial neurotoxin receptor binding domain (H_{C}) may have 20-350, 50-300 or 100-200 amino acid residues of a clostridial neurotoxin receptor binding domain (H_{C}). It is preferred, however, that reference to a clostridial neurotoxin receptor binding domain (H_{C}) is reference to a full-length clostridial neurotoxin receptor binding domain (H_{C}).

Examples of clostridial neurotoxin receptor binding domain (H_{C}) reference sequences include:
BoNT/A—N872-L1296
BoNT/B—E859-E1291
BoNT/C1—N867-E1291
BoNT/D—S863-E1276
BoNT/E—R846-K1252
BoNT/F—K865-E1274
BoNT/G—N864-E1297
TeNT—1880-D1315

For recently-identified BoNT/X, the H_{C} domain has been reported as corresponding to amino acids 893-1306 thereof, with the domain boundary potentially varying by approximately 25 amino acids (e.g. 868-1306 or 918-1306).

A clostridial neurotoxin H-chain (e.g. the H_{C} domain portion) may further comprise a translocation facilitating domain (or a fragment thereof may be translocation facilitating domain fragment). Said domain facilitates delivery of the L-chain into the cytosol of the target cell and are described, for example, in WO 08/008803 and WO 08/008805, each of which is herein incorporated by reference thereto.

By way of example, a translocation facilitating domain may comprise a clostridial neurotoxin H_{CN} domain or a fragment or variant thereof. In more detail, a clostridial neurotoxin H_{CN} translocation facilitating domain may have a length of at least 200 amino acids, at least 225 amino acids, at least 250 amino acids, at least 275 amino acids. In this regard, a clostridial neurotoxin H_{CN} translocation facilitating domain preferably has a length of at most 200 amino acids, at most 225 amino acids, at most 250 amino acids, or at most 275 amino acids. Specific (reference) examples include:
Botulinum type A neurotoxin—amino acid residues (872-1110)
Botulinum type B neurotoxin—amino acid residues (859-1097)
Botulinum type C neurotoxin—amino acid residues (867-1111)
Botulinum type D neurotoxin—amino acid residues (863-1098)
Botulinum type E neurotoxin—amino acid residues (846-1085)
Botulinum type F neurotoxin—amino acid residues (865-1105)
Botulinum type G neurotoxin—amino acid residues (864-1105)
Tetanus neurotoxin—amino acid residues (880-1127)

The above sequence positions may vary a little according to serotype/sub-type, and further examples of suitable (reference) clostridial neurotoxin H_{CN} domains include:
Botulinum type A neurotoxin—amino acid residues (874-1110)
Botulinum type B neurotoxin—amino acid residues (861-1097)
Botulinum type C neurotoxin—amino acid residues (869-1111)
Botulinum type D neurotoxin—amino acid residues (865-1098)
Botulinum type E neurotoxin—amino acid residues (848-1085)
Botulinum type F neurotoxin—amino acid residues (867-1105)
Botulinum type G neurotoxin—amino acid residues (866-1105)
Tetanus neurotoxin—amino acid residues (882-1127)

Suitable clostridial neurotoxin H_{C} domains are described herein.

A clostridial neurotoxin H_{C} domain of the invention may comprise a polypeptide sequence having at least:
(i) 70% (e.g. at least 80%, 90%, 95%, 98%, or 100%) sequence identity to residues 872-1296 of SEQ ID NO: 51;
(ii) 70% (e.g. at least 80%, 90%, 95%, 98%, or 100%) sequence identity to residues 859-1291 of SEQ ID NO: 52;
(iii) 70% (e.g. at least 80%, 90%, 95%, 98%, or 100%) sequence identity to residues 867-1291 of SEQ ID NO: 53;
(iv) 70% (e.g. at least 80%, 90%, 95%, 98%, or 100%) sequence identity to residues 863-1276 of SEQ ID NO: 54;
(v) 70% (e.g. at least 80%, 90%, 95%, 98%, or 100%) sequence identity to residues 846-1251 of SEQ ID NO: 55;
(vi) 70% (e.g. at least 80%, 90%, 95%, 98%, or 100%) sequence identity to residues 865-1277 of SEQ ID NO: 56;
(vii) 70% (e.g. at least 80%, 90%, 95%, 98%, or 100%) sequence identity to residues 864-1297 of SEQ ID NO: 57;
(viii) 70% (e.g. at least 80%, 90%, 95%, 98%, or 100%) sequence identity to residues 868-1306, 893-1306, or 918-1306 of SEQ ID NO: 59; or
(ix) 70% (e.g. at least 80%, 90%, 95%, 98%, or 100%) sequence identity to residues 880-1315 of SEQ ID NO: 58.

A clostridial neurotoxin H_{C} domain of the invention may consist essentially of a polypeptide sequence having at least:
(i) 70% (e.g. at least 80%, 90%, 95%, 98%, or 100%) sequence identity to residues 872-1296 of SEQ ID NO: 51;
(ii) 70% (e.g. at least 80%, 90%, 95%, 98%, or 100%) sequence identity to residues 859-1291 of SEQ ID NO: 52;
(iii) 70% (e.g. at least 80%, 90%, 95%, 98%, or 100%) sequence identity to residues 867-1291 of SEQ ID NO: 53;
(iv) 70% (e.g. at least 80%, 90%, 95%, 98%, or 100%) sequence identity to residues 863-1276 of SEQ ID NO: 54;
(v) 70% (e.g. at least 80%, 90%, 95%, 98%, or 100%) sequence identity to residues 846-1251 of SEQ ID NO: 55;
(vi) 70% (e.g. at least 80%, 90%, 95%, 98%, or 100%) sequence identity to residues 865-1277 of SEQ ID NO: 56;
(vii) 70% (e.g. at least 80%, 90%, 95%, 98%, or 100%) sequence identity to residues 864-1297 of SEQ ID NO: 57;
(viii) 70% (e.g. at least 80%, 90%, 95%, 98%, or 100%) sequence identity to residues 868-1306, 893-1306, or 918-1306 of SEQ ID NO: 59; or
(ix) 70% (e.g. at least 80%, 90%, 95%, 98%, or 100%) sequence identity to residues 880-1315 of SEQ ID NO: 58.

A clostridial neurotoxin H_{C} domain of the invention may consist of a polypeptide sequence having at least:
(i) 70% (e.g. at least 80%, 90%, 95%, 98%, or 100%) sequence identity to residues 872-1296 of SEQ ID NO: 51;
(ii) 70% (e.g. at least 80%, 90%, 95%, 98%, or 100%) sequence identity to residues 859-1291 of SEQ ID NO: 52;
(iii) 70% (e.g. at least 80%, 90%, 95%, 98%, or 100%) sequence identity to residues 867-1291 of SEQ ID NO: 53;
(iv) 70% (e.g. at least 80%, 90%, 95%, 98%, or 100%) sequence identity to residues 863-1276 of SEQ ID NO: 54;
(v) 70% (e.g. at least 80%, 90%, 95%, 98%, or 100%) sequence identity to residues 846-1251 of SEQ ID NO: 55;
(vi) 70% (e.g. at least 80%, 90%, 95%, 98%, or 100%) sequence identity to residues 865-1277 of SEQ ID NO: 56;
(vii) 70% (e.g. at least 80%, 90%, 95%, 98%, or 100%) sequence identity to residues 864-1297 of SEQ ID NO: 57;
(viii) 70% (e.g. at least 80%, 90%, 95%, 98%, or 100%) sequence identity to residues 868-1306, 893-1306, or 918-1306 of SEQ ID NO: 59; or
(ix) 70% (e.g. at least 80%, 90%, 95%, 98%, or 100%) sequence identity to residues 880-1315 of SEQ ID NO: 58.

A clostridial neurotoxin H_{C} domain may comprise a polypeptide sequence having at least 70% sequence identity to any one of SEQ ID NOs: 8, 22, 30, 38, 42, 44, 46, 48 or 50. In one embodiment a clostridial neurotoxin H_{C} domain comprises a polypeptide sequence having at least 80%, 90%, 95% or 98% sequence identity to any one of SEQ ID NOs: 8, 22, 30, 38, 42, 44, 46, 48 or 50. Preferably, a clostridial neurotoxin H_{C} domain comprises (more preferably consists of) a polypeptide sequence comprising any one of SEQ ID NOs: 8, 22, 30, 38, 42, 44, 46, 48 or 50.

A clostridial neurotoxin H_{C} domain may be one encoded by a nucleotide sequence having at least 70% sequence identity to any one of SEQ ID NOs: 7, 21, 29, 37, 41, 43, 45, 47 or 49. In one embodiment a clostridial neurotoxin H_{C} domain is one encoded by a nucleotide sequence having at least 80%, 90%, 95% or 98% sequence identity to any one of SEQ ID NOs: 7, 21, 29, 37, 41, 43, 45, 47 or 49. Preferably, a clostridial neurotoxin H_{C} domain is one encoded by a nucleotide sequence comprising any one of SEQ ID NOs: 7, 21, 29, 37, 41, 43, 45, 47 or 49.

Any of the above-described facilitating domains may be combined with any of the previously described translocation domain peptides that are suitable for use in the present invention. Thus, by way of example, a non-clostridial facilitating domain may be combined with a non-clostridial translocation domain peptide or with clostridial translocation domain peptide. Alternatively, a clostridial neurotoxin H_{CN} translocation facilitating domain may be combined with a non-clostridial translocation domain peptide. Alternatively, a clostridial neurotoxin H_{CN} facilitating domain may be combined with a clostridial translocation domain peptide, examples of which include:
Botulinum type A neurotoxin—amino acid residues (449-1110)
Botulinum type B neurotoxin—amino acid residues (442-1097)
Botulinum type C neurotoxin—amino acid residues (450-1111)
Botulinum type D neurotoxin—amino acid residues (446-1098)
Botulinum type E neurotoxin—amino acid residues (423-1085)
Botulinum type F neurotoxin—amino acid residues (440-1105)
Botulinum type G neurotoxin—amino acid residues (447-1105)
Tetanus neurotoxin—amino acid residues (458-1127)

In some embodiments the polypeptides of the present invention may lack a functional H_{C} domain of a clostridial neurotoxin. In one embodiment, the polypeptides preferably lack the last 50 C-terminal amino acids of a clostridial neurotoxin holotoxin. In another embodiment, the polypeptides preferably lack the last 100, preferably the last 150, more preferably the last 200, particularly preferably the last 250, and most preferably the last 300 C-terminal amino acid residues of a clostridial neurotoxin holotoxin. Alternatively, the H_{C} binding activity may be negated/reduced by mutagenesis—by way of example, referring to BoNT/A for convenience, modification of one or two amino acid residue mutations (W1266 to L and Y1267 to F) in the ganglioside binding pocket causes the H_{C} region to lose its receptor binding function.

Analogous mutations may be made to non-serotype A clostridial peptide components, e.g. a construct based on botulinum B with mutations (W1262 to L and Y1263 to F) or botulinum E (W1224 to L and Y1225 to F). Other mutations to the active site achieve the same ablation of H_{C} receptor binding activity, e.g. Y1267S in botulinum type A toxin and the corresponding highly conserved residue in the other clostridial neurotoxins. Details of this and other mutations are described in Rummel et al (2004) (Molecular Microbiol. 51:631-634), which is hereby incorporated by reference thereto.

The H_{C} peptide of a native clostridial neurotoxin comprises approximately 400-440 amino acid residues, and consists of two functionally distinct domains of approximately 25 kDa each, namely the N-terminal region (commonly referred to as the H_{CN} peptide or domain) and the C-terminal region (commonly referred to as the H_{CC} peptide or domain). This fact is confirmed by the following publications, each of which is herein incorporated in its entirety by reference thereto: Umland T C (1997) Nat. Struct. Biol. 4: 788-792; Herreros J (2000) Biochem. J. 347: 199-204; Halpern J (1993) J. Biol. Chem. 268: 15, pp. 11188-11192; Rummel A (2007) PNAS 104: 359-364; Lacey D B (1998) Nat. Struct. Biol. 5: 898-902; Knapp (1998) Am. Cryst. Assoc. Abstract Papers 25: 90; Swaminathan and Eswaramoorthy (2000) Nat. Struct. Biol. 7: 1751-1759; and Rummel A (2004) Mol. Microbiol. 51(3), 631-643. Moreover, it has been well documented that the C-terminal region (H_{CC}), which constitutes the C-terminal 160-200 amino acid residues, is responsible for binding of a clostridial neurotoxin to its natural cell receptors, namely to nerve terminals at the neuromuscular junction—this fact is also confirmed by the above publications. Thus, reference throughout this specification to a clostridial heavy-chain lacking a functional heavy chain H_{C} peptide (or domain) such that the heavy-chain is incapable of binding to cell surface receptors to which a native clostridial neurotoxin binds means that the clostridial heavy-chain simply lacks a functional H_{CC} peptide. In other words, the Hoc peptide region may be either partially or wholly deleted, or otherwise modified (e.g. through conventional chemical or proteolytic treatment) to reduce its native binding ability for nerve terminals at the neuromuscular junction.

Thus, in one embodiment, a clostridial neurotoxin H_{N} peptide of the present invention lacks part of a C-terminal peptide portion (H_{CC}) of a clostridial neurotoxin and thus lacks the H_{C} binding function of native clostridial neurotoxin. By way of example, in one embodiment, the C-terminally extended clostridial H_{N} peptide lacks the C-terminal 40 amino acid residues, or the C-terminal 60 amino acid residues, or the C-terminal 80 amino acid residues, or the C-terminal 100 amino acid residues, or the C-terminal 120 amino acid residues, or the C-terminal 140 amino acid residues, or the C-terminal 150 amino acid residues, or the C-terminal 160 amino acid residues of a clostridial neurotoxin heavy-chain. In another embodiment, the clostridial H_{N} peptide of the present invention lacks the entire C-terminal peptide portion (H_{CC}) of a clostridial neurotoxin and thus lacks the H_{C} binding function of native clostridial neurotoxin. By way of example, in one embodiment, the clostridial H_{N} peptide lacks the C-terminal 165 amino acid residues, or the C-terminal 170 amino acid residues, or the C-terminal 175 amino acid residues, or the C-terminal 180 amino acid residues, or the C-terminal 185 amino acid residues, or the C-terminal 190 amino acid residues, or the C-terminal 195 amino acid residues of a clostridial neurotoxin heavy-chain. By way of further example, the clostridial H_{N} peptide of the present invention lacks a clostridial H_{CC} reference sequence selected from the group consisting of:
Botulinum type A neurotoxin—amino acid residues (Y1111-L1296)
Botulinum type B neurotoxin—amino acid residues (Y1098-E1291)
Botulinum type C neurotoxin—amino acid residues (Y1112-E1291)
Botulinum type D neurotoxin—amino acid residues (Y1099-E1276)
Botulinum type E neurotoxin—amino acid residues (Y1086-K1252)
Botulinum type F neurotoxin—amino acid residues (Y1106-E1274)
Botulinum type G neurotoxin—amino acid residues (Y1106-E1297)
Tetanus neurotoxin—amino acid residues (Y1128-D1315).

The above-identified reference sequences should be considered a guide as slight variations may occur according to sub-serotypes.

In other embodiments a fragment of an H_{C} domain may comprise an H_{CC} peptide as described herein.

A polypeptide of the invention may comprise a catalytically inactive clostridial neurotoxin L-chain and a clostridial neurotoxin translocation domain (H_{N} domain) and/or a clostridial neurotoxin receptor binding domain (H_{C} domain). For example, a polypeptide may comprise a catalytically inactive clostridial neurotoxin L-chain and a clostridial neurotoxin translocation domain (H_{N}).

Suitable polypeptides comprising a catalytically inactive clostridial neurotoxin L-chain and translocation domain are described herein. Said polypeptide may be referred to as LH_{N}. Such a polypeptide may comprise:
(i) a BoNT/A L-chain and a BoNT/A H_{N} domain with the proviso that the L-chain is catalytically inactive (e.g. the L-chain has been inactivated by modification);
(ii) a BoNT/B L-chain and a BoNT/B H_{N} domain with the proviso that the L-chain is catalytically inactive (e.g. the L-chain has been inactivated by modification);
(iii) a BoNT/C1 L-chain and a BoNT/C1 H_{N} domain with the proviso that the L-chain is catalytically inactive (e.g. the L-chain has been inactivated by modification);
(iv) a BoNT/D L-chain and a BoNT/D H_{N} domain with the proviso that the L-chain is catalytically inactive (e.g. the L-chain has been inactivated by modification);
(v) a BoNT/E L-chain and a BoNT/E H_{N} domain with the proviso that the L-chain is catalytically inactive (e.g. the L-chain has been inactivated by modification);
(vi) a BoNT/F L-chain and a BoNT/F H_{N} domain with the proviso that the L-chain is catalytically inactive (e.g. the L-chain has been inactivated by modification);
(vii) a BoNT/G L-chain and a BoNT/G H_{N} domain with the proviso that the L-chain is catalytically inactive (e.g. the L-chain has been inactivated by modification);
(viii) a BoNT/X L-chain and a BoNT/X H_{N} domain with the proviso that the L-chain is catalytically inactive (e.g. the L-chain has been inactivated by modification); or
(ix) a TeNT L-chain and a TeNT H_{N} domain with the proviso that the L-chain is catalytically inactive (e.g. the L-chain has been inactivated by modification).

Said polypeptide may consist essentially of, preferably consist of, any one of the above.

In one embodiment a polypeptide comprises a polypeptide sequence having at least:
(i) 70% (e.g. at least 80%, 90%, 95%, or 98%) sequence identity to residues 1-871 of SEQ ID NO: 51 with the proviso that the L-chain is catalytically inactive (e.g. the L-chain has been inactivated by modification);
(ii) 70% (e.g. at least 80%, 90%, 95%, or 98%) sequence identity to residues 1-858 of SEQ ID NO: 52 with the proviso that the L-chain is catalytically inactive (e.g. the L-chain has been inactivated by modification);
(iii) 70% (e.g. at least 80%, 90%, 95%, or 98%) sequence identity to residues 1-866 of SEQ ID NO: 53 with the proviso that the L-chain is catalytically inactive (e.g. the L-chain has been inactivated by modification);
(iv) 70% (e.g. at least 80%, 90%, 95%, or 98%) sequence identity to residues 1-862 of SEQ ID NO: 54 with the proviso that the L-chain is catalytically inactive (e.g. the L-chain has been inactivated by modification);
(v) 70% (e.g. at least 80%, 90%, 95%, or 98%) sequence identity to residues 1-845 of SEQ ID NO: 55 with the proviso that the L-chain is catalytically inactive (e.g. the L-chain has been inactivated by modification);
(vi) 70% (e.g. at least 80%, 90%, 95%, or 98%) sequence identity to residues 1-864 of SEQ ID NO: 56 with the proviso that the L-chain is catalytically inactive (e.g. the L-chain has been inactivated by modification);
(vii) 70% (e.g. at least 80%, 90%, 95%, or 98%) sequence identity to residues 1-863 of SEQ ID NO: 57 with the proviso that the L-chain is catalytically inactive (e.g. the L-chain has been inactivated by modification);
(viii) 70% (e.g. at least 80%, 90%, 95%, or 98%) sequence identity to residues 1-867, 1-892, or 1-917 of SEQ ID NO: 59 with the proviso that the L-chain is catalytically inactive (e.g. the L-chain has been inactivated by modification); or
(ix) 70% (e.g. at least 80%, 90%, 95%, or 98%) sequence identity to residues 1-879 of SEQ ID NO: 58 with the proviso that the L-chain is catalytically inactive (e.g. the L-chain has been inactivated by modification).

The polypeptide may comprise a polypeptide sequence having:
(i) residues 1-871 of SEQ ID NO: 51 that has been further modified to catalytically inactivate the L-chain;
(ii) residues 1-858 of SEQ ID NO: 52 that has been further modified to catalytically inactivate the L-chain;
(iii) residues 1-866 of SEQ ID NO: 53 that has been further modified to catalytically inactivate the L-chain;
(iv) residues 1-862 of SEQ ID NO: 54 that has been further modified to catalytically inactivate the L-chain;
(v) residues 1-845 of SEQ ID NO: 55 that has been further modified to catalytically inactivate the L-chain;
(vi) residues 1-864 of SEQ ID NO: 56 that has been further modified to catalytically inactivate the L-chain;
(vii) residues 1-863 of SEQ ID NO: 57 that has been further modified to catalytically inactivate the L-chain;
(viii) residues 1-867, 1-892, or 1-917 of SEQ ID NO: 59 that has been further modified to catalytically inactivate the L-chain; or
(ix) residues 1-879 of SEQ ID NO: 58 that has been further modified to catalytically inactivate the L-chain.

A polypeptide comprising a clostridial neurotoxin L-chain and translocation domain may comprise a polypeptide sequence having at least 70% sequence identity to any one of SEQ ID NOs: 4, 20, 28, 36 or 75 with the proviso that the L-chain is catalytically inactive (e.g. the L-chain has been inactivated by modification). In one a polypeptide comprising a clostridial neurotoxin L-chain and translocation domain comprises a polypeptide sequence having at least 80%, 90%, 95% or 98% sequence identity to any one of SEQ ID NOs: 4, 20, 28, 36 or 75 with the proviso that the L-chain is catalytically inactive (e.g. the L-chain has been inactivated by modification). Preferably, a polypeptide comprising a clostridial neurotoxin L-chain and translocation domain comprises (more preferably consists of) a polypeptide sequence comprising any one of SEQ ID NOs: 4, 20, 28, 36 or 75 that has been modified to catalytically inactivate the L-chain, such as SEQ ID NO: 75.

A polypeptide comprising (or consisting of) a clostridial neurotoxin L-chain and translocation domain may be one encoded by a nucleotide sequence having at least 70% sequence identity to any one of SEQ ID NOs: 3, 19, 27 or 35 with the proviso that the L-chain is catalytically inactive (e.g. the L-chain has been inactivated by modification). In one embodiment a polypeptide comprising (or consisting of) a clostridial neurotoxin L-chain and translocation domain is one encoded by a nucleotide sequence having at least 80%, 90%, 95% or 98% sequence identity to any one of SEQ ID NOs: 3, 19, 27 or 35 with the proviso that the L-chain is catalytically inactive (e.g. the L-chain has been inactivated by modification). Preferably, a polypeptide comprising (or consisting of) a clostridial neurotoxin L-chain and translocation domain is one encoded by a nucleotide sequence comprising any one of SEQ ID NOs: 3, 19, 27 or 35 that has been modified to catalytically inactivate the encoded L-chain.

Preferably, the polypeptide comprises a catalytically inactive clostridial neurotoxin L-chain, a clostridial neurotoxin translocation domain (H_{N} domain), and a clostridial neurotoxin receptor binding domain (H_{C} domain).

In one embodiment, a polypeptide of the invention does not comprise a clostridial neurotoxin receptor binding domain (H_{C}) or at least the C-terminal portion of a clostridial neurotoxin receptor binding domain (H_{CC}). Thus, in one embodiment a polypeptide of the present invention lacks a C-terminal portion of a clostridial neurotoxin receptor binding domain (H_{CC}). Advantageously, such polypeptides lack the endogenous clostridial neurotoxin receptor binding capabilities and may thus exhibit fewer off-target effects in a subject administered said polypeptide.

A polypeptide of the invention may consist essentially of a catalytically inactive clostridial neurotoxin light-chain (L-chain), a clostridial neurotoxin translocation domain (H_{N} domain) and/or a clostridial neurotoxin receptor binding domain (H_{C} domain). For example, a polypeptide may consist essentially of a catalytically inactive clostridial neurotoxin L-chain and a clostridial neurotoxin translocation domain (H_{N}).

In one embodiment a polypeptide consists essentially of a polypeptide sequence having at least:
(i) 70% (e.g. at least 80%, 90%, 95%, or 98%) sequence identity to residues 1-871 of SEQ ID NO: 51 with the proviso that the L-chain is catalytically inactive (e.g. the L-chain has been inactivated by modification);
(ii) 70% (e.g. at least 80%, 90%, 95%, or 98%) sequence identity to residues 1-858 of SEQ ID NO: 52 with the proviso that the L-chain is catalytically inactive (e.g. the L-chain has been inactivated by modification);
(iii) 70% (e.g. at least 80%, 90%, 95%, or 98%) sequence identity to residues 1-866 of SEQ ID NO: 53 with the proviso that the L-chain is catalytically inactive (e.g. the L-chain has been inactivated by modification);
(iv) 70% (e.g. at least 80%, 90%, 95%, or 98%) sequence identity to residues 1-862 of SEQ ID NO: 54 with the proviso that the L-chain is catalytically inactive (e.g. the L-chain has been inactivated by modification);
(v) 70% (e.g. at least 80%, 90%, 95%, or 98%) sequence identity to residues 1-845 of SEQ ID NO: 55 with the proviso that the L-chain is catalytically inactive (e.g. the L-chain has been inactivated by modification);
(vi) 70% (e.g. at least 80%, 90%, 95%, or 98%) sequence identity to residues 1-864 of SEQ ID NO: 56 with the proviso that the L-chain is catalytically inactive (e.g. the L-chain has been inactivated by modification);
(vii) 70% (e.g. at least 80%, 90%, 95%, or 98%) sequence identity to residues 1-863 of SEQ ID NO: 57 with the proviso that the L-chain is catalytically inactive (e.g. the L-chain has been inactivated by modification);
(viii) 70% (e.g. at least 80%, 90%, 95%, or 98%) sequence identity to residues 1-867, 1-892, or 1-917 of SEQ ID NO: 59 with the proviso that the L-chain is catalytically inactive (e.g. the L-chain has been inactivated by modification); or
(ix) 70% (e.g. at least 80%, 90%, 95%, or 98%) sequence identity to residues 1-879 of SEQ ID NO: 58 with the proviso that the L-chain is catalytically inactive (e.g. the L-chain has been inactivated by modification).

The polypeptide may consist essentially of a polypeptide sequence having:
(i) residues 1-871 of SEQ ID NO: 51 that has been further modified to catalytically inactivate the L-chain;
(ii) residues 1-858 of SEQ ID NO: 52 that has been further modified to catalytically inactivate the L-chain;
(iii) residues 1-866 of SEQ ID NO: 53 that has been further modified to catalytically inactivate the L-chain;
(iv) residues 1-862 of SEQ ID NO: 54 that has been further modified to catalytically inactivate the L-chain;
(v) residues 1-845 of SEQ ID NO: 55 that has been further modified to catalytically inactivate the L-chain;
(vi) residues 1-864 of SEQ ID NO: 56 that has been further modified to catalytically inactivate the L-chain;
(vii) residues 1-863 of SEQ ID NO: 57 that has been further modified to catalytically inactivate the L-chain;
(viii) residues 1-867, 1-892, or 1-917 of SEQ ID NO: 59 that has been further modified to catalytically inactivate the L-chain; or
(ix) residues 1-879 of SEQ ID NO: 58 that has been further modified to catalytically inactivate the L-chain.

Preferably, the polypeptide consists essentially of a catalytically inactive clostridial neurotoxin L-chain, a clostridial neurotoxin translocation domain (H_{N} domain), and a clostridial neurotoxin receptor binding domain (H_{C} domain).

The term “consists essentially of” as used in this context means that the polypeptide does not further comprise one or more amino acid residues that confer additional functionality to the polypeptide, e.g. when administered to a subject. In other words, a polypeptide that “consists essentially of” a catalytically inactive clostridial neurotoxin light-chain (L-chain), a clostridial neurotoxin translocation domain (H_{N} domain) and/or a clostridial neurotoxin receptor binding domain (H_{C} domain) may further comprise one or more amino acid residues (to those of the catalytically inactive clostridial neurotoxin light-chain (L-chain), clostridial neurotoxin translocation domain (H_{N} domain) and/or clostridial neurotoxin receptor binding domain (H_{C} domain)) but said one or more further amino acid residues do not confer additional functionality to the polypeptide, e.g. when administered to a subject. Additional functionality may include enzymatic activity, binding activity and/or any physiological activity whatsoever.

A polypeptide of the invention may consist of a catalytically inactive clostridial neurotoxin light-chain (L-chain), a clostridial neurotoxin translocation domain (H_{N} domain) and/or a clostridial neurotoxin receptor binding domain (H_{C} domain). For example, a polypeptide may consist of a catalytically inactive clostridial neurotoxin L-chain and a clostridial neurotoxin translocation domain (H_{N}).

In one embodiment a polypeptide consists of a polypeptide sequence having at least:
(i) 70% (e.g. at least 80%, 90%, 95%, or 98%) sequence identity to residues 1-871 of SEQ ID NO: 51 with the proviso that the L-chain is catalytically inactive (e.g. the L-chain has been inactivated by modification);
(ii) 70% (e.g. at least 80%, 90%, 95%, or 98%) sequence identity to residues 1-858 of SEQ ID NO: 52 with the proviso that the L-chain is catalytically inactive (e.g. the L-chain has been inactivated by modification);
(iii) 70% (e.g. at least 80%, 90%, 95%, or 98%) sequence identity to residues 1-866 of SEQ ID NO: 53 with the proviso that the L-chain is catalytically inactive (e.g. the L-chain has been inactivated by modification);
(iv) 70% (e.g. at least 80%, 90%, 95%, or 98%) sequence identity to residues 1-862 of SEQ ID NO: 54 with the proviso that the L-chain is catalytically inactive (e.g. the L-chain has been inactivated by modification);
(v) 70% (e.g. at least 80%, 90%, 95%, or 98%) sequence identity to residues 1-845 of SEQ ID NO: 55 with the proviso that the L-chain is catalytically inactive (e.g. the L-chain has been inactivated by modification);
(vi) 70% (e.g. at least 80%, 90%, 95%, or 98%) sequence identity to residues 1-864 of SEQ ID NO: 56 with the proviso that the L-chain is catalytically inactive (e.g. the L-chain has been inactivated by modification);
(vii) 70% (e.g. at least 80%, 90%, 95%, or 98%) sequence identity to residues 1-863 of SEQ ID NO: 57 with the proviso that the L-chain is catalytically inactive (e.g. the L-chain has been inactivated by modification);
(viii) 70% (e.g. at least 80%, 90%, 95%, or 98%) sequence identity to residues 1-867, 1-892, or 1-917 of SEQ ID NO: 59 with the proviso that the L-chain is catalytically inactive (e.g. the L-chain has been inactivated by modification); or
(ix) 70% (e.g. at least 80%, 90%, 95%, or 98%) sequence identity to residues 1-879 of SEQ ID NO: 58 with the proviso that the L-chain is catalytically inactive (e.g. the L-chain has been inactivated by modification).

The polypeptide may consist of a polypeptide sequence having:
(i) residues 1-871 of SEQ ID NO: 51 that has been further modified to catalytically inactivate the L-chain;
(ii) residues 1-858 of SEQ ID NO: 52 that has been further modified to catalytically inactivate the L-chain;
(iii) residues 1-866 of SEQ ID NO: 53 that has been further modified to catalytically inactivate the L-chain;
(iv) residues 1-862 of SEQ ID NO: 54 that has been further modified to catalytically inactivate the L-chain;
(v) residues 1-845 of SEQ ID NO: 55 that has been further modified to catalytically inactivate the L-chain;
(vi) residues 1-864 of SEQ ID NO: 56 that has been further modified to catalytically inactivate the L-chain;
(vii) residues 1-863 of SEQ ID NO: 57 that has been further modified to catalytically inactivate the L-chain;
(viii) residues 1-867, 1-892, or 1-917 of SEQ ID NO: 59 that has been further modified to catalytically inactivate the L-chain; or
(ix) residues 1-879 of SEQ ID NO: 58 that has been further modified to catalytically inactivate the L-chain.

Preferably, the polypeptide consists of a catalytically inactive clostridial neurotoxin L-chain, a clostridial neurotoxin translocation domain (H_{N} domain), and a clostridial neurotoxin receptor binding domain (H_{C} domain).

In one embodiment a polypeptide may comprise non-clostridial neurotoxin sequences in addition to any clostridial neurotoxin sequences so long as the non-clostridial neurotoxin sequences do not disrupt the ability of a polypeptide to achieve its therapeutic effect (e.g. to treat pain). Preferably, the non-clostridial neurotoxin sequence is not one having catalytic activity, e.g. enzymatic activity. In one embodiment the polypeptide of the invention does not comprise a catalytically active domain (e.g. a non-clostridial catalytically active domain). In one embodiment, the non-clostridial sequence is not one that binds to a cellular receptor. In other words, in one embodiment, the non-clostridial sequence is not a ligand for a cellular receptor. A cellular receptor may be a proteinaceous cellular receptor, such as an integral membrane protein. Examples of cellular receptors can be found in the IUPHAR Guide to Pharmacology Database, version 2019.4, available at https://www.guidetopharmacology.org/download.jsp#db_reports. Non-clostridial neurotoxin sequences may include tags to aid in purification, such as His-tags. In one embodiment, a polypeptide of the invention does not comprise a label or a site for adding a label, such as a sortase acceptor or donor site.

In a preferred embodiment, a polypeptide of the invention does not comprise a therapeutic or diagnostic agent (e.g. a nucleic acid, protein, peptide or small molecule therapeutic or diagnostic agent) additional to the catalytically inactive clostridial neurotoxin L-chain, H_{N} domain and/or H_{C} domain. For example, in one embodiment, the polypeptide may not comprise a covalently or non-covalently associated therapeutic or diagnostic agent. Thus, a polypeptide of the invention preferably does not function as a delivery vehicle for a further therapeutic or diagnostic agent.

The polypeptide of the invention may comprise (or consist of) a modified clostridial neurotoxin or derivative thereof or modified clostridial neurotoxin fragment or derivative fragment, including but not limited to those described below with the proviso that any L-chain present is catalytically inactive. A modified clostridial neurotoxin or derivative (or modified clostridial neurotoxin fragment or derivative fragment) may contain one or more amino acids that has been modified as compared to the native (unmodified) form of the clostridial neurotoxin (or clostridial neurotoxin fragment), or may contain one or more inserted amino acids that are not present in the native (unmodified) form of the clostridial neurotoxin (or clostridial neurotoxin fragment). By way of example, a modified clostridial neurotoxin (or clostridial neurotoxin fragment) may have modified amino acid sequences in one or more domains relative to the native (unmodified) clostridial neurotoxin sequence (or clostridial neurotoxin fragment). Such modifications may modify functional aspects of the toxin (or toxin fragment). Thus, in one embodiment, the polypeptide of the invention is or comprises a modified clostridial neurotoxin, or a modified clostridial neurotoxin derivative, or a clostridial neurotoxin derivative or a modified clostridial neurotoxin fragment or derivative fragment (e.g. a catalytically inactive L-chain, H_{N} domain and/or H_{C} domain) with the proviso that any L-chain present is catalytically inactive.

A polypeptide of the invention may comprise (or consist of) a modified clostridial neurotoxin or clostridial neurotoxin fragment (e.g. H_{C} domain) having one or more modifications in the amino acid sequence of the heavy chain (such as a modified H_{C} domain), wherein said modified heavy chain binds to target nerve cells with a higher or lower affinity than the native (unmodified) clostridial neurotoxin or clostridial neurotoxin fragment, with the proviso that any L-chain present is catalytically inactive. Such modifications in the H_{C} domain can include modifying residues in the ganglioside binding site of the H_{C} domain or in the protein (SV2 or synaptotagmin) binding site that alter binding to the ganglioside receptor and/or the protein receptor of the target nerve cell. Examples of such modified clostridial neurotoxins are described in WO 2006/027207 and WO 2006/114308, both of which are hereby incorporated by reference in their entirety.

A modified clostridial neurotoxin (or clostridial neurotoxin fragment) may be one that comprises one or more modifications that increases the isoelectric point of the clostridial neurotoxin when compared to an equivalent unmodified clostridial neurotoxin (or clostridial neurotoxin fragment) lacking said one or more modifications, with the proviso that any L-chain present is catalytically inactive. Suitable modified clostridial neurotoxins (with the proviso that any L-chain present has been modified to be catalytically inactive) are described below and in WO 2015/004461 A1 and WO 2016/110662 A1, which are incorporated herein by reference. Exemplary sequences include SEQ ID NOs: 42 and 62 described herein.

In one embodiment, a polypeptide of the invention may comprise a modified BoNT/A or fragment thereof (e.g. a BoNT/A H_{C} domain or fragment thereof). The modified BoNT/A or fragment thereof may be one that comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277.

The modification may be a modification when compared to catalytically inactive BoNT/A shown as SEQ ID NO: 2, wherein the amino acid residue numbering is determined by alignment with SEQ ID NO: 2. As the presence of a methionine residue at position 1 of SEQ ID NO: 2 (as well as the SEQ ID NOs corresponding to modified BoNT/A polypeptides or fragments thereof described herein) is optional, the skilled person will take the presence/absence of the methionine residue into account when determining amino acid residue numbering. For example, where SEQ ID NO: 2 includes a methionine, the position numbering will be as defined above (e.g. ASN 886 will be ASN 886 of SEQ ID NO: 2). Alternatively, where the methionine is absent from SEQ ID NO: 2 the amino acid residue numbering should be modified by −1 (e.g. ASN 886 will be ASN 885 of SEQ ID NO: 2). Similar considerations apply when the methionine at position 1 of the other polypeptide sequences described herein is present/absent, and the skilled person will readily determine the correct amino acid residue numbering using techniques routine in the art.

An alignment described herein for determining amino acid residue numbering may be carried out using any of the methods described herein for determining sequence homology and/or % sequence identity.

The amino acid residue(s) indicated for modification above are surface exposed amino acid residue(s).

A modified BoNT/A or fragment thereof may comprise a modification at one or more amino acid residue(s) selected from: ASN 886, ASN 930, ASN 954, SER 955, GLN 991, ASN 1025, ASN 1026, ASN 1052, ASN 1188, ASP 1213, GLY 1215, ASN 1216, GLN 1229, ASN 1242, ASN 1243, SER 1274 and THR 1277.

The term “one or more amino acid residue(s)” when used in the context of a modified BoNT/A or fragment thereof preferably means at least 2, 3, 4, 5, 6 or 7 of the indicated amino acid residue(s). Thus, a modified BoNT/A or fragment thereof may comprise at least 2, 3, 4, 5, 6 or 7 (preferably 7) modifications at the indicated amino acid residue(s). A modified BoNT/A or fragment thereof may comprise 1-30, 3-20, or 5-10 amino acid modifications. More preferably, the term “one or more amino acid residue(s)” when used in the context of modified BoNT/A or fragment thereof means all of the indicated amino acid residue(s).

Preferably, beyond the one or more amino acid modification(s) at the indicated amino acid residue(s), the modified BoNT/A or fragment thereof does not contain any further amino acid modifications when compared to SEQ ID NO: 2.

The modification may be selected from:
i. substitution of an acidic surface exposed amino acid residue with a basic amino acid residue;
ii. substitution of an acidic surface exposed amino acid residue with an uncharged amino acid residue;
iii. substitution of an uncharged surface exposed amino acid residue with a basic amino acid residue;
iv. insertion of a basic amino acid residue; and
v. deletion of an acidic surface exposed amino acid residue.

A modification as indicated above results in a modified BoNT/A or fragment thereof that has an increased positive surface charge and increased isoelectric point when compared to the corresponding unmodified BoNT/A or fragment thereof.

The isoelectric point (pI) is a specific property of a given protein. As is well known in the art, proteins are made from a specific sequence of amino acids (also referred to when in a protein as amino acid residues). Each amino acid of the standard set of twenty has a different side chain (or R group), meaning that each amino acid residue in a protein displays different chemical properties such as charge and hydrophobicity. These properties may be influenced by the surrounding chemical environment, such as the temperature and pH. The overall chemical characteristics of a protein will depend on the sum of these various factors.

Certain amino acid residues (detailed below) possess ionisable side chains that may display an electric charge depending on the surrounding pH. Whether such a side chain is charged or not at a given pH depends on the pKa of the relevant ionisable moiety, wherein pKa is the negative logarithm of the acid dissociation constant (Ka) for a specified proton from a conjugate base.

For example, acidic residues such as aspartic acid and glutamic acid have side chain carboxylic acid groups with pKa values of approximately 4.1 (precise pKa values may depend on temperature, ionic strength and the microenvironment of the ionisable group). Thus, these side chains exhibit a negative charge at a pH of 7.4 (often referred to as “physiological pH”). At low pH values, these side chains will become protonated and lose their charge.

Conversely, basic residues such as lysine and arginine have nitrogen-containing side chain groups with pKa values of approximately 10-12. These side chains therefore exhibit a positive charge at a pH of 7.4. These side chains will become de-protonated and lose their charge at high pH values.

The overall (net) charge of a protein molecule therefore depends on the number of acidic and basic residues present in the protein (and their degree of surface exposure) and on the surrounding pH. Changing the surrounding pH changes the overall charge on the protein. Accordingly, for every protein there is a given pH at which the number of positive and negative charges is equal and the protein displays no overall net charge. This point is known as the isoelectric point (pI). The isoelectric point is a standard concept in protein biochemistry with which the skilled person would be familiar.

The isoelectric point (pI) is therefore defined as the pH value at which a protein displays a net charge of zero. An increase in pI means that a higher pH value is required for the protein to display a net charge of zero. Thus, an increase in pI represents an increase in the net positive charge of a protein at a given pH. Conversely, a decrease in pI means that a lower pH value is required for the protein to display a net charge of zero. Thus, a decrease in pI represents a decrease in the net positive charge of a protein at a given pH.

Methods of determining the pI of a protein are known in the art and would be familiar to a skilled person. By way of example, the pI of a protein can be calculated from the average pKa values of each amino acid present in the protein (“calculated pI”). Such calculations can be performed using computer programs known in the art, such as the Compute pI/MW Tool from ExPASy (https://web.expasy.org/compute_pi/), which is the preferred method for calculating pI in accordance with the present invention. Comparisons of pI values between different molecules should be made using the same calculation technique/program.

Where appropriate, the calculated pI of a protein can be confirmed experimentally using the technique of isoelectric focusing (“observed pI”). This technique uses electrophoresis to separate proteins according to their pI. Isoelectric focusing is typically performed using a gel that has an immobilised pH gradient. When an electric field is applied, the protein migrates through the pH gradient until it reaches the pH at which it has zero net charge, this point being the pI of the protein. Results provided by isoelectric focusing are typically relatively low-resolution in nature, and thus the present inventors believe that results provided by calculated pI (as described above) are more appropriate to use.

Throughout the present specification, “pI” means “calculated pI” unless otherwise stated.

The pI of a protein may be increased or decreased by altering the number of basic and/or acidic groups displayed on its surface. This can be achieved by modifying one or more amino acids of the protein. For example, an increase in pI may be provided by reducing the number of acidic residues, or by increasing the number of basic residues.

A modified BoNT/A or fragment thereof of the invention may have a pI value that is at least 0.2, 0.4, 0.5 or 1 pI units higher than that of a catalytically inactive BoNT/A (e.g. SEQ ID NO: 2) or fragment thereof. Preferably, a modified BoNT/A or fragment thereof may have a pI of at least 6.6, e.g. at least 6.8.

The properties of the 20 standard amino acids are indicated in the table below:

| Amino Acid | | | Side Chain |
|---|---|---|---|
| Aspartic acid | Asp | D | Charged (acidic) |
| Glutamic acid | Glu | E | Charged (acidic) |
| Arginine | Arg | R | Charged (basic) |
| Lysine | Lys | K | Charged (basic) |
| Histidine | His | H | Uncharged (polar) |
| Asparagine | Asn | N | Uncharged (polar) |
| Glutamine | Gln | Q | Uncharged (polar) |
| Serine | Ser | S | Uncharged (polar) |
| Threonine | Thr | T | Uncharged (polar) |
| Tyrosine | Tyr | Y | Uncharged (polar) |
| Methionine | Met | M | Uncharged (polar) |
| Tryptophan | Trp | W | Uncharged (polar) |
| Cysteine | Cys | C | Uncharged (polar) |
| Alanine | Ala | A | Uncharged (hydrophobic) |
| Glycine | Gly | G | Uncharged (hydrophobic) |
| Valine | Val | V | Uncharged (hydrophobic) |
| Leucine | Leu | L | Uncharged (hydrophobic) |
| Isoleucine | Ile | I | Uncharged (hydrophobic) |
| Proline | Pro | P | Uncharged (hydrophobic) |
| Phenylalanine | Phe | F | Uncharged (hydrophobic) |

The following amino acids are considered charged amino acids: aspartic acid (negative), glutamic acid (negative), arginine (positive), and lysine (positive).

At a pH of 7.4, the side chains of aspartic acid (pKa 3.1) and glutamic acid (pKa 4.1) have a negative charge, while the side chains of arginine (pKa 12.5) and lysine (pKa 10.8) have a positive charge. Aspartic acid and glutamic acid are referred to as acidic amino acid residues. Arginine and lysine are referred to as basic amino acid residues.

The following amino acids are considered uncharged, polar (meaning they can participate in hydrogen bonding) amino acids: asparagine, glutamine, histidine, serine, threonine, tyrosine, cysteine, methionine, and tryptophan.

The following amino acids are considered uncharged, hydrophobic amino acids: alanine, valine, leucine, isoleucine, phenylalanine, proline, and glycine.

In an amino acid insertion, an additional amino acid residue (one that is not normally present) is incorporated into the BoNT/A polypeptide sequence or fragment thereof, thus increasing the total number of amino acid residues in said sequence. In an amino acid deletion, an amino acid residue is removed from the clostridial toxin amino acid sequence, thus reducing the total number of amino acid residues in said sequence.

Preferably, the modification is a substitution, which advantageously maintains the same number of amino acid residues in the modified BoNT/A or fragment thereof. In an amino acid substitution, an amino acid residue that forms part of the BoNT/A polypeptide sequence or fragment thereof is replaced with a different amino acid residue. The replacement amino acid residue may be one of the 20 standard amino acids, as described above. Alternatively, the replacement amino acid in an amino acid substitution may be a non-standard amino acid (an amino acid that is not part of the standard set of 20 described above). By way of example, the replacement amino acid may be a basic non-standard amino acid, e.g. L-Ornithine, L-2-amino-3-guanidinopropionic acid, or D-isomers of Lysine, Arginine and Ornithine). Methods for introducing non-standard amino acids into proteins are known in the art and include recombinant protein synthesis using E. coli auxotrophic expression hosts.

In one embodiment, the substitution is selected from: substitution of an acidic amino acid residue with a basic amino acid residue, substitution of an acidic amino acid residue with an uncharged amino acid residue, and substitution of an uncharged amino acid residue with a basic amino acid residue. In one embodiment, wherein the substitution is a substitution of an acidic amino acid residue with an uncharged amino acid residue, the acidic amino acid residue is replaced with its corresponding uncharged amide amino acid residue (i.e. aspartic acid is replaced with asparagine, and glutamic acid is replaced with glutamine).

Preferably, the basic amino acid residue is a lysine residue or an arginine residue. In other words, the substitution is substitution with lysine or arginine. Most preferably, the modification is substitution with lysine.

Preferably, a modified BoNT/A or fragment thereof for use in the invention comprises between 4 and 40 amino acid modifications located in the clostridial toxin H_{CN} domain. Said modified BoNT/A or fragment thereof preferably also has pI of at least 6.6. Said modified BoNT/A preferably comprises modifications of at least 4 amino acids selected from: ASN 886, ASN 930, ASN 954, SER 955, GLN 991, ASN 1025, ASN 1026, and ASN 1052, wherein said modification comprises substitution of the amino acids with a lysine residue or an arginine residue. For example, said modified BoNT/A or fragment thereof may comprise modifications of at least 5 amino acids selected from: ASN 886, ASN 930, ASN 954, SER 955, GLN 991, ASN 1025, ASN 1026, ASN 1052, and GLN 1229, wherein said modification comprises substitution of the amino acids with a lysine residue or an arginine residue.

Methods for modifying proteins by substitution, insertion or deletion of amino acid residues are known in the art. By way of example, amino acid modifications may be introduced by modification of a DNA sequence encoding a polypeptide (e.g. encoding unmodified BoNT/A or a fragment thereof). This can be achieved using standard molecular cloning techniques, for example by site-directed mutagenesis where short strands of DNA (oligonucleotides) coding for the desired amino acid(s) are used to replace the original coding sequence using a polymerase enzyme, or by inserting/deleting parts of the gene with various enzymes (e.g., ligases and restriction endonucleases). Alternatively, a modified gene sequence can be chemically synthesised.

In one embodiment a polypeptide for use according to the invention comprises a polypeptide sequence having at least 70% sequence identity to SEQ ID NO: 42 and/or a polypeptide sequence that is encoded by a nucleotide sequence having at least 70% sequence identity to SEQ ID NO: 41. In one embodiment a polypeptide for use according to the invention comprises a polypeptide sequence having at least 80%, 90%, 95% or 98% sequence identity to SEQ ID NO: 42. Preferably, a polypeptide for use according to the invention comprises a polypeptide sequence shown as SEQ ID NO: 42. In one embodiment a polypeptide for use according to the invention comprises a polypeptide sequence that is encoded by a nucleotide sequence having at least 80%, 90%, 95% or 98% sequence identity to SEQ ID NO: 41. Preferably, a polypeptide for use according to the invention comprises a polypeptide sequence that is encoded by a nucleotide sequence shown as SEQ ID NO: 41.

In one embodiment a polypeptide for use according to the invention comprises a polypeptide sequence having at least 70% sequence identity to SEQ ID NO: 62. In one embodiment a polypeptide for use according to the invention comprises a polypeptide sequence having at least 80%, 90%, 95% or 98% sequence identity to SEQ ID NO: 62. Preferably, a polypeptide for use according to the invention comprises (more preferably consists of) a polypeptide sequence shown as SEQ ID NO: 62.

SEQ ID NO: 42 is an example of a modified BoNT/A fragment and SEQ ID NO: 62 is an example of a modified BoNT/A polypeptide that is catalytically inactive. Such modified BoNT/A polypeptides and fragments are particularly preferred for use in the present invention. The polypeptides shown as SEQ ID NO: 42 and 62 have a number of amino acid modifications (e.g. substitutions) when compared to wild-type BoNT/A, which increase the isoelectric point of the polypeptide. Without wishing to be bound by theory, it is believed that the increased net positive charge promotes electrostatic interactions between the polypeptide and anionic extracellular components, thereby promoting binding between the polypeptide and cell surface thus increasing retention at a site of administration and/or duration of action. Thus, it is envisaged that treatment using SEQ ID NO: 42 and 62 will be improved compared to equivalent polypeptides lacking said modifications.

In one embodiment a polypeptide comprising a polypeptide sequence having at least 70% sequence identity to SEQ ID NO: 42 or 62 and/or comprising a polypeptide sequence that is encoded by a nucleotide sequence having at least 70% sequence identity to SEQ ID NO: 41 comprises a substitution at one or more (preferably two or more, three or more, four or more, five or more or six or more, more preferably at all) of positions 930, 955, 991, 1026, 1052, 1229, and 886.

Preferably, the polypeptide comprising a polypeptide sequence having at least 70% sequence identity to SEQ ID NO: 42 or 62 and/or comprising a polypeptide sequence that is encoded by a nucleotide sequence having at least 70% sequence identity to SEQ ID NO: 41 comprises lysine or arginine (more preferably lysine) at one or more of positions 930, 955, 991, 1026, 1052, 1229, and 886. In one embodiment, the polypeptide comprises lysine or arginine (more preferably lysine) at least two, three, four, five, six or all of positions 930, 955, 991, 1026, 1052, 1229, and 886. Most preferably, the polypeptide comprises lysine or arginine (more preferably lysine) at all of positions 930, 955, 991, 1026, 1052, 1229, and 886.

In one embodiment a clostridial neurotoxin H_{C} domain for use in the invention is a modified BoNT/A H_{C} domain comprising a modification of one or more amino acids residues selected from Y1117, F1252, H1253, and L1278. For example, a modified BoNT/A H_{C} domain may comprise one or more (preferably two or more) of the following modifications Y1117V, F1252Y, H1253K, and L1278F or L1278H.

In one embodiment a modified BoNT/A H_{C} domain comprises the following modifications: Y1117V and H1253K; or Y1117V, F1252Y, H1253K, and L1278F; or Y1117V, F1252Y, H1253K, and L1278H.

Preferably, a modified BoNT/A H_{C} domain comprises the following modifications: Y1117V and H1253K; or Y1117V, F1252Y, H1253K, and L1278H.

The modification may be a modification when compared to catalytically inactive BoNT/A shown as SEQ ID NO: 2, wherein the amino acid residue numbering is determined by alignment with SEQ ID NO: 2. As the presence of a methionine residue at position 1 of SEQ ID NO: 2 is optional, the skilled person will take the presence/absence of the methionine residue into account when determining amino acid residue numbering. For example, where SEQ ID NO: 2 includes a methionine, the position numbering will be as defined above (e.g. Y1117 will align against Y1117 of SEQ ID NO: 2). Alternatively, where the methionine is absent from SEQ ID NO: 2 the amino acid residue numbering should be modified by −1 (e.g. Y1117 will align against Y1116 of SEQ ID NO: 2). Similar considerations apply when the methionine at position 1 of the other polypeptide sequences described herein is present/absent, and the skilled person will readily determine the correct amino acid residue numbering using techniques routine in the art.

A modified BoNT/A H_{C} domain may comprise a polypeptide sequence having at least 70% sequence identity to any one of SEQ ID NOs: 46, 48 or 50 with the proviso that the modified BoNT/A H_{C} domain comprises a modification as described above. In one embodiment a modified BoNT/A H_{C} domain comprises a polypeptide sequence having at least 80%, 90%, 95% or 98% sequence identity to any one of SEQ ID NOs: 46, 48 or 50 with the proviso that the modified BoNT/A H_{C} domain comprises a modification as described above. In one embodiment a modified BoNT/A H_{C} domain comprises a polypeptide sequence having at least 99% or 99.9% sequence identity to any one of SEQ ID NOs: 46, 48 or 50 with the proviso that the modified BoNT/A H_{C} domain comprises a modification as described above. Preferably, a modified BoNT/A H_{C} domain comprises (more preferably consists of) a polypeptide sequence comprising any one of SEQ ID NOs: 46, 48 or 50.

A modified BoNT/A H_{C} domain may comprise a polypeptide sequence having at least 70% sequence identity to any one of SEQ ID NOs: 46 or 50 with the proviso that the modified BoNT/A H_{C} domain comprises a modification as described above. In one embodiment a modified BoNT/A H_{C} domain comprises a polypeptide sequence having at least 80%, 90%, 95% or 98% sequence identity to any one of SEQ ID NOs: 46 or 50 with the proviso that the modified BoNT/A H_{C} domain comprises a modification as described above. In one embodiment a modified BoNT/A H_{C} domain comprises a polypeptide sequence having at least 99% or 99.9% sequence identity to any one of SEQ ID NOs: 46 or 50 with the proviso that the modified BoNT/A H_{C} domain comprises a modification as described above. Preferably, a modified BoNT/A H_{C} domain comprises (more preferably consists of) a polypeptide sequence comprising any one of SEQ ID NOs: 46 or 50.

A modified BoNT/A H_{C} domain may be one encoded by a nucleotide sequence having at least 70% sequence identity to any one of SEQ ID NOs: 45, 47 or 49 with the proviso that the modified BoNT/A H_{C} domain comprises a modification as described above. In one embodiment a modified BoNT/A H_{C} domain be one encoded by a nucleotide sequence having at least 80%, 90%, 95% or 98% sequence identity to any one of SEQ ID NOs: 45, 47 or 49 with the proviso that the modified BoNT/A H_{C} domain comprises a modification as described above. In one embodiment a modified BoNT/A H_{C} domain be one encoded by a nucleotide sequence having at least 99% or 99.9% sequence identity to any one of SEQ ID NOs: 45, 47 or 49 with the proviso that the modified BoNT/A H_{C} domain comprises a modification as described above. Preferably, a modified BoNT/A H_{C} domain be one encoded by any one of SEQ ID NOs: 45, 47 or 49.

A modified BoNT/A H_{C} domain may be one encoded by a nucleotide sequence having at least 70% sequence identity to any one of SEQ ID NOs: 45 or 49 with the proviso that the modified BoNT/A H_{C} domain comprises a modification as described above. In one embodiment a modified BoNT/A H_{C} domain be one encoded by a nucleotide sequence having at least 80%, 90%, 95% or 98% sequence identity to any one of SEQ ID NOs: 45 or 49 with the proviso that the modified BoNT/A H_{C} domain comprises a modification as described above. In one embodiment a modified BoNT/A H_{C} domain be one encoded by a nucleotide sequence having at least 99% or 99.9% sequence identity to any one of SEQ ID NOs: 45 or 49 with the proviso that the modified BoNT/A H_{C} domain comprises a modification as described above. Preferably, a modified BoNT/A H_{C} domain be one encoded by any one of SEQ ID NOs: 45 or 49.

A polypeptide of the present invention may comprise (or consist of) a hybrid or chimeric clostridial neurotoxin (or a fragment of a hybrid or chimeric clostridial neurotoxin), with the proviso that the L-chain is catalytically inactive (when present). A hybrid clostridial neurotoxin comprises at least a portion of a light chain from one clostridial neurotoxin or subtype thereof, and at least a portion of a heavy chain from another clostridial neurotoxin or clostridial neurotoxin subtype. In one embodiment the hybrid clostridial neurotoxin may contain the entire light chain of a light chain from one clostridial neurotoxin subtype and the heavy chain from another clostridial neurotoxin subtype, with the proviso that the L-chain is catalytically inactive (when present). In another embodiment, a chimeric clostridial neurotoxin may contain a portion (e.g. the binding domain) of the heavy chain of one clostridial neurotoxin subtype, with another portion of the heavy chain being from another clostridial neurotoxin subtype. Similarly or alternatively, the therapeutic element may comprise light chain portions from different clostridial neurotoxins, with the proviso that the L-chain is catalytically inactive (when present). Such hybrid or chimeric clostridial neurotoxins are useful, for example, as a means of delivering the therapeutic benefits of such clostridial neurotoxins to subjects who are immunologically resistant to a given clostridial neurotoxin subtype, to subjects who may have a lower than average concentration of receptors to a given clostridial neurotoxin heavy chain binding domain, or to subjects who may have a protease-resistant variant of the membrane or vesicle toxin substrate (e.g., SNAP-25, VAMP and syntaxin). Hybrid and chimeric clostridial neurotoxins are described in U.S. Pat. No. 8,071,110, which publication is hereby incorporated by reference in its entirety. Thus, in one embodiment, the polypeptide of the invention is or comprises a hybrid clostridial neurotoxin, or a chimeric clostridial neurotoxin with the proviso that the L-chain is catalytically inactive.

In a particularly preferred embodiment, a polypeptide of the invention may be a chimeric clostridial neurotoxin comprising (preferably consisting of) a catalytically inactive BoNT/A light-chain and translocation domain (LH_{N} domain), and a BoNT/B receptor binding domain (H_{C} domain) or a portion thereof. A suitable chimeric and/or hybrid clostridial neurotoxin may be one taught in WO 2017/191315 A1, which is incorporated herein by reference, with the proviso that the L-chain is catalytically inactive (e.g. has been inactivated by a modification). Such preferred sequences include SEQ ID NOs: 44 and 61.

The catalytically inactive BoNT/A LH_{N} domain may be covalently linked to the BoNT/B H_{C} domain. Said chimeric BoNT/A is also referred to herein as “BoNT/AB” or a “BoNT/AB chimera”.

The C-terminal amino acid residue of the LH_{N} domain may correspond to the first amino acid residue of the 3₁₀ helix separating the LH_{N} and H_{C} domains of BoNT/A, and the N-terminal amino acid residue of the H_{C} domain may correspond to the second amino acid residue of the 3₁₀ helix separating the LH_{N} and H_{C} domains in BoNT/B.

Reference herein to the “first amino acid residue of the 3₁₀ helix separating the LH_{N} and H_{C} domains of BoNT/A” means the N-terminal residue of the 3₁₀ helix separating the LH_{N} and H_{C} domains.

Reference herein to the “second amino acid residue of the 3₁₀ helix separating the LH_{N} and H_{C} domains of BoNT/B” means the amino acid residue following the N-terminal residue of the 3₁₀ helix separating the LH_{N} and H_{C} domains.

A “3₁₀ helix” is a type of secondary structure found in proteins and polypeptides, along with α-helices, β-sheets and reverse turns. The amino acids in a 3₁₀ helix are arranged in a right-handed helical structure where each full turn is completed by three residues and ten atoms that separate the intramolecular hydrogen bond between them. Each amino acid corresponds to a 120° turn in the helix (i.e., the helix has three residues per turn), and a translation of 2.0 Å (=0.2 nm) along the helical axis, and has 10 atoms in the ring formed by making the hydrogen bond. Most importantly, the N—H group of an amino acid forms a hydrogen bond with the C═O group of the amino acid three residues earlier; this repeated i+3→i hydrogen bonding defines a 3₁₀ helix. A 3₁₀ helix is a standard concept in structural biology with which the skilled person is familiar.

This 3₁₀ helix corresponds to four residues which form the actual helix and two cap (or transitional) residues, one at each end of these four residues. The term “3₁₀ helix separating the LH_{N} and H_{C} domains” as used herein consists of those 6 residues.

Through carrying out structural analyses and sequence alignments, a 3₁₀ helix separating the LH_{N} and H_{C} domains was identified. This 3₁₀ helix is surrounded by an α-helix at its N-terminus (i.e. at the C-terminal part of the LH_{N} domain) and by a β-strand at its C-terminus (i.e. at the N-terminal part of the H_{C} domain). The first (N-terminal) residue (cap or transitional residue) of the 3₁₀ helix also corresponds to the C-terminal residue of this α-helix.

The 3₁₀ helix separating the LH_{N} and H_{C} domains can be for example determined from publicly available crystal structures of botulinum neurotoxins, for example 3BTA (http://www.rcsb.org/pdb/explore/explore.do?structureId=3BTA) and 1EPW (http://www.rcsb.org/pdb/explore/explore.do?structureId=1EPW) for botulinum neurotoxins A1 and 1 respectively.

In silico modelling and alignment tools which are publicly available can also be used to determine the location of the 3₁₀ helix separating the LH_{N} and H_{C} domains in other neurotoxins, for example the homology modelling servers LOOPP (Learning, Observing and Outputting Protein Patterns, http://loopp.org), PHYRE (Protein Homology/analogY Recognition Engine, http://www.sbg.bio.ic.ac.uk/phyre2/) and Rosetta (https://www.rosettacommons.org/), the protein superposition server SuperPose (http://wishart.biology.ualberta.ca/superpose/), the alignment program Clustal Omega (http://www.clustal.org/omega/), and a number of other tools/services listed at the Internet Resources for Molecular and Cell Biologists (http://molbiol-tools.ca/). In particular that the region around the “H_{N}/H_{CN}” junction is structurally highly conserved which renders it an ideal region to superimpose different serotypes.

For example, the following methodology may be used to determine the sequence of this 3₁₀ helix in other neurotoxins:
1. The structural homology modelling tool LOOP (http://loopp.org) was used to obtain a predicted structure of other BoNT serotypes based on the BoNT/A1 crystal structure (3BTA.pdb);
2. The structural (pdb) files thus obtained were edited to include only the N-terminal end of the H_{CN} domain and about 80 residues before it (which are part of the H_{N} domain), thereby retaining the “H_{N}/H_{CN}” region which is structurally highly conserved;
3. The protein superposition server SuperPose (http://wishart.biology.ualberta.ca/superpose/) was used to superpose each serotype onto the 3BTA.pdb structure;
4. The superposed pdb files were inspected to locate the 3₁₀ helix at the start of the H_{C} domain of BoNT/A1, and corresponding residues in the other serotype were then identified;
5. The other BoNT serotype sequences were aligned with Clustal Omega in order to check that corresponding residues were correct.

Examples of LH_{N}, H_{C} and 3₁₀ helix domains determined by this method are presented below:

| | Accession Number | | | |
|---|---|---|---|---|
| | (Plus Sequence | | | |
| | Version after | | | |
| Neurotoxin | Decimal) | LH_{N} | H_{C} | 3₁₀ helix |
| Catalytically | N/A | 1-872 | 873-1296 | ⁸⁷²NIINTS⁸⁷⁷ |
| inactive | | | | |
| BoNT/A1 | | | | |
| (SEQ ID | | | | |
| NO: 2) | | | | |
| BoNT/A2 | X73423.3 | 1-872 | 873-1296 | ⁸⁷²NIVNTS⁸⁷⁷ |
| BoNT/A3 | DQ185900.1 (aka | 1-872 | 873-1292 | ⁸⁷²NIVNTS⁸⁷⁷ |
| | Q3LRX9.1) | | | |
| BoNT/A4 | EU341307.1 (aka | 1-872 | 873-1296 | ⁸⁷²NITNAS⁸⁷⁷ |
| | Q3LRX8.1) | | | |
| BoNT/A5 | EU679004.1 (aka | 1-872 | 873-1296 | ⁸⁷²NIINTS⁸⁷⁷ |
| | C1IPK2.1) | | | |
| BoNT/A6 | FJ981696.1 | 1-872 | 873-1296 | ⁸⁷²NIINTS⁸⁷⁷ |
| BoNT/A7 | JQ954969.1 (aka | 1-872 | 873-1296 | ⁸⁷²NIINTS⁸⁷⁷ |
| | K4LN57.1) | | | |
| BoNT/A8 | KM233166.1 | 1-872 | 873-1297 | ⁸⁷²NITNTS⁸⁷⁷ |
| BoNT/B1 | B1INP5.1 | 1-859 | 860-1291 | ⁸⁵⁹EILNNI⁸⁶⁴ |
| (a.k.a. SEQ | | | | |
| ID NO: 52) | | | | |
| BoNT/B2 | AB084152.1 (aka | 1-859 | 860-1291 | ⁸⁵⁹EILNNI⁸⁶⁴ |
| | Q8GR96.1) | | | |
| BoNT/B3 | EF028400.1 (aka | 1-859 | 860-1291 | ⁸⁵⁹EILNNI⁸⁶⁴ |
| | A2I2S2.1) | | | |
| BoNT/B4 | EF051570.1 (aka | 1-859 | 860-1291 | ⁸⁵⁹EILNNI⁸⁶⁴ |
| | A2I2W0.1) | | | |
| BoNT/B5 | EF033130.1 (aka | 1-859 | 860-1291 | ⁸⁵⁹DILNNI⁸⁶⁴ |
| | A2I2U6.1) | | | |
| BoNT/B6 | AB302852.1 (aka | 1-859 | 860-1291 | ⁸⁵⁹EILNNI⁸⁶⁴ |
| | A8R089.1) | | | |
| BoNT/B7 | JQ354985.1 (aka | 1-859 | 860-1291 | ⁸⁵⁹EILNNI⁸⁶⁴ |
| | H9CNK9.1) | | | |
| BoNT/B8 | JQ964806.1 (aka | 1-859 | 860-1292 | ⁸⁵⁹EILNNI⁸⁶⁴ |
| | I6Z8G9.1) | | | |

Using structural analysis and sequence alignments, it was found that the β-strand following the 3₁₀ helix separating the LH_{N} and H_{C} domains is a conserved structure in all botulinum and tetanus neurotoxins and starts at the 8^{th} residue when starting from the first residue of the 3₁₀ helix separating the LH_{N} and H_{C} domains (e.g., at residue 879 for BoNT/A1).

A BoNT/AB chimera may comprise an LH_{N} domain from BoNT/A (having a catalytically inactive L-chain) covalently linked to a H_{C} domain from BoNT/B,
wherein the C-terminal amino acid residue of the LH_{N} domain corresponds to the eighth amino acid residue N-terminally to the β-strand located at the beginning (N-term) of the H_{C} domain of BoNT/A, and
wherein the N-terminal amino acid residue of the H_{C} domain corresponds to the seventh amino acid residue N-terminally to the β-strand located at the beginning (N-term) of the H_{C} domain of BoNT/B.

A BoNT/AB chimera may comprise an LH_{N} domain from BoNT/A (having a catalytically inactive L-chain) covalently linked to a H_{C} domain from BoNT/B,
wherein the C-terminal amino acid residue of the LH_{N} domain corresponds to the C-terminal amino acid residue of the α-helix located at the end (C-term) of LH_{N} domain of BoNT/A, and
wherein the N-terminal amino acid residue of the H_{C} domain corresponds to the amino acid residue immediately C-terminal to the C-terminal amino acid residue of the α-helix located at the end (C-term) of LH_{N} domain of BoNT/B.

The rationale of the design process of the BoNT/AB chimera was to try to ensure that the secondary structure was not compromised and thereby minimise any changes to the tertiary structure. Without wishing to be bound by theory, it is hypothesized that by not disrupting the four central amino acid residues of the 3₁₀ helix in the BoNT/AB chimera ensures an optimal conformation for the chimeric neurotoxin.

The catalytically inactive LH_{N} domain from BoNT/A may correspond to amino acid residues 1 to 872 of SEQ ID NO: 2 or 61, or a polypeptide sequence having at least 70% sequence identity thereto. The catalytically inactive LH_{N} domain from BoNT/A may correspond to amino acid residues 1 to 872 of SEQ ID NO: 2 or 61, or a polypeptide sequence having at least 80%, 90% or 95% sequence identity thereto. Preferably, the catalytically inactive LH_{N} domain from BoNT/A corresponds to amino acid residues 1 to 872 of SEQ ID NO: 2 or 61.

The H_{C} domain from BoNT/B may correspond to amino acid residues 860 to 1291 of SEQ ID NO: 52, or a polypeptide sequence having at least 70% sequence identity thereto. The H_{C} domain from BoNT/B may correspond to amino acid residues 860 to 1291 of SEQ ID NO: 52, or a polypeptide sequence having at least 80%, 90% or 95% sequence identity thereto. Preferably, the H_{C} domain from BoNT/B corresponds to amino acid residues 860 to 1291 of SEQ ID NO: 52.

Preferably, the catalytically inactive LH_{N} domain corresponds to amino acid residues 1 to 872 of BoNT/A (SEQ ID NO: 2 or 61) and the H_{C} domain corresponds to amino acid residues 860 to 1291 of BoNT/B (SEQ ID NO: 52).

Preferably, a BoNT/B H_{C} domain further comprises at least one amino acid residue substitution, addition or deletion in the H_{CC} subdomain which has the effect of increasing the binding affinity of BoNT/B neurotoxin for human Syt II as compared to the natural BoNT/B sequence. Suitable amino acid residue substitution, addition or deletion in the BoNT/B H_{CC} subdomain have been disclosed in WO 2013/180799 and in WO 2016/154534 (both herein incorporated by reference).

Suitable amino acid residue substitution, addition or deletion in the BoNT/B H_{CC} subdomain include substitution mutations selected from the group consisting of: V1118M; Y1183M; E1191M; E11911; E1191Q; E1191T; S1199Y; S1199F; S1199L; S1201V; E1191C, E1191V, E1191L, E1191Y, S1199W, S1199E, S1199H, W1178Y, W1178Q, W1178A, W1178S, Y1183C, Y1183P and combinations thereof.

Suitable amino acid residue substitution, addition or deletion in the BoNT/B H_{CC} subdomain further include combinations of two substitution mutations selected from the group consisting of: E1191M and S1199L, E1191M and S1199Y, E1191M and S1199F, E1191Q and S1199L, E1191Q and S1199Y, E1191Q and S1199F, E1191M and S1199W, E1191M and W1178Q, E1191C and S1199W, E1191C and S1199Y, E1191C and W1178Q, E1191Q and S1199W, E1191V and S1199W, E1191V and S1199Y, or E1191V and W1178Q.

Suitable amino acid residue substitution, addition or deletion in the BoNT/B H_{CC} subdomain also include a combination of three substitution mutations which are E1191M, S1199W and W1178Q.

Preferably, the suitable amino acid residue substitution, addition or deletion in the BoNT/B H_{CC} subdomain includes a combination of two substitution mutations which are E1191M and S1199Y.

The modification may be a modification when compared to unmodified BoNT/B shown as SEQ ID NO: 52, wherein the amino acid residue numbering is determined by alignment with SEQ ID NO: 52. As the presence of a methionine residue at position 1 of SEQ ID NO: 52 is optional, the skilled person will take the presence/absence of the methionine residue into account when determining amino acid residue numbering. For example, where SEQ ID NO: 52 includes a methionine, the position numbering will be as defined above (e.g. E1191 will be E1191 of SEQ ID NO: 52). Alternatively, where the methionine is absent from SEQ ID NO: 52 the amino acid residue numbering should be modified by −1 (e.g. E1191 will be E1190 of SEQ ID NO: 52). Similar considerations apply when the methionine at position 1 of the other polypeptide sequences described herein is present/absent, and the skilled person will readily determine the correct amino acid residue numbering using techniques routine in the art.

In one embodiment a polypeptide for use according to the invention comprises a polypeptide sequence having at least 70% sequence identity to SEQ ID NO: 61. In one embodiment a polypeptide for use according to the invention comprises a polypeptide sequence having at least 80%, 90%, 95% or 98% sequence identity to SEQ ID NO: 61. Preferably, a polypeptide for use according to the invention comprises (more preferably consists of) a polypeptide sequence shown as SEQ ID NO: 61.

A chimeric and/or hybrid clostridial neurotoxin for use in the present invention may comprise a portion of a BoNT/A polypeptide and a portion of a BoNT/B polypeptide, an example of which includes the polypeptide described herein as SEQ ID NO: 44.

In one embodiment a polypeptide for use according to the invention comprises a polypeptide sequence having at least 70% sequence identity to SEQ ID NO: 44 and/or a polypeptide sequence that is encoded by a nucleotide sequence having at least 70% sequence identity to SEQ ID NO: 43. In one embodiment a polypeptide for use according to the invention comprises a polypeptide sequence having at least 80%, 90%, 95% or 98% sequence identity to SEQ ID NO: 44. Preferably, a polypeptide for use according to the invention comprises a polypeptide sequence shown as SEQ ID NO: 44. In one embodiment a polypeptide for use according to the invention comprises a polypeptide sequence that is encoded by a nucleotide sequence having at least 80%, 90%, 95% or 98% sequence identity to SEQ ID NO: 43. Preferably, a polypeptide for use according to the invention comprises a polypeptide sequence that is encoded by a nucleotide sequence shown as SEQ ID NO: 43.

Suitable chimeric clostridial neurotoxins may include BoNT/FA, with the proviso that any L-chain present is catalytically inactive. Thus, a polypeptide of the invention may comprise BoNT/FA or a fragment thereof, with the proviso that any L-chain present is catalytically inactive. Catalytically inactive forms of BoNT/FA are described herein as SEQ ID NO: 26 and 34. Suitable fragments of BoNT/FA are also described herein as SEQ ID NOs: 28, 30, and 32.

In another preferred embodiment, a polypeptide of the invention may be a chimeric clostridial neurotoxin comprising a catalytically inactive BoNT/X light-chain and translocation domain (LH_{N} domain), and a receptor binding domain (H_{C} domain) or a portion thereof from a different (i.e. non-BoNT/X) clostridial neurotoxin. A suitable chimeric and/or hybrid clostridial neurotoxin may be one taught in WO 2020/065336 A1, which is incorporated herein by reference, with the proviso that the L-chain is catalytically inactive (e.g. has been inactivated by a modification). Such preferred sequences include SEQ ID NOs: 63-70 described herein.

A chimeric clostridial neurotoxin may comprise a catalytically inactive BoNT/X light-chain and translocation domain (LH_{N} domain), and:
(i) a BoNT/A receptor binding domain (H_{C} domain) or a portion thereof; or
(ii) a BoNT/B receptor binding domain (H_{C} domain) or a portion thereof; or
(iii) a BoNT/C receptor binding domain (H_{C} domain) or a portion thereof; or
(iv) a BoNT/D receptor binding domain (H_{C} domain) or a portion thereof; or
(v) a BoNT/E receptor binding domain (H_{C} domain) or a portion thereof; or
(vi) a BoNT/F receptor binding domain (H_{C} domain) or a portion thereof; or
(vii) a BoNT/G receptor binding domain (H_{C} domain) or a portion thereof; or
(viii) a TeNT receptor binding domain (H_{C} domain) or a portion thereof.

In one embodiment, the receptor binding domain (H_{C} domain) or portion thereof from a different (i.e. non-BoNT/X) clostridial neurotoxin domain may be one that binds to synaptotagmin I and/or II (Syt I/II).

Preferably, a chimeric clostridial neurotoxin may comprise a catalytically inactive BoNT/X light-chain and translocation domain (LH_{N} domain), and a BoNT/B receptor binding domain (H_{C} domain) or a portion thereof.

A polypeptide comprising a catalytically inactive BoNT/X light-chain and translocation domain (LH_{N} domain), and a receptor binding domain (H_{C} domain) or a portion thereof from a different (i.e. non-BoNT/X) clostridial neurotoxin may comprise a polypeptide sequence having at least 70% sequence identity to any one of SEQ ID NOs: 63-70. In one embodiment a polypeptide comprising a catalytically inactive BoNT/X light-chain and translocation domain (LH_{N} domain), and a receptor binding domain (H_{C} domain) or a portion thereof from a different (i.e. non-BoNT/X) clostridial neurotoxin comprises a polypeptide sequence having at least 80%, 90%, 95% or 98% sequence identity to any one of SEQ ID NOs: 63-70. Preferably, a polypeptide comprising a catalytically inactive BoNT/X light-chain and translocation domain (LH_{N} domain), and a receptor binding domain (H_{C} domain) or a portion thereof from a different (i.e. non-BoNT/X) clostridial neurotoxin (more preferably consists of) any one of SEQ ID NOs: 63-70. Of those polypeptides, SEQ ID NOs: 63-66 are most preferred.

The polypeptide comprising a catalytically inactive BoNT/X light-chain and translocation domain (LH_{N} domain), and a receptor binding domain (H_{C} domain) or a portion thereof from a different (i.e. non-BoNT/X) clostridial neurotoxin may comprise the following N-terminal amino acid sequence MGS. Where SEQ ID NOs: 63-66 contain said N-terminal amino acid sequence, said sequence is optional. In one embodiment SEQ ID NOs: 63-66 lack an N-terminal amino acid sequence shown as MGS. In one embodiment SEQ ID NOs: 63-66 comprise an N-terminal amino acid sequence shown as MGS.

The catalytically inactive LH_{N} domain from BoNT/X may correspond to amino acid residues 1 to 899 of SEQ ID NO: 63, or a polypeptide sequence having at least 70% sequence identity thereto. The catalytically inactive LH_{N} domain from BoNT/X may correspond to amino acid residues 1 to 899 of SEQ ID NO: 63, or a polypeptide sequence having at least 80%, 90% or 95% sequence identity thereto. Preferably, the catalytically inactive LH_{N} domain from BoNT/X may correspond to amino acid residues 1 to 899 of SEQ ID NO: 63.

The catalytically inactive LH_{N} domain from BoNT/X may correspond to amino acid residues 4 to 899 of SEQ ID NO: 63, or a polypeptide sequence having at least 70% sequence identity thereto. The catalytically inactive LH_{N} domain from BoNT/X may correspond to amino acid residues 4 to 899 of SEQ ID NO: 63, or a polypeptide sequence having at least 80%, 90% or 95% sequence identity thereto. Preferably, the catalytically inactive LH_{N} domain from BoNT/X may correspond to amino acid residues 4 to 899 of SEQ ID NO: 63.

The catalytically inactive LH_{N} domain from BoNT/X may correspond to amino acid residues 1 to 866 of SEQ ID NO: 65, or a polypeptide sequence having at least 70% sequence identity thereto. The catalytically inactive LH_{N} domain from BoNT/X may correspond to amino acid residues 1 to 866 of SEQ ID NO: 65, or a polypeptide sequence having at least 80%, 90% or 95% sequence identity thereto. Preferably, the catalytically inactive LH_{N} domain from BoNT/X may correspond to amino acid residues 1 to 866 of SEQ ID NO: 65.

The catalytically inactive LH_{N} domain from BoNT/X may correspond to amino acid residues 4 to 866 of SEQ ID NO: 65, or a polypeptide sequence having at least 70% sequence identity thereto. The catalytically inactive LH_{N} domain from BoNT/X may correspond to amino acid residues 4 to 866 of SEQ ID NO: 65, or a polypeptide sequence having at least 80%, 90% or 95% sequence identity thereto. Preferably, the catalytically inactive LH_{N} domain from BoNT/X may correspond to amino acid residues 4 to 866 of SEQ ID NO: 65.

The H_{C} domain from BoNT/B may correspond to amino acid residues 860 to 1291 of SEQ ID NO: 52, or a polypeptide sequence having at least 70% sequence identity thereto. The H_{C} domain from BoNT/B may correspond to amino acid residues 860 to 1291 of SEQ ID NO: 52, or a polypeptide sequence having at least 80%, 90% or 95% sequence identity thereto. Preferably, the H_{C} domain from BoNT/B corresponds to amino acid residues 860 to 1291 of SEQ ID NO: 52.

Preferably, a BoNT/B H_{C} domain further comprises at least one amino acid residue substitution, addition or deletion in the H_{CC} subdomain which has the effect of increasing the binding affinity of BoNT/B neurotoxin for human Syt II as compared to the natural BoNT/B sequence. Suitable amino acid residue substitution, addition or deletion in the BoNT/B H_{CC} subdomain have been disclosed in WO 2013/180799 and in WO 2016/154534 (both herein incorporated by reference).

Suitable amino acid residue substitution, addition or deletion in the BoNT/B H_{CC} subdomain include substitution mutations selected from the group consisting of: V1118M; Y1183M; E1191M; E11911; E1191Q; E1191T; S1199Y; S1199F; S1199L; S1201V; E1191C, E1191V, E1191L, E1191Y, S1199W, S1199E, S1199H, W1178Y, W1178Q, W1178A, W1178S, Y1183C, Y1183P and combinations thereof.

Suitable amino acid residue substitution, addition or deletion in the BoNT/B H_{CC} subdomain further include combinations of two substitution mutations selected from the group consisting of: E1191M and S1199L, E1191M and S1199Y, E1191M and S1199F, E1191Q and S1199L, E1191Q and S1199Y, E1191Q and S1199F, E1191M and S1199W, E1191M and W1178Q, E1191C and S1199W, E1191C and S1199Y, E1191C and W1178Q, E1191Q and S1199W, E1191V and S1199W, E1191V and S1199Y, or E1191V and W1178Q.

Suitable amino acid residue substitution, addition or deletion in the BoNT/B H_{CC} subdomain also include a combination of three substitution mutations which are E1191M, S1199W and W1178Q.

Preferably, the suitable amino acid residue substitution, addition or deletion in the BoNT/B H_{CC} subdomain includes a combination of two substitution mutations which are E1191M and S1199Y.

The modification may be a modification when compared to unmodified BoNT/B shown as SEQ ID NO: 52, wherein the amino acid residue numbering is determined by alignment with SEQ ID NO: 52. As the presence of a methionine residue at position 1 of SEQ ID NO: 52 is optional, the skilled person will take the presence/absence of the methionine residue into account when determining amino acid residue numbering. For example, where SEQ ID NO: 52 includes a methionine, the position numbering will be as defined above (e.g. E1191 will be E1191 of SEQ ID NO: 52). Alternatively, where the methionine is absent from SEQ ID NO: 52 the amino acid residue numbering should be modified by −1 (e.g. E1191 will be E1190 of SEQ ID NO: 52). Similar considerations apply when the methionine at position 1 of the other polypeptide sequences described herein is present/absent, and the skilled person will readily determine the correct amino acid residue numbering using techniques routine in the art.

The H_{C} domain from BoNT/A may correspond to amino acid residues 873 to 1296 of SEQ ID NO: 60, or a polypeptide sequence having at least 70% sequence identity thereto. The H_{C} domain from BoNT/A may correspond to amino acid residues 873 to 1296 of SEQ ID NO: 60, or a polypeptide sequence having at least 80%, 90% or 95% sequence identity thereto. Preferably, the H_{C} domain from BoNT/B corresponds to amino acid residues 873 to 1296 of SEQ ID NO: 60.

In one embodiment, where the polypeptide is for use in treating an inflammatory disorder, the polypeptide used does not comprise a catalytically inactive BoNT/X L-chain, a BoNT/X translocation domain (H_{N} domain), and a BoNT/A receptor binding domain (H_{C} domain). Thus, in one embodiment, where a polypeptide is for use in treating an inflammatory disorder, the polypeptide may comprise a catalytically inactive BoNT/X L-chain, a BoNT/X translocation domain (H_{N} domain) and: (i) a BoNT/B receptor binding domain (H_{C} domain); (ii) a BoNT/D receptor binding domain (H_{C} domain); or (iii) a BoNT/F receptor binding (H_{C} domain).

Similarly, in one embodiment, where the polypeptide is for use in treating pain, the polypeptide used does not comprise a catalytically inactive BoNT/X L-chain, a BoNT/X translocation domain (H_{N} domain), and a BoNT/A receptor binding domain (H_{C} domain). Thus, in one embodiment, where a polypeptide is for use in treating pain, the polypeptide may comprise a catalytically inactive BoNT/X L-chain, a BoNT/X translocation domain (H_{N} domain) and: (i) a BoNT/B receptor binding domain (H_{C} domain); (ii) a BoNT/D receptor binding domain (H_{C} domain); or (iii) a BoNT/F receptor binding (H_{C} domain).

The H_{C} domain from BoNT/D may correspond to amino acid residues 865 to 1276 of SEQ ID NO: 54, or a polypeptide sequence having at least 70% sequence identity thereto. The H_{C} domain from BoNT/D may correspond to amino acid residues 865 to 1276 of SEQ ID NO: 54, or a polypeptide sequence having at least 80%, 90% or 95% sequence identity thereto. Preferably, the H_{C} domain from BoNT/D corresponds to amino acid residues 865 to 1276 of SEQ ID NO: 54.

The H_{C} domain from BoNT/F may correspond to amino acid residues 866 to 1278 of SEQ ID NO: 56, or a polypeptide sequence having at least 70% sequence identity thereto. The H_{C} domain from BoNT/F may correspond to amino acid residues 866 to 1278 of SEQ ID NO: 56, or a polypeptide sequence having at least 80%, 90% or 95% sequence identity thereto. Preferably, the H_{C} domain from BoNT/F corresponds to amino acid residues 866 to 1278 of SEQ ID NO: 56 having a histidine to lysine substitution at position 1241 (H1241K).

Preferably, the chimeric clostridial neurotoxin may comprise (preferably consist of) a catalytically inactive BoNT/X light-chain and translocation domain (LH_{N} domain), and a receptor binding domain (H_{C} domain) or a portion thereof from a different (i.e. non-BoNT/X) clostridial neurotoxin and Cys-(Xaa)ₐ-Ile-Asp/Glu-Gly-Arg-(Yaa)_{b}-Cys (SEQ ID NO: 71), wherein a=1-10 and b=4-15. SEQ ID NO: 71 is an activation loop consensus sequence based on the BoNT/C1 activation loop.

Moreover, any polypeptide of the invention may comprise Cys-(Xaa)ₐ-Ile-Asp/Glu-Gly-Arg-(Yaa)_{b}-Cys (SEQ ID NO: 71), wherein a=1-10 and b=4-15. Said activation loop may suitably replace any activation loop sequences present in either a clostridial neurotoxin L-chain and/or translocation domain (H_{N} domain) present in a polypeptide described herein.

Xaa or Yaa when used in the context of SEQ ID NO: 71 can be any amino acid. The number of amino acids at position Xaa and Yaa are indicated by the letters ‘a’ and ‘b’, respectively. In one embodiment ‘a’ and ‘b’ can be any integer that allows for proteolytic cleavage of the activation loop and yields an active di-chain clostridial neurotoxin. In one embodiment ‘a’ is at least 1, 2, 3, 4, 5, 6, 7, 8, 9 or 10. In one embodiment ‘b’ is at least 1, 2, 3, 4, 5, 6, 7, 8, 9, 10, 11, 12, 13, 14 or 15. In one embodiment ‘a’ is ≤12, ≤11, ≤10, ≤9, ≤8, ≤7, ≤6, ≤5 or ≤4. In one embodiment ‘b’ is ≤20, ≤19, ≤18, ≤17, ≤16, ≤15, ≤14, ≤13, ≤12, ≤11, ≤10 or ≤9.

In one embodiment ‘a’ is 1-12, for example 1-10. Preferably ‘a’ is 1-7, such as 2-4. More preferably ‘a’ is 3. In one embodiment ‘b’ is 1-20, for example 4-15. Preferably ‘b’ is 6-10. More preferably ‘b’ is 8.

It is not intended that Xaa or Yaa be limited to only one type of amino acid. Thus, one or more residues present at position Xaa may be independently selected from the standard amino acids: aspartic acid, glutamic acid, arginine, lysine, histidine, asparagine, glutamine, serine, threonine, tyrosine, methionine, tryptophan, cysteine, alanine, glycine, valine, leucine, isoleucine, proline, and phenylalanine. One or more residues present at position Yaa may be independently selected from the standard amino acids: aspartic acid, glutamic acid, arginine, lysine, histidine, asparagine, glutamine, serine, threonine, tyrosine, methionine, tryptophan, cysteine, alanine, glycine, valine, leucine, isoleucine, proline, and phenylalanine. Preferably an amino acid at position Yaa (more preferably immediately C-terminal to the Arg residue of SEQ ID NO: 71) is not proline.

Alternatively/additionally, one or more residues present at position Xaa or Yaa may be independently selected from a non-standard amino acid (an amino acid that is not part of the standard set of 20 described above). By way of example, non-standard amino acids may include 4-hydroxyproline, 6-N-methyl lysine, 2-aminoisobutyric acid, isovaline, α-methyl serine, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, L-Ornithine, L-2-amino-3-guanidinopropionic acid, or D-isomers of Lysine, Arginine and/or Ornithine, and 4-fluorophenylalanine. Methods for introducing non-standard amino acids into proteins are known in the art, and include recombinant protein synthesis using E. coli auxotrophic expression hosts.

The sequence Ile-Asp/Glu-Gly-Arg comprised in SEQ ID NO: 71 refers to a site surprisingly found in WO 2020/065336 A1 to be recognised by enterokinase (as well as factor Xa). Said document describes suitable methods for cleaving at Ile-Asp/Glu-Gly-Arg, thereby producing a di-chain polypeptide. Preferably the sequence is Ile-Asp-Gly-Arg, e.g. Cys-(Xaa)ₐ-Ile-Asp-Gly-Arg-(Yaa)_{b}-Cys. It is believed that enterokinase and factor Xa hydrolyse a peptide bond immediately C-terminal to Arg of SEQ ID NO: 71 (i.e. the peptide bond between Arg and Yaa).

In one embodiment an amino acid residue at Xaa immediately N-terminal to lie of SEQ ID NO: 71 is an uncharged hydrophobic amino acid, preferably alanine. In some embodiments ‘a’ is at least 2, and Xaa comprises at least a C-terminal uncharged polar amino acid and a charged basic amino acid immediately N-terminal thereto. The charged basic amino acid is preferably lysine. Thus in embodiments where ‘a’ is at least 2, Xaa may comprise at least Lys-Ala, wherein Ala is immediately N-terminal to lie of SEQ ID NO: 71.

In one embodiment Xaa comprises or consists of the sequence HKA.

In one embodiment an amino acid residue at Yaa immediately C-terminal to Arg of SEQ ID NO: 71 is an uncharged polar amino acid, preferably serine. In some embodiments ‘b’ is at least 2, and Yaa comprises at least an N-terminal uncharged polar amino acid and an uncharged hydrophobic amino acid immediately C-terminal thereto. The uncharged hydrophobic amino acid is preferably leucine. Thus in embodiments where ‘b’ is at least 2, Yaa may comprise at least Ser-Leu, wherein Ser is immediately C-terminal to Arg of SEQ ID NO: 71.

In one embodiment Yaa comprises or consists of the sequence SLYNKTLDC.

In some embodiments a polypeptide herein comprises an activation loop having at least 70% sequence identity to SEQ ID NO: 72. In one embodiment a polypeptide herein comprises an activation loop having at least 80%, 85% or 90% sequence identity to SEQ ID NO: 72. Preferably a polypeptide herein comprises an activation loop having at least 95% sequence identity to SEQ ID NO: 72. More preferably, a polypeptide herein comprises an activation loop having at least 99% sequence identity to SEQ ID NO: 72.

In a particularly preferred embodiment a polypeptide herein comprises an activation loop comprising SEQ ID NO: 72, more preferably consisting of SEQ ID NO: 72.

The activation loop may also be a variant of SEQ ID NO: 72, such as SEQ ID NO:73 or a sequence having at least 70% sequence identity thereto. SEQ ID NO: 73 is a variant of SEQ ID NO: 72 in which the enterokinase recognition site IDGR has been mutated to IEGR. In one embodiment a polypeptide herein comprises an activation loop having at least 70% sequence identity to SEQ ID NO: 73. In one embodiment a polypeptide herein comprises an activation loop having at least 80%, 85% or 90% sequence identity to SEQ ID NO: 73. Preferably a polypeptide herein comprises an activation loop having at least 95% sequence identity to SEQ ID NO: 73. More preferably, a polypeptide herein comprises an activation loop having at least 99% sequence identity to SEQ ID NO: 73.

In a particularly preferred embodiment a polypeptide herein comprises an activation loop comprising SEQ ID NO: 73, more preferably consisting of SEQ ID NO: 73.

In one embodiment an activation loop described herein (e.g. SEQ ID NO: 71, 72 or 73) may be modified to include an additional or alternative protease site. For example, a protease site shown as SEQ ID NO: 77. An example of such a modified activation loop is shown as SEQ ID NO: 78. Thus, in one embodiment a polypeptide herein comprises an activation loop having at least 70% sequence identity to SEQ ID NO: 78. In one embodiment a polypeptide herein comprises an activation loop having at least 80%, 85% or 90% sequence identity to SEQ ID NO: 78. Preferably a polypeptide herein comprises an activation loop having at least 95% sequence identity to SEQ ID NO: 78. More preferably, a polypeptide herein comprises an activation loop having at least 99% sequence identity to SEQ ID NO: 78. In a particularly preferred embodiment a polypeptide herein comprises an activation loop comprising SEQ ID NO: 78, more preferably consisting of SEQ ID NO: 78.

In one embodiment, a polypeptide of the invention may comprise (or consist of) a re-targeted clostridial neurotoxin, with the proviso that any L-chain present is catalytically inactive. In a re-targeted clostridial neurotoxin, the clostridial neurotoxin is modified to include an exogenous ligand known as a Targeting Moiety (TM). The TM is selected to provide binding specificity for a desired target cell, and as part of the re-targeting process the native binding portion of the clostridial neurotoxin (e.g. the H_{C} domain, or the H_{CC} domain) may be removed. Re-targeting technology is described, for example, in: EP-B-0689459; WO 1994/021300; EP-B-0939818; U.S. Pat. Nos. 6,461,617; 7,192,596; WO 1998/007864; EP-B-0826051; U.S. Pat. Nos. 5,989,545; 6,395,513; 6,962,703; WO 1996/033273; EP-B-0996468; U.S. Pat. No. 7,052,702; WO 1999/017806; EP-B-1107794; U.S. Pat. No. 6,632,440; WO 2000/010598; WO 2001/21213; WO 2006/059093; WO 2000/62814; WO 2000/04926; WO 1993/15766; WO 2000/61192; and WO 1999/58571; all of which are hereby incorporated by reference in their entirety. Thus, in one embodiment, the polypeptide of the invention is a re-targeted clostridial neurotoxin, with the proviso that any L-chain present is catalytically inactive. The polypeptide of the present invention may lack a functional H_{C} domain of a clostridial neurotoxin and also lack any functionally equivalent TM.

In embodiments where a polypeptide described herein has a tag for purification (e.g. a His-tag) and/or a linker, said tag and/or linker are optional.

The polypeptides of the present invention may be free from the complexing proteins that are present in a naturally occurring clostridial neurotoxin complex.

The polypeptides of the present invention can be produced using recombinant nucleic acid technologies. Thus, in one embodiment, a polypeptide (as described above) is a recombinant polypeptide.

In one embodiment a nucleic acid (for example, a DNA) comprising a nucleic acid sequence encoding a polypeptide is provided. In one embodiment, the nucleic acid sequence is prepared as part of a DNA vector comprising a promoter and a terminator. The nucleic acid sequence may be selected from any of the nucleic acid sequences described herein.

In a preferred embodiment, the vector has a promoter selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |

In another preferred embodiment, the vector has a promoter selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |
| T5-lac operator | IPTG | 0.2 mM (0.05-2.0 mM) |

The nucleic acid molecules may be made using any suitable process known in the art. Thus, the nucleic acid molecules may be made using chemical synthesis techniques. Alternatively, the nucleic acid molecules of the invention may be made using molecular biology techniques.

The DNA construct of the present invention is preferably designed in silico, and then synthesised by conventional DNA synthesis techniques.

The above-mentioned nucleic acid sequence information is optionally modified for codon-biasing according to the ultimate host cell (e.g. E. coli) expression system that is to be employed.

The terms “nucleotide sequence” and “nucleic acid” are used synonymously herein. Preferably the nucleotide sequence is a DNA sequence.

A polypeptide of the invention (and especially any clostridial neurotoxin portion thereof) may be present as a single-chain or as a di-chain. However, it is preferred that the polypeptide is present as a di-chain in which the catalytically inactive L-chain is linked to the H-chain (or component thereof, e.g. the H_{N} domain) via a di-sulphide bond.

The invention provides a method of producing a single-chain polypeptide having a catalytically inactive light chain and a heavy chain, the method comprising expressing a nucleic acid described herein in an expression host, lysing the host cell to provide a host cell homogenate containing the single-chain polypeptide, and isolating the single-chain polypeptide. In one aspect, the present invention provides a method of proteolytically processing a polypeptide described herein, the method comprising contacting the polypeptide with a protease that hydrolyses a peptide bond in the activation loop of the polypeptide, thereby converting the (single-chain) polypeptide into a corresponding di-chain polypeptide (e.g. wherein the catalytically inactive light chain and heavy chain are joined together by a disulphide bond).

The present invention therefore provides a di-chain polypeptide obtainable by a method of the invention.

A “subject” as used herein may be a mammal, such as a human or other mammal. Preferably “subject” means a human subject.

The term “disorder” as used herein also encompasses a “disease”. In one embodiment the disorder is a disease.

The term “treat” or “treating” as used herein encompasses prophylactic treatment (e.g. to prevent onset of a disorder [e.g. pain]) as well as corrective treatment (treatment of a subject already suffering from a disorder [e.g. pain]). Preferably “treat” or “treating” as used herein means corrective treatment.

The term “treat” or “treating” as used herein refers to the disorder (e.g. pain) and/or a symptom thereof. In the context of treating a lower urinary tract disorder, it is preferred that the polypeptide of the invention treats a symptom thereof, such as pain and/or the urge to urinate (a.k.a. urgency). Preferably, a polypeptide of the invention treats at least pain associated with a lower urinary tract disorder.

Therefore a polypeptide of the invention may be administered to a subject in a therapeutically effective amount or a prophylactically effective amount. Preferably a polypeptide of the invention is administered to a subject in a therapeutically effective amount.

A “therapeutically effective amount” is any amount of the polypeptide, which when administered alone or in combination with another agent to a subject for treating said disorder (e.g. pain) (or a symptom thereof) is sufficient to effect such treatment of the disorder, or symptom thereof.

A “prophylactically effective amount” is any amount of the polypeptide that, when administered alone or in combination with another agent to a subject inhibits or delays the onset or reoccurrence of a disorder (e.g. pain) (or a symptom thereof). In some embodiments, the prophylactically effective amount prevents the onset or reoccurrence of a disorder (e.g. pain) entirely. “Inhibiting” the onset means either lessening the likelihood of a disorder's onset (e.g. where the disorder is pain) (or symptom thereof), or preventing the onset entirely.

In some embodiments, the polypeptide of the invention may be administered to a subject prior to the onset of pain (e.g. via a noxious insult [such as UV or a chemical insult] or traumatic insult [such as surgery]). For example, the polypeptide of the invention may be administered at least 1 hour, 6 hours, 12 hours, or 24 hours prior to onset of pain.

The polypeptides of the invention may be formulated in any suitable manner for administration to a subject, for example as part of a pharmaceutical composition. Thus, in one aspect, the invention provides a pharmaceutical composition comprising a polypeptide of the invention and a pharmaceutically acceptable carrier, excipient, adjuvant, propellant and/or salt.

The polypeptides of the present invention may be formulated for oral, parenteral, continuous infusion, inhalation or topical application. Compositions suitable for injection may be in the form of solutions, suspensions or emulsions, or dry powders which are dissolved or suspended in a suitable vehicle prior to use.

In the case of a polypeptide that is to be delivered locally, the polypeptide may be formulated as a cream (e.g. for topical application), or for sub-dermal injection.

Local delivery means may include an aerosol, or other spray (e.g. a nebuliser). In this regard, an aerosol formulation of a polypeptide enables delivery to the lungs and/or other nasal and/or bronchial or airway passages.

Polypeptides of the invention may be administered to a subject by intrathecal or epidural injection in the spinal column at the level of the spinal segment involved in the innervation of an affected organ.

A route of administration may be via laproscopic and/or localised injection. In one embodiment a polypeptide of the invention is administered at or near to a site to be treated, preferably at a site to be treated. For example, the polypeptide may be administered intrathecally or intraspinally. In one embodiment the route of administration of a polypeptide of the invention may be perineural, intraneural, intraspinal, and/or intrathecal.

In one embodiment a polypeptide of the invention may be administered peripherally. In one embodiment, the polypeptide may be administered intradermally subcutaneously or intramuscularly. Preferably, a polypeptide of the invention is administered intradermally.

The dosage ranges for administration of the polypeptides of the present invention are those to produce the desired therapeutic and/or prophylactic effect. It will be appreciated that the dosage range required depends on the precise nature of the clostridial neurotoxin or composition, the route of administration, the nature of the formulation, the age of the subject, the nature, extent or severity of the subject's condition, contraindications, if any, and the judgement of the attending physician. Variations in these dosage levels can be adjusted using standard empirical routines for optimisation.

In one embodiment a dosage of the polypeptide is a flat dose. A flat dose may be in the range of 50 pg to 250 μg, preferably 100 pg to 100 μg. In one embodiment a flat dose may be at least 50 pg, 100 pg, 500 pg, 1 ng, 50 ng, 100 ng, 500 ng, 1 μg or 50 μg. Said dose may be a single flat dose.

In a preferred embodiment, a polypeptide may be dosed in an amount of greater than 250 μg. In one embodiment, a polypeptide of the invention may be dosed in an amount of greater than 500 μg, 1 mg, 10 mg, 100 mg, 500 mg, 1 g or 5 g. In one embodiment, a polypeptide of the invention may be dosed in an amount equal to or less than 10 g, 5 g, 1 g, 500 mg, 100 mg, 10 mg or 1 mg. Preferably, a polypeptide of the invention is dosed at an amount of 251 μg to 10 g, 251 μg to 5 g, 251 μg to 1 g, 251 μg to 500 mg, 251 μg to 100 mg, 251 μg to 10 mg, or 251 μg to 1000 μg, e.g. 251 μg to 500 μg. In one embodiment, a polypeptide of the invention is dosed in an amount of 500 μg to 5 g, e.g. 1 mg to 1 g or 1 g to 3 g. This is made possible by the non-toxic (e.g. substantially non-toxic) nature of the polypeptides of the invention.

Fluid dosage forms are typically prepared utilising the polypeptide and a pyrogen-free sterile vehicle. The clostridial neurotoxin, depending on the vehicle and concentration used, can be either dissolved or suspended in the vehicle. In preparing solutions the polypeptide can be dissolved in the vehicle, the solution being made isotonic if necessary by addition of sodium chloride and sterilised by filtration through a sterile filter using aseptic techniques before filling into suitable sterile vials or ampoules and sealing. Alternatively, if solution stability is adequate, the solution in its sealed containers may be sterilised by autoclaving. Advantageously additives such as buffering, solubilising, stabilising, preservative or bactericidal, suspending or emulsifying agents and or local anaesthetic agents may be dissolved in the vehicle.

Dry powders, which are dissolved or suspended in a suitable vehicle prior to use, may be prepared by filling pre-sterilised ingredients into a sterile container using aseptic technique in a sterile area. Alternatively the ingredients may be dissolved into suitable containers using aseptic technique in a sterile area. The product is then freeze dried and the containers are sealed aseptically.

Parenteral suspensions, suitable for an administration route described herein, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, e.g. by gamma irradiation.

Advantageously, a suspending agent for example polyvinylpyrrolidone is included in the composition(s) to facilitate uniform distribution of the components.

Administration in accordance with the present invention may take advantage of a variety of delivery technologies including microparticle encapsulation, or high-pressure aerosol impingement.

The polypeptides of the invention are preferably administered iteratively (e.g. up to 5, 10, 15 or 20 times) as part of a treatment regimen. Iterative administration means administration at least two times, e.g. at least 5, 10, 15 or 20 times. Thus, in one embodiment, polypeptides of the invention may be administered two or more times to treat a subject (e.g. to treat pain in a subject). This is particularly pertinent for the treatment of chronic conditions, such as chronic pain, where ongoing treatment is typically necessary. In one embodiment a polypeptide of the invention may be administered weekly, twice monthly, monthly, every two months, every six months or annually, preferably at least twice annually or annually. In one embodiment, a polypeptide of the invention is administered two or more times in a period of 10 years, 5 years, 2 years or 1 year. Preferably, a polypeptide of the invention is administered two or more times in a period of 1 year. Treatment may continue for at least 6 months, 1 year, 2 years, 3 years, 5 years, 10 years, 15 years, 20 years, 25 years or 30 years.

It is preferred that the polypeptide is not administered together with a further therapeutic or diagnostic agent (e.g. a nucleic acid, protein, peptide or small molecule therapeutic or diagnostic agent) additional to the catalytically inactive clostridial neurotoxin L-chain, H_{N} domain and/or H_{C} domain. For example, in one embodiment the polypeptide is not administered with a further analgesic and/or anti-inflammatory. In one embodiment a polypeptide of the invention is not administered together with a covalently associated therapeutic agent. In one embodiment a polypeptide of the invention is not administered together with a non-covalently associated therapeutic agent.

The polypeptides described herein may be used to treat a subject suffering from one or more types of pain. The pain may be chronic or acute pain. The pain may be one or more selected from the following four categories of pain: nociceptive pain; neuropathic pain; mixed pain; and pain of an unknown origin. Nociceptive pain may be caused by a known noxious stimulus to a nociceptor (pain receptor) and may be somatic or visceral. Neuropathic pain may be pain initiated or caused by a primary lesion or dysfunction in the nervous system. Mixed pain may be a combination of nociceptive pain and neuropathic pain.

Examples of pain (e.g. of chronic pain) treated by the present invention include neuropathic pain, inflammatory pain, headache pain, somatic pain, visceral pain, referred pain, allodynia, mixed pain, and post-operative pain.

The term “pain” as used here, means any unpleasant sensory experience, usually associated with a physical disorder. The physical disorder may or may not be apparent to a clinician. Pain is of two types: chronic and acute. An “acute pain” is a pain of short duration having a sudden onset. One type of acute pain, for example, is cutaneous pain felt on injury to the skin or other superficial tissues, such as caused by a cut or a burn. Cutaneous nociceptors terminate just below the skin, and due to the high concentration of nerve endings, produce a well-defined, localized pain of short duration. “Chronic pain” is a pain other than an acute pain.

The polypeptides of the invention may be used to treat pain caused by or otherwise associated with any of the following neuropathic pain conditions. “Neuropathic pain” means abnormal sensory input, resulting in discomfort, from the peripheral nervous system, central nervous systems, or both. Symptoms of neuropathic pain can involve persistent, spontaneous pain, as well as allodynia (a painful response to a stimulus that normally is not painful), hyperalgesia (an accentuated response to a painful stimulus that usually causes only a mild discomfort, such as a pin prick), or hyperpathia (where a short discomfort becomes a prolonged severe pain). Neuropathic pain may be caused by any of the following:
1. A traumatic insult, such as, for example, a nerve compression injury (e.g., a nerve crush, a nerve stretch, a nerve entrapment or an incomplete nerve transsection); a spinal cord injury (e.g., a hemisection of the spinal cord); a limb amputation; a contusion; an inflammation (e.g., an inflammation of the spinal cord); or a surgical procedure.
2. An ischemic event, including, for example, a stroke and heart attack.
3. An infectious agent.
4. Exposure to a toxic agent, including, for example, a drug, an alcohol, a heavy metal (e.g., lead, arsenic, mercury), an industrial agent (e.g., a solvent, fumes from a glue) or nitrous oxide.
5. A disease, including, for example, an inflammatory disorder, a neoplastic tumour, an acquired immune deficiency syndrome (AIDS), Lymes disease, a leprosy, a metabolic disease, a peripheral nerve disorder, like neuroma, a mononeuropathy or a polyneuropathy.

Types of neuropathic pain include the following:

### 1. Neuralgia.

A neuralgia is a pain that radiates along the course of one or more specific nerves usually without any demonstrable pathological change in the nerve structure. The causes of neuralgia are varied. Chemical irritation, inflammation, trauma (including surgery), compression by nearby structures (for instance, tumours), and infections may all lead to neuralgia. In many cases, however, the cause is unknown or unidentifiable. Neuralgia is most common in elderly persons, but it may occur at any age. A neuralgia, includes, without limitation, a trigeminal neuralgia, a post-herpetic neuralgia, a postherpetic neuralgia, a glossopharyngeal neuralgia, a sciatica and an atypical facial pain.

Neuralgia is pain in the distribution of a nerve or nerves. Examples are trigeminal neuralgia, atypical facial pain, and postherpetic neuralgia (caused by shingles or herpes). The affected nerves are responsible for sensing touch, temperature, and pressure in the facial area from the jaw to the forehead. The disorder generally causes short episodes of excruciating pain, usually for less than two minutes and on only one side of the face. The pain can be described in a variety of ways such as “stabbing,” “sharp,” “like lightning,” “burning,” and even “itchy”. In the atypical form of TN, the pain can also present as severe or merely aching and last for extended periods. The pain associated with TN is recognized as one the most excruciating pains that can be experienced.

Simple stimuli such as eating, talking, washing the face, or any light touch or sensation can trigger an attack (even the sensation of a gentle breeze). The attacks can occur in clusters or as an isolated attack.

Symptoms include sharp, stabbing pain or constant, burning pain located anywhere, usually on or near the surface of the body, in the same location for each episode; pain along the path of a specific nerve; impaired function of an affected body part due to pain, or muscle weakness due to concomitant motor nerve damage; increased sensitivity of the skin or numbness of the affected skin area (feeling similar to a local anaesthetic such as a Novocaine shot); and any touch or pressure is interpreted as pain. Movement may also be painful.

Trigeminal neuralgia is the most common form of neuralgia. It affects the main sensory nerve of the face, the trigeminal nerve (“trigeminal” literally means “three origins”, referring to the division of the nerve into 3 branches). This condition involves sudden and short attacks of severe pain on the side of the face, along the area supplied by the trigeminal nerve on that side. The pain attacks may be severe enough to cause a facial grimace, which is classically referred to as a painful tic (tic douloureux). Sometimes, the cause of trigeminal neuralgia is a blood vessel or small tumour pressing on the nerve. Disorders such as multiple sclerosis (an inflammatory disease affecting the brain and spinal cord), certain forms of arthritis, and diabetes (high blood sugar) may also cause trigeminal neuralgia, but a cause is not always identified. In this condition, certain movements such as chewing, talking, swallowing, or touching an area of the face may trigger a spasm of excruciating pain.

A related but rather uncommon neuralgia affects the glosso-pharyngeal nerve, which provides sensation to the throat. Symptoms of this neuralgia are short, shock-like episodes of pain located in the throat.

Neuralgia may occur after infections such as shingles, which is caused by the varicella-zoster virus, a type of herpesvirus. This neuralgia produces a constant burning pain after the shingles rash has healed. The pain is worsened by movement of or contact with the affected area. Not all of those diagnosed with shingles go on to experience postherpetic neuralgia, which can be more painful than shingles. The pain and sensitivity can last for months or even years. The pain is usually in the form of an intolerable sensitivity to any touch but especially light touch. Postherpetic neuralgia is not restricted to the face; it can occur anywhere on the body but usually occurs at the location of the shingles rash. Depression is not uncommon due to the pain and social isolation during the illness.

Postherpetic neuralgia may be debilitating long after signs of the original herpes infection have disappeared. Other infectious diseases that may cause neuralgia are syphilis and Lyme disease.

Diabetes is another common cause of neuralgia. This very common medical problem affects almost 1 out of every 20 Americans during adulthood. Diabetes damages the tiny arteries that supply circulation to the nerves, resulting in nerve fibre malfunction and sometimes nerve loss. Diabetes can produce almost any neuralgia, including trigeminal neuralgia, carpal tunnel syndrome (pain and numbness of the hand and wrist), and meralgia paresthetica (numbness and pain in the thigh due to damage to the lateral femoral cutaneous nerve). Strict control of blood sugar may prevent diabetic nerve damage and may accelerate recovery in subjects who do develop neuralgia.

Other medical conditions that may be associated with neuralgias are chronic renal insufficiency and porphyria—a hereditary disease in which the body cannot rid itself of certain substances produced after the normal breakdown of blood in the body. Certain drugs may also cause this problem.

### 2. Deafferentation.

Deafferentation indicates a loss of the sensory input from a portion of the body, and can be caused by interruption of either peripheral sensory fibres or nerves from the central nervous system. A deafferentation pain syndrome, includes, without limitation, an injury to the brain or spinal cord, a post-stroke pain, a phantom pain, a paraplegia, a brachial plexus avulsion injuries, lumbar radiculopathies.

### 3. Complex Regional Pain Syndromes (CRPSs)

CRPS is a chronic pain syndrome resulting from sympathetically-maintained pain, and presents in two forms. CRPS 1 currently replaces the term “reflex sympathetic dystrophy syndrome”. It is a chronic nerve disorder that occurs most often in the arms or legs after a minor or major injury. CRPS 1 is associated with severe pain; changes in the nails, bone, and skin; and an increased sensitivity to touch in the affected limb. CRPS 2 replaces the term causalgia, and results from an identified injury to the nerve. A CRPS, includes, without limitation, a CRPS Type I (reflex sympathetic dystrophy) and a CRPS Type II (causalgia).

### 4. Neuropathy.

A neuropathy is a functional or pathological change in a nerve and is characterized clinically by sensory or motor neuron abnormalities.

Central neuropathy is a functional or pathological change in the central nervous system.

Peripheral neuropathy is a functional or pathological change in one or more peripheral nerves. The peripheral nerves relay information from your central nervous system (brain and spinal cord) to muscles and other organs and from your skin, joints, and other organs back to your brain. Peripheral neuropathy occurs when these nerves fail to carry information to and from the brain and spinal cord, resulting in pain, loss of sensation, or inability to control muscles. In some cases, the failure of nerves that control blood vessels, intestines, and other organs results in abnormal blood pressure, digestion problems, and loss of other basic body processes. Risk factors for neuropathy include diabetes, heavy alcohol use, and exposure to certain chemicals and drugs. Some people have a hereditary predisposition for neuropathy. Prolonged pressure on a nerve is another risk for developing a nerve injury. Pressure injury may be caused by prolonged immobility (such as a long surgical procedure or lengthy illness) or compression of a nerve by casts, splints, braces, crutches, or other devices. Polyneuropathy implies a widespread process that usually affects both sides of the body equally. The symptoms depend on which type of nerve is affected. The three main types of nerves are sensory, motor, and autonomic. Neuropathy can affect any one or a combination of all three types of nerves. Symptoms also depend on whether the condition affects the whole body or just one nerve (as from an injury). The cause of chronic inflammatory polyneuropathy is an abnormal immune response. The specific antigens, immune processes, and triggering factors are variable and in many cases are unknown. It may occur in association with other conditions such as HIV, inflammatory bowel disease, lupus erythematosis, chronic active hepatitis, and blood cell abnormalities.

Peripheral neuropathy may involve a function or pathological change to a single nerve or nerve group (mononeuropathy) or a function or pathological change affecting multiple nerves (polyneuropathy).

Peripheral neuropathies may include the following:

### Hereditary Disorders

Charcot-Marie-Tooth disease
Friedreich's ataxia

### Systemic or Metabolic Disorders

Diabetes (diabetic neuropathy)
Dietary deficiencies (especially vitamin B-12)
Excessive alcohol use (alcoholic neuropathy)
Uremia (from kidney failure)
Cancer

### Infectious or Inflammatory Conditions

AIDS
Hepatitis
Colorado tick fever
diphtheria
Guillain-Barre syndrome
HIV infection without development of AIDS
leprosy
Lyme
polyarteritis nodosa
rheumatoid arthritis
sarcoidosis
Sjogren syndrome
syphilis
systemic lupus erythematosus
amyloid

### Exposure to Toxic Compounds

sniffing glue or other toxic compounds
nitrous oxide
industrial agents—especially solvents
heavy metals (lead, arsenic, mercury, etc.)
Neuropathy secondary to drugs like analgesic nephropathy

### Miscellaneous Causes

ischemia (decreased oxygen/decreased blood flow)
prolonged exposure to cold temperature
   a. Polyneuropathy

Polyneuropathy is a peripheral neuropathy involving the loss of movement or sensation to an area caused by damage or destruction to multiple peripheral nerves. Polyneuropathic pain, includes, without limitation, post-polio syndrome, postmastectomy syndrome, diabetic neuropathy, alcohol neuropathy, amyloid, toxins, AIDS, hypothyroidism, uremia, vitamin deficiencies, chemotherapy-induced pain, 2′,3′-didexoycytidine (ddC) treatment, Guillain-Barre syndrome or Fabry's disease.

b. Mononeuropathy

Mononeuropathy is a peripheral neuropathy involving loss of movement or sensation to an area caused by damage or destruction to a single peripheral nerve or nerve group. Mononeuropathy is most often caused by damage to a local area resulting from injury or trauma, although occasionally systemic disorders may cause isolated nerve damage (as with mononeuritis multiplex). The usual causes are direct trauma, prolonged pressure on the nerve, and compression of the nerve by swelling or injury to nearby body structures. The damage includes destruction of the myelin sheath (covering) of the nerve or of part of the nerve cell (the axon). This damage slows or prevents conduction of impulses through the nerve. Mononeuropathy may involve any part of the body. Mononeuropathic pain, includes, without limitation, a sciatic nerve dysfunction, a common peroneal nerve dysfunction. a radial nerve dysfunction, an ulnar nerve dysfunction, a cranial mononeuropathy VI, a cranial mononeuropathy VII, a cranial mononeuropathy Ill (compression type), a cranial mononeuropathy Ill (diabetic type), an axillary nerve dysfunction, a carpal tunnel syndrome, a femoral nerve dysfunction, a tibial nerve dysfunction, a Bell's palsy, a thoracic outlet syndrome, a carpal tunnel syndrome and a sixth (abducent) nerve palsy

c. Generalized Peripheral Neuropathies

Generalized peripheral neuropathies are symmetrical, and usually due to various systematic illnesses and disease processes that affect the peripheral nervous system in its entirety. They are further subdivided into several categories:
i. Distal axonopathies are the result of some metabolic or toxic derangement of neurons. They may be caused by metabolic diseases such as diabetes, renal failure, deficiency syndromes such as malnutrition and alcoholism, or the effects of toxins or drugs. Distal axonopathy (aka dying back neuropathy) is a type of peripheral neuropathy that results from some metabolic or toxic derangement of peripheral nervous system (PNS) neurons. It is the most common response of nerves to metabolic or toxic disturbances, and as such may be caused by metabolic diseases such as diabetes, renal failure, deficiency syndromes such as malnutrition and alcoholism, or the effects of toxins or drugs. The most common cause of distal axonopathy is diabetes, and the most common distal axonopathy is diabetic neuropathy.
ii. Myelinopathies are due to a primary attack on myelin causing an acute failure of impulse conduction. The most common cause is acute inflammatory demyelinating polyneuropathy (AIDP; aka Guillain-Barre syndrome), though other causes include chronic inflammatory demyelinating syndrome (CIDP), genetic metabolic disorders (e.g., leukodystrophy), or toxins. Myelinopathy is due to primary destruction of myelin or the myelinating Schwann cells, which leaves the axon intact, but causes an acute failure of impulse conduction. This demyelination slows down or completely blocks the conduction of electical impulses through the nerve. The most common cause is acute inflammatory demyelinating polyneuropathy (AIDP, better known as Guillain-Barre syndrome), though other causes include chronic inflammatory demyelinating polyneuropathy (CIDP), genetic metabolic disorders (e.g., leukodystrophy or Charcot-Marie-Tooth disease), or toxins.
iii. Neuronopathies are the result of destruction of peripheral nervous system (PNS) neurons. They may be caused by motor neurone diseases, sensory neuronopathies (e.g., Herpes zoster), toxins or autonomic dysfunction. Neurotoxins may cause neuronopathies, such as the chemotherapy agent vincristine. Neuronopathy is dysfunction due to damage to neurons of the peripheral nervous system (PNS), resulting in a peripheral neuropathy. It may be caused by motor neurone diseases, sensory neuronopathies (e.g., Herpes zoster), toxic substances or autonomic dysfunction. A person with neuronopathy may present in different ways, depending on the cause, the way it affects the nerve cells, and the type of nerve cell that is most affected.
iv. Focal entrapment neuropathies (e.g., carpal tunnel syndrome).

Where the pain is neuropathic pain, in one embodiment, the polypeptide used does not comprise a catalytically inactive BoNT/X L-chain, a BoNT/X translocation domain (H_{N} domain) and/or a BoNT/X receptor binding domain (H_{C} domain). In one embodiment, where the pain is neuropathic pain the polypeptide used does not comprise a catalytically inactive BoNT/X L-chain and translocation domain (H_{N} domain), optionally in combination with an H_{C} domain from a different (i.e. non-BoNT/X) clostridial neurotoxin (e.g. an H_{C} domain from BoNT/B). Preferably, where the pain is neuropathic pain the polypeptide used does not comprise a catalytically inactive BoNT/X L-chain and translocation domain (H_{N} domain), and a BoNT/B H_{C} domain.

The polypeptides of the invention may be used to treat pain caused by or otherwise associated with any of the following inflammatory conditions. Similarly, a polypeptide of the invention may be used to treat one or more of the following inflammatory conditions.

### A. Arthritic Disorder

Arthritic disorders include, for example, a rheumatoid arthritis; a juvenile rheumatoid arthritis; a systemic lupus erythematosus (SLE); a gouty arthritis; a scleroderma; an osteoarthritis; a psoriatic arthritis; an ankylosing spondylitis; a Reiter's syndrome (reactive arthritis); an adult Still's disease; an arthritis from a viral infection; an arthritis from a bacterial infection, such as, e.g., a gonococcal arthritis and a non-gonococcal bacterial arthritis (septic arthritis); a Tertiary Lyme disease; a tuberculous arthritis; and an arthritis from a fungal infection, such as, e.g. a blastomycosis

### B. Autoimmune Diseases

Autoimmune diseases include, for example, a Guillain-Barre syndrome, a Hashimoto's thyroiditis, a pernicious anemia, an Addison's disease, a type I diabetes, a systemic lupus erythematosus, a dermatomyositis, a Sjogren's syndrome, a lupus erythematosus, a multiple sclerosis, a myasthenia gravis, a Reiter's syndrome and a Grave's disease.

### C. Connective Tissue Disorder

Connective tissue disorders include, for example, a spondyloarthritis a dermatomyositis, and a fibromyalgia.

### D. Injury

Inflammation caused by injury, including, for example, a crush, puncture, stretch of a tissue or joint, may cause chronic inflammatory pain.

### E. Infection

Inflammation caused by infection, including, for example, a tuberculosis or an interstitial keratitis may cause chronic inflammatory pain.

### F. Neuritis

Neuritis is an inflammatory process affecting a nerve or group of nerves. Symptoms depend on the nerves involved, but may include pain, paresthesias, paresis, or hypesthesia (numbness).

### Examples Include:

a. Brachial neuritis
b. Retrobulbar neuropathy, an inflammatory process affecting the part of the optic nerve lying immediately behind the eyeball.
c. Optic neuropathy, an inflammatory process affecting the optic nerve causing sudden, reduced vision in the affected eye. The cause of optic neuritis is unknown. The sudden inflammation of the optic nerve (the nerve connecting the eye and the brain) leads to swelling and destruction of the myelin sheath. The inflammation may occasionally be the result of a viral infection, or it may be caused by autoimmune diseases such as multiple sclerosis. Risk factors are related to the possible causes.
d. Vestibular neuritis, a viral infection causing an inflammatory process affecting the vestibular nerve.

### G. Joint Inflammation

Inflammation of the joint, such as that caused by bursitis or tendonitis, for example, may cause chronic inflammatory pain.

H. Sunburn and/or UV-Induced Damage

The polypeptides of the invention may be used to treat pain caused by or otherwise associated with any of the following headache conditions. A headache (medically known as cephalgia) is a condition of mild to severe pain in the head; sometimes neck or upper back pain may also be interpreted as a headache. It may indicate an underlying local or systemic disease or be a disorder in itself.

### A. Muscular/Myogenic Headache

Muscular/myogenic headaches appear to involve the tightening or tensing of facial and neck muscles; they may radiate to the forehead. Tension headache is the most common form of myogenic headache.

A tension headache is a condition involving pain or discomfort in the head, scalp, or neck, usually associated with muscle tightness in these areas. Tension headaches result from the contraction of neck and scalp muscles. One cause of this muscle contraction is a response to stress, depression or anxiety. Any activity that causes the head to be held in one position for a long time without moving can cause a headache. Such activities include typing or use of computers, fine work with the hands, and use of a microscope. Sleeping in a cold room or sleeping with the neck in an abnormal position may also trigger this type of headache. A tension-type headache, includes, without limitation, an episodic tension headache and a chronic tension headache.

### B. Vascular Headache

The most common type of vascular headache is migraine. Other kinds of vascular headaches include cluster headaches, which cause repeated episodes of intense pain, and headaches resulting from high blood pressure

### 1. Migraine

A migraine is a heterogeneous disorder that generally involves recurring headaches. Migraines are different from other headaches because they occur with other symptoms, such as, e.g., nausea, vomiting, or sensitivity to light. In most people, a throbbing pain is felt only on one side of the head. Clinical features such as type of aura symptoms, presence of prodromes, or associated symptoms such as vertigo, may be seen in subgroups of subjects with different underlying pathophysiological and genetic mechanisms. A migraine headache, includes, without limitation, a migraine without aura (common migraine), a migraine with aura (classic migraine), a menstrual migraine, a migraine equivalent (acephalic headache), a complicated migraine, an abdominal migraine and a mixed tension migraine.

### 2. Cluster Headache

Cluster headaches affect one side of the head (unilateral) and may be associated with tearing of the eyes and nasal congestion. They occurs in clusters, happening repeatedly every day at the same time for several weeks and then remitting.

### D. High Blood Pressure Headache

### E. Traction and Inflammatory Headache

Traction and inflammatory headaches are usually symptoms of other disorders, ranging from stroke to sinus infection.

### F. Hormone Headache

### G. Rebound Headache

Rebound headaches, also known as medication overuse headaches, occur when medication is taken too frequently to relieve headache. Rebound headaches frequently occur daily and can be very painful.

### H. Chronic Sinusitis Headache

Sinusitis is inflammation, either bacterial, fungal, viral, allergic or autoimmune, of the paranasal sinuses. Chronic sinusitis is one of the most common complications of the common cold. Symptoms include: Nasal congestion; facial pain; headache; fever; general malaise; thick green or yellow discharge; feeling of facial ‘fullness’ worsening on bending over. In a small number of cases, chronic maxillary sinusitis can also be brought on by the spreading of bacteria from a dental infection. Chronic hyperplastic eosinophilic sinusitis is a noninfective form of chronic sinusitis.

### I. An Organic Headache

### J. Ictal Headaches

Ital headaches are headaches associated with seizure activity.

The polypeptides of the invention may be used to treat pain caused by or otherwise associated with any of the following somatic pain conditions. Somatic pain originates from ligaments, tendons, bones, blood vessels, and even nerves themselves. It is detected with somatic nociceptors. The scarcity of pain receptors in these areas produces a dull, poorly-localized pain of longer duration than cutaneous pain; examples include sprains and broken bones. Additional examples include the following.

### A. Excessive Muscle Tension

Excessive muscle tension can be caused, for example, by a sprain or a strain.

### B. Repetitive Motion Disorders

Repetitive motion disorders can result from overuse of the hands, wrists, elbows, shoulders, neck, back, hips, knees, feet, legs, or ankles.

### C. Muscle Disorders

Muscle disorders causing somatic pain include, for example, a polymyositis, a dermatomyositis, a lupus, a fibromyalgia, a polymyalgia rheumatica, and a rhabdomyolysis.

### D. Myalgia

Myalgia is muscle pain and is a symptom of many diseases and disorders. The most common cause for myalgia is either overuse or over-stretching of a muscle or group of muscles. Myalgia without a traumatic history is often due to viral infections. Longer-term myalgias may be indicative of a metabolic myopathy, some nutritional deficiencies or chronic fatigue syndrome.

### E. Infection

Infection can cause somatic pain. Examples of such infection include, for example, an abscess in the muscle, a trichinosis, an influenza, a Lyme disease, a malaria, a Rocky Mountain spotted fever, Avian influenza, the common cold, community-acquired pneumonia, meningitis, monkeypox, Severe Acute Respiratory Syndrome, toxic shock syndrome, trichinosis, typhoid fever, and upper respiratory tract infection.

### F. Drugs

Drugs can cause somatic pain. Such drugs include, for example, cocaine, a statin for lowering cholesterol (such as atorvastatin, simvastatin, and lovastatin), and an ACE inhibitor for lowering blood pressure (such as enalapril and captopril)

The polypeptides of the invention may be used to treat pain caused by or otherwise associated with any of the following visceral pain conditions. Visceral pain originates from body's viscera, or organs. Visceral nociceptors are located within body organs and internal cavities. The even greater scarcity of nociceptors in these areas produces pain that is usually more aching and of a longer duration than somatic pain. Visceral pain is extremely difficult to localise, and several injuries to visceral tissue exhibit “referred” pain, where the sensation is localised to an area completely unrelated to the site of injury. Examples of visceral pain include the following.

A. Functional Visceral Pain Functional visceral pain includes, for example, an irritable bowel syndrome and a chronic functional abdominal pain (CFAP), a functional constipation and a functional dyspepsia, a non-cardiac chest pain (NCCP) and a chronic abdominal pain.

### B. Chronic Gastrointestinal Inflammation

Chronic gastrointestinal inflammation includes, for example, a gastritis, an inflammatory bowel disease, like, e.g., a Crohn's disease, an ulcerative colitis, a microscopic colitis, a diverticulitis and a gastroenteritis; an interstitial cystitis; an intestinal ischemia; a cholecystitis; an appendicitis; a gastroesophageal reflux; an ulcer, a nephrolithiasis, an urinary tract infection, a pancreatitis and a hernia.

### C. Autoimmune Pain

Autoimmune pain includes, for example, a sarcoidosis and a vasculitis.

### D. Organic Visceral Pain

Organic visceral pain includes, for example, pain resulting from a traumatic, inflammatory or degenerative lesion of the gut or produced by a tumour impinging on sensory innervation.

### E. Treatment-Induced Visceral Pain

Treatment-induced visceral pain includes, for example, a pain attendant to chemotherapy therapy or a pain attendant to radiation therapy.

The polypeptides of the invention may be used to treat pain caused by or otherwise associated with any of the following referred pain conditions.

Referred pain arises from pain localized to an area separate from the site of pain stimulation. Often, referred pain arises when a nerve is compressed or damaged at or near its origin. In this circumstance, the sensation of pain will generally be felt in the territory that the nerve serves, even though the damage originates elsewhere. A common example occurs in intervertebral disc herniation, in which a nerve root arising from the spinal cord is compressed by adjacent disc material. Although pain may arise from the damaged disc itself, pain will also be felt in the region served by the compressed nerve (for example, the thigh, knee, or foot). Relieving the pressure on the nerve root may ameliorate the referred pain, provided that permanent nerve damage has not occurred. Myocardial ischaemia (the loss of blood flow to a part of the heart muscle tissue) is possibly the best known example of referred pain; the sensation can occur in the upper chest as a restricted feeling, or as an ache in the left shoulder, arm or even hand.

The polypeptides of the invention may be used to treat post-operative pain.

Post-operative (e.g. post-surgical) pain is an unpleasant sensation that results from a surgical procedure. Post-operative pain may be caused by damage to tissue by an incision, the procedure itself, the closing of the wound, and any force that is applied during the procedure. Pain after surgery (e.g. post-operative pain) can also stem from factors that accompany surgery. For example, a subject may suffer back pain due to the way the subject was positioned on the surgical table, or chest pain may be due to an incision in the chest area. Throat pain may also occur after general anesthesia because the insertion of the breathing tube can cause irritation. However, most common is post-operative pain caused by cutting into the skin and muscle from a surgical incision.

For example, the surgical procedure (or more particularly, surgical incision) may represent a ‘noxious stimulus’ causing pain. Noxious stimuli, stimuli which can elicit tissue damage, can activate the release of neurotransmitters from nociceptive afferent terminals and the release of neuropeptides such as Substance P and Calcitonin gene related peptide (CGRP) from sensory terminals. The noxious information is then transduced from the peripheral nervous system to the central nervous system, where pain is perceived by the individual.

Post-operative pain can be caused by the combination of inflammation and neural tissue damage. For example, degranulation of activated mast cells in response to tissue injury can result in the release of various substances including proteases, cytokines, serotonin and extracellular space. These substances can sensitize (activate at a lower threshold) primary afferent neurons to produce pain hypersensitivity. As tissue is extensively innervated, any region of the body is susceptible to nerve damage from surgery.

Reference to surgery means a medical procedure involving the treatment of an injury or disease in a subject comprising subjecting a part of the body to an incision (optionally removing or repairing a damaged part of the body). Although the level of invasiveness (e.g. level of surgical incision required) may vary amongst surgery types, surgery having a level of invasiveness that causes pain in the subject once surgery is complete is intended to be encompassed.

The surgery may comprise an incision to skin and/or fascia and/or muscle. Preferably, the surgery comprises an incision to the skin.

The surgery is not limited to that which may be carried out by a physician, but also includes for example dental surgery. Non-limiting examples of surgery include appendectomy, breast biopsy, breast augmentation or reduction, facelift, cholecystectomy, coronary artery bypass, debridement (e.g. of a wound, a burn, or infection), skin graft, organ transplant and tonsillectomy.

Preferably, “post-operative” may refer to a time period beginning at most one day subsequent to surgery (e.g. post-surgery). In other words, the term “post-operative” may refer to a time period beginning not greater than one day post-surgery. For example, the term “post-operative” may refer to a time point beginning 1-20 hours post-surgery; optionally 2-15 hours post-surgery; optionally 5-10 hours post-surgery. Such time may represent a time period beginning at the chronological interface at which the analgesic effects from a surgical anaesthetic administered to a subject diminish (e.g. taper) and thus the subject begins to perceive pain.

Furthermore, the term “post-operative” may be used interchangeably with the term “post-surgical”, as ‘operative’ is used in the sense of ‘surgery’ herein.

Similarly, the term “post-operative pain” may refer to pain that is perceived (or more particularly, begins to be perceived) for a time period beginning at most one day subsequent to surgery (e.g. post-surgery). In other words, the term “post-operative pain” may refer to pain that is perceived by a subject for a time period beginning not greater than one day post-surgery. For example, the term “post-operative pain” may refer to pain that is perceived for a time period beginning 1-20 hours post-surgery; optionally 2-15 hours post-surgery; optionally 5-10 hours post-surgery.

Said time period may be 1-50 weeks; for example 5-45 weeks, 10-40 weeks or 10-35 weeks post-surgery.

This contrasts with the term “peri-operative”, which may refer, for example, to a time period at or around the time that a subject is undergoing surgery (e.g. the time when the subject is in the operating theatre), suitably a period beginning at least 1 hour pre-surgery and/or ending less than 1 hour post-surgery.

The present invention addresses a wide range of pain conditions, e.g. chronic pain conditions. In some embodiments, the polypeptides of the invention are for treating cancerous and non-cancerous pain.

Preferably, polypeptides of the invention are used to treat neuropathic pain. The neuropathic pain may be acute or chronic. In one embodiment the neuropathic pain is injury-induced neuropathic pain (neuropathic pain associated with an injury). In one embodiment the neuropathic pain is chemotherapy-induced neuropathic pain (neuropathic pain associated with chemotherapy).

Preferably, polypeptides of the invention are used to treat inflammatory pain. The inflammatory pain may be acute or chronic. In one embodiment the inflammatory pain may be a burn. For example, the inflammatory pain may be caused by UV damage (e.g. UV-B damage).

Most preferably, the polypeptides of the invention are used to treat bladder pain syndrome, phantom limb pain, or migraine pain. The bladder pain syndrome may be caused by or associated with interstitial cystitis.

Treating pain preferably means reducing pain. In other words, in one embodiment, administration of a polypeptide of the invention reduces pain in a subject.

In more detail, reference to “reduced” or “reducing” (in terms of pain) preferably means a lower level of pain is perceived by the subject after administration with a polypeptide of the invention (post-administration) when compared with a level of pain perceived by the subject prior to administration (pre-administration). For example, the level of pain perceived may be reduced by at least 15%, 25%, 35%, 45%, 55%, 65%, 75%, 85% or 95% post-administration relative to pre-administration. For example, the level of pain perceived may be reduced by at least 75%; preferably at least 85%; more preferably at least 95% post-administration.

Thus, pain perception by a subject may be reduced following treatment in accordance with the invention. The pain perception (e.g. bladder pain perception) may comprise allodynia and/or hyperalgesia, such as allodynia and hyperalgesia. For example, the level of pain perception by the subject may be reduced or abolished following treatment with a polypeptide of the invention compared with a level of pain perception in a (control) subject that was not administered the polypeptide. Pain perception may be reduced for at least 1 day, 5 days, 1 week, 2 weeks, 1 month, 2 months, 3 months, 4 months, 5 months or 6 months after administration. Pain perception may be reduced for up to 1 year, 6 months, 5 months, 4 months, 3 months, 2 months, 1 month, 2 weeks, 1 week, 5 days or 1 day after administration. For example, pain perception may be reduced for 1 day to 1 year, 5 days to 6 months or 1 week to 3 months after administration. Pain perception may be reduced by at least 1 hour, 12 hours, 1 day, 2 days or 3 days following administration of the polypeptide.

A variety of means for assessing pain perception are known to those skilled in the art. For example, evaluation of mechanical allodynia (either static or dynamic) is routinely used in human pain studies as described in Pogatzki-Zahn et. al. (Pain Rep. 2017 March; 2(2): e588), incorporated herein by reference.

A suitable (albeit non-limiting) method for assessing pain perception in a subject includes the following: Numerical Rating Scale (NRS) score; although the skilled person is aware of other methods which may be used additionally or alternatively such as sensory threshold, pain perception threshold, static mechanical allodynia, dynamic mechanical allodynia, temporal summation, pressure pain threshold, conditioned pain modulation, and temperature threshold.

Other non-limiting examples of pain perception measures include: change from baseline in SF-36 scores at each scheduled time point; amount of rescue medication taken during the study and time to first intake of rescue medication. These may be considered “exploratory” endpoints or pain perception assessment measures.

Thus, in a preferred embodiment, following the administration of a polypeptide of the invention, pain perception may assessed by one or more of: (a) a Numerical Rating Scale (NRS); (b) stimulus-evoked NRS; (c) temperature of the painful area; (d) size of the painful area; (e) time to onset of analgesic effect; (f) peak analgesic effect; (g) time to peak analgesic effect; (h) duration of analgesic effect; and (i) SF-36 quality of life.

The skilled person is aware of such methods for assessing pain perception. For convenience, further description of the Numerical Rating Score and Quality of Life questionnaire Short Form-36 are provided below.

Numerical Rating Scale (NRS): Typically pain perception according to the present invention uses the Numerical Rating Scale (NRS). The NRS is an 11-point scale to assess subject pain perception. Subjects are asked to give a number between 0 and 10 that fits best to their pain intensity. Zero represents ‘no pain at all’ whereas the upper limit, 10, represents ‘the worst pain possible’.

The NRS can be used to assess numerous facets of pain, including spontaneous average pain, spontaneous worst pain and spontaneous current pain. Spontaneous average pain is assessed by asking a subject to select a number that best describes the subject's average pain (e.g. perceived pain) over a period of time, for example at least 6 hours, 12 hours, 24 hours, or at least 48 hours. Spontaneous worst pain is assessed by asking a subject to select a number that best describes the subject's pain at its worst during a specified period, e.g. at least the previous 6 hours, 12 hours, 24 hours or previous 48 hours. Spontaneous current pain is assessed by asking a subject to select a number that best describes how much pain the subject is in at the time of assessment.

The NRS can also be used to assess a subject's pain perception in response to a variety of different stimuli. To assess pain perception in response to a stimulus, the subject will be submitted to stimuli of various nature applied to the painful area. Subjects will be asked what are their current NRS scores pre-dose and post-stimulus.

Examples of stimuli used include: (i) light touch (which can be assessed by measuring pain on the surface of the painful area on radial spokes following application of a von Frey filament as described herein); (ii) pressure (pressure pain threshold), which can be assessed by asking the subject to give a NRS score as increasing pressure is applied using a pressure algometer; and (iii) temperature (which can be assessed by asking the subject for an NRS score for warm, cold and hot stimulation using a thermode applied to the painful area).

Preferably, administration of a polypeptide of the invention reduces the subject's NRS score post-administration (e.g. from a rating of ≥7 to a rating of ≤6) when compared with the subject's NRS score pre-administration.

Quality of Life questionnaire Short Form-36 (SF-36): The SF-36 quality of life questionnaire may be used to assess a subject's pain perception. The SF-36 is a 36-item, subject-reported survey of subject health. The SF-36 consists of eight scaled scores (vitality, physical functioning, bodily pain, general health perceptions, physical role functioning, emotional role functioning, social role functioning and mental health). Each scale is directly transformed into a 0-100 scale on the assumption that each question carries equal weight. The higher the score recorded in the SF-36, the less disability.

Relevant parameters commonly tested in clinical trials for the treatment of pain are known in the art and could be readily selected by one of ordinary skill in the art. Examples of such parameters include, but are not limited to NRS; stimulus-evoked NRS; temperature of the painful area; size of the painful area; time to onset of analgesic effect; peak analgesic effect; time to peak analgesic effect; duration of analgesic effect; and/or SF-36 quality of life as described herein. Methods for assessing these parameters are also known in the art and can be carried out by one of ordinary skill using routine methods and procedures.

Preferably, administration of a polypeptide of the invention increases the subject's SF-36 score post-administration (e.g. from a score of ≤50 to a score of ≥50) when compared with the subject's SF-36 score pre-administration.

In one embodiment the nociceptive threshold of a subject is increased post administration of the polypeptide of the invention. The term “nociceptive threshold” may refer to the level of noxious stimuli required for a subject to perceive pain.

An inflammatory disorder treated by a polypeptide of the invention may an inflammatory disorder of: the nervous system, the cardiovascular system, the respiratory system, the digestion system, the integumentary system, the musculoskeletal system, the urinary system, the reproductive system, the endocrine system, or the lymphatic system.

An inflammatory disorder of the nervous system may be one or more selected from the group consisting of: central nervous system inflammation (e.g. encephalitis, myelitis, meningitis, or arachnoiditis), peripheral nervous system inflammation (e.g. neuritis), eye inflammation (e.g. dacryoadenitis, scleritis, episcleritis, keratitis, retinitis, chorioretinitis, blepharitis, conjunctivitis, or uveitis), and ear inflammation (e.g. otitis externa, otitis media, labyrinthitis, and mastoiditis).

An inflammatory disorder of the cardiovascular system may be one or more selected from the group consisting of: carditis (e.g. endocarditis, myocarditis or pericarditis) and vasculitis (e.g. arteritis, phlebitis or capillaritis).

An inflammatory disorder of the respiratory system may be one or more selected from the group consisting of: an upper respiratory system inflammatory disorder (e.g. sinusitis, rhinitis, pharyngitis or laryngitis), a lower respiratory system inflammatory disorder (e.g. tracheitis, bronchitis, bronchiolitis, pneumonitis or pleuritis), and mediastinitis.

An inflammatory disorder of the digestion system may be one or more selected from the group consisting of: mouth inflammation (e.g. stomatitis, gingivitis, gingivostomatitis, glossitis, tonsillitis, sialadenitis/parotitis, cheilitis, pulpitis or gnathitis), gastrointestinal tract inflammation (e.g. esophagitis, gastritis, gastroenteritis, enteritis, colitis, enterocolitis, duodenitis, ileitis, caecitis, appendicitis or proctitis), and inflammation of the accessory digestive organs (e.g. hepatitis, ascending cholangitis, cholecystitis, pancreatitis or peritonitis).

An inflammatory disorder of the integumentary system may be one or more selected from the group consisting of: dermatitis (e.g. folliculitis), cellulitis, and hidradenitis.

An inflammatory disorder of the musculoskeletal system may be one or more selected from the group consisting of: arthritis, dermatomyositis, soft tissue inflammation (e.g. myositis, synovitis/tenosynovitis, bursitis, enthesitis, fasciitis, capsulitis, epicondylitis, tendinitis or panniculitis), osteochondritis, osteitis/osteomyelitis, spondylitis, periostitis, and chondritis.

An inflammatory disorder of the urinary system may be one or more selected from the group consisting of: nephritis (e.g. glomerulonephritis or pyelonephritis), ureteritis, cystitis, and urethritis.

An inflammatory disorder of the reproductive system may be one or more selected from the group consisting of: inflammation of the female reproductive system (e.g. oophoritis, salpingitis, endometritis, parametritis, cervicitis, vaginitis, vulvitis or mastitis), inflammation of the male reproductive system (e.g. orchitis, epididymitis, prostatitis, seminal vesiculitis, balanitis, posthitis or balanoposthitis), and inflammation associated with pregnancy, birth and/or the new-born (e.g. chorioamnionitis, funisitis or omphalitis).

An inflammatory disorder of the endocrine system may be one or more selected from the group consisting of: insulitis, hypophysitis, thyroiditis, parathyroiditis, and adrenalitis.

An inflammatory disorder of the lymphatic system may be one or more selected from the group consisting of: lymphangitis and lymphadenitis.

Preferably, an inflammatory disorder is one or more selected from: complex regional pain syndrome, endometriosis, rheumatoid arthritis, cystitis, and neuritis. The cystitis is preferably interstitial cystitis. The neuritis is preferably peripheral neuritis.

Polypeptides Comprising a Catalytically Active Clostridial Neurotoxin Light-Chain and Lacking a Functional H_{CC} Domain or H_{C} Domain

The main focus of the present invention is on the utility of polypeptides wherein, when they comprise a clostridial neurotoxin L-chain, the L-chain is catalytically inactive. However, the inventors have also shown that a polypeptide comprising a catalytically active clostridial neurotoxin L-chain, but lacking a functional H_{CC} domain or H_{C} domain of a clostridial neurotoxin, can also be used in the present invention for treating pain (preferably bladder pain), lower urinary tract disorders, and/or inflammatory disorders. Thus, the following aspects are provided.

In one aspect, the invention provides a polypeptide for use in treating pain, wherein the polypeptide comprises a clostridial neurotoxin light-chain (L-chain) and lacks a functional H_{CC} domain or H_{C} domain of a clostridial neurotoxin. In a related aspect, the invention provides a method for treating pain, the method comprising administering a polypeptide to a subject, wherein the polypeptide comprises a clostridial neurotoxin light-chain (L-chain) and lacks a functional H_{CC} domain or H_{C} domain of a clostridial neurotoxin. In a related aspect, the invention provides the use of a polypeptide in the manufacture of a medicament for treating pain, wherein the polypeptide comprises a clostridial neurotoxin light-chain (L-chain) and lacks a functional H_{CC} domain or H_{C} domain of a clostridial neurotoxin.

Thus, in a preferred aspect, the invention provides a polypeptide for use in treating bladder pain, wherein the polypeptide comprises a clostridial neurotoxin light-chain (L-chain) and lacks a functional H_{CC} domain or H_{C} domain of a clostridial neurotoxin. In a related preferred aspect, the invention provides a method for treating bladder pain, the method comprising administering a polypeptide to a subject, wherein the polypeptide comprises a clostridial neurotoxin light-chain (L-chain) and lacks a functional H_{CC} domain or H_{C} domain of a clostridial neurotoxin. In a related preferred aspect, the invention provides the use of a polypeptide in the manufacture of a medicament for treating bladder pain, wherein the polypeptide comprises a clostridial neurotoxin light-chain (L-chain) and lacks a functional H_{CC} domain or H_{C} domain of a clostridial neurotoxin.

In one aspect, the invention provides a polypeptide for use in treating a lower urinary tract disorder, wherein the polypeptide comprises a clostridial neurotoxin light-chain (L-chain) and lacks a functional H_{CC} domain or H_{C} domain of a clostridial neurotoxin. In a related aspect, the invention provides a method for treating a lower urinary tract disorder, the method comprising administering a polypeptide to a subject, wherein the polypeptide comprises a clostridial neurotoxin light-chain (L-chain) and lacks a functional H_{CC} domain or H_{C} domain of a clostridial neurotoxin. In a related aspect, the invention provides the use of a polypeptide in the manufacture of a medicament for treating a lower urinary tract disorder, wherein the polypeptide comprises a clostridial neurotoxin light-chain (L-chain) and lacks a functional H_{CC} domain or H_{C} domain of a clostridial neurotoxin.

In one aspect, the invention provides a polypeptide for use in treating an inflammatory disorder, wherein the polypeptide comprises a clostridial neurotoxin light-chain (L-chain) and lacks a functional H_{CC} domain or H_{C} domain of a clostridial neurotoxin. In a related aspect, the invention provides a method for treating an inflammatory disorder, the method comprising administering a polypeptide to a subject, wherein the polypeptide comprises a clostridial neurotoxin light-chain (L-chain) and lacks a functional H_{CC} domain or H_{C} domain of a clostridial neurotoxin. In a related aspect, the invention provides the use of a polypeptide in the manufacture of a medicament for treating an inflammatory disorder, wherein the polypeptide comprises a clostridial neurotoxin light-chain (L-chain) and lacks a functional H_{CC} domain or H_{C} domain of a clostridial neurotoxin.

The embodiments described above in respect of the polypeptide wherein, when said polypeptide comprises a clostridial neurotoxin L-chain, the chain is catalytically inactive apply equally to aspects employing the polypeptide comprising a clostridial neurotoxin light-chain (L-chain) (i.e. a catalytically active L-chain) and wherein the polypeptide lacks a functional H_{CC} domain or H_{C} domain of a clostridial neurotoxin. Of course, any reference in said embodiments to the L-chain being catalytically inactive and/or any reference to a lack of non-cytotoxic protease activity of the L-chain does not apply to the above-mentioned aspects comprising a clostridial neurotoxin light-chain (L-chain) (i.e. a catalytically active L-chain), and wherein the polypeptide lacks a functional H_{CC} domain or H_{C} domain of a clostridial neurotoxin.

Preferably, the H_{CC} domain or H_{C} domain of a clostridial neurotoxin is absent from a clostridial neurotoxin according to the foregoing aspects.

In the foregoing aspects, it is preferred that the polypeptide lacks a functional H_{CC} domain or H_{C} domain of a clostridial neurotoxin and also lacks any functionally equivalent exogenous ligand Targeting Moiety (TM).

A clostridial neurotoxin L-chain (or polypeptide) may comprise:
(i) a BoNT/A L-chain;
(ii) a BoNT/B L-chain;
(iii) a BoNT/C1 L-chain;
(iv) a BoNT/D L-chain;
(v) a BoNT/E L-chain;
(vi) a BoNT/F L-chain;
(vii) a BoNT/G L-chain;
(viii) a BoNT/X L-chain; or
(ix) a TeNT L-chain.

Said clostridial neurotoxin L-chain (or polypeptide) may consist essentially of, preferably consist of, any one of the above.

In one embodiment a polypeptide comprises a polypeptide sequence having at least:
(i) 70% (e.g. at least 80%, 90%, 95%, 98%, or 100%) sequence identity to residues 1-448 of SEQ ID NO: 51;
(ii) 70% (e.g. at least 80%, 90%, 95%, 98%, or 100%) sequence identity to residues 1-440 of SEQ ID NO: 52;
(iii) 70% (e.g. at least 80%, 90%, 95%, 98%, or 100%) sequence identity to residues 1-441 of SEQ ID NO: 53;
(iv) 70% (e.g. at least 80%, 90%, 95%, 98%, or 100%) sequence identity to residues 1-445 of SEQ ID NO: 54;
(v) 70% (e.g. at least 80%, 90%, 95%, 98%, or 100%) sequence identity to residues 1-422 of SEQ ID NO: 55;
(vi) 70% (e.g. at least 80%, 90%, 95%, 98%, or 100%) sequence identity to residues 1-439 of SEQ ID NO: 56;
(vii) 70% (e.g. at least 80%, 90%, 95%, 98%, or 100%) sequence identity to residues 1-441 of SEQ ID NO: 57;
(viii) 70% (e.g. at least 80%, 90%, 95%, 98%, or 100%) sequence identity to residues 1-414, 1-439, or 1-464 of SEQ ID NO: 59; or
(ix) 70% (e.g. at least 80%, 90%, 95%, 98%, or 100%) sequence identity to residues 1-457 of SEQ ID NO: 58.

The polypeptide may consist essentially of a polypeptide sequence having at least:
(i) 70% (e.g. at least 80%, 90%, 95%, 98%, or 100%) sequence identity to residues 1-448 of SEQ ID NO: 51;
(ii) 70% (e.g. at least 80%, 90%, 95%, 98%, or 100%) sequence identity to residues 1-440 of SEQ ID NO: 52;
(iii) 70% (e.g. at least 80%, 90%, 95%, 98%, or 100%) sequence identity to residues 1-441 of SEQ ID NO: 53;
(iv) 70% (e.g. at least 80%, 90%, 95%, 98%, or 100%) sequence identity to residues 1-445 of SEQ ID NO: 54;
(v) 70% (e.g. at least 80%, 90%, 95%, 98%, or 100%) sequence identity to residues 1-422 of SEQ ID NO: 55;
(vi) 70% (e.g. at least 80%, 90%, 95%, 98%, or 100%) sequence identity to residues 1-439 of SEQ ID NO: 56;
(vii) 70% (e.g. at least 80%, 90%, 95%, 98%, or 100%) sequence identity to residues 1-441 of SEQ ID NO: 57;
(viii) 70% (e.g. at least 80%, 90%, 95%, 98%, or 100%) sequence identity to residues 1-414, 1-439, or 1-464 of SEQ ID NO: 59; or
(ix) 70% (e.g. at least 80%, 90%, 95%, 98%, or 100%) sequence identity to residues 1-457 of SEQ ID NO: 58.

The polypeptide may consist of a polypeptide sequence having at least:
(i) 70% (e.g. at least 80%, 90%, 95%, 98%, or 100%) sequence identity to residues 1-448 of SEQ ID NO: 51;
(ii) 70% (e.g. at least 80%, 90%, 95%, 98%, or 100%) sequence identity to residues 1-440 of SEQ ID NO: 52;
(iii) 70% (e.g. at least 80%, 90%, 95%, 98%, or 100%) sequence identity to residues 1-441 of SEQ ID NO: 53;
(iv) 70% (e.g. at least 80%, 90%, 95%, 98%, or 100%) sequence identity to residues 1-445 of SEQ ID NO: 54;
(v) 70% (e.g. at least 80%, 90%, 95%, 98%, or 100%) sequence identity to residues 1-422 of SEQ ID NO: 55;
(vi) 70% (e.g. at least 80%, 90%, 95%, 98%, or 100%) sequence identity to residues 1-439 of SEQ ID NO: 56;
(vii) 70% (e.g. at least 80%, 90%, 95%, 98%, or 100%) sequence identity to residues 1-441 of SEQ ID NO: 57;
(viii) 70% (e.g. at least 80%, 90%, 95%, 98%, or 100%) sequence identity to residues 1-414, 1-439, or 1-464 of SEQ ID NO: 59; or
(ix) 70% (e.g. at least 80%, 90%, 95%, 98%, or 100%) sequence identity to residues 1-457 of SEQ ID NO: 58.

A clostridial neurotoxin L-chain may comprise a polypeptide sequence having at least 70% sequence identity to any one of SEQ ID NOs: 6, 24, 32, or 40. In one embodiment a clostridial neurotoxin L-chain comprises a polypeptide sequence having at least 80%, 90%, 95% or 98% sequence identity to any one of SEQ ID NOs: 6, 24, 32, or 40. Preferably, a clostridial neurotoxin L-chain comprises (more preferably consists of) any one of SEQ ID NOs: 6, 24, 32 or 40.

A clostridial neurotoxin L-chain may be one encoded by a nucleotide sequence having at least 70% sequence identity to any one of SEQ ID NOs: 5, 23, 31 or 39. In one embodiment a clostridial neurotoxin L-chain is one encoded by a nucleotide sequence having at least 80%, 90%, 95% or 98% sequence identity to any one of SEQ ID NOs: 5, 23, 31 or 39. Preferably, a clostridial neurotoxin L-chain is one encoded by a nucleotide sequence comprising any one of SEQ ID NOs: 5, 23, 31 or 39.

Where a catalytically active L-chain is employed it is preferred that the polypeptide comprises a clostridial neurotoxin translocation domain (H_{N} domain). For example, the polypeptide may comprise a clostridial neurotoxin L-chain and H_{N} domain, and may lack a functional H_{CC} domain or H_{C} domain of a clostridial neurotoxin, said polypeptide may be referred to as LH_{N}. Such a polypeptide may comprise:
(i) a BoNT/A L-chain and a BoNT/A H_{N} domain;
(ii) a BoNT/B L-chain and a BoNT/B H_{N} domain;
(iii) a BoNT/C1 L-chain and a BoNT/C1 H_{N} domain;
(iv) a BoNT/D L-chain and a BoNT/D H_{N} domain;
(v) a BoNT/E L-chain and a BoNT/E H_{N} domain;
(vi) a BoNT/F L-chain and a BoNT/F H_{N} domain;
(vii) a BoNT/G L-chain and a BoNT/G H_{N} domain;
(viii) a BoNT/X L-chain and a BoNT/X H_{N} domain; or
(ix) a TeNT L-chain and a TeNT H_{N} domain.

Said polypeptide may consist essentially of, preferably consist of, any one of the above.

In one embodiment a polypeptide comprises a polypeptide sequence having at least:
(i) 70% (e.g. at least 80%, 90%, 95%, 98%, or 100%) sequence identity to residues 1-871 of SEQ ID NO: 51;
(ii) 70% (e.g. at least 80%, 90%, 95%, 98%, or 100%) sequence identity to residues 1-858 of SEQ ID NO: 52;
(iii) 70% (e.g. at least 80%, 90%, 95%, 98%, or 100%) sequence identity to residues 1-866 of SEQ ID NO: 53;
(iv) 70% (e.g. at least 80%, 90%, 95%, 98%, or 100%) sequence identity to residues 1-862 of SEQ ID NO: 54;
(v) 70% (e.g. at least 80%, 90%, 95%, 98%, or 100%) sequence identity to residues 1-845 of SEQ ID NO: 55;
(vi) 70% (e.g. at least 80%, 90%, 95%, 98%, or 100%) sequence identity to residues 1-864 of SEQ ID NO: 56;
(vii) 70% (e.g. at least 80%, 90%, 95%, 98%, or 100%) sequence identity to residues 1-863 of SEQ ID NO: 57;
(viii) 70% (e.g. at least 80%, 90%, 95%, 98%, or 100%) sequence identity to residues 1-867, 1-892, or 1-917 of SEQ ID NO: 59; or
(ix) 70% (e.g. at least 80%, 90%, 95%, 98%, or 100%) sequence identity to residues 1-879 of SEQ ID NO: 58.

Preferably, the polypeptide consists essentially of a clostridial neurotoxin L-chain and H_{N} domain. In one embodiment a polypeptide consists essentially of a polypeptide sequence having at least:
(i) 70% (e.g. at least 80%, 90%, 95%, 98%, or 100%) sequence identity to residues 1-871 of SEQ ID NO: 51;
(ii) 70% (e.g. at least 80%, 90%, 95%, 98%, or 100%) sequence identity to residues 1-858 of SEQ ID NO: 52;
(iii) 70% (e.g. at least 80%, 90%, 95%, 98%, or 100%) sequence identity to residues 1-866 of SEQ ID NO: 53;
(iv) 70% (e.g. at least 80%, 90%, 95%, 98%, or 100%) sequence identity to residues 1-862 of SEQ ID NO: 54;
(v) 70% (e.g. at least 80%, 90%, 95%, 98%, or 100%) sequence identity to residues 1-845 of SEQ ID NO: 55;
(vi) 70% (e.g. at least 80%, 90%, 95%, 98%, or 100%) sequence identity to residues 1-864 of SEQ ID NO: 56;
(vii) 70% (e.g. at least 80%, 90%, 95%, 98%, or 100%) sequence identity to residues 1-863 of SEQ ID NO: 57;
(viii) 70% (e.g. at least 80%, 90%, 95%, 98%, or 100%) sequence identity to residues 1-867, 1-892, or 1-917 of SEQ ID NO: 59; or
(ix) 70% (e.g. at least 80%, 90%, 95%, 98%, or 100%) sequence identity to residues 1-879 of SEQ ID NO: 58.

More preferably, the polypeptide consists of a clostridial neurotoxin L-chain and H_{N} domain. In one embodiment a polypeptide consists of a polypeptide sequence having at least:
(i) 70% (e.g. at least 80%, 90%, 95%, 98%, or 100%) sequence identity to residues 1-871 of SEQ ID NO: 51;
(ii) 70% (e.g. at least 80%, 90%, 95%, 98%, or 100%) sequence identity to residues 1-858 of SEQ ID NO: 52;
(iii) 70% (e.g. at least 80%, 90%, 95%, 98%, or 100%) sequence identity to residues 1-866 of SEQ ID NO: 53;
(iv) 70% (e.g. at least 80%, 90%, 95%, 98%, or 100%) sequence identity to residues 1-862 of SEQ ID NO: 54;
(v) 70% (e.g. at least 80%, 90%, 95%, 98%, or 100%) sequence identity to residues 1-845 of SEQ ID NO: 55;
(vi) 70% (e.g. at least 80%, 90%, 95%, 98%, or 100%) sequence identity to residues 1-864 of SEQ ID NO: 56;
(vii) 70% (e.g. at least 80%, 90%, 95%, 98%, or 100%) sequence identity to residues 1-863 of SEQ ID NO: 57;
(viii) 70% (e.g. at least 80%, 90%, 95%, 98%, or 100%) sequence identity to residues 1-867, 1-892, or 1-917 of SEQ ID NO: 59; or
(ix) 70% (e.g. at least 80%, 90%, 95%, 98%, or 100%) sequence identity to residues 1-879 of SEQ ID NO: 58.

A polypeptide comprising a clostridial neurotoxin L-chain and translocation domain may comprise a polypeptide sequence having at least 70% sequence identity to any one of SEQ ID NOs: 4, 20, 28, or 36. In one a polypeptide comprising a clostridial neurotoxin L-chain and translocation domain comprises a polypeptide sequence having at least 80%, 90%, 95% or 98% sequence identity to any one of SEQ ID NOs: 4, 20, 28, or 36. Preferably, a polypeptide comprising a clostridial neurotoxin L-chain and translocation domain comprises (more preferably consists of) any one of SEQ ID NOs: 4, 20, 28, or 36.

A polypeptide comprising (or consisting of) a clostridial neurotoxin L-chain and translocation domain may be one encoded by a nucleotide sequence having at least 70% sequence identity to any one of SEQ ID NOs: 3, 19, 27, or 35. In one embodiment a polypeptide comprising (or consisting of) a clostridial neurotoxin L-chain and translocation domain is one encoded by a nucleotide sequence having at least 80%, 90%, 95% or 98% sequence identity to any one of SEQ ID NOs: 3, 19, 27, or 35. Preferably, a polypeptide comprising (or consisting of) a clostridial neurotoxin L-chain and translocation domain is one encoded by a nucleotide sequence comprising any one of SEQ ID NOs: 3, 19, 27, or 35.

Embodiments related to the various therapeutic uses of the invention are intended to be applied equally to methods of treatment, and vice versa.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes); preferably this method is used to align a sequence with a SEQ ID NO described herein to define amino acid position numbering, as described herein.

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\begin{matrix}[ length of the longer sequence plus \\ the number of gaps introduced into the longer \\ sequence in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

Basic: arginine
   lysine
   histidine
Acidic: glutamic acid
   aspartic acid
Polar: glutamine
   asparagine
Hydrophobic: leucine
   isoleucine
   valine
Aromatic: phenylalanine
   tryptophan
   tyrosine
Small: glycine
   alanine
   serine
   threonine
   methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5 to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a clostridial neurotoxin” includes a plurality of such candidate agents and reference to “the clostridial neurotoxin” includes reference to one or more clostridial neurotoxins and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1 shows % SNAP25 cleavage in human neuronal cells administered BoNT/A or BoNT/A(0).

FIG. 2 shows % SNAP25 cleavage in rat neuronal cells administered BoNT/A or BoNT/A(0).

FIG. 3 (A) shows the characteristic startle response of mice suspended by the tail. (B) shows the scoring used in the Digit Abduction Score (DAS) assay.

FIG. 4 (A) presents an experimental schematic for a study using a chronic constriction injury (CCI) model of chronic, neuropathic pain in adult, male Sprague-Dawley rats (220-250 g). Days (D) pre- and post-administration of BoNT/A(0) (60 pg/kg i.pl. administration), BoNT/A (30 pg/kg intraplantar (i.pl.) administration), BoNT/A (60 pg/kg i.pl. administration), vehicle (Gelatine Phosphate Buffer (GPB) i.pl. administration—negative control) or gabapentin (100 mg/kg p.o. administration—positive control) are indicated. Administration occurred on day 0 (DO) and CCI surgery was carried out at D−14. vF indicates that the von Frey test was carried out on the days indicated. (B) shows mechanical sensitivity (measured via the von Frey test) in the ipsilateral paw (i.e. the paw to which control compositions, BoNT/A or BoNT/A(0) were administered) over time for animals administered as described in (A). (C) shows mechanical sensitivity (measured via the von Frey test) in the contralateral paw (i.e. the paw to which control compositions, BoNT/A or BoNT/A(0) were not administered) over time for animals administered as described in (A). (D) shows the bodyweight change for rats administered as described in (A) over time.

FIG. 5 (A) presents an experimental schematic for a study using a model of acute oxaliplatin-induced neuropathic pain in adult, male Sprague-Dawley rats. Oxaliplatin (10 mg/kg intraperitoneal (i.p.) administration) and BoNT/A(0) (1000 pg/kg i.pl. administration), BoNT/A (50 pg/kg i.pl. administration), BoNT/A (100 pg/kg i.pl. administration), BoNT/A (160 pg/kg i.pl. administration), or vehicle (GPB i.pl. administration—negative control) were administered on day 0 (D0). As a further negative control for oxaliplatin treatment, a subset of rats were administered 5% glucose i.p. and GBP (i.pl. administration). Duloxetine (100 mg/kg p.o. administration—positive control) was administered 1 h before testing on D3. Days and hours post-administration are shown. PI indicates that the paw immersion (cold) test was carried out on the days indicated. (B) shows cold sensitivity (measured via the paw immersion test) in the ipsilateral paw (i.e. the paw to which control compositions, BoNT/A or BoNT/A(0) were administered) over time for animals administered as described in (A). (C) shows cold sensitivity (measured via the paw immersion test) in the contralateral paw (i.e. the paw to which control compositions, BoNT/A or BoNT/A(0) were not administered) over time for animals administered as described in (A).

FIG. 6 (A) presents an experimental schematic for a study using a model of chronic oxaliplatin-induced neuropathic pain in adult, male Sprague-Dawley rats (180-210 g). Oxaliplatin was administered at day −2 (D−2). BoNT/A(0) (100 pg/kg i.pl. administration), BoNT/A (100 pg/kg i.pl. administration), or vehicle (GPB i.pl. administration—negative control) was administered on day 0 (DO). Pregabalin (30 mg/kg p.o. administration—positive control) on day 3. Days pre- and post-administration are shown. vF and CP indicate that the von Frey and cold plate tests (respectively) were carried out on the days indicated. (B) shows mechanical sensitivity (measured via the von Frey test) in the ipsilateral paw (i.e. the paw to which control compositions, BoNT/A or BoNT/A(0) were administered) over time for animals administered as described in (A). (C) shows mechanical sensitivity (measured via the von Frey test) in the contralateral paw (i.e. the paw to which control compositions, BoNT/A or BoNT/A(0) were not administered) over time for animals administered as described in (A). (D) shows thermal sensitivity (measured via the cold plate test) over time for animals administered as described in (A).

FIG. 7 (A) presents an experimental schematic for a study using a model of acute ultraviolet-B (UV-B)-burn induced inflammatory pain in adult, male Wistar rats (180-210 g). BoNT/A(0) (100 pg/kg i.pl. administration), BoNT/A (100 pg/kg i.pl. administration), or vehicle (GPB i.pl. administration—negative control) was administered on day 0 (DO). Indomethacin (5 mg/kg p.o. administration—positive control) was administered 1 h before the readout on D3. Rats were exposed to UV-B (500 mJ/cm²) at day 1 (D1). vF indicates that the von Frey test was carried out on the day indicated. (B) shows mechanical sensitivity (measured via the von Frey test) for animals administered as described in (A).

FIG. 8 shows mechanical sensitivity (measured via the von Frey test) for mice administered vehicle, catalytically active chimeric BoNT/XB (0.3 ng/kg, n=10), catalytically active chimeric BoNT/XB (30 ng/kg, n=10), catalytically inactive chimeric BoNT/XB(0) (0.3 ng/kg, n=10), catalytically inactive chimeric BoNT/XB(0) (30 ng/kg, n=10), BoNT/A (160 pg/kg, n=10) or indomethacin (10 mg/kg, n=9). Sensitivity is shown in non-treated animals (baseline), 2 days after administration of BoNTs or vehicle and prior to Complete Freund's adjuvant (CFA) administration (Day 0 CFA, Day 2) as well as 1 day after CFA administration (Day 1 CFA, Day 3). **P<0.1, ***P<0.01 (Dunnett's multiple comparison vs vehicle after Repeated Measure two-way ANOVA).

FIG. 9 shows afferent discharge (area under the curve of normalised data) in a bladder afferent nerve firing assay where bladders were treated with phosphate buffered saline (PBS) control, or 3 pM of: recombinant BoNT/A (rBoNT/A); Dysport (holotoxin); non-complexed BoNT/A isolated from clostridia (nBoNT/A), catalytically-active BoNT/A L-chain and translocation domain without the H_{C} domain (LH_{N}/A), catalytically inactive BoNT/A (BoNT/A(0)), or catalytically inactive BoNT/A L-chain (LC/A(0)). Statistical significance was calculated using 1-way ANOVA followed by a Turkey's multiple comparison test: *P<0.05 versus control, ****P<0.0001 versus control, ####P<0.0001 versus all groups except any other catalytically inactive molecule; $$$P<0.001 pairwise.

FIG. 10 shows the nociceptive threshold (A), allodynia (B), and hyperalgesia (C) in control rats (white bars), CYP animals (light grey bars) and CYP animals treated with BoNT/AB(0) (dark grey bars), at day 12. Unpaired Student's t test, **p<0.001, ***p<0.0001 vs control rats; ##p<0.001, ###p<0.0001 vs CYP-saline rats.

### SEQUENCE LISTING

Where an initial Met amino acid residue or a corresponding initial codon is indicated in any of the following SEQ ID NOs, said residue/codon is optional.
SEQ ID NO: 1—Nucleotide Sequence of Recombinant Catalytically Inactive BoNT/A (rBoNT/A(0))
SEQ ID NO: 2—Polypeptide Sequence of rBoNT/A(0)
SEQ ID NO: 3—Nucleotide Sequence of rLH_{N}/A (light-chain plus translocation domain only).
SEQ ID NO: 4—Polypeptide Sequence of rLH_{N}/A
SEQ ID NO: 5—Nucleotide Sequence of rL/A (light-chain only)
SEQ ID NO: 6—Polypeptide Sequence of rL/A
SEQ ID NO: 7—Nucleotide Sequence of rH_{C}/A
SEQ ID NO: 8—Polypeptide Sequence of rH_{C}/A
SEQ ID NO: 9—Nucleotide Sequence of rBoNT/B(0)
SEQ ID NO: 10—Polypeptide Sequence of rBoNT/B(0)
SEQ ID NO: 11—Nucleotide Sequence of rBoNT/C(0)
SEQ ID NO: 12—Polypeptide Sequence of rBoNT/C(0)
SEQ ID NO: 13—Nucleotide Sequence of rBoNT/E(0)
SEQ ID NO: 14—Polypeptide Sequence of rBoNT/E(0)
SEQ ID NO: 15—Nucleotide Sequence of rBoNT/F(0)
SEQ ID NO: 16—Polypeptide Sequence of rBoNT/F(0)
SEQ ID NO: 17—Nucleotide Sequence of rBoNT/A(0) (His-tagged)
SEQ ID NO: 18—Polypeptide Sequence of rBoNT/A(0) (His-tagged)
SEQ ID NO: 19—Nucleotide Sequence of rLH_{N}/A (His-tagged)
SEQ ID NO: 20—Polypeptide Sequence of rLH_{N}/A (His-tagged)
SEQ ID NO: 21—Nucleotide Sequence of rH_{C}/A (His-tagged)
SEQ ID NO: 22—Polypeptide Sequence of rH_{C}/A (His-tagged)
SEQ ID NO: 23—Nucleotide Sequence of rLC/A (His-tagged)
SEQ ID NO: 24—Polypeptide Sequence of rLC/A (His-tagged)
SEQ ID NO: 25—Nucleotide Sequence of rBoNT/FA(0) (His-tagged)
SEQ ID NO: 26—Polypeptide Sequence of rBoNT/FA(0) (His-tagged)
SEQ ID NO: 27—Nucleotide Sequence of rLH_{N}/FA (His-tagged)
SEQ ID NO: 28—Polypeptide Sequence of rLH_{N}/FA (His-tagged)
SEQ ID NO: 29—Nucleotide Sequence of rH_{C}/FA (His-tagged)
SEQ ID NO: 30—Polypeptide Sequence of rH_{C}/FA (His-tagged)
SEQ ID NO: 31—Nucleotide Sequence of rLC/FA (His-tagged)
SEQ ID NO: 32—Polypeptide Sequence of rLC/FA (His-tagged)
SEQ ID NO: 33—Nucleotide Sequence of rBoNT/F(0) (His-tagged)
SEQ ID NO: 34—Polypeptide Sequence of rBoNT/F(0) (His-tagged)
SEQ ID NO: 35—Nucleotide Sequence of rL_{H}N/F (His-tagged)
SEQ ID NO: 36—Polypeptide Sequence of rL_{H}N/F (His-tagged)
SEQ ID NO: 37—Nucleotide Sequence of rH_{C}/F (His-tagged)
SEQ ID NO: 38—Polypeptide Sequence of rH_{C}/F (His-tagged)
SEQ ID NO: 39—Nucleotide Sequence of rLC/F (His-tagged)
SEQ ID NO: 40—Polypeptide Sequence of rLC/F (His-tagged)
SEQ ID NO: 41—Nucleotide Sequence of Cationic rH_{C}/A (His-tagged)
SEQ ID NO: 42—Polypeptide Sequence of Cationic rH_{C}/A (His-tagged)
SEQ ID NO: 43—Nucleotide Sequence of rH_{C}/AB (His-tagged)
SEQ ID NO: 44—Polypeptide Sequence of rH_{C}/AB (His-tagged)
SEQ ID NO: 45—Nucleotide Sequence of rH_{C}/A Variant Y1117V H1253K (His-tagged)
SEQ ID NO: 46—Polypeptide Sequence of rH_{C}/A Variant Y1117V H1253K (His-tagged)
SEQ ID NO: 47—Nucleotide Sequence of rH_{C}/A Variant Y1117V F1252Y H1253K L1278F (His-tagged)
SEQ ID NO: 48—Polypeptide Sequence of rH_{C}/A Variant Y1117V F1252Y H1253K L1278F (His-tagged)
SEQ ID NO: 49—Nucleotide Sequence of rH_{C}/A Variant Y1117V F1252Y H1253K L1278H (His-tagged)
SEQ ID NO: 50—Polypeptide Sequence of rH_{C}/A Variant Y1117V F1252Y H1253K L1278H (His-tagged)
SEQ ID NO: 51—Polypeptide Sequence of BoNT/A—UniProt P10845
SEQ ID NO: 52—Polypeptide Sequence of BoNT/B—UniProt P10844
SEQ ID NO: 53—Polypeptide Sequence of BoNT/C—UniProt P18640
SEQ ID NO: 54—Polypeptide Sequence of BoNT/D—UniProt P19321
SEQ ID NO: 55—Polypeptide Sequence of BoNT/E—UniProt Q00496
SEQ ID NO: 56—Polypeptide Sequence of BoNT/F—UniProt A7GBG3
SEQ ID NO: 57—Polypeptide Sequence of BoNT/G—UniProt Q60393
SEQ ID NO: 58—Polypeptide Sequence of TeNT—UniProt P04958
SEQ ID NO: 59—Polypeptide Sequence of BoNT/X
SEQ ID NO: 60—Polypeptide Sequence of Unmodified BoNT/A1
SEQ ID NO: 61—Polypeptide Sequence of mrBoNT/AB(0)
SEQ ID NO: 62—Polypeptide Sequence of mrBoNT/A(0)
SEQ ID NO: 63—Polypeptide Sequence of BoNT/XB(0) (His-tagged)
SEQ ID NO: 64—Polypeptide Sequence of BoNT/XB(0)
SEQ ID NO: 65—Polypeptide Sequence of BoNT/XB(0) Variant (His-tagged)
SEQ ID NO: 66—Polypeptide Sequence of BoNT/XB(0) Variant
SEQ ID NO: 67—Polypeptide Sequence of BoNT/XA(0)
SEQ ID NO: 68—Polypeptide Sequence of BoNT/XA(0) Variant
SEQ ID NO: 69—Polypeptide Sequence of BoNT/XD(0)
SEQ ID NO: 70—Polypeptide Sequence of BoNT/XF(0)
SEQ ID NO: 71—C1 Activation Loop Consensus Sequence
SEQ ID NO: 72—C1 Activation Loop
SEQ ID NO: 73—C1 Activation Loop Variant
SEQ ID NO: 74—Polypeptide Sequence of rLC/A(0) (His-tagged)
SEQ ID NO: 75—Polypeptide Sequence of rLH_{N}/A(0) (His-tagged)
SEQ ID NO: 76—Polypeptide Sequence of rLC/X(0)
SEQ ID NO: 77—PreScission Protease Site
SEQ ID NO: 78—C1 Activation Loop Variant 2

SEQ ID NO: 1-Nucleotide Sequence of rBoNT/A(0)
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATCAACTG
ATCtACGCAGGCCACCGCCTGTACGGCATTGCCATCAACCCAAACCGTGTGTTCAAGGTTAATACGAATGCATAC
TACGAGATGAGCGGCCTGGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGAGATTTACACC
GAGGACAACTTTGTGAAATTCTTCAAAGTGTTGAATCGTAAAACCTATCTGAATTTTGACAAAGCGGTTTTCAAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGTGCGTGCGCGGTATCATCACCAGCAAAACCAAAAGCCTGGACAAAGGCTACAACAAGGCGCTG
AATGACCTGTGCATTAAGGTAAACAATTGGGATCTGTTCTTTTCGCCATCCGAAGATAATTTTACCAACGACCTG
AACAAGGGTGAAGAAATCACCAGCGATACGAATATTGAAGCAGCGGAAGAGAATATCAGCCTGGATCTGATCCAG
CAGTACTATCTGACCTTTAACTTCGACAATGAACCGGAGAACATTAGCATTGAGAATCTGAGCAGCGACATTATC
GGTCAGCTGGAACTGATGCCGAATATCGAACGTTTCCCGAACGGCAAAAAGTACGAGCTGGACAAGTACACTATG
TTCCATTACCTGCGTGCACAGGAGTTTGAACACGGTAAAAGCCGTATCGCGCTGACCAACAGCGTTAACGAGGCC
CTGCTGAACCCGAGCCGTGTCTATACCTTCTTCAGCAGCGACTATGTTAAGAAAGTGAACAAAGCCACTGAGGCC
GCGATGTTCCTGGGCTGGGTGGAACAGCTGGTATATGACTTCACGGACGAGACGAGCGAAGTGAGCACTACCGAC
AAAATTGCTGATATTACCATCATTATCCCGTATATTGGTCCGGCACTGAACATTGGCAACATGCTGTACAAAGAC
GATTTTGTGGGTGCCCTGATCTTCTCCGGTGCCGTGATTCTGCTGGAGTTCATTCCGGAGATTGCGATCCCGGTG
TTGGGTACCTTCGCGCTGGTGTCCTACATCGCGAATAAGGTTCTGACGGTTCAGACCATCGATAACGCGCTGTCG
AAACGTAATGAAAAATGGGACGAGGTTTACAAATACATTGTTACGAATTGGCTGGCGAAAGTCAATACCCAGATC
GACCTGATCCGTAAGAAAATGAAAGAGGCGCTGGAGAATCAGGCGGAGGCCACCAAAGCAATTATCAACTACCAA
TACAACCAGTACACGGAAGAAGAGAAGAATAACATTAACTTCAATATCGATGATTTGAGCAGCAAGCTGAATGAA
TCTATCAACAAAGCGATGATCAATATCAACAAGTTTTTGAATCAGTGTAGCGTTTCGTACCTGATGAATAGCATG
ATTCCGTATGGCGTCAAACGTCTGGAGGACTTCGACGCCAGCCTGAAAGATGCGTTGCTGAAATACATTTACGAC
AATCGTGGTACGCTGATTGGCCAAGTTGACCGCTTGAAAGACAAAGTTAACAATACCCTGAGCACCGACATCCCA
TTTCAACTGAGCAAGTATGTTGATAATCAACGTCTGTTGAGCACTTTCACCGAGTATATCAAAAACATCATCAAT
ACTAGCATTCTGAACCTGCGTTACGAGAGCAATCATCTGATTGATCTGAGCCGTTATGCAAGCAAGATCAACATC
GGTAGCAAGGTCAATTTTGACCCGATCGATAAGAACCAGATCCAGCTGTTTAATCTGGAATCGAGCAAAATTGAG
GTTATCCTGAAAAACGCCATTGTCTACAACTCCATGTACGAGAATTTCTCCACCAGCTTCTGGATTCGCATCCCG
AAATACTTCAACAGCATTAGCCTGAACAACGAGTATACTATCATCAACTGTATGGAGAACAACAGCGGTTGGAAG
GTGTCTCTGAACTATGGTGAGATCATTTGGACCTTGCAGGACACCCAAGAGATCAAGCAGCGCGTCGTGTTCAAG
TACTCTCAAATGATCAACATTTCCGATTACATTAATCGTTGGATCTTCGTGACCATTACGAATAACCGTCTGAAT
AACAGCAAGATTTACATCAATGGTCGCTTGATCGATCAGAAACCGATTAGCAACCTGGGTAATATCCACGCAAGC
AACAACATTATGTTCAAATTGGACGGTTGCCGCGATACCCATCGTTATATCTGGATCAAGTATTTCAACCTGTTT
GATAAAGAACTGAATGAGAAGGAGATCAAAGATTTGTATGACAACCAATCTAACAGCGGCATTTTGAAGGACTTC
TGGGGCGATTATCTGCAATACGATAAGCCGTACTATATGCTGAACCTGTATGATCCGAACAAATATGTGGATGTC
AATAATGTGGGTATTCGTGGTTACATGTATTTGAAGGGTCCGCGTGGCAGCGTTATGACGACCAACATTTACCTG
AACTCTAGCCTGTACCGTGGTACGAAATTCATCATTAAGAAATATGCCAGCGGCAACAAAGATAACATTGTGCGT
AATAACGATCGTGTCTACATCAACGTGGTCGTGAAGAATAAAGAGTACCGTCTGGCGACCAACGCTTCGCAGGCG
GGTGTTGAGAAAATTCTGAGCGCGTTGGAGATCCCTGATGTCGGTAATCTGAGCCAAGTCGTGGTTATGAAGAGC
AAGAACGACCAGGGTATCACTAACAAGTGCAAGATGAACCTGCAAGACAACAATGGTAACGACATCGGCTTTATT
GGTTTCCACCAGTTCAACAATATTGCTAAACTGGTAGCGAGCAATTGGTACAATCGTCAGATTGAGCGCAGCAGC
CGTACTTTGGGCTGTAGCTGGGAGTTTATCCCGGTCGATGATGGTTGGGGCGAACGTCCGCTG
SEQ ID NO: 2-Polypeptide Sequence of rBoNT/A(0)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHQL
IYAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVEK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYENSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSS
RTLGCSWEFIPVDDGWGERPL
SEQ ID NO: 3-Nucleotide Sequence of rLH_{N}/A
atggagttcgttaacaaacagttcaactataaagacccagttaacggtgttgacattgcttacatcaaaatcccg
aacgctggccagatgcagccggtaaaggcattcaaaatccacaacaaaatctgggttatcccggaacgtgatacc
tttactaacccggaagaaggtgacctgaacccgccaccggaagcgaaacaggtgccggtatcttactatgactcc
acctacctgtctaccgataacgaaaaggacaactacctgaaaggtgttactaaactgttcgagcgtatttactcc
accgacctgggccgtatgctgctgactagcatcgttcgcggtatcccgttctggggcggttctaccatcgatacc
gaactgaaagtaatcgacactaactgcatcaacgttattcagccggacggttcctatcgttccgaagaactgaac
ctggtgatcatcggcccgtctgctgatatcatccagttcgagtgtaagagctttggtcacgaagttctgaacctc
acccgtaacggctacggttccactcagtacatccgtttctctccggacttcaccttcggttttgaagaatccctg
gaagtagacacgaacccactgctgggcgctggtaaattcgcaactgatcctgcggttaccctggctcacgaactg
attcatgcaggccaccgcctgtacggtatcgccatcaatccgaaccgtgtcttcaaagttaacaccaacgcgtat
tacgagatgtccggtctggaagttagcttcgaagaactgcgtacttttggcggtcacgacgctaaattcatcgac
tctctgcaagaaaacgagttccgtctgtactactataacaagttcaaagatatcgcatccaccctgaacaaagcg
aaatccatcgtgggtaccactgcttctctccagtacatgaagaacgtttttaaagaaaaatacctgctcagcgaa
gacacctccggcaaattctctgtagacaagttgaaattcgataaactttacaaaatgctgactgaaatttacacc
gaagacaacttcgttaagttctttaaagttctgaaccgcaaaacctatctgaacttcgacaaggcagtattcaaa
atcaacatcgtgccgaaagttaactacactatctacgatggtttcaacctgcgtaacaccaacctggctgctaat
tttaacggccagaacacggaaatcaacaacatgaacttcacaaaactgaaaaacttcactggtctgttcgagttt
tacaagctgctgtgcGTCGACGGCATCATTACCTCCAAAACTAAATCTGACGATGACGATAAAAACAAAGCGCTG
AACCTGCAGtgtatcaaggttaacaactgggatttattcttcagcccgagtgaagacaacttcaccaacgacctg
aacaaaggtgaagaaatcacctcagatactaacatcgaagcagccgaagaaaacatctcgctggacctgatccag
cagtactacctgacctttaatttcgacaacgagccggaaaacatttctatcgaaaacctgagctctgatatcatc
ggccagctggaactgatgccgaacatcgaacgtttcccaaacggtaaaaagtacgagctggacaaatataccatg
ttccactacctgcgcgcgcaggaatttgaacacggcaaatcccgtatcgcactgactaactccgttaacgaagct
ctgctcaacccgtcccgtgtatacaccttcttctctagcgactacgtgaaaaaggtcaacaaagcgactgaagct
gcaatgttcttgggttgggttgaacagcttgtttatgattttaccgacgagacgtccgaagtatctactaccgac
aaaattgcggatatcactatcatcatcccgtacatcggtccggctctgaacattggcaacatgctgtacaaagac
gacttcgttggcgcactgatcttctccggtgcggtgatcctgctggagttcatcccggaaatcgccatcccggta
ctgggcacctttgctctggtttcttacattgcaaacaaggttctgactgtacaaaccatcgacaacgcgctgagc
aaacgtaacgaaaaatgggatgaagtttacaaatatatcgtgaccaactggctggctaaggttaatactcagatc
gacctcatccgcaaaaaaatgaaagaagcactggaaaaccaggcggaagctaccaaggcaatcattaactaccag
tacaaccagtacaccgaggaagaaaaaaacaacatcaacttcaacatcgacgatctgtcctctaaactgaacgaa
tccatcaacaaagctatgatcaacatcaacaagttcctgaaccagtgctctgtaagctatctgatgaactccatg
atcccgtacggtgttaaacgtctggaggacttcgatgcgtctctgaaagacgccctgctgaaatacatttacgac
aaccgtggcactctgatcggtcaggttgatcgtctgaaggacaaagtgaacaataccttatcgaccgacatccct
tttcagctcagtaaatatgtcgataaccaacgccttttgtccactctagaagcaCACCATCATCACcaccatcac
catcaccat
SEQ ID NO: 4-Polypeptide Sequence of rLH_{N}/A
MEFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVEK
INIVPKVNYTIYDGFNLRNTNLAANFNGQNTEINNMNFTKLKNFTGLFEFYKLLCVDGIITSKTKSDDDDKNKAL
NLQCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTLEAHHHHHHHHHH
SEQ ID NO: 5-Nucleotide Sequence of rL/A
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATGAACTG
ATCCACGCAGGCCACCGCCTGTACGGCATTGCCATCAACCCAAACCGTGTGTTCAAGGTTAATACGAATGCATAC
TACGAGATGAGCGGCCTGGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGAGATTTACACC
GAGGACAACTTTGTGAAATTCTTCAAAGTGTTGAATCGTAAAACCTATCTGAATTTTGACAAAGCGGTTTTCAAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGggtctagaagcaCACCATCATCACcaccatcaccatcaccat
SEQ ID NO: 6-Polypeptide Sequence of rL_{C}/A
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLGLEAHHHHHHHHHH
SEQ ID NO: 7-Nucleotide Sequence of rH_{C}/A
ATGCATCATCACCATCACCACAAAAACATCATCAATACTAGCATTCTGAACCTGCGTTACGAGAGCAATCATCTG
ATTGATCTGAGCCGTTATGCAAGCAAGATCAACATCGGTAGCAAGGTCAATTTTGACCCGATCGATAAGAACCAG
ATCCAGCTGTTTAATCTGGAATCGAGCAAAATTGAGGTTATCCTGAAAAACGCCATTGTCTACAACTCCATGTAC
GAGAATTTCTCCACCAGCTTCTGGATTCGCATCCCGAAATACTTCAACAGCATTAGCCTGAACAACGAGTATACT
ATCATCAACTGTATGGAGAACAACAGCGGTTGGAAGGTGTCTCTGAACTATGGTGAGATCATTTGGACCTTGCAG
GACACCCAAGAGATCAAGCAGCGCGTCGTGTTCAAGTACTCTCAAATGATCAACATTTCCGATTACATTAATCGT
TGGATCTTCGTGACCATTACGAATAACCGTCTGAATAACAGCAAGATTTACATCAATGGTCGCTTGATCGATCAG
AAACCGATTAGCAACCTGGGTAATATCCACGCAAGCAACAACATTATGTTCAAATTGGACGGTTGCCGCGATACC
CATCGTTATATCTGGATCAAGTATTTCAACCTGTTTGATAAAGAACTGAATGAGAAGGAGATCAAAGATTTGTAT
GACAACCAATCTAACAGCGGCATTTTGAAGGACTTCTGGGGCGATTATCTGCAATACGATAAGCCGTACTATATG
CTGAACCTGTATGATCCGAACAAATATGTGGATGTCAATAATGTGGGTATTCGTGGTTACATGTATTTGAAGGGT
CCGCGTGGCAGCGTTATGACGACCAACATTTACCTGAACTCTAGCCTGTACCGTGGTACGAAATTCATCATTAAG
AAATATGCCAGCGGCAACAAAGATAACATTGTGCGTAATAACGATCGTGTCTACATCAACGTGGTCGTGAAGAAT
AAAGAGTACCGTCTGGCGACCAACGCTTCGCAGGCGGGTGTTGAGAAAATTCTGAGCGCGTTGGAGATCCCTGAT
GTCGGTAATCTGAGCCAAGTCGTGGTTATGAAGAGCAAGAACGACCAGGGTATCACTAACAAGTGCAAGATGAAC
CTGCAAGACAACAATGGTAACGACATCGGCTTTATTGGTTTCCACCAGTTCAACAATATTGCTAAACTGGTAGCG
AGCAATTGGTACAATCGTCAGATTGAGCGCAGCAGCCGTACTTTGGGCTGTAGCTGGGAGTTTATCCCGGTCGAT
GATGGTTGGGGCGAACGTCCGCTG
SEQ ID NO: 8-Polypeptide Sequence of rH_{C}/A
MHHHHHHKNIINTSILNLRYESNHLIDLSRYASKINIGSKVNFDPIDKNQIQLENLESSKIEVILKNAIVYNSMY
ENFSTSFWIRIPKYENSISLNNEYTIINCMENNSGWKVSLNYGEIIWTLQDTQEIKQRVVFKYSQMINISDYINR
WIFVTITNNRLNNSKIYINGRLIDQKPISNLGNIHASNNIMFKLDGCRDTHRYIWIKYENLFDKELNEKEIKDLY
DNQSNSGILKDFWGDYLQYDKPYYMLNLYDPNKYVDVNNVGIRGYMYLKGPRGSVMTTNIYLNSSLYRGTKFIIK
KYASGNKDNIVRNNDRVYINVVVKNKEYRLATNASQAGVEKILSALEIPDVGNLSQVVVMKSKNDQGITNKCKMN
LQDNNGNDIGFIGFHQFNNIAKLVASNWYNRQIERSSRTLGCSWEFIPVDDGWGERPL
SEQ ID NO: 9-Nucleotide Sequence of rBoNT/B(0)
ATGCCGGTGACGATTAACAACTTCAACTACAACGACCCGATTGACAACAACAACATTATCATGATGGAACCGCCG
TTTGCACGCGGCACGGGCCGTTATTACAAAGCGTTTAAAATCACCGATCGTATTTGGATTATCCCGGAACGCTAC
ACGTTTGGTTATAAACCGGAAGACTTCAACAAAAGCTCTGGCATCTTCAACCGTGATGTTTGCGAATACTACGAT
CCGGACTACCTGAACACCAACGATAAGAAAAACATTTTTCTGCAAACGATGATCAAACTGTTCAATCGCATTAAA
AGCAAACCGCTGGGTGAAAAACTGCTGGAAATGATTATCAATGGCATTCCGTATCTGGGTGATCGTCGCGTGCCG
CTGGAAGAATTTAACACCAATATCGCGAGTGTTACGGTCAACAAACTGATTTCCAATCCGGGTGAAGTCGAACGT
AAAAAAGGCATCTTCGCCAACCTGATCATCTTCGGCCCGGGTCCGGTGCTGAACGAAAATGAAACCATTGATATC
GGTATTCAGAACCATTTTGCCTCACGCGAAGGCTTCGGCGGTATTATGCAAATGAAATTTTGCCCGGAATATGTG
TCGGTTTTCAACAATGTTCAGGAAAACAAAGGTGCAAGCATCTTTAATCGTCGCGGCTATTTCTCTGATCCGGCT
CTGATCCTGATGCACCAACTGATTtATGTGCTGCACGGCCTGTATGGTATCAAAGTGGATGACCTGCCGATCGTT
CCGAACGAGAAAAAATTTTTCATGCAGAGCACCGACGCAATTCAAGCTGAAGAACTGTATACGTTTGGCGGTCAG
GACCCGTCTATTATCACCCCGAGCACCGACAAAAGCATCTACGATAAAGTGCTGCAAAACTTTCGTGGCATTGTT
GACCGCCTGAATAAAGTCCTGGTGTGTATCTCTGATCCGAACATCAACATCAACATCTACAAAAACAAATTCAAA
GACAAATACAAATTCGTTGAAGATTCTGAAGGCAAATATAGTATTGACGTCGAATCCTTTGATAAACTGTACAAA
AGTCTGATGTTCGGTTTCACCGAAACGAACATCGCGGAAAACTACAAAATCAAAACCCGCGCCTCCTATTTCAGC
GACTCTCTGCCGCCGGTTAAAATCAAAAATCTGCTGGATAACGAAATTTATACGATCGAAGAAGGTTTCAACATC
AGCGATAAAGACATGGAAAAAGAATACCGTGGCCAGAATAAAGCAATCAACAAACAGGCGTATGAAGAAATTAGT
AAAGAACATCTGGCGGTCTACAAAATTCAGATGTGCAAATCCGTGAAAGCCCCGGGTATTTGTATCGATGTTGAC
AATGAAGACCTGTTTTTCATCGCCGATAAAAACAGTTTTTCCGATGACCTGTCAAAAAATGAACGCATCGAATAC
AACACCCAATCGAACTACATCGAAAACGATTTCCCGATCAACGAACTGATTCTGGATACGGACCTGATTAGTAAA
ATCGAACTGCCGTCAGAAAACACCGAATCGCTGACGGACTTTAATGTTGATGTCCCGGTGTATGAAAAACAGCCG
GCAATTAAGAAAATTTTTACCGATGAAAACACGATCTTCCAGTACCTGTACAGCCAAACCTTTCCGCTGGACATT
CGCGATATCTCTCTGACGAGTTCCTTTGATGACGCACTGCTGTTCAGCAACAAAGTGTACTCCTTTTTCTCAATG
GATTACATCAAAACCGCTAACAAAGTGGTTGAAGCGGGCCTGTTTGCCGGTTGGGTGAAACAGATCGTTAACGAT
TTCGTCATCGAAGCCAACAAAAGTAACACGATGGATAAAATTGCTGATATCTCCCTGATTGTCCCGTATATTGGC
CTGGCACTGAATGTGGGTAACGAAACGGCGAAAGGCAATTTTGAAAACGCCTTCGAAATTGCAGGCGCTTCAATC
CTGCTGGAATTTATTCCGGAACTGCTGATCCCGGTCGTGGGTGCGTTCCTGCTGGAATCTTACATCGACAACAAA
AACAAAATCATCAAAACCATTGATAACGCGCTGACGAAACGTAACGAAAAATGGTCAGATATGTACGGCCTGATT
GTTGCCCAGTGGCTGAGCACCGTCAACACGCAATTTTACACCATCAAAGAAGGTATGTACAAAGCGCTGAATTAT
CAGGCGCAAGCCCTGGAAGAAATCATCAAATACCGCTACAACATCTACAGCGAAAAAGAAAAATCTAACATCAAC
ATCGACTTTAATGATATCAACAGCAAACTGAACGAAGGTATCAACCAGGCAATCGATAACATCAACAACTTCATC
AACGGCTGCTCAGTGTCGTATCTGATGAAGAAAATGATCCCGCTGGCTGTTGAAAAACTGCTGGATTTTGACAAC
ACCCTGAAGAAAAACCTGCTGAACTACATCGATGAAAACAAACTGTACCTGATCGGCTCAGCCGAATACGAAAAA
TCGAAAGTGAACAAATACCTGAAAACCATCATGCCGTTTGACCTGAGTATTTACACCAACGATACGATCCTGATC
GAAATGTTCAACAAATACAACTCCGAAATTCTGAACAATATTATCCTGAACCTGCGTTACAAAGACAACAATCTG
ATCGATCTGAGCGGCTATGGTGCAAAAGTTGAAGTCTACGACGGTGTCGAACTGAACGATAAAAACCAGTTCAAA
CTGACCTCATCGGCTAACTCAAAAATTCGTGTGACGCAGAACCAAAACATCATCTTCAACTCGGTCTTTCTGGAC
TTCAGCGTGTCTTTCTGGATTCGCATCCCGAAATATAAAAATGATGGCATCCAGAACTACATCCATAACGAATAC
ACCATCATCAACTGTATGAAAAACAACAGTGGTTGGAAAATTTCCATCCGTGGCAACCGCATTATCTGGACCCTG
ATTGATATCAATGGTAAAACGAAAAGCGTGTTTTTCGAATACAACATCCGTGAAGATATCTCTGAATACATCAAT
CGCTGGTTTTTCGTGACCATTACGAACAATCTGAACAATGCGAAAATCTATATCAACGGCAAACTGGAAAGTAAT
ACCGACATCAAAGATATTCGTGAAGTTATCGCCAACGGTGAAATCATCTTCAAACTGGATGGCGACATCGATCGC
ACCCAGTTCATTTGGATGAAATACTTCTCCATCTTCAACACGGAACTGAGTCAGTCCAATATCGAAGAACGCTAC
AAAATCCAATCATACTCGGAATACCTGAAAGATTTCTGGGGTAACCCGCTGATGTACAACAAAGAATACTACATG
TTCAACGCGGGCAACAAAAACTCATACATCAAACTGAAAAAAGATTCGCCGGTGGGTGAAATCCTGACCCGTAGC
AAATACAACCAGAACTCTAAATACATCAACTATCGCGATCTGTACATTGGCGAAAAATTTATTATCCGTCGCAAA
AGCAACTCTCAGAGTATTAATGATGACATCGTGCGTAAAGAAGACTACATCTATCTGGATTTCTTTAATCTGAAC
CAAGAATGGCGCGTTTATACCTACAAATACTTCAAAAAAGAAGAAGAGAAACTGTTCCTGGCCCCGATTAGCGAC
AGCGATGAATTTTACAACACCATCCAGATCAAAGAATACGATGAACAGCCGACGTATAGTTGCCAACTGCTGTTC
AAAAAAGACGAAGAATCCACCGATGAAATTGGCCTGATTGGTATCCACCGTTTCTATGAAAGCGGTATCGTTTTC
GAAGAATACAAAGATTACTTCTGTATCTCTAAATGGTATCTGAAAGAAGTCAAACGCAAACCGTACAACCTGAAA
CTGGGCTGCAACTGGCAATTTATCCCGAAAGACGAAGGCTGGACCGAA
SEQ ID NO: 10-Polypeptide Sequence of rBoNT/B(0)
MPVTINNFNYNDPIDNNNIIMMEPPFARGTGRYYKAFKITDRIWIIPERYTFGYKPEDENKSSGIFNRDVCEYYD
PDYLNTNDKKNIFLQTMIKLFNRIKSKPLGEKLLEMIINGIPYLGDRRVPLEEFNTNIASVTVNKLISNPGEVER
KKGIFANLIIFGPGPVLNENETIDIGIQNHFASREGFGGIMQMKFCPEYVSVENNVQENKGASIFNRRGYFSDPA
LILMHQLIYVLHGLYGIKVDDLPIVPNEKKFFMQSTDAIQAEELYTFGGQDPSIITPSTDKSIYDKVLQNERGIV
DRLNKVLVCISDPNININIYKNKFKDKYKFVEDSEGKYSIDVESFDKLYKSLMFGFTETNIAENYKIKTRASYFS
DSLPPVKIKNLLDNEIYTIEEGENISDKDMEKEYRGQNKAINKQAYEEISKEHLAVYKIQMCKSVKAPGICIDVD
NEDLFFIADKNSFSDDLSKNERIEYNTQSNYIENDFPINELILDTDLISKIELPSENTESLTDFNVDVPVYEKQP
AIKKIFTDENTIFQYLYSQTFPLDIRDISLTSSFDDALLESNKVYSFFSMDYIKTANKVVEAGLFAGWVKQIVND
FVIEANKSNTMDKIADISLIVPYIGLALNVGNETAKGNFENAFEIAGASILLEFIPELLIPVVGAFLLESYIDNK
NKIIKTIDNALTKRNEKWSDMYGLIVAQWLSTVNTQFYTIKEGMYKALNYQAQALEEIIKYRYNIYSEKEKSNIN
IDFNDINSKLNEGINQAIDNINNFINGCSVSYLMKKMIPLAVEKLLDFDNTLKKNLLNYIDENKLYLIGSAEYEK
SKVNKYLKTIMPFDLSIYINDTILIEMENKYNSEILNNIILNLRYKDNNLIDLSGYGAKVEVYDGVELNDKNQFK
LTSSANSKIRVTQNQNIIFNSVFLDFSVSFWIRIPKYKNDGIQNYIHNEYTIINCMKNNSGWKISIRGNRIIWTL
IDINGKTKSVFFEYNIREDISEYINRWFFVTITNNLNNAKIYINGKLESNTDIKDIREVIANGEIIFKLDGDIDR
TQFIWMKYFSIFNTELSQSNIEERYKIQSYSEYLKDFWGNPLMYNKEYYMFNAGNKNSYIKLKKDSPVGEILTRS
KYNQNSKYINYRDLYIGEKFIIRRKSNSQSINDDIVRKEDYIYLDFFNLNQEWRVYTYKYFKKEEEKLFLAPISD
SDEFYNTIQIKEYDEQPTYSCQLLFKKDEESTDEIGLIGIHRFYESGIVFEEYKDYFCISKWYLKEVKRKPYNLK
LGCNWQFIPKDEGWTE
SEQ ID NO: 11-Nucleotide Sequence of rBoNT/C(0)
ATGCCGATCACGATTAATAATTTCAACTATAGCGATCCGGTGGACAATAAGAATATTCTGTATCTGGATACTCAT
CTGAATACGCTGGCTAACGAACCGGAGAAAGCGTTCCGCATCACAGGCAACATCTGGGTTATTCCCGATCGCTTT
TCACGCAACAGCAACCCTAATCTGAACAAACCTCCTCGTGTCACCAGTCCTAAATCCGGTTATTACGACCCAAAC
TATCTGAGTACGGATAGCGATAAAGATCCCTTTCTGAAAGAGATCATTAAGCTGTTCAAACGCATTAACTCTCGC
GAAATTGGGGAAGAGCTGATCTATCGGCTTTCGACAGATATCCCGTTCCCAGGTAACAATAATACCCCGATTAAT
ACTTTCGACTTTGATGTTGATTTCAATTCTGTGGATGTGAAAACGCGTCAAGGCAATAATTGGGTGAAAACTGGT
AGCATTAACCCGAGTGTAATTATCACAGGTCCCCGTGAGAACATCATCGACCCGGAAACCTCTACCTTCAAGCTG
ACGAACAACACGTTTGCTGCACAGGAAGGGTTTGGTGCCCTGTCAATCATTTCCATCTCACCGCGTTTCATGTTA
ACCTACTCCAATGCCACAAATGATGTTGGCGAAGGACGTTTTAGCAAATCAGAATTTTGCATGGACCCAATTCTC
ATTCTGATGggCacGCTGAACaATGCGATGCACAACTTGTATGGCATTGCTATTCCAAACGATCAAACCATTAGC
TCCGTTACCAGTAATATCTTCTATAGCCAGTATAATGTCAAATTGGAGTATGCCGAAATTTACGCCTTTGGAGGC
CCGACCATTGACCTGATTCCGAAATCTGCACGCAAATACTTCGAAGAAAAGGCGTTAGATTACTATCGCAGCATC
GCGAAACGCCTGAACTCGATTACCACGGCCAATCCGTCGTCGTTCAACAAATACATTGGTGAATATAAACAGAAA
CTGATTCGCAAATATCGGTTTGTCGTAGAAAGCTCTGGTGAAGTGACTGTAAACCGCAACAAATTTGTCGAACTC
TACAACGAGTTGACCCAAATCTTTACCGAGTTTAACTACGCAAAGATCTATAACGTACAGAACCGCAAGATTTAT
CTTAGCAATGTATACACACCGGTTACTGCGAACATCTTAGACGACAATGTGTATGATATTCAGAATGGCTTTAAC
ATCCCGAAATCAAATCTGAACGTTCTGTTTATGGGCCAGAACCTGAGTCGTAATCCAGCACTGCGTAAAGTGAAC
CCGGAAAATATGCTCTACTTGTTTACCAAATTTTGCCACAAAGCGATTGATGGCCGCTCTCTCTATAACAAAACG
CTGGATTGTCGTGAGTTACTTGTGAAGAACACTGATTTACCGTTCATTGGGGATATCTCCGACGTGAAAACCGAT
ATCTTCCTGCGCAAAGACATTAATGAAGAAACGGAAGTCATCTATTACCCCGACAATGTGAGCGTTGATCAGGTC
ATTTTATCGAAGAACACCTCCGAACATGGTCAGTTGGATTTGCTGTACCCTAGCATTGACTCGGAGAGTGAAATC
CTTCCGGGCGAAAATCAAGTGTTTTACGACAACCGTACCCAAAATGTTGATTATTTGAATTCTTATTACTACCTG
GAATCTCAGAAATTGAGCGACAATGTGGAAGATTTCACGTTCACACGCTCCATTGAGGAAGCGCTGGATAATAGC
GCGAAAGTGTATACGTATTTCCCTACCTTGGCGAATAAAGTAAATGCTGGTGTCCAGGGAGGCTTATTTCTGATG
TGGGCGAATGATGTGGTAGAAGATTTTACGACCAATATTTTGCGTAAGGACACCTTAGATAAAATTAGCGATGTT
AGCGCCATCATCCCCTATATTGGCCCAGCACTGAATATCTCGAACTCTGTGCGTCGCGGAAACTTCACCGAAGCA
TTTGCGGTGACCGGGGTTACTATTCTGTTGGAAGCCTTTCCGGAGTTTACTATTCCGGCGCTGGGTGCGTTTGTG
ATTTATTCGAAAGTACAAGAACGCAATGAAATTATCAAAACCATCGATAATTGCCTGGAACAACGCATTAAACGC
TGGAAGGATTCTTATGAATGGATGATGGGCACCTGGTTATCCCGTATTATCACACAGTTTAACAACATCTCGTAT
CAGATGTACGATTCACTGAACTACCAAGCAGGGGCGATCAAAGCCAAGATCGACTTAGAATACAAGAAATATTCA
GGTAGCGATAAAGAGAATATTAAAAGCCAGGTTGAAAACCTGAAGAACTCTCTGGATGTCAAAATTTCAGAGGCT
ATGAACAACATTAACAAATTTATCCGCGAATGTAGCGTCACGTATCTGTTTAAAAACATGCTCCCGAAAGTGATT
GATGAGCTCAACGAGTTTGATCGCAACACAAAGGCCAAACTGATTAACCTGATTGATAGTCACAATATTATTTTA
GTCGGTGAAGTTGACAAGCTGAAGGCTAAGGTCAATAACAGCTTTCAGAACACTATTCCGTTTAATATTTTCTCC
TATACGAACAATAGTCTGCTGAAAGACATTATCAACGAATACTTCAACAATATTAATGACAGCAAAATTCTGAGC
CTGCAGAATCGTAAGAATACGCTGGTAGATACCAGTGGATATAATGCGGAAGTCTCAGAAGAGGGTGATGTACAG
CTGAACCCGATCTTTCCGTTCGACTTTAAACTGGGGTCTAGTGGTGAAGATCGCGGTAAAGTGATCGTTACCCAA
AACGAGAACATTGTGTATAACAGCATGTACGAGAGTTTCTCAATTTCTTTCTGGATTCGCATCAATAAATGGGTT
TCTAATTTGCCTGGCTATACCATCATTGATAGCGTCAAAAACAACTCGGGCTGGTCGATTGGCATTATTAGCAAC
TTTCTGGTGTTTACCCTGAAACAGAATGAGGATTCGGAACAGAGCATTAACTTCTCCTACGACATCAGCAACAAT
GCACCAGGGTATAACAAATGGTTCTTCGTAACGGTGACGAACAATATGATGGGCAATATGAAAATCTACATTAAC
GGGAAACTTATCGACACCATTAAAGTGAAAGAGCTTACTGGGATCAATTTTAGTAAAACCATTACCTTTGAGATC
AACAAAATTCCGGACACGGGTCTGATTACCTCCGATTCGGATAATATCAATATGTGGATTCGCGACTTTTATATC
TTCGCCAAAGAACTTGATGGCAAAGATATCAACATTTTGTTTAATTCCCTGCAGTATACCAATGTCGTTAAGGAC
TATTGGGGCAATGATCTCCGCTACAATAAAGAATACTACATGGTTAACATCGACTATCTCAATCGCTACATGTAT
GCTAACTCGCGTCAAATTGTGTTTAACACACGTCGTAACAACAACGATTTTAACGAAGGTTATAAAATCATTATC
AAACGGATCCGCGGCAATACGAACGATACTCGTGTTCGTGGCGGTGACATTCTGTATTTCGACATGACGATTAAT
AATAAAGCGTACAATCTGTTCATGAAGAACGAAACCATGTACGCCGATAACCATTCCACTGAAGATATCTACGCA
ATCGGACTTCGCGAACAGACCAAAGACATTAACGACAACATCATCTTTCAGATTCAACCGATGAATAATACCTAC
TACTATGCCTCCCAGATCTTCAAAAGTAATTTCAACGGCGAAAACATTTCAGGCATTTGCTCAATCGGCACTTAT
CGGTTCCGGTTAGGTGGTGATTGGTATCGTCACAACTACCTTGTTCCCACAGTGAAACAAGGCAACTATGCATCG
CTCTTAGAAAGCACATCTACGCATTGGGGTTTTGTGCCAGTCAGTGAA
SEQ ID NO: 12-Polypeptide Sequence of rBoNT/C(0)
MPITINNFNYSDPVDNKNILYLDTHLNTLANEPEKAFRITGNIWVIPDRESRNSNPNLNKPPRVTSPKSGYYDPN
YLSTDSDKDPFLKEIIKLFKRINSREIGEELIYRLSTDIPFPGNNNTPINTFDFDVDENSVDVKTRQGNNWVKTG
SINPSVIITGPRENIIDPETSTFKLTNNTFAAQEGFGALSIISISPRFMLTYSNATNDVGEGRFSKSEFCMDPIL
ILMGTLNNAMHNLYGIAIPNDQTISSVTSNIFYSQYNVKLEYAEIYAFGGPTIDLIPKSARKYFEEKALDYYRSI
AKRLNSITTANPSSENKYIGEYKQKLIRKYRFVVESSGEVTVNRNKEVELYNELTQIFTEFNYAKIYNVQNRKIY
LSNVYTPVTANILDDNVYDIQNGFNIPKSNLNVLFMGQNLSRNPALRKVNPENMLYLFTKFCHKAIDGRSLYNKT
LDCRELLVKNTDLPFIGDISDVKTDIFLRKDINEETEVIYYPDNVSVDQVILSKNTSEHGQLDLLYPSIDSESEI
LPGENQVFYDNRTQNVDYLNSYYYLESQKLSDNVEDFTFTRSIEEALDNSAKVYTYFPTLANKVNAGVQGGLFLM
WANDVVEDFTTNILRKDTLDKISDVSAIIPYIGPALNISNSVRRGNFTEAFAVTGVTILLEAFPEFTIPALGAFV
IYSKVQERNEIIKTIDNCLEQRIKRWKDSYEWMMGTWLSRIITQFNNISYQMYDSLNYQAGAIKAKIDLEYKKYS
GSDKENIKSQVENLKNSLDVKISEAMNNINKFIRECSVTYLFKNMLPKVIDELNEFDRNTKAKLINLIDSHNIIL
VGEVDKLKAKVNNSFQNTIPFNIFSYTNNSLLKDIINEYENNINDSKILSLQNRKNTLVDTSGYNAEVSEEGDVQ
LNPIFPFDFKLGSSGEDRGKVIVTQNENIVYNSMYESFSISFWIRINKWVSNLPGYTIIDSVKNNSGWSIGIISN
FLVFTLKQNEDSEQSINFSYDISNNAPGYNKWFFVTVTNNMMGNMKIYINGKLIDTIKVKELTGINFSKTITFEI
NKIPDTGLITSDSDNINMWIRDFYIFAKELDGKDINILFNSLQYTNVVKDYWGNDLRYNKEYYMVNIDYLNRYMY
ANSRQIVENTRRNNNDFNEGYKIIIKRIRGNTNDTRVRGGDILYFDMTINNKAYNLFMKNETMYADNHSTEDIYA
IGLREQTKDINDNIIFQIQPMNNTYYYASQIFKSNENGENISGICSIGTYRFRLGGDWYRHNYLVPTVKQGNYAS
LLESTSTHWGFVPVSE
SEQ ID NO: 13-Nucleotide Sequence of rBoNT/E(0)
atgccgaaaatcaactctttcaactacaacgacccggttaacgaccgtaccatcctgtatatcaaaccgggtggt
tgccaggagttctacaaatctttcaacatcatgaaaaacatctggatcatcccggaacgtaacgttatcggtacc
accccgcaggacttccacccgccgacctctctgaaaaacggtgactcttcttactacgacccgaactacctccag
tctgacgaagaaaaagaccgtttcctgaaaatcgttaccaaaatcttcaaccgtatcaacaacaacctgtctggt
ggtatcctgctggaagaactgtctaaagctaacccgtacctgggtaacgacaacaccccggacaaccagttccac
atcggtgacgcttctgctgttgaaatcaaattctctaacggttctcaggacatcctgctgccgaacgttatcatc
atgggtgctgaaccggacctgttcgaaaccaactcttctaacatctctctgcgtaacaactacatgccgtctaac
cacggtttcggttctatcgctatcgttaccttctctccggaatactctttccgtttcaacgacaacagcatgaac
gagttcatccaggacccggctctgaccctgatgcaccaactgatctactctctgcacggtctgtacggtgctaaa
ggtatcaccaccaaatacaccatcacccagaaacagaacccgctgatcaccaacatccgtggtaccaacatcgaa
gagttcctgaccttcggtggtaccgacctgaacatcatcacctctgctcagtctaacgacatctacaccaacctg
ctggctgactacaaaaaaatcgcttctaaactgtctaaagttcaggtttctaacccgctgctgaacccgtacaaa
gacgttttcgaagctaaatacggtctggacaaagacgcttctggtatctactctgttaacatcaacaaattcaac
gacatcttcaaaaaactgtactctttcaccgagttcgacctggcgaccaaattccaggttaaatgccgtcagacc
tacatcggtcagtacaaatacttcaaactgtctaacctgctgaacgactctatctacaacatctctgaaggttac
aacatcaacaacctgaaagttaacttccgtggtcagaacgctaacctgaacccgcgtatcatcaccccgatcacc
ggtcgtggtctggttaaaaaaatcatccgtttctgcAAGAATATTGTAAGCGTTAAAGGAATAAGAAAAAGTATC
tgcatcgaaatcaacaacggtgaactgttcttcgttgcttctgaaaactcttacaacgacgacaacatcaacacc
ccgaaagaaatcgacgacaccgttacctctaacaacaactacgaaaacgacctggaccaggttatcctgaacttc
aactctgaatctgctccgggtctgtctgacgaaaaactgaacctgaccatccagaacgacgcttacatcccgaaa
tacgactctaacggtacctctgacatcgaacagcacgacgttaacgaactgaacgttttcttctacctggacgct
cagaaagttccggaaggtgaaaacaacgttaacctgacctcttctatcgacaccgctctgctggaacagccgaaa
atctacaccttcttctcttctgagttcatcaacaacgttaacaaaccggttcaggctgctctgttcgtttcttgg
attcagcaggttctggttgacttcaccaccgaagctaaccagaaatctaccgttgacaaaatcgctgacatctct
atcgttgttccgtacatcggtctggctctgaacatcggtaacgaagctcagaaaggtaacttcaaagacgctctg
gaactgctgggtgctggtatcctgctggagttcgaaccggaactgctgatcccgaccatcctggttttcaccatc
aaatctttcctgggttcttctgacaacaaaaacaaagttatcaaagctatcaacaacgctctgaaagaacgtgac
gaaaaatggaaagaagtttactctttcatcgtttctaactggatgaccaaaatcaacacccagttcaacaaacgt
aaagaacagatgtaccaggctctccagaaccaggttaacgctatcaaaaccatcatcgaatctaaatacaactct
tacaccctggaagaaaaaaacgaactgaccaacaaatacgacatcaaacagatcgaaaacgaactgaaccagaaa
gtttctatcgctatgaacaacatcgaccgtttcctgaccgaatcttctatctcttacctgatgaaactcatcaac
gaagttaaaatcaacaaactgcgtgaatacgacgaaaacgttaaaacctacctgctgaactacatcatccagcac
ggttctatcctgggtgaatctcagcaggaactgaactctatggttaccgacaccctgaacaactctatcccgttc
aaactgtcttcttacaccgacgacaaaatcctGATCTCTTACTTCAACAAATTCTTTAAAcgcATTAAGAGTTCA
TCGGTTctgaatATGCGGTACAAAAATGATAAAtatGTCGATACTTCTGGATATgatAGCAATATCAACATTAAC
GGCGACGTGTATAAATATccgACAAATAAAAACCAGTTTGGGATATATAACGACAAGctgTCGGAGGTCAATatt
TCTCAAAACGACtatATCattTACGATAATaaaTATAAAAACTTTAGCATTAGTtttTGGGTTcgtATACCTAAT
tatGACAATaaaattGTAAATGTGAATAACGAGTATACCATTATAAACTGTATGcgcGACAATAACAGTGGTTGG
AAGGTATCGctgAACCATAATGAGATTATCTGGACCctgcagGATAATgcaGGTATAAACCAGAAACTGGCTTTT
AACTATGGAAACGCAAATGGGATCTCAGATTACATTaataaaTGGatttttGTTaccATTACGAACGATcgcTTA
GGCGACTCAAAACTTTATATTAATggcAATctgATAGATCAGAAATCAATCTTAAATTTGGGCAATATTCATGTC
TCTgatAACATCTTGTTCAAGATCGTTAATTGCAGTTACACTcgtTATATTGGCATTCGTTACTTTAATATCTTC
gataaaGAActgGACGAGACGGAAATCcagACTCTGTATTCAAACGAGCCCAATACTAATATATTGAAAGATTTT
TGGGGTAACTATCTTTTATATGATAAAGAATACTATCTCCTGaatGTATTGAAGCCAAACAATTTCATAGATAGA
CGCAAGGATAGCACATTAAGTATCAACAATATCAGATCTACTATActgttaGCAAATCGCCTcTACTCCggtATT
AAAGTGAAGATTcagCGGGTTAATAACTCCAGTACCAATGATAATCTGGTCCGTAAGAACGATCAGGTATACATC
aatTTCGTCGCGAGCAAAACTcatCTCTTCCCGCTTTACGCCgatACAGCTACGACAAACAAGGAAAAAACCATA
AAAATTTCCAGCTCCGGAAACAGATTCAATCAAGTAGTTGTAATGAACTCTGTGGGTaatAATTGTACGATGAAC
TTTaagAATAACAATGGGAACAATattGGACTTTTGGGCTTCAAAGCCGACACAGTGGTGGCGTCCACCTGGTAT
TACACGcacATGcggGACCATACGAATTCGAACGGTTGCTTCTGGAACTTTATCTCGGAAgaaCACGGGTGGCAA
GAAAAA
SEQ ID NO: 14-Polypeptide Sequence of rBoNT/E(0)
MPKINSFNYNDPVNDRTILYIKPGGCQEFYKSENIMKNIWIIPERNVIGTTPQDFHPPTSLKNGDSSYYDPNYLQ
SDEEKDRFLKIVTKIFNRINNNLSGGILLEELSKANPYLGNDNTPDNQFHIGDASAVEIKFSNGSQDILLPNVII
MGAEPDLFETNSSNISLRNNYMPSNHGFGSIAIVTFSPEYSFRENDNSMNEFIQDPALTLMHQLIYSLHGLYGAK
GITTKYTITQKQNPLITNIRGTNIEEFLTFGGTDLNIITSAQSNDIYTNLLADYKKIASKLSKVQVSNPLLNPYK
DVFEAKYGLDKDASGIYSVNINKENDIFKKLYSFTEFDLATKFQVKCRQTYIGQYKYFKLSNLLNDSIYNISEGY
NINNLKVNFRGQNANLNPRIITPITGRGLVKKIIRFCKNIVSVKGIRKSICIEINNGELFFVASENSYNDDNINT
PKEIDDTVTSNNNYENDLDQVILNENSESAPGLSDEKLNLTIQNDAYIPKYDSNGTSDIEQHDVNELNVFFYLDA
QKVPEGENNVNLTSSIDTALLEQPKIYTFFSSEFINNVNKPVQAALFVSWIQQVLVDETTEANQKSTVDKIADIS
IVVPYIGLALNIGNEAQKGNFKDALELLGAGILLEFEPELLIPTILVFTIKSFLGSSDNKNKVIKAINNALKERD
EKWKEVYSFIVSNWMTKINTQFNKRKEQMYQALQNQVNAIKTIIESKYNSYTLEEKNELTNKYDIKQIENELNQK
VSIAMNNIDRFLTESSISYLMKLINEVKINKLREYDENVKTYLLNYIIQHGSILGESQQELNSMVTDTLNNSIPF
KLSSYTDDKILISYFNKFFKRIKSSSVLNMRYKNDKYVDTSGYDSNININGDVYKYPTNKNQFGIYNDKLSEVNI
SQNDYIIYDNKYKNFSISFWVRIPNYDNKIVNVNNEYTIINCMRDNNSGWKVSLNHNEIIWTLQDNAGINQKLAF
NYGNANGISDYINKWIFVTITNDRLGDSKLYINGNLIDQKSILNLGNIHVSDNILFKIVNCSYTRYIGIRYFNIF
DKELDETEIQTLYSNEPNTNILKDFWGNYLLYDKEYYLLNVLKPNNFIDRRKDSTLSINNIRSTILLANRLYSGI
KVKIQRVNNSSTNDNLVRKNDQVYINFVASKTHLFPLYADTATTNKEKTIKISSSGNRFNQVVVMNSVGNNCTMN
FKNNNGNNIGLLGFKADTVVASTWYYTHMRDHTNSNGCFWNFISEEHGWQEK
SEQ ID NO: 15-Nucleotide Sequence of rBoNT/F(0)
ATGCCGGTGGTCATCAACAGCTTCAACTACAACGACCCAGTAAACGACGACACGATCCTGTATATGCAAATCCCG
TATGAAGAGAAGAGCAAGAAGTACTATAAGGCCTTTGAAATCATGCGCAATGTGTGGATTATTCCGGAGCGTAAT
ACGATTGGTACTGACCCAAGCGACTTCGATCCACCTGCGTCTTTGGAAAACGGCTCGTCCGCATATTACGACCCG
AATTACCTGACCACCGATGCGGAGAAAGATCGTTATTTGAAAACCACCATCAAGCTGTTCAAACGCATTAACAGC
AATCCGGCAGGTGAGGTCCTGCTGCAAGAGATTAGCTACGCAAAGCCTTATCTGGGTAATGAGCATACGCCTATT
AACGAGTTTCACCCGGTTACCCGCACTACCAGCGTTAACATCAAGTCCTCGACCAACGTGAAGTCTAGCATTATC
CTGAACCTGCTGGTTCTGGGTGCCGGTCCGGACATCTTCGAAAACTCTAGCTACCCGGTGCGTAAACTGATGGAT
AGCGGCGGTGTTTATGACCCGAGCAATGACGGTTTTGGCAGCATCAATATCGTGACGTTTAGCCCGGAGTACGAG
TACACCTTCAATGATATCAGCGGTGGTTACAATTCTTCTACCGAGAGCTTCATCGCCGACCCGGCGATCAGCCTG
GCACACCAACTGATCTATGCATTGCATGGCTTGTACGGTGCCCGTGGTGTGACGTATAAAGAGACTATCAAGGTT
AAGCAGGCACCTCTGATGATTGCGGAAAAGCCGATTCGCCTGGAAGAGTTCCTGACCTTCGGCGGTCAAGATTTG
AACATCATTACCTCGGCCATGAAAGAGAAAATCTATAACAATTTGCTGGCCAACTATGAAAAGATTGCAACGCGC
TTGTCTCGTGTTAACTCCGCTCCGCCGGAATACGACATTAATGAGTACAAAGACTACTTTCAATGGAAATATGGC
CTGGACAAAAATGCGGATGGTTCTTATACCGTGAATGAAAACAAATTCAATGAAATCTACAAGAAACTGTACAGC
TTCACCGAAATCGATCTGGCGAACAAGTTCAAAGTCAAATGTCGTAATACCTACTTCATCAAATATGGCTTCCTG
AAAGTCCCGAACCTGCTGGACGATGACATCTATACCGTCAGCGAAGGCTTCAACATCGGCAATCTGGCCGTGAAT
AATCGTGGTCAGAACATCAAACTGAATCCGAAAATCATTGACTCCATCCCAGACAAGGGCCTGGTTGAGAAAATC
GTGAAGTTCTGCAAAAGCGTTATTCCGCGTAAAGGTACGAAAGCACCGCCTCGCCTGTGCATTCGCGTTAACAAC
CGTGAGTTGTTCTTTGTGGCATCTGAAAGCAGCTACAACGAGAACGACATCAACACCCCTAAAGAAATTGATGAT
ACCACGAACCTGAATAACAATTATCGCAACAATCTGGACGAGGTGATCCTGGATTACAATTCGGAAACCATTCCG
CAAATTAGCAATCAGACGCTGAACACCCTGGTTCAGGACGATAGCTACGTTCCGCGTTACGACTCCAATGGTACT
AGCGAGATTGAAGAACACAACGTAGTGGACTTGAACGTTTTCTTTTATCTGCACGCCCAGAAGGTTCCGGAGGGC
GAAACCAATATTAGCCTGACCAGCTCGATCGACACCGCGCTGTCTGAGGAGAGCCAAGTCTACACCTTTTTCAGC
AGCGAGTTTATCAACACTATTAACAAGCCAGTTCATGCTGCATTGTTTATCTCTTGGATTAACCAGGTGATTCGC
GACTTTACGACGGAGGCGACCCAGAAGTCTACCTTCGACAAAATTGCAGACATCTCCCTGGTCGTCCCATACGTC
GGCCTGGCGTTGAATATTGGCAATGAAGTTCAAAAAGAGAACTTCAAAGAAGCGTTCGAGCTGCTGGGTGCAGGC
ATCCTGCTGGAGTTCGTGCCGGAACTGTTGATCCCGACCATCCTGGTGTTCACCATTAAGAGCTTCATTGGATCC
TCCGAGAATAAGAACAAGATCATCAAGGCGATCAATAACAGCCTGATGGAGCGTGAAACGAAGTGGAAAGAAATC
TATAGCTGGATTGTTAGCAATTGGCTGACTCGTATTAACACGCAATTCAACAAGCGTAAAGAGCAAATGTACCAA
GCCCTGCAAAACCAAGTTGACGCCATCAAAACGGTAATTGAATACAAGTACAACAATTACACGAGCGATGAGCGC
AACCGCCTGGAAAGCGAATACAACATCAACAACATTCGCGAAGAATTGAACAAGAAAGTGAGCCTGGCGATGGAG
AACATTGAGCGTTTTATCACCGAAAGCAGCATCTTTTACCTGATGAAATTGATTAATGAGGCGAAAGTCTCGAAA
CTGCGTGAGTACGACGAAGGTGTGAAAGAGTATCTGCTGGATTACATTAGCGAGCACCGTAGCATCTTGGGTAAC
TCGGTTCAGGAGCTGAACGATCTGGTGACCTCTACCCTGAACAATAGCATCCCGTTCGAACTGAGCAGCTATACC
AATGACAAGATTCTGATTCTGTATTTCAATAAACTGTATAAGAAGATCAAGGATAACAGCATTCTGGATATGCGT
TACGAAAACAATAAGTTTATCGACATTTCTGGTTACGGCAGCAACATTTCCATCAATGGCGATGTCTACATCTAC
AGCACCAATCGCAACCAGTTCGGCATCTACTCTAGCAAACCGAGCGAAGTTAACATCGCACAGAACAATGATATT
ATTTATAACGGTCGTTATCAAAACTTCTCTATCAGCTTTTGGGTCCGTATCCCGAAGTACTTCAATAAAGTCAAT
CTGAATAATGAATACACGATCATCGACTGCATTCGCAATAACAACAGCGGTTGGAAAATCAGCCTGAATTACAAC
AAAATTATTTGGACCCTGCAAGATACGGCGGGTAACAATCAGAAACTGGTGTTTAACTACACGCAAATGATCAGC
ATTTCTGACTATATCAACAAGTGGATCTTTGTTACCATCACCAATAATCGTCTGGGCAATAGCCGTATTTACATC
AACGGTAACCTGATTGATGAGAAAAGCATCAGCAACCTGGGCGATATTCACGTCAGCGACAACATTCTGTTCAAA
ATTGTTGGTTGTAACGATACCCGTTACGTCGGCATCCGTTATTTCAAGGTTTTCGATACGGAGCTGGGTAAAACG
GAAATCGAAACGTTGTACTCCGATGAACCAGATCCGAGCATTCTGAAGGACTTTTGGGGTAACTACTTGCTGTAC
AATAAACGTTACTATCTGCTGAATCTGTTGCGCACCGACAAGAGCATTACCCAAAACAGCAATTTCCTGAACATT
AATCAGCAACGCGGCGTATACCAAAAACCGAACATCTTCAGCAATACGCGCCTGTATACTGGTGTTGAAGTGATC
ATTCGTAAGAACGGTAGCACCGACATTAGCAACACGGACAATTTCGTCCGTAAGAATGACCTGGCGTACATTAAC
GTCGTGGACCGTGATGTCGAGTATCGTCTGTACGCAGACATCAGCATTGCGAAACCGGAAAAGATTATCAAGCTG
ATCCGTACCAGCAACAGCAACAACAGCCTGGGTCAGATCATTGTGATGGACAGCATTGGTAATAACTGCACGATG
AACTTCCAGAACAACAATGGTGGTAATATCGGTCTGCTGGGTTTTCACAGCAATAATCTGGTTGCTTCCAGCTGG
TACTACAATAACATTCGTAAAAACACGTCTAGCAATGGTTGTTTTTGGAGCTTTATCAGCAAAGAGCACGGCTGG
CAAGAAAAT
SEQ ID NO: 16-Polypeptide Sequence of rBoNT/F(0)
MPVVINSFNYNDPVNDDTILYMQIPYEEKSKKYYKAFEIMRNVWIIPERNTIGTDPSDFDPPASLENGSSAYYDP
NYLTTDAEKDRYLKTTIKLFKRINSNPAGEVLLQEISYAKPYLGNEHTPINEFHPVTRTTSVNIKSSTNVKSSII
LNLLVLGAGPDIFENSSYPVRKLMDSGGVYDPSNDGFGSINIVTFSPEYEYTENDISGGYNSSTESFIADPAISL
AHQLIYALHGLYGARGVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKENEIYKKLYSFTEIDLANKFKVKCRNTYFIKYGEL
KVPNLLDDDIYTVSEGFNIGNLAVNNRGQNIKLNPKIIDSIPDKGLVEKIVKFCKSVIPRKGTKAPPRLCIRVNN
RELFFVASESSYNENDINTPKEIDDTTNLNNNYRNNLDEVILDYNSETIPQISNQTLNTLVQDDSYVPRYDSNGT
SEIEEHNVVDLNVFFYLHAQKVPEGETNISLTSSIDTALSEESQVYTFFSSEFINTINKPVHAALFISWINQVIR
DFTTEATQKSTFDKIADISLVVPYVGLALNIGNEVQKENFKEAFELLGAGILLEFVPELLIPTILVFTIKSFIGS
SENKNKIIKAINNSLMERETKWKEIYSWIVSNWLTRINTQFNKRKEQMYQALQNQVDAIKTVIEYKYNNYTSDER
NRLESEYNINNIREELNKKVSLAMENIERFITESSIFYLMKLINEAKVSKLREYDEGVKEYLLDYISEHRSILGN
SVQELNDLVTSTLNNSIPFELSSYTNDKILILYENKLYKKIKDNSILDMRYENNKFIDISGYGSNISINGDVYIY
STNRNQFGIYSSKPSEVNIAQNNDIIYNGRYQNFSISFWVRIPKYFNKVNLNNEYTIIDCIRNNNSGWKISLNYN
KIIWTLQDTAGNNQKLVFNYTQMISISDYINKWIFVTITNNRLGNSRIYINGNLIDEKSISNLGDIHVSDNILFK
IVGCNDTRYVGIRYFKVFDTELGKTEIETLYSDEPDPSILKDFWGNYLLYNKRYYLLNLLRTDKSITQNSNELNI
NQQRGVYQKPNIFSNTRLYTGVEVIIRKNGSTDISNTDNFVRKNDLAYINVVDRDVEYRLYADISIAKPEKIIKL
IRTSNSNNSLGQIIVMDSIGNNCTMNFQNNNGGNIGLLGFHSNNLVASSWYYNNIRKNTSSNGCFWSFISKEHGW
QEN
SEQ ID NO: 17-Nucleotide Sequence of rBoNT/A(0) (His-tagged)
ATGCCGTTTGTGAACAAGCAGTTCAACTATAAAGATCCGGTTAATGGTGTGGATATCGCCTATATCAAAATTCCG
AATGCAGGTCAGATGCAGCCGGTTAAAGCCTTTAAAATCCATAACAAAATTTGGGTGATTCCGGAACGTGATACC
TTTACCAATCCGGAAGAAGGTGATCTGAATCCGCCTCCGGAAGCAAAACAGGTTCCGGTTAGCTATTATGATAGC
ACCTATCTGAGCACCGATAACGAGAAAGATAACTATCTGAAAGGTGTGACCAAACTGTTTGAACGCATTTATAGT
ACCGATCTGGGTCGTATGCTGCTGACCAGCATTGTTCGTGGTATTCCGTTTTGGGGTGGTAGCACCATTGATACC
GAACTGAAAGTTATTGACACCAACTGCATTAATGTGATTCAGCCGGATGGTAGCTATCGTAGCGAAGAACTGAAT
CTGGTTATTATTGGTCCGAGCGCAGATATCATTCAGTTTGAATGTAAAAGCTTTGGCCACGAAGTTCTGAATCTG
ACCCGTAATGGTTATGGTAGTACCCAGTATATTCGTTTCAGTCCGGATTTTACCTTTGGCTTTGAAGAAAGCCTG
GAAGTTGATACAAATCCGCTGTTAGGTGCAGGTAAATTTGCAACCGATCCGGCAGTTACCCTGGCACACCAGCTG
ATTTATGCCGGTCATCGTCTGTATGGTATTGCCATTAATCCGAATCGTGTGTTCAAAGTGAATACCAACGCCTAT
TATGAAATGAGCGGTCTGGAAGTGAGTTTTGAAGAACTGCGTACCTTTGGTGGTCATGATGCCAAATTTATCGAT
AGCCTGCAAGAAAATGAATTTCGCCTGTACTACTATAACAAATTCAAGGATATTGCGAGCACCCTGAATAAAGCC
AAAAGCATTGTTGGCACCACCGCAAGCCTGCAGTATATGAAAAATGTGTTTAAAGAAAAATATCTGCTGAGCGAA
GATACCAGCGGTAAATTTAGCGTTGACAAACTGAAATTCGATAAACTGTACAAGATGCTGACCGAGATTTATACC
GAAGATAACTTCGTGAAGTTTTTCAAAGTGCTGAACCGCAAAACCTACCTGAACTTTGATAAAGCCGTGTTCAAA
ATCAACATCGTGCCGAAAGTGAACTATACCATCTATGATGGTTTTAACCTGCGCAATACCAATCTGGCAGCAAAC
TTTAATGGTCAGAACACCGAAATCAACAACATGAACTTTACCAAACTGAAGAACTTCACCGGTCTGTTCGAATTT
TACAAACTGCTGTGTGTTCGTGGCATTATTACCAGCAAAACCAAAAGTCTGGATAAAGGCTACAATAAAGCCCTG
AATGATCTGTGCATTAAGGTGAATAATTGGGACCTGTTTTTTAGCCCGAGCGAGGATAATTTCACCAACGATCTG
AACAAAGGCGAAGAAATTACCAGCGATACCAATATTGAAGCAGCCGAAGAAAACATTAGCCTGGATCTGATTCAG
CAGTATTATCTGACCTTCAACTTCGATAATGAGCCGGAAAATATCAGCATTGAAAACCTGAGCAGCGATATTATT
GGCCAGCTGGAACTGATGCCGAATATTGAACGTTTTCCGAACGGCAAAAAATACGAGCTGGATAAATACACCATG
TTCCATTATCTGCGTGCCCAAGAATTTGAACATGGTAAAAGCCGTATTGCACTGACCAATAGCGTTAATGAAGCA
CTGCTGAACCCGAGCCGTGTTTATACCTTTTTTAGCAGCGATTACGTGAAAAAGGTTAACAAAGCAACCGAAGCA
GCCATGTTTTTAGGTTGGGTTGAACAGCTGGTTTATGATTTCACCGATGAAACCAGCGAAGTTAGCACCACCGAT
AAAATTGCAGATATTACCATCATCATCCCGTATATCGGTCCGGCACTGAATATTGGCAATATGCTGTATAAAGAC
GATTTTGTGGGTGCCCTGATTTTTAGCGGTGCAGTTATTCTGCTGGAATTTATTCCGGAAATTGCCATTCCGGTT
CTGGGCACCTTTGCACTGGTGAGCTATATTGCAAATAAAGTTCTGACCGTGCAGACCATCGATAATGCACTGAGC
AAACGTAACGAAAAATGGGATGAAGTGTACAAGTATATCGTGACCAATTGGCTGGCAAAAGTTAACACCCAGATT
GACCTGATTCGCAAGAAGATGAAAGAAGCACTGGAAAATCAGGCAGAAGCAACCAAAGCCATTATCAACTATCAG
TATAACCAGTACACCGAAGAAGAGAAAAATAACATCAACTTCAACATCGACGATCTGTCCAGCAAACTGAACGAA
AGCATCAACAAAGCCATGATTAACATTAACAAATTTCTGAACCAGTGCAGCGTGAGCTATCTGATGAATAGCATG
ATTCCGTATGGTGTGAAACGTCTGGAAGATTTTGATGCAAGCCTGAAAGATGCCCTGCTGAAATATATCTATGAT
AATCGTGGCACCCTGATTGGTCAGGTTGATCGTCTGAAAGATAAAGTGAACAACACCCTGAGTACCGATATTCCT
TTTCAGCTGAGCAAATATGTGGATAATCAGCGTCTGCTGTCAACCTTTACCGAATACATTAAGAACATCATCAAC
ACCAGCATTCTGAACCTGCGTTATGAAAGCAATCATCTGATTGATCTGAGCCGTTATGCCAGCAAAATCAATATA
GGCAGCAAGGTTAACTTCGACCCGATTGACAAAAATCAGATACAGCTGTTTAATCTGGAAAGCAGCAAAATTGAG
GTGATCCTGAAAAACGCCATTGTGTATAATAGCATGTACGAGAATTTCTCGACCAGCTTTTGGATTCGTATCCCG
AAATACTTTAATAGCATCAGCCTGAACAACGAGTACACCATTATTAACTGCATGGAAAACAATAGCGGCTGGAAA
GTTAGCCTGAATTATGGCGAAATTATCTGGACCCTGCAGGATACCCAAGAAATCAAACAGCGTGTGGTTTTCAAA
TACAGCCAGATGATTAATATCAGCGACTATATCAACCGCTGGATTTTTGTGACCATTACCAATAATCGCCTGAAT
AACAGCAAGATCTATATTAACGGTCGTCTGATTGACCAGAAACCGATTAGTAATCTGGGTAATATTCATGCGAGC
AACAACATCATGTTTAAACTGGATGGTTGTCGTGATACCCATCGTTATATTTGGATCAAGTACTTCAACCTGTTC
GATAAAGAGTTGAACGAAAAAGAAATTAAAGACCTGTATGATAACCAGAGCAACAGCGGTATTCTGAAGGATTTT
TGGGGAGATTATCTGCAGTATGACAAACCGTATTATATGCTGAATCTGTACGACCCGAATAAATACGTGGATGTG
AATAATGTTGGCATCCGTGGTTATATGTACCTGAAAGGTCCGCGTGGTAGCGTTATGACCACAAACATTTATCTG
AATAGCAGCCTGTATCGCGGAACCAAATTCATCATTAAAAAGTATGCCAGCGGCAACAAGGATAATATTGTGCGT
AATAATGATCGCGTGTACATTAACGTTGTGGTGAAGAATAAAGAATATCGCCTGGCAACCAATGCAAGCCAGGCA
GGCGTTGAAAAAATTCTGAGTGCCCTGGAAATTCCGGATGTTGGTAATCTGAGCCAGGTTGTTGTGATGAAAAGC
AAAAATGATCAGGGCATCACCAACAAGTGCAAAATGAATCTGCAGGACAATAACGGCAACGATATTGGTTTTATT
GGCTTCCACCAGTTCAACAATATTGCGAAACTGGTTGCAAGCAATTGGTATAATCGTCAGATTGAACGTAGCAGT
CGTACCCTGGGTTGTAGCTGGGAATTTATCCCTGTGGATGATGGTTGGGGTGAACGTCCGCTGGAAAACCTGTAT
TTTCAAGGTGCAAGTCATCATCACCATCACCACCATCATTAA
SEQ ID NO: 18-Polypeptide Sequence of rBoNT/A(0) (His-tagged)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHQL
IYAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLENLESSKIEVILKNAIVYNSMYENFSTSFWIRIPKYENSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLEDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSS
RTLGCSWEFIPVDDGWGERPLENLYFQGASHHHHHHHH
SEQ ID NO: 19-Nucleotide Sequence of rLHN/A (His-tagged)
ATGCCGTTTGTGAACAAGCAGTTCAACTATAAAGATCCGGTTAATGGTGTGGATATCGCCTATATCAAAATTCCG
AATGCAGGTCAGATGCAGCCGGTTAAAGCCTTTAAAATCCATAACAAAATTTGGGTGATTCCGGAACGTGATACC
TTTACCAATCCGGAAGAAGGTGATCTGAATCCGCCTCCGGAAGCAAAACAGGTTCCGGTTAGCTATTATGATAGC
ACCTATCTGAGCACCGATAACGAGAAAGATAACTATCTGAAAGGTGTGACCAAACTGTTTGAACGCATTTATAGT
ACCGATCTGGGTCGTATGCTGCTGACCAGCATTGTTCGTGGTATTCCGTTTTGGGGTGGTAGCACCATTGATACC
GAACTGAAAGTTATTGACACCAACTGCATTAATGTGATTCAGCCGGATGGTAGCTATCGTAGCGAAGAACTGAAT
CTGGTTATTATTGGTCCGAGCGCAGATATCATTCAGTTTGAATGTAAATCCTTTGGCCACGAAGTTCTGAATCTG
ACCCGTAATGGTTATGGTAGTACCCAGTATATTCGTTTCAGTCCGGATTTTACCTTTGGCTTTGAAGAAAGCCTG
GAAGTTGATACAAATCCGCTGTTAGGTGCAGGTAAATTTGCAACCGATCCGGCAGTTACCCTGGCACATGAACTG
ATTCATGCCGGTCATCGTCTGTATGGTATTGCAATTAATCCGAACCGTGTGTTCAAAGTGAATACCAACGCATAT
TATGAAATGAGCGGTCTGGAAGTGTCATTTGAAGAACTGCGTACCTTTGGTGGTCATGATGCCAAATTTATCGAT
AGCCTGCAAGAAAATGAATTTCGCCTGTACTACTATAACAAATTCAAGGATATTGCGAGCACCCTGAATAAAGCC
AAAAGCATTGTTGGCACCACCGCAAGCCTGCAGTATATGAAAAATGTGTTTAAAGAAAAATATCTGCTGAGCGAA
GATACCAGCGGTAAATTTAGCGTTGACAAACTGAAATTCGATAAACTGTACAAGATGCTGACCGAGATTTATACC
GAAGATAACTTCGTGAAGTTTTTCAAAGTGCTGAACCGCAAAACCTACCTGAACTTTGATAAAGCCGTGTTCAAA
ATCAACATCGTGCCGAAAGTGAACTATACCATCTATGATGGTTTTAACCTGCGCAATACCAATCTGGCAGCAAAC
TTTAATGGTCAGAACACCGAAATCAACAACATGAACTTTACCAAACTGAAGAACTTCACCGGTCTGTTCGAATTT
TACAAACTGCTGTGTGTTCGTGGCATTATTACCAGCAAAACCAAAAGTCTGGATAAAGGCTACAATAAAGCCCTG
AATGATCTGTGCATTAAGGTGAATAATTGGGACCTGTTTTTTAGCCCGAGCGAGGATAATTTCACCAACGATCTG
AACAAAGGCGAAGAAATTACCAGCGATACCAATATTGAAGCAGCCGAAGAAAACATTAGCCTGGATCTGATTCAG
CAGTATTATCTGACCTTCAACTTCGATAATGAGCCGGAAAATATCAGCATTGAAAACCTGAGCAGCGATATTATT
GGCCAGCTGGAACTGATGCCGAATATTGAACGTTTTCCGAACGGCAAAAAATACGAGCTGGATAAATACACCATG
TTCCATTATCTGCGTGCCCAAGAATTTGAACATGGTAAAAGCCGTATTGCACTGACCAATAGCGTTAATGAAGCA
CTGCTGAACCCGAGCCGTGTTTATACCTTTTTTAGCAGCGATTACGTGAAAAAGGTTAACAAAGCAACCGAAGCA
GCCATGTTTTTAGGTTGGGTTGAACAGCTGGTTTATGATTTCACCGATGAAACCAGCGAAGTTAGCACCACCGAT
AAAATTGCAGATATTACCATCATCATCCCGTATATCGGTCCGGCACTGAATATTGGCAATATGCTGTATAAAGAC
GATTTTGTGGGTGCCCTGATTTTTAGCGGTGCAGTTATTCTGCTGGAATTTATTCCGGAAATTGCCATTCCGGTT
CTGGGCACCTTTGCACTGGTGAGCTATATTGCAAATAAAGTTCTGACCGTGCAGACCATCGATAATGCACTGAGC
AAACGTAACGAAAAATGGGATGAAGTGTACAAGTATATCGTGACCAATTGGCTGGCAAAAGTTAACACCCAGATT
GACCTGATTCGCAAGAAGATGAAAGAAGCACTGGAAAATCAGGCAGAAGCAACCAAAGCCATTATCAACTATCAG
TATAACCAGTACACCGAAGAAGAGAAAAATAACATCAACTTCAACATCGACGATCTGTCCAGCAAACTGAACGAA
AGCATCAACAAAGCCATGATTAACATTAACAAATTTCTGAACCAGTGCAGCGTGAGCTATCTGATGAATAGCATG
ATTCCGTATGGTGTGAAACGTCTGGAAGATTTTGATGCAAGCCTGAAAGATGCCCTGCTGAAATATATCTATGAT
AATCGTGGCACCCTGATTGGTCAGGTTGATCGTCTGAAAGATAAAGTGAACAACACCCTGAGTACCGATATTCCT
TTTCAGCTGAGCAAATATGTGGATAATCAGCGTCTGCTGTCAACCGAAAATCTGTATTTCCAGGGTGCAAGTCAT
CATCACCATCACCACCATCATTAA
SEQ ID NO: 20-Polypeptide Sequence of rLHN/A (His-tagged)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVEK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTENLYFQGASHHHHHHHH
SEQ ID NO: 21-Nucleotide Sequence of rHc/A (His-tagged)
ATGCATCATCACCATCACCACGAAAATCTATACTTCCAAGGAAAAAACATCATCAATACTAGCATTCTGAACCTG
CGTTACGAGAGCAATCATCTGATTGATCTGAGCCGTTATGCAAGCAAGATCAACATCGGTAGCAAGGTCAATTTT
GACCCGATCGATAAGAACCAGATCCAGCTGTTTAATCTGGAATCGAGCAAAATTGAGGTTATCCTGAAAAACGCC
ATTGTCTACAACTCCATGTACGAGAATTTCTCCACCAGCTTCTGGATTCGCATCCCGAAATACTTCAACAGCATT
AGCCTGAACAACGAGTATACTATCATCAACTGTATGGAGAACAACAGCGGTTGGAAGGTGTCTCTGAACTATGGT
GAGATCATTTGGACCTTGCAGGACACCCAAGAGATCAAGCAGCGCGTCGTGTTCAAGTACTCTCAAATGATCAAC
ATTTCCGATTACATTAATCGTTGGATCTTCGTGACCATTACGAATAACCGTCTGAATAACAGCAAGATTTACATC
AATGGTCGCTTGATCGATCAGAAACCGATTAGCAACCTGGGTAATATCCACGCAAGCAACAACATTATGTTCAAA
TTGGACGGTTGCCGCGATACCCATCGTTATATCTGGATCAAGTATTTCAACCTGTTTGATAAAGAACTGAATGAG
AAGGAGATCAAAGATTTGTATGACAACCAATCTAACAGCGGCATTTTGAAGGACTTCTGGGGCGATTATCTGCAA
TACGATAAGCCGTACTATATGCTGAACCTGTATGATCCGAACAAATATGTGGATGTCAATAATGTGGGTATTCGT
GGTTACATGTATTTGAAGGGTCCGCGTGGCAGCGTTATGACGACCAACATTTACCTGAACTCTAGCCTGTACCGT
GGTACGAAATTCATCATTAAGAAATATGCCAGCGGCAACAAAGATAACATTGTGCGTAATAACGATCGTGTCTAC
ATCAACGTGGTCGTGAAGAATAAAGAGTACCGTCTGGCGACCAACGCTTCGCAGGCGGGTGTTGAGAAAATTCTG
AGCGCGTTGGAGATCCCTGATGTCGGTAATCTGAGCCAAGTCGTGGTTATGAAGAGCAAGAACGACCAGGGTATC
ACTAACAAGTGCAAGATGAACCTGCAAGACAACAATGGTAACGACATCGGCTTTATTGGTTTCCACCAGTTCAAC
AATATTGCTAAACTGGTAGCGAGCAATTGGTACAATCGTCAGATTGAGCGCAGCAGCCGTACTTTGGGCTGTAGC
TGGGAGTTTATCCCGGTCGATGATGGTTGGGGCGAACGTCCGCTGTAA
SEQ ID NO: 22-Polypeptide Sequence of rH_{C}/A (His-tagged)
MHHHHHHENLYFQGKNIINTSILNLRYESNHLIDLSRYASKINIGSKVNEDPIDKNQIQLENLESSKIEVILKNA
IVYNSMYENFSTSFWIRIPKYENSISLNNEYTIINCMENNSGWKVSLNYGEIIWTLQDTQEIKQRVVFKYSQMIN
ISDYINRWIFVTITNNRLNNSKIYINGRLIDQKPISNLGNIHASNNIMFKLDGCRDTHRYIWIKYFNLEDKELNE
KEIKDLYDNQSNSGILKDFWGDYLQYDKPYYMLNLYDPNKYVDVNNVGIRGYMYLKGPRGSVMTTNIYLNSSLYR
GTKFIIKKYASGNKDNIVRNNDRVYINVVVKNKEYRLATNASQAGVEKILSALEIPDVGNLSQVVVMKSKNDQGI
TNKCKMNLQDNNGNDIGFIGFHQFNNIAKLVASNWYNRQIERSSRTLGCSWEFIPVDDGWGERPL
SEQ ID NO: 23-Nucleotide Sequence of rL_{C}/A (His-tagged)
ATGCCGTTTGTGAACAAGCAGTTCAACTATAAAGATCCGGTTAATGGTGTGGATATCGCCTATATCAAAATTCCG
AATGCAGGTCAGATGCAGCCGGTTAAAGCCTTTAAAATCCATAACAAAATTTGGGTGATTCCGGAACGTGATACC
TTTACCAATCCGGAAGAAGGTGATCTGAATCCGCCTCCGGAAGCAAAACAGGTTCCGGTTAGCTATTATGATAGC
ACCTATCTGAGCACCGATAACGAGAAAGATAACTATCTGAAAGGTGTGACCAAACTGTTTGAACGCATTTATAGT
ACCGATCTGGGTCGTATGCTGCTGACCAGCATTGTTCGTGGTATTCCGTTTTGGGGTGGTAGCACCATTGATACC
GAACTGAAAGTTATTGACACCAACTGCATTAATGTGATTCAGCCGGATGGTAGCTATCGTAGCGAAGAACTGAAT
CTGGTTATTATTGGTCCGAGCGCAGATATCATTCAGTTTGAATGTAAATCCTTTGGCCACGAAGTTCTGAATCTG
ACCCGTAATGGTTATGGTAGTACCCAGTATATTCGTTTCAGTCCGGATTTTACCTTTGGCTTTGAAGAAAGCCTG
GAAGTTGATACAAATCCGCTGTTAGGTGCAGGTAAATTTGCAACCGATCCGGCAGTTACCCTGGCACATGAACTG
ATTCATGCCGGTCATCGTCTGTATGGTATTGCAATTAATCCGAACCGTGTGTTCAAAGTGAATACCAACGCATAT
TATGAAATGAGCGGTCTGGAAGTGTCATTTGAAGAACTGCGTACCTTTGGTGGTCATGATGCCAAATTTATCGAT
AGCCTGCAAGAAAATGAATTTCGCCTGTACTACTATAACAAATTCAAGGATATTGCGAGCACCCTGAATAAAGCC
AAAAGCATTGTTGGCACCACCGCAAGCCTGCAGTATATGAAAAATGTGTTTAAAGAAAAATATCTGCTGAGCGAA
GATACCAGCGGTAAATTTAGCGTTGACAAACTGAAATTCGATAAACTGTACAAGATGCTGACCGAGATTTATACC
GAAGATAACTTCGTGAAGTTTTTCAAAGTGCTGAACCGCAAAACCTACCTGAACTTTGATAAAGCCGTGTTCAAA
ATCAACATCGTGCCGAAAGTGAACTATACCATCTATGATGGTTTTAACCTGCGCAATACCAATCTGGCAGCAAAC
TTTAATGGTCAGAACACCGAAATCAACAACATGAACTTTACCAAACTGAAGAACTTCACCGGTCTGTTTGAAGAG
AATCTGTATTTCCAGGGTGCAAGTCATCATCACCATCACCACCATCATTAA
SEQ ID NO: 24-Polypeptide Sequence of rLC/A (His-tagged)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVEK
INIVPKVNYTIYDGFNLRNTNLAANFNGQNTEINNMNFTKLKNFTGLFEENLYFQGASHHHHHHHH
SEQ ID NO: 25-Nucleotide Sequence of rBoNT/FA(0) (His-tagged)
ATGCCGGTTGTGATTAACAGCTTCAATTATGATGATCCGGTGAACGATAACACCATCATTTATATCCGTCCGCCT
TATTATGAAACCAGCAACACCTATTTCAAAGCCTTCCAGATTATGGATAACGTGTGGATTATTCCGGAACGTTAT
CGTCTGGGTATTGATCCGAGCCTGTTTAATCCGCCTGTTAGCCTGAAAGCAGGTAGTGATGGTTATTTTGATCCG
AATTATCTGAGCACCAACACCGAGAAAAACAAATACCTGCAGATTATGATCAAGCTGTTCAAACGCATTAATAGC
AAACCGGCAGGTCAGATTCTGCTGGAAGAAATCAAAAATGCAATTCCGTATCTGGGCAACAGCTATACCCAAGAA
GAACAGTTTACCACCAATAATCGTACCGTGAGCTTTAATGTTAAACTGGCCAATGGTAATATCGTTCAGCAGATG
GCAAATCTGATTATTTGGGGTCCGGGTCCTGATCTGACCACAAATAAAACCGGTGGTATCATCTATAGCCCGTAT
CAGAGCATGGAAGCAACCCCGTATAAAGATGGTTTTGGTAGCATTATGACCGTGGAATTTAGTCCGGAATATGCA
ACCGCCTTTAACGATATTTCAATTGCAAGCCATAGTCCGTCGCTGTTTATCAAAGATCCGGCACTGATTCTGATG
CACCAGCTGATTTATGTTCTGCATGGTCTGTATGGCACCTATATCACCGAATACAAAATTACCCCGAATGTGGTT
CAGAGCTATATGAAAGTTACCAAACCGATTACCAGCGCAGAATTTCTGACCTTTGGTGGTCGTGATCGCAATATT
GTTCCGCAGAGCATTCAGAGCCAGCTGTATAACAAAGTTCTGAGCGATTATAAACGTATTGCCAGCCGTCTGAAT
AAAGTTAATACCGCAACCGCACTGATCAACATCGATGAATTCAAAAACCTGTACGAGTGGAAATACCAGTTTGCC
AAAGATAGCAATGGTGTGTATAGCGTGGATCTGAACAAATTTGAGCAGCTGTACAAAAAAATCTATAGCTTCACC
GAATTCAACCTGGCCTATGAGTTTAAAATCAAAACCCGTCTGGGTTATCTGGCCGAAAATTTTGGTCCGTTTTAT
CTGCCGAATCTGCTGGATGATAGCATTTATACCGAAGTGGATGGTTTTAACATTGGTGCACTGAGCATTAACTAT
CAGGGTCAGAATATTGGCAGCGATATCAACAGCATCAAAAAACTGCAAGGTCAGGGTGTTGTTAGCCGTGTTGTT
CGTCTGTGTAGCAATAGCAATACCAAAAACAGCCTGTGCATTACCGTTAATAATCGCGACCTGTTTTTTATCGCA
AGCCAAGAAAGCTATGGCGAGAATACCATTAACACCTATAAAGAGATTGACGATACCACCACACTGGATCCGAGC
TTTGAAGATATTCTGGATAAAGTGATCCTGAACTTCAACGAACAGGTTATTCCGCAGATGCCGAATCGTAATGTT
AGCACCGATATTCAGAAAGACAACTACATCCCGAAATACGATTATAACCGCACCGACATTATCGATAGCTATGAA
GTTGGTCGCAACTACAACACCTTTTTCTATCTGAATGCCCAGAAATTTAGCCCGAACGAAAGCAATATTACCCTG
ACCAGCAGCTTTGATACAGGTCTGTTAGAAGGTAGCAAAGTGTATACCTTTTTCAGCAGCGATTTCATTAACAAC
ATCAACAAACCGGTTCAGGCCCTGCTGTTTATTGAATGGGTTAAACAGGTGATTCGCGATTTTACCACCGAAGCA
ACCAAAACCTCAACCGTTGATAAACTGAAAGATATTAGCCTGGTGGTGCCGTATATTGGTCTGGCACTGAATATT
GGTGATGAGATCTACAAACAGCATTTTGCAGAAGCAGTTGAACTGGTTGGTGCAGGTCTGCTGCTGGAATTTTCA
CCGGAATTTCTTATTCCGACGCTGCTGATTTTTACCATCAAAGGTTATCTGACCGGTAGCATTCGCGATAAAGAC
AAAATCATTAAAACCCTGGATAACGCCCTGAATGTTCGTGATCAGAAATGGAAAGAACTGTATCGTTGGGTTGTT
AGCAAATGGCTGACCACCATTAATACGCAGTTCAACAAACGCAAAGAACAAATGTACAAAGCCCTGAAAAATCAG
GCCACCGCCATTAAAAAGATCATCGAGAACAAATATAACAACTATACCACCGATGAAAAAAGCAAGATCGATAGC
AGCTATAACATCAACGAAATTGAACGCACCCTGAACGAAAAAATCAATCTGGCCATGAAAAACATCGAGCAGTTT
ATTACCGAAAGCAGCATTGCCTATCTGATCAATATCATCAACAACGAAACGATCCAGAAACTGAAAAGCTATGAT
GACCTGGTTCGTCGTTATCTGCTGGGTTATATTCGTAATCATAGCAGCATTCTGGGCAATAGCGTTGAAGAACTG
AATTCCAAAGTGAACAACCATCTGGATAATGGCATTCCGTTTGAACTGAGCAGTTATACCAATGATAGCCTGCTG
ATCCGCTACTTCAATAAAAACTATGGCGAACTGAAGTACAACTGCATTCTGAACATCAAATATGAGATGGATCGT
GACAAACTGGTTGATAGCAGCGGTTATCGTAGCCGTATCAATATTGGTACAGGCGTCAAATTTAGCGAGATCGAT
AAAAATCAAGTGCAGCTGAGCAATCTGGAATCCAGCAAAATTGAAGTCATTCTGAATAACGGCGTCATCTATAAC
AGCATGTATGAAAACTTTTCGACCAGCTTTTGGATTCGCATTCCGAAATACTTTCGCAACATCAATAACGAGTAC
AAGATCATCAGCTGTATGCAGAATAATAGCGGTTGGGAAGTGAGCCTGAATTTTAGCAATATGAACTCGAAAATC
ATCTGGACCCTGCAGGATACCGAAGGTATCAAAAAAACCGTTGTGTTTCAGTACACCCAGAACATTAACATTAGC
GACTATATCAACCGCTGGATCTTTGTGACCATTACAAATAATCGTCTGAGCAACAGCAAAATCTACATTAATGGT
CGCCTGATCAACGAAGAAAGCATTAGCGATCTGGGTAATATCCATGCCAGCAACAACATTATGTTTAAACTGGAT
GGTTGCCGTGATCCGCATCGTTATATCTGGATTAAATACTTTAACCTGTTTGACAAAGAGCTGAACAAGAAAGAA
ATTAAAGATCTGTACGACAACCAGAGCAATAGCGGTATTCTGAAAGATTTCTGGGGTGATTATCTGCAGTATGAC
AAACCGTATTATATGCTGAATCTGTATGACCCGAATAAGTATCTGGATGTGAATAATGTTGGCATCCGTGGCTAT
ATGTATCTGAAAGGTCCGCGTGGTCGTATTGTGACCACCAACATTTATCTGAATAGCACCCTGTATATGGGCACC
AAATTCATCATTAAGAAATATGCCAGCGGCAACAAAGATAACATTGTGCGTAATAATGATCGCGTGTATATTAAC
GTGGTGGTGAAGAATAAAGAATATCGCCTGGCAACCAATGCAAGCCAGGCAGGCGTTGAAAAAATTCTGAGCGCA
GTTGAAATCCCGGATGTTGGTAATCTGAGCCAGGTTGTTGTGATGAAAAGCGAAAATGATCAGGGCATTCGCAAC
AAGTGTAAAATGAATCTGCAAGACAATAACGGCAACGATATTGGCTTTATCGGCTTTCACCAGTTTAATAACATT
GCAAAACTGGTGGCCAGCAACTGGTATAACCGTCAGATTGGTAAAGCAAGCCGTACCTTTGGTTGTAGCTGGGAA
TTTATCCCGGTTGATGATGGTTGGGGTGAAAGCAGCCTGGAAAATCTGTATTTCCAGGGTGCCAGTCATCATCAC
CACCATCACCATCACTGA
SEQ ID NO: 26-Polypeptide Sequence of rBoNT/FA(0) (His-tagged)
MPVVINSFNYDDPVNDNTIIYIRPPYYETSNTYFKAFQIMDNVWIIPERYRLGIDPSLFNPPVSLKAGSDGYFDP
NYLSTNTEKNKYLQIMIKLFKRINSKPAGQILLEEIKNAIPYLGNSYTQEEQFTTNNRTVSFNVKLANGNIVQQM
ANLIIWGPGPDLTTNKTGGIIYSPYQSMEATPYKDGFGSIMTVEFSPEYATAFNDISIASHSPSLFIKDPALILM
HQLIYVLHGLYGTYITEYKITPNVVQSYMKVTKPITSAEFLTFGGRDRNIVPQSIQSQLYNKVLSDYKRIASRLN
KVNTATALINIDEFKNLYEWKYQFAKDSNGVYSVDLNKFEQLYKKIYSFTEFNLAYEFKIKTRLGYLAENFGPFY
LPNLLDDSIYTEVDGFNIGALSINYQGQNIGSDINSIKKLQGQGVVSRVVRLCSNSNTKNSLCITVNNRDLFFIA
SQESYGENTINTYKEIDDTTTLDPSFEDILDKVILNFNEQVIPQMPNRNVSTDIQKDNYIPKYDYNRTDIIDSYE
VGRNYNTFFYLNAQKFSPNESNITLTSSFDTGLLEGSKVYTFFSSDFINNINKPVQALLFIEWVKQVIRDETTEA
TKTSTVDKLKDISLVVPYIGLALNIGDEIYKQHFAEAVELVGAGLLLEFSPEFLIPTLLIFTIKGYLTGSIRDKD
KIIKTLDNALNVRDQKWKELYRWVVSKWLTTINTQFNKRKEQMYKALKNQATAIKKIIENKYNNYTTDEKSKIDS
SYNINEIERTLNEKINLAMKNIEQFITESSIAYLINIINNETIQKLKSYDDLVRRYLLGYIRNHSSILGNSVEEL
NSKVNNHLDNGIPFELSSYTNDSLLIRYFNKNYGELKYNCILNIKYEMDRDKLVDSSGYRSRINIGTGVKFSEID
KNQVQLSNLESSKIEVILNNGVIYNSMYENFSTSFWIRIPKYFRNINNEYKIISCMQNNSGWEVSLNFSNMNSKI
IWTLQDTEGIKKTVVFQYTQNINISDYINRWIFVTITNNRLSNSKIYINGRLINEESISDLGNIHASNNIMFKLD
GCRDPHRYIWIKYFNLFDKELNKKEIKDLYDNQSNSGILKDFWGDYLQYDKPYYMLNLYDPNKYLDVNNVGIRGY
MYLKGPRGRIVTTNIYLNSTLYMGTKFIIKKYASGNKDNIVRNNDRVYINVVVKNKEYRLATNASQAGVEKILSA
VEIPDVGNLSQVVVMKSENDQGIRNKCKMNLQDNNGNDIGFIGFHQFNNIAKLVASNWYNRQIGKASRTFGCSWE
FIPVDDGWGESSLENLYFQGASHHHHHHHH
SEQ ID NO: 27-Nucleotide Sequence of rLH_{N}/FA (His-tagged)
ATGCCGGTTGTGATTAACAGCTTCAATTATGATGATCCGGTGAACGATAACACCATCATTTATATCCGTCCGCCT
TATTATGAAACCAGCAACACCTATTTCAAAGCCTTCCAGATTATGGATAACGTGTGGATTATTCCGGAACGTTAT
CGTCTGGGTATTGATCCGAGCCTGTTTAATCCGCCTGTTAGCCTGAAAGCAGGTAGTGATGGTTATTTTGATCCG
AATTATCTGAGCACCAACACCGAGAAAAACAAATACCTGCAGATTATGATCAAGCTGTTCAAACGCATTAATAGC
AAACCGGCAGGTCAGATTCTGCTGGAAGAAATCAAAAATGCAATTCCGTATCTGGGCAACAGCTATACCCAAGAA
GAACAGTTTACCACCAATAATCGTACCGTGAGCTTTAATGTTAAACTGGCCAATGGTAATATCGTTCAGCAGATG
GCAAATCTGATTATTTGGGGTCCGGGTCCTGATCTGACCACAAATAAAACCGGTGGTATCATCTATAGCCCGTAT
CAGAGCATGGAAGCAACCCCGTATAAAGATGGTTTTGGTAGCATTATGACCGTGGAATTTAGTCCGGAATATGCA
ACCGCCTTTAACGATATTTCAATTGCAAGCCATAGTCCGTCGCTGTTTATCAAAGATCCGGCACTGATTCTGATG
CATGAACTGATTCATGTTCTGCATGGTCTGTATGGCACCTATATTACCGAATACAAAATTACCCCGAATGTGGTG
CAGAGCTATATGAAAGTTACCAAACCGATTACCAGCGCAGAATTTCTGACCTTTGGTGGTCGTGATCGCAATATT
GTTCCGCAGAGCATTCAGAGCCAGCTGTATAACAAAGTTCTGAGCGATTATAAACGTATTGCCAGCCGTCTGAAT
AAAGTTAATACCGCAACCGCACTGATCAACATCGATGAATTCAAAAACCTGTACGAGTGGAAATACCAGTTTGCC
AAAGATAGCAATGGTGTGTATAGCGTGGATCTGAACAAATTTGAGCAGCTGTACAAAAAAATCTATAGCTTCACC
GAATTCAACCTGGCCTATGAGTTTAAAATCAAAACCCGTCTGGGTTATCTGGCCGAAAATTTTGGTCCGTTTTAT
CTGCCGAATCTGCTGGATGATAGCATTTATACCGAAGTGGATGGTTTTAACATTGGTGCACTGAGCATTAACTAT
CAGGGTCAGAATATTGGCAGCGATATCAACAGCATCAAAAAACTGCAAGGTCAGGGTGTTGTTAGCCGTGTTGTT
CGTCTGTGTAGCAATAGCAATACCAAAAACAGCCTGTGCATTACCGTTAATAATCGCGACCTGTTTTTTATCGCA
AGCCAAGAAAGCTATGGCGAGAATACCATTAACACCTATAAAGAGATTGACGATACCACCACACTGGATCCGAGC
TTTGAAGATATTCTGGATAAAGTGATCCTGAACTTCAACGAACAGGTTATTCCGCAGATGCCGAATCGTAATGTT
AGCACCGATATTCAGAAAGACAACTACATCCCGAAATACGATTATAACCGCACCGACATTATCGATAGCTATGAA
GTTGGTCGCAACTACAACACCTTTTTCTATCTGAATGCCCAGAAATTTAGCCCGAACGAAAGCAATATTACCCTG
ACCAGCAGCTTTGATACAGGTCTGTTAGAAGGTAGCAAAGTGTATACCTTTTTCAGCAGCGATTTCATTAACAAC
ATCAACAAACCGGTTCAGGCCCTGCTGTTTATTGAATGGGTTAAACAGGTGATTCGCGATTTTACCACCGAAGCA
ACCAAAACCTCAACCGTTGATAAACTGAAAGATATTAGCCTGGTGGTGCCGTATATTGGTCTGGCACTGAATATT
GGTGATGAGATCTACAAACAGCATTTTGCAGAAGCAGTTGAACTGGTTGGTGCAGGTCTGCTGCTGGAATTTTCA
CCGGAATTTCTTATTCCGACGCTGCTGATTTTTACCATCAAAGGTTATCTGACCGGTAGCATTCGCGATAAAGAC
AAAATCATTAAAACCCTGGATAACGCCCTGAATGTTCGTGATCAGAAATGGAAAGAACTGTATCGTTGGGTTGTT
AGCAAATGGCTGACCACCATTAATACGCAGTTCAACAAACGCAAAGAACAAATGTACAAAGCCCTGAAAAATCAG
GCCACCGCCATTAAAAAGATCATCGAGAACAAATATAACAACTATACCACCGATGAAAAAAGCAAGATCGATAGC
AGCTATAACATCAACGAAATTGAACGCACCCTGAACGAAAAAATCAATCTGGCCATGAAAAACATCGAGCAGTTT
ATTACAGAAAGCAGCATTGCCTACCTGATCAATATCATCAACAACGAAACCATTCAGAAACTGAAAAGCTATGAT
GACCTGGTTCGTCGTTATCTGCTGGGTTATATTCGTAATCATAGCAGCATTCTGGGCAATAGCGTTGAAGAACTG
AATTCCAAAGTGAACAACCATCTGGATAATGGCATTCCGTTTGAACTGAGCAGTTATACCAATGATAGCCTGCTG
ATCCGCTACTTCAATAAAAACTATGGCGAAGAGAACCTGTATTTCCAGGGTGCCAGTCATCATCACCACCATCAC
CATCACTGA
SEQ ID NO: 28-Polypeptide Sequence of rLH_{N}/FA (His-tagged)
MPVVINSFNYDDPVNDNTIIYIRPPYYETSNTYFKAFQIMDNVWIIPERYRLGIDPSLENPPVSLKAGSDGYFDP
NYLSTNTEKNKYLQIMIKLFKRINSKPAGQILLEEIKNAIPYLGNSYTQEEQFTTNNRTVSFNVKLANGNIVQQM
ANLIIWGPGPDLTTNKTGGIIYSPYQSMEATPYKDGFGSIMTVEFSPEYATAFNDISIASHSPSLFIKDPALILM
HELIHVLHGLYGTYITEYKITPNVVQSYMKVTKPITSAEFLTFGGRDRNIVPQSIQSQLYNKVLSDYKRIASRLN
KVNTATALINIDEFKNLYEWKYQFAKDSNGVYSVDLNKFEQLYKKIYSFTEFNLAYEFKIKTRLGYLAENFGPFY
LPNLLDDSIYTEVDGFNIGALSINYQGQNIGSDINSIKKLQGQGVVSRVVRLCSNSNTKNSLCITVNNRDLFFIA
SQESYGENTINTYKEIDDTTTLDPSFEDILDKVILNFNEQVIPQMPNRNVSTDIQKDNYIPKYDYNRTDIIDSYE
VGRNYNTFFYLNAQKFSPNESNITLTSSFDTGLLEGSKVYTFFSSDFINNINKPVQALLFIEWVKQVIRDETTEA
TKTSTVDKLKDISLVVPYIGLALNIGDEIYKQHFAEAVELVGAGLLLEFSPEFLIPTLLIFTIKGYLTGSIRDKD
KIIKTLDNALNVRDQKWKELYRWVVSKWLTTINTQFNKRKEQMYKALKNQATAIKKIIENKYNNYTTDEKSKIDS
SYNINEIERTLNEKINLAMKNIEQFITESSIAYLINIINNETIQKLKSYDDLVRRYLLGYIRNHSSILGNSVEEL
NSKVNNHLDNGIPFELSSYTNDSLLIRYENKNYGEENLYFQGASHHHHHHHH
SEQ ID NO: 29-Nucleotide Sequence of rH_{C}/FA (His-tagged)
ATGCTGAAGTATAACTGCATCCTGAACATCAAATATGAGATGGATCGTGATAAACTGGTTGATAGCAGCGGTTAT
CGTAGCCGTATCAATATTGGCACCGGTGTGAAATTTAGCGAGATCGATAAAAATCAGGTGCAGCTGAGCAATCTG
GAAAGCAGCAAAATTGAAGTGATTCTGAATAACGGCGTGATCTACAATAGCATGTATGAAAACTTTTCGACCAGC
TTCTGGATTCGCATTCCGAAATACTTTCGCAACATCAACAACGAGTACAAGATTATCAGCTGTATGCAGAATAAT
AGCGGTTGGGAAGTTAGCCTGAATTTCAGCAATATGAACAGCAAAATCATTTGGACCCTGCAGGATACCGAAGGT
ATCAAAAAAACCGTTGTGTTTCAGTACACCCAGAACATTAACATCAGCGATTACATTAACCGCTGGATCTTTGTG
ACCATTACCAATAATCGTCTGAGCAACAGCAAGATCTATATTAACGGTCGCCTGATTAACGAAGAGAGCATTAGC
GATCTGGGTAATATTCATGCCAGCAACAACATCATGTTTAAACTGGATGGTTGTCGTGATCCGCATCGTTATATT
TGGATCAAATACTTCAACCTGTTTGATAAAGAACTGAACAAAAAAGAAATCAAAGACCTGTATGATAACCAGAGC
AATAGCGGCATTCTGAAAGATTTTTGGGGTGATTATCTGCAGTATGACAAACCGTATTACATGCTGAATCTGTAC
GATCCGAACAAATATCTGGATGTGAATAATGTGGGTATCCGTGGCTATATGTATCTGAAAGGTCCGCGTGGTCGT
ATTGTTACCACCAACATTTATCTGAATAGCACCCTGTATATGGGCACCAAATTCATCATTAAAAAGTATGCCAGC
GGCAACAAAGATAACATTGTGCGTAATAATGATCGCGTGTATATCAATGTGGTGGTGAAGAATAAAGAATATCGT
CTGGCCACCAATGCAAGCCAGGCAGGCGTTGAAAAAATTCTGAGCGCAGTTGAAATTCCGGATGTTGGTAATCTG
AGCCAGGTTGTTGTTATGAAAAGCGAAAATGATCAGGGCATTCGCAACAAATGCAAAATGAATCTGCAGGACAAT
AACGGCAACGATATTGGTTTTATTGGCTTCCACCAGTTCAACAACATTGCAAAACTGGTGGCGAGCAATTGGTAT
AATCGTCAGATTGGTAAAGCAAGCCGTACCTTTGGTTGTAGCTGGGAATTTATTCCGGTTGATGATGGTTGGGGT
GAAAGCAGCCTGGAAAATCTGTATTTTCAGGGTGCAAGTCATCATCACCACCATCACCATCATTAA
SEQ ID NO: 30-Polypeptide Sequence of rH_{C}/FA (His-tagged)
MLKYNCILNIKYEMDRDKLVDSSGYRSRINIGTGVKFSEIDKNQVQLSNLESSKIEVILNNGVIYNSMYENESTS
FWIRIPKYFRNINNEYKIISCMQNNSGWEVSLNFSNMNSKIIWTLQDTEGIKKTVVFQYTQNINISDYINRWIFV
TITNNRLSNSKIYINGRLINEESISDLGNIHASNNIMFKLDGCRDPHRYIWIKYFNLFDKELNKKEIKDLYDNQS
NSGILKDFWGDYLQYDKPYYMLNLYDPNKYLDVNNVGIRGYMYLKGPRGRIVTTNIYLNSTLYMGTKFIIKKYAS
GNKDNIVRNNDRVYINVVVKNKEYRLATNASQAGVEKILSAVEIPDVGNLSQVVVMKSENDQGIRNKCKMNLQDN
NGNDIGFIGFHQFNNIAKLVASNWYNRQIGKASRTFGCSWEFIPVDDGWGESSLENLYFQGASHHHHHHHH
SEQ ID NO: 31-Nucleotide Sequence of rLC/FA (His-tagged)
ATGCCGGTTGTGATTAACAGCTTCAATTATGATGATCCGGTGAACGATAACACCATCATTTATATCCGTCCGCCT
TATTATGAAACCAGCAACACCTATTTCAAAGCCTTCCAGATTATGGATAACGTGTGGATTATTCCGGAACGTTAT
CGTCTGGGTATTGATCCGAGCCTGTTTAATCCGCCTGTTAGCCTGAAAGCAGGTAGTGATGGTTATTTTGATCCG
AATTATCTGAGCACCAACACCGAGAAAAACAAATACCTGCAGATTATGATCAAGCTGTTCAAACGCATTAATAGC
AAACCGGCAGGTCAGATTCTGCTGGAAGAAATCAAAAATGCAATTCCGTATCTGGGCAACAGCTATACCCAAGAA
GAACAGTTTACCACCAATAATCGTACCGTGAGCTTTAATGTTAAACTGGCCAATGGTAATATCGTTCAGCAGATG
GCAAATCTGATTATTTGGGGTCCGGGTCCTGATCTGACCACAAATAAAACCGGTGGTATCATCTATAGCCCGTAT
CAGAGCATGGAAGCAACCCCGTATAAAGATGGTTTTGGTAGCATTATGACCGTGGAATTTAGTCCGGAATATGCA
ACCGCCTTTAACGATATTTCAATTGCAAGCCATAGTCCGTCGCTGTTTATCAAAGATCCGGCACTGATTCTGATG
CATGAACTGATTCATGTTCTGCATGGTCTGTATGGCACCTATATTACCGAATACAAAATTACCCCGAATGTGGTG
CAGAGCTATATGAAAGTTACCAAACCGATTACCAGCGCAGAATTTCTGACCTTTGGTGGTCGTGATCGCAATATT
GTTCCGCAGAGCATTCAGAGCCAGCTGTATAACAAAGTTCTGAGCGATTATAAACGTATTGCCAGCCGTCTGAAT
AAAGTTAATACCGCAACCGCACTGATCAACATCGATGAATTCAAAAACCTGTACGAGTGGAAATACCAGTTTGCC
AAAGATAGCAATGGTGTGTATAGCGTGGATCTGAACAAATTTGAGCAGCTGTACAAAAAAATCTATAGCTTCACC
GAATTCAACCTGGCCTATGAGTTTAAAATCAAAACCCGTCTGGGTTATCTGGCCGAAAATTTTGGTCCGTTTTAT
CTGCCGAATCTGCTGGATGATAGCATTTATACCGAAGTGGATGGTTTTAACATTGGTGCACTGAGCATTAACTAT
CAGGGTCAGAATATTGGCAGCGATATCAACAGCATCAAAAAACTGCAAGGTCAGGGTGTTGTTAGCCGTGTTGTT
CGTCTGTGTAGCAATAGCGAAAATCTGTATTTTCAGGGTGCCAGTCATCATCACCACCATCACCATCACTGA
SEQ ID NO: 32-Polypeptide Sequence of rLC/FA (His-tagged)
MPVVINSFNYDDPVNDNTIIYIRPPYYETSNTYFKAFQIMDNVWIIPERYRLGIDPSLFNPPVSLKAGSDGYFDP
NYLSTNTEKNKYLQIMIKLFKRINSKPAGQILLEEIKNAIPYLGNSYTQEEQFTTNNRTVSFNVKLANGNIVQQM
ANLIIWGPGPDLTTNKTGGIIYSPYQSMEATPYKDGFGSIMTVEFSPEYATAFNDISIASHSPSLFIKDPALILM
HELIHVLHGLYGTYITEYKITPNVVQSYMKVTKPITSAEFLTFGGRDRNIVPQSIQSQLYNKVLSDYKRIASRLN
KVNTATALINIDEFKNLYEWKYQFAKDSNGVYSVDLNKFEQLYKKIYSFTEFNLAYEFKIKTRLGYLAENFGPFY
LPNLLDDSIYTEVDGFNIGALSINYQGQNIGSDINSIKKLQGQGVVSRVVRLCSNSENLYFQGASHHHHHHHH
SEQ ID NO: 33-Nucleotide Sequence of rBoNT/F(0) (His-tagged)
ATGCCGGTTGTGATTAACAGCTTCAATTATAACGATCCGGTGAACGATGATACCATCCTGTATATGCAGATTCCG
TATGAAGAGAAAAGCAAAAAGTACTACAAAGCCTTTGAGATCATGCGCAACGTTTGGATTATTCCGGAACGTAAT
ACCATTGGCACCGATCCGAGCGATTTTGATCCGCCTGCAAGCCTGGAAAATGGTAGCAGCGCATATTATGATCCG
AATTATCTGACCACCGATGCCGAAAAAGATCGTTATCTGAAAACCACCATCAAACTGTTCAAACGCATTAATAGC
AATCCGGCAGGCGAAGTTCTGCTGCAAGAAATTAGCTATGCAAAACCGTATCTGGGCAATGAACATACCCCGATT
AATGAATTTCATCCGGTTACACGTACCACGAGCGTTAACATTAAAAGCAGCACCAATGTGAAGTCCAGCATTATT
CTGAATCTGCTGGTTTTAGGTGCAGGTCCGGATATTTTTGAAAATTCAAGCTATCCGGTGCGCAAACTGATGGAT
AGCGGTGGTGTGTATGATCCGTCAAATGATGGTTTTGGCAGCATTAACATTGTGACCTTTAGTCCGGAATATGAA
TACACCTTCAACGATATTAGCGGTGGCTATAATAGCAGCACCGAAAGTTTTATTGCAGATCCGGCAATTAGCCTG
GCACACCAGCTGATTTATGCACTGCATGGTCTGTATGGTGCACGTGGTGTTACCTATAAAGAAACCATTAAAGTT
AAACAGGCACCGCTGATGATTGCGGAAAAACCGATTCGTCTGGAAGAATTTCTGACCTTTGGTGGTCAGGATCTG
AACATTATTACCAGCGCAATGAAAGAGAAAATCTATAATAACCTGCTGGCCAACTATGAGAAAATTGCAACCCGT
CTGAGCCGTGTTAATAGCGCACCTCCTGAATATGATATCAACGAGTATAAAGACTATTTTCAGTGGAAATACGGC
CTGGATAAAAATGCAGATGGTAGCTATACCGTGAACGAGAACAAATTTAACGAGATCTACAAAAAACTGTATAGC
TTCACCGAAATCGATCTGGCCAACAAATTCAAAGTGAAATGCCGCAACACCTACTTCATCAAATATGGCTTTCTG
AAAGTTCCGAACCTGCTTGATGATGATATCTATACCGTTAGCGAAGGCTTTAACATTGGTAATCTGGCCGTTAAT
AATCGCGGTCAGAACATTAAACTGAACCCGAAAATTATCGATAGCATCCCGGATAAAGGCCTGGTTGAAAAAATT
GTGAAATTCTGCAAAAGCGTGATTCCGCGTAAAGGCACCAAAGCACCGCCTCGTCTGTGTATTCGTGTGAATAAT
CGTGAACTGTTTTTTGTTGCAAGCGAGAGCAGCTATAACGAGAATGATATTAACACCCCGAAAGAGATTGACGAT
ACCACCAATCTGAATAACAACTATCGCAACAATCTGGATGAAGTGATCCTGGATTATAACAGCGAAACCATTCCG
CAGATTAGCAATCAGACCCTGAATACCCTGGTTCAGGATGATAGCTATGTTCCGCGTTATGATAGCAATGGCACC
AGCGAAATTGAAGAACATAATGTGGTTGATCTGAACGTGTTCTTTTATCTGCATGCACAGAAAGTGCCGGAAGGT
GAAACCAATATTAGCCTGACCAGCAGCATTGATACCGCACTGAGCGAAGAAAGCCAGGTTTATACCTTTTTTAGC
AGCGAATTCATCAACACCATTAACAAACCGGTTCATGCAGCACTGTTTATTAGCTGGATTAATCAGGTGATTCGC
GATTTTACCACCGAAGCAACCCAGAAAAGCACCTTTGATAAAATTGCCGATATTAGTCTGGTGGTGCCGTATGTT
GGTCTGGCACTGAATATTGGTAATGAAGTGCAGAAAGAGAACTTTAAAGAAGCCTTCGAACTGTTAGGTGCCGGT
ATTCTGCTGGAATTTGTGCCGGAACTGCTGATTCCGACCATTCTGGTTTTTACCATTAAGAGCTTTATTGGCAGC
AGCGAGAACAAGAACAAAATCATTAAAGCCATCAACAACAGCCTGATGGAACGCGAAACCAAATGGAAAGAAATT
TACAGCTGGATTGTGAGCAATTGGCTGACCCGTATCAATACCCAGTTTAACAAACGCAAAGAACAAATGTATCAG
GCCCTGCAGAATCAGGTTGATGCAATTAAAACCGTGATCGAATACAAATACAACAACTATACCAGCGACGAACGT
AATCGCCTGGAAAGCGAATACAACATTAATAACATTCGCGAAGAACTGAACAAAAAAGTGAGCCTGGCAATGGAA
AACATCGAACGTTTTATTACCGAAAGCAGCATCTTCTACCTGATGAAACTGATTAACGAAGCCAAAGTTAGCAAA
CTGCGCGAATATGATGAAGGCGTTAAAGAATATCTGCTGGACTATATTAGCGAACATCGTAGCATTCTGGGTAAT
AGCGTTCAAGAGCTGAATGATCTGGTTACCAGCACACTGAATAATAGCATTCCGTTTGAACTGAGCAGCTACACC
AACGATAAAATCCTGATCCTGTACTTCAACAAACTGTACAAGAAGATCAAGGACAACAGCATACTGGATATGCGC
TATGAAAACAACAAGTTCATTGATATCAGCGGCTATGGTAGCAACATTAGCATTAATGGTGATGTGTATATCTAC
AGCACCAACCGCAATCAGTTTGGTATTTATAGCAGCAAACCGAGCGAAGTTAATATTGCGCAGAATAACGATATC
ATCTACAACGGTCGCTATCAGAACTTTAGCATTAGCTTTTGGGTTCGCATTCCGAAATACTTTAACAAGGTGAAC
CTGAACAACGAGTACACCATTATTGATTGCATTCGCAATAATAACAGCGGCTGGAAAATCAGCCTGAACTATAAC
AAAATTATCTGGACCCTGCAGGATACCGCAGGTAATAATCAGAAACTGGTGTTTAACTACACCCAGATGATTAGC
ATCAGCGACTATATCAACAAATGGATCTTTGTGACCATTACCAACAATCGTCTGGGTAACAGCCGCATTTATATC
AATGGCAATCTGATCGACGAAAAAAGCATTTCAAATCTGGGCGATATTCACGTGAGCGATAACATTCTGTTCAAA
ATTGTTGGCTGCAACGATACCCGTTATGTTGGTATTCGTTACTTCAAAGTGTTTGATACGGAACTGGGCAAAACG
GAAATTGAAACCCTGTATAGTGATGAACCGGATCCGAGCATTCTGAAAGATTTTTGGGGTAATTATCTGCTGTAC
AACAAACGCTACTATCTGCTGAACCTGCTGCGTACCGATAAAAGCATTACACAGAATAGCAACTTTCTGAACATC
AATCAGCAGCGTGGTGTTTATCAGAAACCGAACATTTTTAGCAACACCCGTCTGTATACCGGTGTGGAAGTTATT
ATTCGTAAAAACGGTAGCACCGATATCAGCAACACCGATAACTTTGTGCGTAAAAATGACCTGGCCTATATTAAC
GTTGTTGATCGTGATGTTGAGTATCGTCTGTATGCGGATATTAGCATTGCCAAACCGGAAAAGATTATCAAACTG
ATCCGTACCAGCAACAGCAATAATTCACTGGGTCAGATTATCGTGATGGACAGCATTGGTAACAATTGCACCATG
AATTTCCAGAACAATAACGGTGGTAATATTGGCCTGCTGGGCTTTCATAGCAATAATCTGGTTGCAAGCAGCTGG
TATTACAACAACATCCGTAAAAATACCAGCAGTAATGGTTGCTTTTGGAGCTTTATCAGTAAAGAACATGGCTGG
CAAGAAAACGAGAACCTGTATTTTCAGGGTGCAAGTCATCATCACCATCACCACCATCATTAA
SEQ ID NO: 34-Polypeptide Sequence of rBoNT/F(0) (His-tagged)
MPVVINSFNYNDPVNDDTILYMQIPYEEKSKKYYKAFEIMRNVWIIPERNTIGTDPSDFDPPASLENGSSAYYDP
NYLTTDAEKDRYLKTTIKLFKRINSNPAGEVLLQEISYAKPYLGNEHTPINEFHPVTRTTSVNIKSSTNVKSSII
LNLLVLGAGPDIFENSSYPVRKLMDSGGVYDPSNDGFGSINIVTFSPEYEYTENDISGGYNSSTESFIADPAISL
AHQLIYALHGLYGARGVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKFKVKCRNTYFIKYGEL
KVPNLLDDDIYTVSEGENIGNLAVNNRGQNIKLNPKIIDSIPDKGLVEKIVKFCKSVIPRKGTKAPPRLCIRVNN
RELFFVASESSYNENDINTPKEIDDTTNLNNNYRNNLDEVILDYNSETIPQISNQTLNTLVQDDSYVPRYDSNGT
SEIEEHNVVDLNVFFYLHAQKVPEGETNISLTSSIDTALSEESQVYTFFSSEFINTINKPVHAALFISWINQVIR
DFTTEATQKSTFDKIADISLVVPYVGLALNIGNEVQKENFKEAFELLGAGILLEFVPELLIPTILVFTIKSFIGS
SENKNKIIKAINNSLMERETKWKEIYSWIVSNWLTRINTQFNKRKEQMYQALQNQVDAIKTVIEYKYNNYTSDER
NRLESEYNINNIREELNKKVSLAMENIERFITESSIFYLMKLINEAKVSKLREYDEGVKEYLLDYISEHRSILGN
SVQELNDLVTSTLNNSIPFELSSYTNDKILILYENKLYKKIKDNSILDMRYENNKFIDISGYGSNISINGDVYIY
STNRNQFGIYSSKPSEVNIAQNNDIIYNGRYQNFSISFWVRIPKYFNKVNLNNEYTIIDCIRNNNSGWKISLNYN
KIIWTLQDTAGNNQKLVENYTQMISISDYINKWIFVTITNNRLGNSRIYINGNLIDEKSISNLGDIHVSDNILEK
IVGCNDTRYVGIRYFKVEDTELGKTEIETLYSDEPDPSILKDFWGNYLLYNKRYYLLNLLRTDKSITQNSNFLNI
NQQRGVYQKPNIFSNTRLYTGVEVIIRKNGSTDISNTDNFVRKNDLAYINVVDRDVEYRLYADISIAKPEKIIKL
IRTSNSNNSLGQIIVMDSIGNNCTMNFQNNNGGNIGLLGFHSNNLVASSWYYNNIRKNTSSNGCFWSFISKEHGW
QENENLYFQGASHHHHHHHH
SEQ ID NO: 35-Nucleotide Sequence of rL_{H}N/F (His-tagged)
ATGCCGGTTGTGATTAACAGCTTCAATTATAACGATCCGGTGAACGATGATACCATCCTGTATATGCAGATTCCG
TATGAAGAGAAAAGCAAAAAGTACTACAAAGCCTTTGAGATCATGCGCAACGTTTGGATTATTCCGGAACGTAAT
ACCATTGGCACCGATCCGAGCGATTTTGATCCGCCTGCAAGCCTGGAAAATGGTAGCAGCGCATATTATGATCCG
AATTATCTGACCACCGATGCCGAAAAAGATCGTTATCTGAAAACCACCATCAAACTGTTCAAACGCATTAATAGC
AATCCGGCAGGCGAAGTTCTGCTGCAAGAAATTAGCTATGCAAAACCGTATCTGGGCAATGAACATACCCCGATT
AATGAATTTCATCCGGTTACACGTACCACGAGCGTTAACATTAAAAGCAGCACCAATGTGAAGTCCAGCATTATT
CTGAATCTGCTGGTTTTAGGTGCAGGTCCGGATATTTTTGAAAATTCAAGCTATCCGGTGCGCAAACTGATGGAT
AGCGGTGGTGTGTATGATCCGTCAAATGATGGTTTTGGCAGCATTAACATTGTGACCTTTAGTCCGGAATATGAA
TACACCTTCAACGATATTAGCGGTGGCTATAATAGCAGCACCGAAAGTTTTATTGCAGATCCGGCAATTAGCCTG
GCACATGAACTGATTCATGCACTGCATGGTCTGTATGGTGCACGTGGTGTTACCTATAAAGAAACCATTAAAGTT
AAACAGGCACCGCTGATGATTGCGGAAAAACCGATTCGTCTGGAAGAATTTCTGACCTTTGGTGGTCAGGATCTG
AACATTATTACCAGCGCAATGAAAGAGAAAATCTATAATAACCTGCTGGCCAACTATGAGAAAATTGCAACCCGT
CTGAGCCGTGTTAATAGCGCACCTCCTGAATATGATATCAACGAGTATAAAGACTATTTTCAGTGGAAATACGGC
CTGGATAAAAATGCAGATGGTAGCTATACCGTGAACGAGAACAAATTTAACGAGATCTACAAAAAACTGTATAGC
TTCACCGAAATCGATCTGGCCAACAAATTCAAAGTGAAATGCCGCAACACCTACTTCATCAAATATGGCTTTCTG
AAAGTTCCGAACCTGCTTGATGATGATATCTATACCGTTAGCGAAGGCTTTAACATTGGTAATCTGGCCGTTAAT
AATCGCGGTCAGAACATTAAACTGAACCCGAAAATTATCGATAGCATCCCGGATAAAGGCCTGGTTGAAAAAATT
GTGAAATTCTGCAAAAGCGTGATTCCGCGTAAAGGCACCAAAGCACCGCCTCGTCTGTGTATTCGTGTGAATAAT
CGTGAACTGTTTTTTGTTGCAAGCGAGAGCAGCTATAACGAGAATGATATTAACACCCCGAAAGAGATTGACGAT
ACCACCAATCTGAATAACAACTATCGCAACAATCTGGATGAAGTGATCCTGGATTATAACAGCGAAACCATTCCG
CAGATTAGCAATCAGACCCTGAATACCCTGGTTCAGGATGATAGCTATGTTCCGCGTTATGATAGCAATGGCACC
AGCGAAATTGAAGAACATAATGTGGTTGATCTGAACGTGTTCTTTTATCTGCATGCACAGAAAGTGCCGGAAGGT
GAAACCAATATTAGCCTGACCAGCAGCATTGATACCGCACTGAGCGAAGAAAGCCAGGTTTATACCTTTTTTAGC
AGCGAATTCATCAACACCATTAACAAACCGGTTCATGCAGCACTGTTTATTAGCTGGATTAATCAGGTGATTCGC
GATTTTACCACCGAAGCAACCCAGAAAAGCACCTTTGATAAAATTGCCGATATTAGTCTGGTGGTGCCGTATGTT
GGTCTGGCACTGAATATTGGTAATGAAGTGCAGAAAGAGAACTTTAAAGAAGCCTTCGAACTGTTAGGTGCCGGT
ATTCTGCTGGAATTTGTGCCGGAACTGCTGATTCCGACCATTCTGGTTTTTACCATTAAGAGCTTTATTGGCAGC
AGCGAGAACAAGAACAAAATCATTAAAGCCATCAACAACAGCCTGATGGAACGCGAAACCAAATGGAAAGAAATT
TACAGCTGGATTGTGAGCAATTGGCTGACCCGTATCAATACCCAGTTTAACAAACGCAAAGAACAAATGTATCAG
GCCCTGCAGAATCAGGTTGATGCAATTAAAACCGTGATCGAATACAAATACAACAACTATACCAGCGACGAACGT
AATCGCCTGGAAAGCGAATACAACATTAATAACATTCGCGAAGAACTGAACAAAAAAGTGAGCCTGGCAATGGAA
AACATCGAACGTTTTATTACCGAAAGCAGCATCTTCTACCTGATGAAACTGATTAACGAAGCCAAAGTTAGCAAA
CTGCGCGAATATGATGAAGGCGTTAAAGAATATCTGCTGGACTATATTAGCGAACATCGTAGCATTCTGGGTAAT
AGCGTTCAAGAGCTGAATGATCTGGTTACCAGCACACTGAATAATAGCATTCCGTTTGAACTGAGCAGCTACACC
AACGATAAAATCCTGATCCTGTACTTCAACAAACTGTACAAGAAAGAAAACCTGTATTTTCAGGGTGCAAGCCAT
CATCACCACCATCACCATCATTAA
SEQ ID NO: 36-Polypeptide Sequence of rL_{H}N/F (His-tagged)
MPVVINSFNYNDPVNDDTILYMQIPYEEKSKKYYKAFEIMRNVWIIPERNTIGTDPSDFDPPASLENGSSAYYDP
NYLTTDAEKDRYLKTTIKLFKRINSNPAGEVLLQEISYAKPYLGNEHTPINEFHPVTRTTSVNIKSSTNVKSSII
LNLLVLGAGPDIFENSSYPVRKLMDSGGVYDPSNDGFGSINIVTFSPEYEYTENDISGGYNSSTESFIADPAISL
AHELIHALHGLYGARGVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKFKVKCRNTYFIKYGFL
KVPNLLDDDIYTVSEGFNIGNLAVNNRGQNIKLNPKIIDSIPDKGLVEKIVKFCKSVIPRKGTKAPPRLCIRVNN
RELFFVASESSYNENDINTPKEIDDTTNLNNNYRNNLDEVILDYNSETIPQISNQTLNTLVQDDSYVPRYDSNGT
SEIEEHNVVDLNVFFYLHAQKVPEGETNISLTSSIDTALSEESQVYTFFSSEFINTINKPVHAALFISWINQVIR
DFTTEATQKSTEDKIADISLVVPYVGLALNIGNEVQKENFKEAFELLGAGILLEFVPELLIPTILVFTIKSFIGS
SENKNKIIKAINNSLMERETKWKEIYSWIVSNWLTRINTQFNKRKEQMYQALQNQVDAIKTVIEYKYNNYTSDER
NRLESEYNINNIREELNKKVSLAMENIERFITESSIFYLMKLINEAKVSKLREYDEGVKEYLLDYISEHRSILGN
SVQELNDLVTSTLNNSIPFELSSYTNDKILILYFNKLYKKENLYFQGASHHHHHHHH
SEQ ID NO: 37-Nucleotide Sequence of rH_{C}/F (His-tagged)
ATGATCAAGGATAACAGCATTCTGGATATGCGCTATGAGAACAACAAATTCATTGATATTAGCGGCTATGGCAGC
AACATTAGCATTAATGGTGATGTGTATATCTACAGCACCAACCGTAATCAGTTTGGCATTTATAGCAGCAAACCG
AGCGAAGTTAATATTGCCCAGAACAACGATATCATCTATAACGGTCGCTATCAGAACTTCAGCATTAGCTTTTGG
GTTCGCATTCCGAAATACTTCAATAAGGTGAACCTGAACAACGAGTATACCATCATTGATTGCATTCGCAATAAT
AACAGCGGCTGGAAAATTAGCCTGAACTACAACAAAATTATCTGGACCCTGCAGGATACCGCAGGTAATAATCAG
AAACTGGTGTTTAACTACACCCAGATGATTAGCATCAGCGACTATATCAACAAATGGATCTTTGTGACCATTACC
AATAATCGCCTGGGTAATAGCCGCATTTATATCAATGGTAACCTGATCGATGAGAAAAGCATTAGCAATCTGGGT
GATATTCATGTGAGCGATAACATCCTGTTTAAAATCGTGGGTTGTAACGATACCCGTTATGTTGGTATTCGCTAC
TTCAAAGTGTTTGATACCGAACTGGGTAAAACCGAAATTGAAACCCTGTATAGTGATGAACCGGATCCGAGCATT
CTGAAAGATTTTTGGGGTAATTATCTGCTGTACAACAAACGCTACTATCTGCTGAATCTGCTGCGTACCGATAAA
TCAATTACCCAGAATAGCAACTTCCTGAACATTAATCAGCAGCGTGGTGTTTATCAGAAACCGAACATTTTTAGC
AACACCCGTCTGTATACCGGTGTGGAAGTTATTATTCGTAAAAATGGCAGCACCGATATCAGCAACACCGATAAC
TTTGTTCGCAAAAATGATCTGGCGTATATCAACGTTGTTGATCGTGATGTTGAATATCGTCTGTATGCCGATATT
AGCATTGCCAAACCGGAAAAAATCATCAAACTGATCCGTACCAGCAACAGCAATAATTCACTGGGTCAGATTATT
GTGATGGATAGCATTGGTAATAACTGCACCATGAACTTTCAGAACAATAACGGTGGTAATATTGGTCTGCTGGGC
TTTCATAGTAATAATCTGGTTGCAAGCAGCTGGTATTATAACAACATCCGTAAAAATACCAGCAGCAATGGTTGC
TTTTGGAGCTTTATTAGCAAAGAACATGGCTGGCAAGAAAACGAGAATCTGTATTTTCAGGGTGCAAGTCATCAT
CACCACCATCACCATCATTAA
SEQ ID NO: 38-Polypeptide Sequence of rH_{C}/F (His-tagged)
MIKDNSILDMRYENNKFIDISGYGSNISINGDVYIYSTNRNQFGIYSSKPSEVNIAQNNDIIYNGRYQNFSISFW
VRIPKYFNKVNLNNEYTIIDCIRNNNSGWKISLNYNKIIWTLQDTAGNNQKLVENYTQMISISDYINKWIFVTIT
NNRLGNSRIYINGNLIDEKSISNLGDIHVSDNILFKIVGCNDTRYVGIRYFKVEDTELGKTEIETLYSDEPDPSI
LKDFWGNYLLYNKRYYLLNLLRTDKSITQNSNFLNINQQRGVYQKPNIFSNTRLYTGVEVIIRKNGSTDISNTDN
FVRKNDLAYINVVDRDVEYRLYADISIAKPEKIIKLIRTSNSNNSLGQIIVMDSIGNNCTMNFQNNNGGNIGLLG
FHSNNLVASSWYYNNIRKNTSSNGCFWSFISKEHGWQENENLYFQGASHHHHHHHH
SEQ ID NO: 39-Nucleotide Sequence of rLC/F (His-tagged)
ATGCCGGTTGTGATTAACAGCTTCAATTATAACGATCCGGTGAACGATGATACCATCCTGTATATGCAGATTCCG
TATGAAGAGAAAAGCAAAAAGTACTACAAAGCCTTTGAGATCATGCGCAACGTTTGGATTATTCCGGAACGTAAT
ACCATTGGCACCGATCCGAGCGATTTTGATCCGCCTGCAAGCCTGGAAAATGGTAGCAGCGCATATTATGATCCG
AATTATCTGACCACCGATGCCGAAAAAGATCGTTATCTGAAAACCACCATCAAACTGTTCAAACGCATTAATAGC
AATCCGGCAGGCGAAGTTCTGCTGCAAGAAATTAGCTATGCAAAACCGTATCTGGGCAATGAACATACCCCGATT
AATGAATTTCATCCGGTTACACGTACCACGAGCGTTAACATTAAAAGCAGCACCAATGTGAAGTCCAGCATTATT
CTGAATCTGCTGGTTTTAGGTGCAGGTCCGGATATTTTTGAAAATTCAAGCTATCCGGTGCGCAAACTGATGGAT
AGCGGTGGTGTGTATGATCCGTCAAATGATGGTTTTGGCAGCATTAACATTGTGACCTTTAGTCCGGAATATGAA
TACACCTTCAACGATATTAGCGGTGGCTATAATAGCAGCACCGAAAGTTTTATTGCAGATCCGGCAATTAGCCTG
GCACATGAACTGATTCATGCACTGCATGGTCTGTATGGTGCACGTGGTGTTACCTATAAAGAAACCATTAAAGTT
AAACAGGCACCGCTGATGATTGCGGAAAAACCGATTCGTCTGGAAGAATTTCTGACCTTTGGTGGTCAGGATCTG
AACATTATTACCAGCGCAATGAAAGAGAAAATCTATAATAACCTGCTGGCCAACTATGAGAAAATTGCAACCCGT
CTGAGCCGTGTTAATAGCGCACCTCCTGAATATGATATCAACGAGTATAAAGACTATTTTCAGTGGAAATACGGC
CTGGATAAAAATGCAGATGGTAGCTATACCGTGAACGAGAACAAATTTAACGAGATCTACAAAAAACTGTATAGC
TTCACCGAAATCGATCTGGCCAACAAATTCAAAGTGAAATGCCGCAACACCTACTTCATCAAATATGGCTTTCTG
AAAGTTCCGAACCTGCTTGATGATGATATCTATACCGTTAGCGAAGGCTTTAACATTGGTAATCTGGCCGTTAAT
AATCGCGGTCAGAACATTAAACTGAACCCGAAAATTATCGATAGCATCCCGGATAAAGGCCTGGTTGAAAAAATT
GTGAAATTCTGCAAAAGCGAGAACCTGTATTTTCAGGGTGCAAGTCATCATCACCATCACCACCATCATTAA
SEQ ID NO: 40-Polypeptide Sequence of rLC/F (His-tagged)
MPVVINSFNYNDPVNDDTILYMQIPYEEKSKKYYKAFEIMRNVWIIPERNTIGTDPSDFDPPASLENGSSAYYDP
NYLTTDAEKDRYLKTTIKLFKRINSNPAGEVLLQEISYAKPYLGNEHTPINEFHPVTRTTSVNIKSSTNVKSSII
LNLLVLGAGPDIFENSSYPVRKLMDSGGVYDPSNDGFGSINIVTFSPEYEYTENDISGGYNSSTESFIADPAISL
AHELIHALHGLYGARGVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKFKVKCRNTYFIKYGFL
KVPNLLDDDIYTVSEGFNIGNLAVNNRGQNIKLNPKIIDSIPDKGLVEKIVKFCKSENLYFQGASHHHHHHHH
SEQ ID NO: 41-Nucleotide Sequence of Cationic rH_{C}/A (His-tagged)
ATGATCATCAACACCAGCATTCTGAACCTGCGTTATGAAAGCAAACATCTGATTGATCTGAGCCGTTATGCCAGC
AAAATCAATATAGGCAGCAAGGTTAACTTCGACCCGATTGACAAAAATCAGATACAGCTGTTTAATCTGGAAAGC
AGCAAAATTGAGGTGATCCTGAAAAAAGCGATCGTGTATAATAGCATGTACGAGAATTTTTCGACCAGCTTTTGG
ATTCGCATCCCGAAATACTTTAACAAGATTAGCCTGAACAACGAGTATACCATCATTAACTGCATGGAAAACAAT
AGCGGTTGGAAAGTCAGCCTGAATTATGGCGAAATTATCTGGACCCTGCAGGATACCAAAGAAATCAAACAGCGT
GTGGTGTTCAAATACAGCCAGATGATTAATATCAGCGACTATATCAACCGCTGGATTTTTGTGACCATTACCAAT
AATCGGCTGAACAAGAGCAAGATCTATATTAACGGTCGTCTGATTGACCAGAAACCGATTAGTAATCTGGGTAAT
ATTCATGCGAGCAACAAAATCATGTTTAAACTGGATGGTTGCCGTGATACCCATCGTTATATTTGGATCAAATAC
TTCAACCTGTTCGATAAAGAGTTGAACGAAAAAGAAATTAAAGACCTGTACGATAACCAGAGCAATAGCGGCATA
CTGAAAGATTTTTGGGGAGATTATCTGCAGTATGACAAACCGTATTATATGCTGAATCTGTACGACCCGAATAAA
TACGTGGATGTTAATAATGTGGGCATCCGTGGTTATATGTACCTGAAAGGTCCGCGTGGTAGCGTTATGACCACA
AACATTTATCTGAATAGCAGCCTGTATCGCGGAACCAAATTCATCATTAAAAAGTATGCCAGCGGCAACAAGGAT
AATATTGTGCGTAATAATGATCGCGTGTACATTAACGTTGTGGTGAAGAATAAAGAATATCGCCTGGCAACCAAT
GCAAGCCAGGCAGGCGTTGAAAAAATTCTGAGTGCCCTGGAAATTCCGGATGTTGGTAATCTGAGCCAGGTTGTT
GTGATGAAAAGCAAAAACGATAAAGGCATCACCAACAAATGCAAGATGAATCTGCAGGACAATAACGGCAATGAT
ATTGGCTTCATTGGCTTTCACCAGTTTAACAACATTGCAAAACTGGTTGCGAGCAATTGGTATAATCGTCAGATT
GAACGTAGCAGTCGTACCCTGGGTTGTAGCTGGGAATTTATCCCTGTGGATGATGGTTGGGGTGAACGTCCGCTG
AAGCTTGCGGCCGCACTCGAGCACCACCACCACCACCACTGA
SEQ ID NO: 42-Polypeptide Sequence of Cationic rH_{C}/A (His-tagged)
MIINTSILNLRYESKHLIDLSRYASKINIGSKVNFDPIDKNQIQLENLESSKIEVILKKAIVYNSMYENFSTSFW
IRIPKYFNKISLNNEYTIINCMENNSGWKVSLNYGEIIWTLQDTKEIKQRVVFKYSQMINISDYINRWIFVTITN
NRLNKSKIYINGRLIDQKPISNLGNIHASNKIMFKLDGCRDTHRYIWIKYFNLFDKELNEKEIKDLYDNQSNSGI
LKDFWGDYLQYDKPYYMLNLYDPNKYVDVNNVGIRGYMYLKGPRGSVMTTNIYLNSSLYRGTKFIIKKYASGNKD
NIVRNNDRVYINVVVKNKEYRLATNASQAGVEKILSALEIPDVGNLSQVVVMKSKNDKGITNKCKMNLQDNNGND
IGFIGFHQFNNIAKLVASNWYNRQIERSSRTLGCSWEFIPVDDGWGERPLKLAAALEHHHHHH
SEQ ID NO: 43-Nucleotide Sequence of rH_{C}/AB (His-tagged)
ATGATTCTGAACAATATTATCCTGAACCTGCGTTACAAAGACAACAATCTGATCGATCTGAGCGGCTATGGTGCA
AAAGTTGAAGTCTACGACGGTGTCGAACTGAACGATAAAAACCAGTTCAAACTGACCTCATCGGCTAACTCAAAA
ATTCGTGTGACGCAGAACCAAAACATCATCTTCAACTCGGTCTTTCTGGACTTCAGCGTGTCTTTCTGGATTCGC
ATCCCGAAATATAAAAATGATGGCATCCAGAACTACATCCATAACGAATACACCATCATCAACTGTATGAAAAAC
AACAGTGGTTGGAAAATTTCCATCCGTGGCAACCGCATTATCTGGACCCTGATTGATATCAATGGTAAAACGAAA
AGCGTGTTTTTCGAATACAACATCCGTGAAGATATCTCTGAATACATCAATCGCTGGTTTTTCGTGACCATTACG
AACAATCTGAACAATGCGAAAATCTATATCAACGGCAAACTGGAAAGTAATACCGACATCAAAGATATTCGTGAA
GTTATCGCCAACGGTGAAATCATCTTCAAACTGGATGGCGACATCGATCGCACCCAGTTCATTTGGATGAAATAC
TTCTCCATCTTCAACACGGAACTGAGTCAGTCCAATATCGAAGAACGCTACAAAATCCAATCATACTCGGAATAC
CTGAAAGATTTCTGGGGTAACCCGCTGATGTACAACAAAGAATACTACATGTTCAACGCGGGCAACAAAAACTCA
TACATCAAACTGAAAAAAGATTCGCCGGTGGGTGAAATCCTGACCCGTAGCAAATACAACCAGAACTCTAAATAC
ATCAACTATCGCGATCTGTACATTGGCGAAAAATTTATTATCCGTCGCAAAAGCAACTCTCAGAGTATTAATGAT
GACATCGTGCGTAAAGAAGACTACATCTATCTGGATTTCTTTAATCTGAACCAAGAATGGCGCGTTTATACCTAC
AAATACTTCAAAAAAGAAGAAATGAAACTGTTCCTGGCCCCGATTTACGACAGCGATGAATTTTACAACACCATC
CAGATCAAAGAATACGATGAACAGCCGACGTATAGTTGCCAACTGCTGTTCAAAAAAGACGAAGAATCCACCGAT
GAAATTGGCCTGATTGGTATCCACCGTTTCTATGAAAGCGGTATCGTTTTCGAAGAATACAAAGATTACTTCTGT
ATCTCTAAATGGTATCTGAAAGAAGTCAAACGCAAACCGTACAACCTGAAACTGGGCTGCAACTGGCAATTTATC
CCGAAAGACGAAGGCTGGACCGAAAAGCTTGCGGCCGCACTCGAGCACCACCACCACCACCACTGA
SEQ ID NO: 44-Polypeptide Sequence of rH_{C}/AB (His-tagged)
MILNNIILNLRYKDNNLIDLSGYGAKVEVYDGVELNDKNQFKLTSSANSKIRVTQNQNIIFNSVFLDFSVSFWIR
IPKYKNDGIQNYIHNEYTIINCMKNNSGWKISIRGNRIIWTLIDINGKTKSVFFEYNIREDISEYINRWFFVTIT
NNLNNAKIYINGKLESNTDIKDIREVIANGEIIFKLDGDIDRTQFIWMKYFSIFNTELSQSNIEERYKIQSYSEY
LKDFWGNPLMYNKEYYMFNAGNKNSYIKLKKDSPVGEILTRSKYNQNSKYINYRDLYIGEKFIIRRKSNSQSIND
DIVRKEDYIYLDFFNLNQEWRVYTYKYFKKEEMKLFLAPIYDSDEFYNTIQIKEYDEQPTYSCQLLFKKDEESTD
EIGLIGIHRFYESGIVFEEYKDYFCISKWYLKEVKRKPYNLKLGCNWQFIPKDEGWTEKLAAALEHHHHHH
SEQ ID NO: 45-Nucleotide Sequence of rH_{C}/A Variant Y1117V H1253K (His-tagged)
ATGATCATCAATACTAGCATTCTGAACCTGCGTTACGAGAGCAATCATCTGATTGATCTGAGCCGTTATGCAAGC
AAGATCAACATCGGTAGCAAGGTCAATTTTGACCCGATCGATAAGAACCAGATCCAGCTGTTTAATCTGGAATCG
AGCAAAATTGAGGTTATCCTGAAAAACGCCATTGTCTACAACTCCATGTACGAGAATTTCTCCACCAGCTTCTGG
ATTCGCATCCCGAAATACTTCAACAGCATTAGCCTGAACAACGAGTATACTATCATCAACTGTATGGAGAACAAC
AGCGGTTGGAAGGTGTCTCTGAACTATGGTGAGATCATTTGGACCTTGCAGGACACCCAAGAGATCAAGCAGCGC
GTCGTGTTCAAGTACTCTCAAATGATCAACATTTCCGATTACATTAATCGTTGGATCTTCGTGACCATTACGAAT
AACCGTCTGAATAACAGCAAGATTTACATCAATGGTCGCTTGATCGATCAGAAACCGATTAGCAACCTGGGTAAT
ATCCACGCAAGCAACAACATTATGTTCAAATTGGACGGTTGCCGCGATACCCATCGTTATATCTGGATCAAGTAT
TTCAACCTGTTTGATAAAGAACTGAATGAGAAGGAGATCAAAGATTTGTATGACAACCAATCTAACAGCGGCATT
TTGAAGGACTTCTGGGGCGATTATCTGCAATACGATAAGCCGTACTATATGCTGAACCTGgtTGATCCGAACAAA
TATGTGGATGTCAATAATGTGGGTATTCGTGGTTACATGTATTTGAAGGGTCCGCGTGGCAGCGTTATGACGACC
AACATTTACCTGAACTCTAGCCTGTACCGTGGTACGAAATTCATCATTAAGAAATATGCCAGCGGCAACAAAGAT
AACATTGTGCGTAATAACGATCGTGTCTACATCAACGTGGTCGTGAAGAATAAAGAGTACCGTCTGGCGACCAAC
GCTTCGCAGGCGGGTGTTGAGAAAATTCTGAGCGCGTTGGAGATCCCTGATGTCGGTAATCTGAGCCAAGTCGTG
GTTATGAAGAGCAAGAACGACCAGGGTATCACTAACAAGTGCAAGATGAACCTGCAAGACAACAATGGTAACGAC
ATCGGCTTTATTGGTTTCaAaCAGTTCAACAATATTGCTAAACTGGTAGCGAGCAATTGGTACAATCGTCAGATT
GAGCGCAGCAGCCGTACTTTGGGCTGTAGCTGGGAGTTTATCCCGGTCGATGATGGTTGGGGCGAACGTCCGCTG
CACCATCACCATCACCATCACCATCACCATT
SEQ ID NO: 46-Polypeptide Sequence of rH_{C}/A Variant Y1117V H1253K (His-tagged)
MIINTSILNLRYESNHLIDLSRYASKINIGSKVNFDPIDKNQIQLENLESSKIEVILKNAIVYNSMYENFSTSFW
IRIPKYFNSISLNNEYTIINCMENNSGWKVSLNYGEIIWTLQDTQEIKQRVVFKYSQMINISDYINRWIFVTITN
NRLNNSKIYINGRLIDQKPISNLGNIHASNNIMFKLDGCRDTHRYIWIKYFNLFDKELNEKEIKDLYDNQSNSGI
LKDFWGDYLQYDKPYYMLNLVDPNKYVDVNNVGIRGYMYLKGPRGSVMTTNIYLNSSLYRGTKFIIKKYASGNKD
NIVRNNDRVYINVVVKNKEYRLATNASQAGVEKILSALEIPDVGNLSQVVVMKSKNDQGITNKCKMNLQDNNGND
IGFIGFKQFNNIAKLVASNWYNRQIERSSRTLGCSWEFIPVDDGWGERPLHHHHHHHHHH
SEQ ID NO: 47-Nucleotide Sequence of rHc/A Variant Y1117V F1252Y H1253K L1278F
(His-tagged)
ATGATCATCAATACTAGCATTCTGAACCTGCGTTACGAGAGCAATCATCTGATTGATCTGAGCCGTTATGCAAGC
AAGATCAACATCGGTAGCAAGGTCAATTTTGACCCGATCGATAAGAACCAGATCCAGCTGTTTAATCTGGAATCG
AGCAAAATTGAGGTTATCCTGAAAAACGCCATTGTCTACAACTCCATGTACGAGAATTTCTCCACCAGCTTCTGG
ATTCGCATCCCGAAATACTTCAACAGCATTAGCCTGAACAACGAGTATACTATCATCAACTGTATGGAGAACAAC
AGCGGTTGGAAGGTGTCTCTGAACTATGGTGAGATCATTTGGACCTTGCAGGACACCCAAGAGATCAAGCAGCGC
GTCGTGTTCAAGTACTCTCAAATGATCAACATTTCCGATTACATTAATCGTTGGATCTTCGTGACCATTACGAAT
AACCGTCTGAATAACAGCAAGATTTACATCAATGGTCGCTTGATCGATCAGAAACCGATTAGCAACCTGGGTAAT
ATCCACGCAAGCAACAACATTATGTTCAAATTGGACGGTTGCCGCGATACCCATCGTTATATCTGGATCAAGTAT
TTCAACCTGTTTGATAAAGAACTGAATGAGAAGGAGATCAAAGATTTGTATGACAACCAATCTAACAGCGGCATT
TTGAAGGACTTCTGGGGCGATTATCTGCAATACGATAAGCCGTACTATATGCTGAACCTGgtTGATCCGAACAAA
TATGTGGATGTCAATAATGTGGGTATTCGTGGTTACATGTATTTGAAGGGTCCGCGTGGCAGCGTTATGACGACC
AACATTTACCTGAACTCTAGCCTGTACCGTGGTACGAAATTCATCATTAAGAAATATGCCAGCGGCAACAAAGAT
AACATTGTGCGTAATAACGATCGTGTCTACATCAACGTGGTCGTGAAGAATAAAGAGTACCGTCTGGCGACCAAC
GCTTCGCAGGCGGGTGTTGAGAAAATTCTGAGCGCGTTGGAGATCCCTGATGTCGGTAATCTGAGCCAAGTCGTG
GTTATGAAGAGCAAGAACGACCAGGGTATCACTAACAAGTGCAAGATGAACCTGCAAGACAACAATGGTAACGAC
ATCGGCTTTATTGGTTaCaAaCAGTTCAACAATATTGCTAAACTGGTAGCGAGCAATTGGTACAATCGTCAGATT
GAGCGCAGCAGCCGTACTTTtGGCTGTAGCTGGGAGTTTATCCCGGTCGATGATGGTTGGGGCGAACGTCCGCTG
CACCATCACCATCACCATCACCATCACCATTAA
SEQ ID NO: 48-Polypeptide Sequence of rH_{C}/A Variant Y1117V F1252Y H1253K L1278F
(His-tagged)
MIINTSILNLRYESNHLIDLSRYASKINIGSKVNFDPIDKNQIQLFNLESSKIEVILKNAIVYNSMYENFSTSFW
IRIPKYFNSISLNNEYTIINCMENNSGWKVSLNYGEIIWTLQDTQEIKQRVVFKYSQMINISDYINRWIFVTITN
NRLNNSKIYINGRLIDQKPISNLGNIHASNNIMFKLDGCRDTHRYIWIKYFNLEDKELNEKEIKDLYDNQSNSGI
LKDFWGDYLQYDKPYYMLNLVDPNKYVDVNNVGIRGYMYLKGPRGSVMTTNIYLNSSLYRGTKFIIKKYASGNKD
NIVRNNDRVYINVVVKNKEYRLATNASQAGVEKILSALEIPDVGNLSQVVVMKSKNDQGITNKCKMNLQDNNGND
IGFIGYKQFNNIAKLVASNWYNRQIERSSRTFGCSWEFIPVDDGWGERPLHHHHHHHHHH
SEQ ID NO: 49-Nucleotide Sequence of rH_{C}/A Variant Y1117V F1252Y H1253K L1278H
(His-tagged)
ATGATCATCAATACTAGCATTCTGAACCTGCGTTACGAGAGCAATCATCTGATTGATCTGAGCCGTTATGCAAGC
AAGATCAACATCGGTAGCAAGGTCAATTTTGACCCGATCGATAAGAACCAGATCCAGCTGTTTAATCTGGAATCG
AGCAAAATTGAGGTTATCCTGAAAAACGCCATTGTCTACAACTCCATGTACGAGAATTTCTCCACCAGCTTCTGG
ATTCGCATCCCGAAATACTTCAACAGCATTAGCCTGAACAACGAGTATACTATCATCAACTGTATGGAGAACAAC
AGCGGTTGGAAGGTGTCTCTGAACTATGGTGAGATCATTTGGACCTTGCAGGACACCCAAGAGATCAAGCAGCGC
GTCGTGTTCAAGTACTCTCAAATGATCAACATTTCCGATTACATTAATCGTTGGATCTTCGTGACCATTACGAAT
AACCGTCTGAATAACAGCAAGATTTACATCAATGGTCGCTTGATCGATCAGAAACCGATTAGCAACCTGGGTAAT
ATCCACGCAAGCAACAACATTATGTTCAAATTGGACGGTTGCCGCGATACCCATCGTTATATCTGGATCAAGTAT
TTCAACCTGTTTGATAAAGAACTGAATGAGAAGGAGATCAAAGATTTGTATGACAACCAATCTAACAGCGGCATT
TTGAAGGACTTCTGGGGCGATTATCTGCAATACGATAAGCCGTACTATATGCTGAACCTGgtTGATCCGAACAAA
TATGTGGATGTCAATAATGTGGGTATTCGTGGTTACATGTATTTGAAGGGTCCGCGTGGCAGCGTTATGACGACC
AACATTTACCTGAACTCTAGCCTGTACCGTGGTACGAAATTCATCATTAAGAAATATGCCAGCGGCAACAAAGAT
AACATTGTGCGTAATAACGATCGTGTCTACATCAACGTGGTCGTGAAGAATAAAGAGTACCGTCTGGCGACCAAC
GCTTCGCAGGCGGGTGTTGAGAAAATTCTGAGCGCGTTGGAGATCCCTGATGTCGGTAATCTGAGCCAAGTCGTG
GTTATGAAGAGCAAGAACGACCAGGGTATCACTAACAAGTGCAAGATGAACCTGCAAGACAACAATGGTAACGAC
ATCGGCTTTATTGGTTaCaAaCAGTTCAACAATATTGCTAAACTGGTAGCGAGCAATTGGTACAATCGTCAGATT
GAGCGCAGCAGCCGTACTcatGGCTGTAGCTGGGAGTTTATCCCGGTCGATGATGGTTGGGGCGAACGTCCGCTG
CACCATCACCATCACCAT
SEQ ID NO: 50-Polypeptide Sequence of rH_{C}/A Variant Y1117V F1252Y H1253K L1278H
(His-tagged)
MIINTSILNLRYESNHLIDLSRYASKINIGSKVNFDPIDKNQIQLENLESSKIEVILKNAIVYNSMYENFSTSFW
IRIPKYENSISLNNEYTIINCMENNSGWKVSLNYGEIIWTLQDTQEIKQRVVFKYSQMINISDYINRWIFVTITN
NRLNNSKIYINGRLIDQKPISNLGNIHASNNIMFKLDGCRDTHRYIWIKYENLEDKELNEKEIKDLYDNQSNSGI
LKDFWGDYLQYDKPYYMLNLVDPNKYVDVNNVGIRGYMYLKGPRGSVMTTNIYLNSSLYRGTKFIIKKYASGNKD
NIVRNNDRVYINVVVKNKEYRLATNASQAGVEKILSALEIPDVGNLSQVVVMKSKNDQGITNKCKMNLQDNNGND
IGFIGYKQFNNIAKLVASNWYNRQIERSSRTHGCSWEFIPVDDGWGERPLHHHHHH
SEQ ID NO: 51-Polypeptide Sequence of BoNT/A-UniProt P10845
MPFVNKQFNYKDPVNGVDIAYIKIPNVGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNEVKFFKV
LNRKTYLNFDKAVFKINIVPKVNYTIYDGENLRNTNLAANENGQNTEINNMNFTKLKNFT
GLFEFYKLLCVRGIITSKTKSLDKGYNKALNDLCIKVNNWDLFFSPSEDNFTNDLNKGEE
ITSDTNIEAAEENISLDLIQQYYLTENEDNEPENISIENLSSDIIGQLELMPNIEREPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNIINTSILNLRYESNHLIDLSRYASKINI
GSKVNFDPIDKNQIQLENLESSKIEVILKNAIVYNSMYENFSTSFWIRIPKYFNSISLNN
EYTIINCMENNSGWKVSLNYGEIIWTLQDTQEIKQRVVFKYSQMINISDYINRWIFVTIT
NNRLNNSKIYINGRLIDQKPISNLGNIHASNNIMFKLDGCRDTHRYIWIKYENLEDKELN
EKEIKDLYDNQSNSGILKDFWGDYLQYDKPYYMLNLYDPNKYVDVNNVGIRGYMYLKGPR
GSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAK
LVASNWYNRQIERSSRTLGCSWEFIPVDDGWGERPL
SEQ ID NO: 52-Polypeptide Sequence of BoNT/B-UniProt P10844
MPVTINNENYNDPIDNNNIIMMEPPFARGTGRYYKAFKITDRIWIIPERYTFGYKPEDEN
KSSGIFNRDVCEYYDPDYLNTNDKKNIFLQTMIKLENRIKSKPLGEKLLEMIINGIPYLG
DRRVPLEEFNTNIASVTVNKLISNPGEVERKKGIFANLIIFGPGPVLNENETIDIGIQNH
FASREGFGGIMQMKFCPEYVSVENNVQENKGASIFNRRGYFSDPALILMHELIHVLHGLY
GIKVDDLPIVPNEKKFFMQSTDAIQAEELYTFGGQDPSIITPSTDKSIYDKVLQNERGIV
DRLNKVLVCISDPNININIYKNKFKDKYKFVEDSEGKYSIDVESFDKLYKSLMFGFTETN
IAENYKIKTRASYFSDSLPPVKIKNLLDNEIYTIEEGFNISDKDMEKEYRGQNKAINKQA
YEEISKEHLAVYKIQMCKSVKAPGICIDVDNEDLFFIADKNSESDDLSKNERIEYNTQSN
YIENDEPINELILDTDLISKIELPSENTESLTDFNVDVPVYEKQPAIKKIFTDENTIFQY
LYSQTFPLDIRDISLTSSFDDALLFSNKVYSFFSMDYIKTANKVVEAGLFAGWVKQIVND
FVIEANKSNTMDKIADISLIVPYIGLALNVGNETAKGNFENAFEIAGASILLEFIPELLI
PVVGAFLLESYIDNKNKIIKTIDNALTKRNEKWSDMYGLIVAQWLSTVNTQFYTIKEGMY
KALNYQAQALEEIIKYRYNIYSEKEKSNINIDENDINSKLNEGINQAIDNINNFINGCSV
SYLMKKMIPLAVEKLLDFDNTLKKNLLNYIDENKLYLIGSAEYEKSKVNKYLKTIMPEDL
SIYTNDTILIEMENKYNSEILNNIILNLRYKDNNLIDLSGYGAKVEVYDGVELNDKNQFK
LTSSANSKIRVTQNQNIIFNSVELDFSVSFWIRIPKYKNDGIQNYIHNEYTIINCMKNNS
GWKISIRGNRIIWTLIDINGKTKSVFFEYNIREDISEYINRWFFVTITNNLNNAKIYING
KLESNTDIKDIREVIANGEIIFKLDGDIDRTQFIWMKYESIENTELSQSNIEERYKIQSY
SEYLKDFWGNPLMYNKEYYMFNAGNKNSYIKLKKDSPVGEILTRSKYNQNSKYINYRDLY
IGEKFIIRRKSNSQSINDDIVRKEDYIYLDFFNLNQEWRVYTYKYFKKEEEKLFLAPISD
SDEFYNTIQIKEYDEQPTYSCQLLFKKDEESTDEIGLIGIHRFYESGIVFEEYKDYFCIS
KWYLKEVKRKPYNLKLGCNWQFIPKDEGWTE
SEQ ID NO: 53-Polypeptide Sequence of BoNT/C-UniProt P18640
MPITINNENYSDPVDNKNILYLDTHLNTLANEPEKAFRITGNIWVIPDRESRNSNPNLNK
PPRVTSPKSGYYDPNYLSTDSDKDPFLKEIIKLFKRINSREIGEELIYRLSTDIPFPGNN
NTPINTFDFDVDFNSVDVKTRQGNNWVKTGSINPSVIITGPRENIIDPETSTEKLINNTF
AAQEGFGALSIISISPREMLTYSNATNDVGEGRESKSEFCMDPILILMHELNHAMHNLYG
IAIPNDQTISSVTSNIFYSQYNVKLEYAEIYAFGGPTIDLIPKSARKYFEEKALDYYRSI
AKRLNSITTANPSSENKYIGEYKQKLIRKYRFVVESSGEVTVNRNKEVELYNELTQIFTE
FNYAKIYNVQNRKIYLSNVYTPVTANILDDNVYDIQNGENIPKSNLNVLEMGQNLSRNPA
LRKVNPENMLYLFTKFCHKAIDGRSLYNKTLDCRELLVKNTDLPFIGDISDVKTDIFLRK
DINEETEVIYYPDNVSVDQVILSKNTSEHGQLDLLYPSIDSESEILPGENQVFYDNRTQN
VDYLNSYYYLESQKLSDNVEDFTFTRSIEEALDNSAKVYTYFPTLANKVNAGVQGGLELM
WANDVVEDFTTNILRKDTLDKISDVSAIIPYIGPALNISNSVRRGNFTEAFAVTGVTILL
EAFPEFTIPALGAFVIYSKVQERNEIIKTIDNCLEQRIKRWKDSYEWMMGTWLSRIITQF
NNISYQMYDSLNYQAGAIKAKIDLEYKKYSGSDKENIKSQVENLKNSLDVKISEAMNNIN
KFIRECSVTYLFKNMLPKVIDELNEEDRNTKAKLINLIDSHNIILVGEVDKLKAKVNNSF
QNTIPFNIFSYTNNSLLKDIINEYENNINDSKILSLQNRKNTLVDTSGYNAEVSEEGDVQ
LNPIFPFDEKLGSSGEDRGKVIVTQNENIVYNSMYESFSISFWIRINKWVSNLPGYTIID
SVKNNSGWSIGIISNFLVFTLKQNEDSEQSINFSYDISNNAPGYNKWFFVTVTNNMMGNM
KIYINGKLIDTIKVKELTGINFSKTITFEINKIPDTGLITSDSDNINMWIRDFYIFAKEL
DGKDINILENSLQYTNVVKDYWGNDLRYNKEYYMVNIDYLNRYMYANSRQIVENTRRNNN
DENEGYKIIIKRIRGNTNDTRVRGGDILYFDMTINNKAYNLFMKNETMYADNHSTEDIYA
IGLREQTKDINDNIIFQIQPMNNTYYYASQIFKSNENGENISGICSIGTYRFRLGGDWYR
HNYLVPTVKQGNYASLLESTSTHWGFVPVSE
SEQ ID NO: 54-Polypeptide Sequence of BoNT/D-UniProt P19321
MTWPVKDFNYSDPVNDNDILYLRIPQNKLITTPVKAFMITQNIWVIPERFSSDTNPSLSK
PPRPTSKYQSYYDPSYLSTDEQKDTFLKGIIKLFKRINERDIGKKLINYLVVGSPFMGDS
STPEDTFDFTRHTTNIAVEKFENGSWKVTNIITPSVLIFGPLPNILDYTASLTLQGQQSN
PSFEGFGTLSILKVAPEFLLTESDVTSNQSSAVLGKSIFCMDPVIALMHELTHSLHQLYG
INIPSDKRIRPQVSEGFFSQDGPNVQFEELYTFGGLDVEIIPQIERSQLREKALGHYKDI
AKRLNNINKTIPSSWISNIDKYKKIFSEKYNEDKDNTGNFVVNIDKENSLYSDLTNVMSE
VVYSSQYNVKNRTHYFSRHYLPVFANILDDNIYTIRDGENLINKGENIENSGQNIERNPA
LQKLSSESVVDLFTKVCLRLTKNSRDDSTCIKVKNNRLPYVADKDSISQEIFENKIITDE
TNVQNYSDKFSLDESILDGQVPINPEIVDPLLPNVNMEPLNLPGEEIVFYDDITKYVDYL
NSYYYLESQKLSNNVENITLTTSVEEALGYSNKIYTFLPSLAEKVNKGVQAGLELNWANE
VVEDFTTNIMKKDTLDKISDVSVIIPYIGPALNIGNSALRGNENQAFATAGVAFLLEGEP
EFTIPALGVFTFYSSIQEREKIIKTIENCLEQRVKRWKDSYQWMVSNWLSRITTQENHIN
YQMYDSLSYQADAIKAKIDLEYKKYSGSDKENIKSQVENLKNSLDVKISEAMNNINKFIR
ECSVTYLFKNMLPKVIDELNKFDLRTKTELINLIDSHNIILVGEVDRLKAKVNESFENTM
PFNIFSYTNNSLLKDIINEYENSINDSKILSLQNKKNALVDTSGYNAEVRVGDNVQLNTI
YTNDFKLSSSGDKIIVNLNNNILYSAIYENSSVSFWIKISKDLTNSHNEYTIINSIEQNS
GWKLCIRNGNIEWILQDVNRKYKSLIFDYSESLSHTGYTNKWFFVTITNNIMGYMKLYIN
GELKQSQKIEDLDEVKLDKTIVEGIDENIDENQMLWIRDENIESKELSNEDINIVYEGQI
LRNVIKDYWGNPLKEDTEYYIINDNYIDRYIAPESNVLVLVQYPDRSKLYTGNPITIKSV
SDKNPYSRILNGDNIILHMLYNSRKYMIIRDTDTIYATQGGECSQNCVYALKLQSNLGNY
GIGIFSIKNIVSKNKYCSQIFSSFRENTMLLADIYKPWRESEKNAYTPVAVTNYETKLLS
TSSFWKFISRDPGWVE
SEQ ID NO: 55-Polypeptide Sequence of BoNT/E-UniProt Q00496
MPKINSFNYNDPVNDRTILYIKPGGCQEFYKSFNIMKNIWIIPERNVIGTTPQDFHPPTS
LKNGDSSYYDPNYLQSDEEKDRFLKIVTKIFNRINNNLSGGILLEELSKANPYLGNDNTP
DNQFHIGDASAVEIKESNGSQDILLPNVIIMGAEPDLFETNSSNISLRNNYMPSNHRFGS
IAIVTESPEYSFRENDNCMNEFIQDPALTLMHELIHSLHGLYGAKGITTKYTITQKQNPL
ITNIRGTNIEEFLTFGGTDLNIITSAQSNDIYTNLLADYKKIASKLSKVQVSNPLLNPYK
DVFEAKYGLDKDASGIYSVNINKENDIFKKLYSFTEFDLRTKFQVKCRQTYIGQYKYFKL
SNLLNDSIYNISEGYNINNLKVNFRGQNANLNPRIITPITGRGLVKKIIRFCKNIVSVKG
IRKSICIEINNGELFFVASENSYNDDNINTPKEIDDTVTSNNNYENDLDQVILNENSESA
PGLSDEKLNLTIQNDAYIPKYDSNGTSDIEQHDVNELNVEFYLDAQKVPEGENNVNLTSS
IDTALLEQPKIYTFFSSEFINNVNKPVQAALFVSWIQQVLVDETTEANQKSTVDKIADIS
IVVPYIGLALNIGNEAQKGNEKDALELLGAGILLEFEPELLIPTILVFTIKSFLGSSDNK
NKVIKAINNALKERDEKWKEVYSFIVSNWMTKINTQFNKRKEQMYQALQNQVNAIKTIIE
SKYNSYTLEEKNELINKYDIKQIENELNQKVSIAMNNIDRELTESSISYLMKIINEVKIN
KLREYDENVKTYLLNYIIQHGSILGESQQELNSMVTDTLNNSIPFKLSSYTDDKILISYF
NKFFKRIKSSSVLNMRYKNDKYVDTSGYDSNININGDVYKYPTNKNQFGIYNDKLSEVNI
SQNDYIIYDNKYKNFSISFWVRIPNYDNKIVNVNNEYTIINCMRDNNSGWKVSLNHNEII
WTFEDNRGINQKLAFNYGNANGISDYINKWIFVTITNDRLGDSKLYINGNLIDQKSILNL
GNIHVSDNILFKIVNCSYTRYIGIRYENIFDKELDETEIQTLYSNEPNTNILKDEWGNYL
LYDKEYYLLNVLKPNNFIDRRKDSTLSINNIRSTILLANRLYSGIKVKIQRVNNSSTNDN
LVRKNDQVYINFVASKTHLFPLYADTATTNKEKTIKISSSGNRFNQVVVMNSVGNCTMNE
KNNNGNNIGLLGFKADTVVASTWYYTHMRDHTNSNGCFWNFISEEHGWQEK
SEQ ID NO: 56-Polypeptide Sequence of BoNT/F-UniProt A7GBG3
MPVVINSFNYNDPVNDDTILYMQIPYEEKSKKYYKAFEIMRNVWIIPERNTIGTDPSDED
PPASLENGSSAYYDPNYLTTDAEKDRYLKTTIKLFKRINSNPAGEVLLQEISYAKPYLGN
EHTPINEFHPVTRTTSVNIKSSTNVKSSIILNLLVLGAGPDIFENSSYPVRKLMDSGGVY
DPSNDGFGSINIVTFSPEYEYTENDISGGYNSSTESFIADPAISLAHELIHALHGLYGAR
GVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKE
KVKCRNTYFIKYGFLKVPNLLDDDIYTVSEGFNIGNLAVNNRGQNIKLNPKIIDSIPDKG
LVEKIVKFCKSVIPRKGTKAPPRLCIRVNNRELFFVASESSYNENDINTPKEIDDTTNLN
NNYRNNLDEVILDYNSETIPQISNQTLNTLVQDDSYVPRYDSNGTSEIEEHNVVDLNVFF
YLHAQKVPEGETNISLTSSIDTALSEESQVYTFFSSEFINTINKPVHAALFISWINQVIR
DFTTEATQKSTEDKIADISLVVPYVGLALNIGNEVQKENFKEAFELLGAGILLEFVPELL
IPTILVFTIKSFIGSSENKNKIIKAINNSLMERETKWKEIYSWIVSNWLTRINTQFNKRK
EQMYQALQNQVDAIKTVIEYKYNNYTSDERNRLESEYNINNIREELNKKVSLAMENIERF
ITESSIFYLMKLINEAKVSKLREYDEGVKEYLLDYISEHRSILGNSVQELNDLVTSTLNN
SIPFELSSYTNDKILILYENKLYKKIKDNSILDMRYENNKFIDISGYGSNISINGDVYIY
STNRNQFGIYSSKPSEVNIAQNNDIIYNGRYQNFSISFWVRIPKYFNKVNLNNEYTIIDC
IRNNNSGWKISLNYNKIIWTLQDTAGNNQKLVENYTQMISISDYINKWIFVTITNNRLGN
SRIYINGNLIDEKSISNLGDIHVSDNILFKIVGCNDTRYVGIRYFKVFDTELGKTEIETL
YSDEPDPSILKDFWGNYLLYNKRYYLLNLLRTDKSITQNSNFLNINQQRGVYQKPNIFSN
TRLYTGVEVIIRKNGSTDISNTDNFVRKNDLAYINVVDRDVEYRLYADISIAKPEKIIKL
IRTSNSNNSLGQIIVMDSIGNNCTMNFQNNNGGNIGLLGFHSNNLVASSWYYNNIRKNTS
SNGCFWSFISKEHGWQEN
SEQ ID NO: 57-Polypeptide Sequence of BoNT/G-UniProt Q60393
MPVNIKXFNYNDPINNDDIIMMEPENDPGPGTYYKAFRIIDRIWIVPERFTYGFQPDQEN
ASTGVESKDVYEYYDPTYLKTDAEKDKELKTMIKLENRINSKPSGQRLLDMIVDAIPYLG
NASTPPDKFAANVANVSINKKIIQPGAEDQIKGLMTNLIIFGPGPVLSDNFTDSMIMNGH
SPISEGFGARMMIRFCPSCLNVENNVQENKDTSIFSRRAYFADPALTLMHELIHVLHGLY
GIKISNLPITPNTKEFFMQHSDPVQAEELYTFGGHDPSVISPSTDMNIYNKALQNFQDIA
NRLNIVSSAQGSGIDISLYKQIYKNKYDFVEDPNGKYSVDKDKEDKLYKALMFGFTETNL
AGEYGIKTRYSYFSEYLPPIKTEKLLDNTIYTQNEGENIASKNLKTEFNGQNKAVNKEAY
EEISLEHLVIYRIAMCKPVMYKNTGKSEQCIIVNNEDLFFIANKDSFSKDLAKAETIAYN
TQNNTIENNESIDQLILDNDLSSGIDLPNENTEPFTNEDDIDIPVYIKQSALKKIFVDGD
SLFEYLHAQTFPSNIENLQLTNSLNDALRNNNKVYTFFSTNLVEKANTVVGASLFVNWVK
GVIDDFTSESTQKSTIDKVSDVSIIIPYIGPALNVGNETAKENEKNAFEIGGAAILMEFI
PELIVPIVGFFTLESYVGNKGHIIMTISNALKKRDQKWTDMYGLIVSQWLSTVNTQFYTI
KERMYNALNNQSQAIEKIIEDQYNRYSEEDKMNINIDENDIDFKLNQSINLAINNIDDFI
NQCSISYLMNRMIPLAVKKLKDEDDNLKRDLLEYIDTNELYLLDEVNILKSKVNRHLKDS
IPFDLSLYTKDTILIQVENNYISNISSNAILSLSYRGGRLIDSSGYGATMNVGSDVIEND
IGNGQFKLNNSENSNITAHQSKFVVYDSMFDNESINFWVRTPKYNNNDIQTYLQNEYTII
SCIKNDSGWKVSIKGNRIIWTLIDVNAKSKSIFFEYSIKDNISDYINKWFSITITNDRLG
NANIYINGSLKKSEKILNLDRINSSNDIDFKLINCTDTTKFVWIKDENIFGRELNATEVS
SLYWIQSSTNTLKDFWGNPLRYDTQYYLENQGMQNIYIKYFSKASMGETAPRTNENNAAI
NYQNLYLGLRFIIKKASNSRNINNDNIVREGDYIYLNIDNISDESYRVYVLVNSKEIQTQ
LFLAPINDDPTFYDVLQIKKYYEKTTYNCQILCEKDTKTFGLFGIGKFVKDYGYVWDTYD
NYFCISQWYLRRISENINKLRLGCNWQFIPVDEGWTE
SEQ ID NO: 58-Polypeptide Sequence of TeNT-UniProt P04958
MPITINNFRYSDPVNNDTIIMMEPPYCKGLDIYYKAFKITDRIWIVPERYEFGTKPEDEN
PPSSLIEGASEYYDPNYLRTDSDKDRFLQTMVKLENRIKNNVAGEALLDKIINAIPYLGN
SYSLLDKFDTNSNSVSFNLLEQDPSGATTKSAMLTNLIIFGPGPVLNKNEVRGIVLRVDN
KNYFPCRDGFGSIMQMAFCPEYVPTFDNVIENITSLTIGKSKYFQDPALLLMHELIHVLH
GLYGMQVSSHEIIPSKQEIYMQHTYPISAEELFTFGGQDANLISIDIKNDLYEKTLNDYK
AIANKLSQVTSCNDPNIDIDSYKQIYQQKYQFDKDSNGQYIVNEDKFQILYNSIMYGFTE
IELGKKFNIKTRLSYFSMNHDPVKIPNLLDDTIYNDTEGENIESKDLKSEYKGQNMRVNT
NAFRNVDGSGLVSKLIGLCKKIIPPTNIRENLYNRTASLTDLGGELCIKIKNEDLTFIAE
KNSFSEEPFQDEIVSYNTKNKPLNFNYSLDKIIVDYNLQSKITLPNDRTTPVTKGIPYAP
EYKSNAASTIEIHNIDDNTIYQYLYAQKSPTTLQRITMTNSVDDALINSTKIYSYFPSVI
SKVNQGAQGILFLQWVRDIIDDFTNESSQKTTIDKISDVSTIVPYIGPALNIVKQGYEGN
FIGALETTGVVLLLEYIPEITLPVIAALSIAESSTQKEKIIKTIDNFLEKRYEKWIEVYK
LVKAKWLGTVNTQFQKRSYQMYRSLEYQVDAIKKIIDYEYKIYSGPDKEQIADEINNLKN
KLEEKANKAMININIFMRESSRSFLVNQMINEAKKQLLEFDTQSKNILMQYIKANSKFIG
ITELKKLESKINKVFSTPIPFSYSKNLDCWVDNEEDIDVILKKSTILNLDINNDIISDIS
GENSSVITYPDAQLVPGINGKAIHLVNNESSEVIVHKAMDIEYNDMENNFTVSFWLRVPK
VSASHLEQYGTNEYSIISSMKKHSLSIGSGWSVSLKGNNLIWTLKDSAGEVRQITFRDLP
DKFNAYLANKWVFITITNDRLSSANLYINGVLMGSAEITGLGAIREDNNITLKLDRCNNN
NQYVSIDKFRIFCKALNPKEIEKLYTSYLSITFLRDFWGNPLRYDTEYYLIPVASSSKDV
QLKNITDYMYLTNAPSYTNGKLNIYYRRLYNGLKFIIKRYTPNNEIDSFVKSGDFIKLYV
SYNNNEHIVGYPKDGNAFNNLDRILRVGYNAPGIPLYKKMEAVKLRDLKTYSVQLKLYDD
KNASLGLVGTHNGQIGNDPNRDILIASNWYFNHLKDKILGCDWYFVPTDEGWTND
SEQ ID NO: 59-Polypeptide Sequence of BoNT/X
MKLEINKENYNDPIDGINVITMRPPRHSDKINKGKGPFKAFQVIKNIWIVPERYNFTNNT
NDLNIPSEPIMEADAIYNPNYLNTPSEKDEFLQGVIKVLERIKSKPEGEKLLELISSSIP
LPLVSNGALTLSDNETIAYQENNNIVSNLQANLVIYGPGPDIANNATYGLYSTPISNGEG
TLSEVSFSPFYLKPFDESYGNYRSLVNIVNKFVKREFAPDPASTLMHELVHVTHNLYGIS
NRNFYYNFDTGKIETSRQQNSLIFEELLTFGGIDSKAISSLIIKKIIETAKNNYTTLISE
RLNTVTVENDLLKYIKNKIPVQGRLGNFKLDTAEFEKKLNTILFVLNESNLAQRFSILVR
KHYLKERPIDPIYVNILDDNSYSTLEGENISSQGSNDFQGQLLESSYFEKIESNALRAFI
KICPRNGLLYNAIYRNSKNYLNNIDLEDKKTTSKTNVSYPCSLLNGCIEVENKDLFLISN
KDSLNDINLSEEKIKPETTVFFKDKLPPQDITLSNYDFTEANSIPSISQQNILERNEELY
EPIRNSLFEIKTIYVDKLTTFHFLEAQNIDESIDSSKIRVELTDSVDEALSNPNKVYSPF
KNMSNTINSIETGITSTYIFYQWLRSIVKDESDETGKIDVIDKSSDTLAIVPYIGPLLNI
GNDIRHGDFVGAIELAGITALLEYVPEFTIPILVGLEVIGGELAREQVEAIVNNALDKRD
QKWAEVYNITKAQWWGTIHLQINTRLAHTYKALSRQANAIKMNMEFQLANYKGNIDDKAK
IKNAISETEILLNKSVEQAMKNTEKFMIKLSNSYLTKEMIPKVQDNLKNEDLETKKTLDK
FIKEKEDILGTNLSSSLRRKVSIRLNKNIAFDINDIPESEFDDLINQYKNEIEDYEVLNL
GAEDGKIKDLSGTTSDINIGSDIELADGRENKAIKIKGSENSTIKIAMNKYLRFSATDNE
SISFWIKHPKPTNLLNNGIEYTLVENFNQRGWKISIQDSKLIWYLRDHNNSIKIVTPDYI
AFNGWNLITITNNRSKGSIVYVNGSKIEEKDISSIWNTEVDDPIIFRLKNNRDTQAFTLL
DQFSIYRKELNQNEVVKLYNYYENSNYIRDIWGNPLQYNKKYYLQTQDKPGKGLIREYWS
SFGYDYVILSDSKTITFPNNIRYGALYNGSKVLIKNSKKLDGLVRNKDFIQLEIDGYNMG
ISADRFNEDTNYIGTTYGTTHDLTTDFEIIQRQEKYRNYCQLKTPYNIFHKSGLMSTETS
KPTFHDYRDWVYSSAWYFQNYENLNLRKHTKTNWYFIPKDEGWDED
SEQ ID NO: 60-Polypeptide Sequence of Unmodified BoNT/A1
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTINKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLENLESSKIEVILKNAIVYNSMYENFSTSFWIRIPKYENSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSS
RTLGCSWEFIPVDDGWGERPL
SEQ ID NO: 61-Polypeptide Sequence of mrBoNT/AB(0)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHQL
IYAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVEK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTENEDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKYKNDGIQNYIHNEYTIINCMKNNSGW
KISIRGNRIIWTLIDINGKTKSVFFEYNIREDISEYINRWFFVTITNNLNNAKIYINGKLESNTDIKDIREVIAN
GEIIFKLDGDIDRTQFIWMKYFSIFNTELSQSNIEERYKIQSYSEYLKDFWGNPLMYNKEYYMFNAGNKNSYIKL
KKDSPVGEILTRSKYNQNSKYINYRDLYIGEKFIIRRKSNSQSINDDIVRKEDYIYLDFFNLNQEWRVYTYKYFK
KEEMKLFLAPIYDSDEFYNTIQIKEYDEQPTYSCQLLFKKDEESTDEIGLIGIHRFYESGIVFEEYKDYFCISKW
YLKEVKRKPYNLKLGCNWQFIPKDEGWTE
SEQ ID NO: 62-Polypeptide Sequence of mrBoNT/A(0)
MPFVNKQFNYKDPVNGVDIAYIKIPNAGQMQPVKAFKIHNKIWVIPERDT
FTNPEEGDLNPPPEAKQVPVSYYDSTYLSTDNEKDNYLKGVTKLFERIYS
TDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESL
EVDTNPLLGAGKFATDPAVTLAHQLIYAGHRLYGIAINPNRVFKVNTNAY
YEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYT
EDNFVKFFKVLNRKTYLNFDKAVFKINIVPKVNYTIYDGFNLRNTNLAAN
FNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQ
QYYLTFNFDNEPENISIENLSSDIIGQLELMPNIERFPNGKKYELDKYTM
FHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALS
KRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSM
IPYGVKRLEDFDASLKDALLKYIYDNRGTLIGQVDRLKDKVNNTLSTDIP
FQLSKYVDNQRLLSTFTEYIKNIINTSILNLRYESKHLIDLSRYASKINI
GSKVNFDPIDKNQIQLENLESSKIEVILKKAIVYNSMYENFSTSFWIRIP
KYFNKISLNNEYTIINCMENNSGWKVSLNYGEIIWTLQDTKEIKQRVVFK
YSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDF
WGDYLQYDKPYYMLNLYDPNKYVDVNNVGIRGYMYLKGPRGSVMTTNIYL
NSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFI
GFHQFNNIAKLVASNWYNRQIERSSRTLGCSWEFIPVDDGWGERPL
SEQ ID NO: 63-Polypeptide Sequence of BoNT/XB(0) (His-tagged)
MGSMKLEINKFNYNDPIDGINVITMRPPRH SDKINKGKGPFKAFQVIKNIWIVPERYNFT
NNTNDLNIPSEPIMEADAIYNPNYLNTPSE KDEFLQGVIKVLERIKSKPEGEKLLELISS
SIPLPLVSNGALTLSDNETIAYQENNNIVS NLQANLVIYGPGPDIANNATYGLYSTPISN
GEGTLSEVSFSPFYLKPFDESYGNYRSLVN IVNKFVKREFAPDPASTLMHQLVYVTHNLY
GISNRNFYYNFDTGKIETSRQQNSLIFEEL LTFGGIDSKAISSLIIKKIIETAKNNYTTL
ISERLNTVTVENDLLKYIKNKIPVQGRLGN FKLDTAEFEKKLNTILFVLNESNLAQRFSI
LVRKHYLKERPIDPIYVNILDDNSYSTLEG FNISSQGSNDFQGQLLESSYFEKIESNALR
AFIKICHKAIDGRSLYNKTLDCIEVENKDL FLISNKDSLNDINLSEEKIKPETTVFFKDK
LPPQDITLSNYDFTEANSIPSISQQNILER NEELYEPIRNSLFEIKTIYVDKLTTFHFLE
AQNIDESIDSSKIRVELTDSVDEALSNPNK VYSPFKNMSNTINSIETGITSTYIFYQWLR
SIVKDFSDETGKIDVIDKSSDTLAIVPYIG PLLNIGNDIRHGDFVGAIELAGITALLEYV
PEFTIPILVGLEVIGGELAREQVEAIVNNA LDKRDQKWAEVYNITKAQWWGTIHLQINTR
LAHTYKALSRQANAIKMNMEFQLANYKGNI DDKAKIKNAISETEILLNKSVEQAMKNTEK
FMIKLSNSYLTKEMIPKVQDNLKNEDLETK KTLDKFIKEKEDILGTNLSSSLRRKVSIRL
NKNIAFDINDIPFSEFDDLINQYKNEIEDY EVLNLGAEDGKIKDLSGTTSDINIGSDIEI
LNNIILNLRYKDNNLIDLSGYGAKVEVYDG VELNDKNQFKLTSSANSKIRVTQNQNIIEN
SVFLDFSVSFWIRIPKYKNDGIQNYIHNEY TIINCMKNNSGWKISIRGNRIIWTLIDING
KTKSVFFEYNIREDISEYINRWFFVTITNN LNNAKIYINGKLESNTDIKDIREVIANGEI
IFKLDGDIDRTQFIWMKYFSIFNTELSQSN IEERYKIQSYSEYLKDFWGNPLMYNKEYYM
FNAGNKNSYIKLKKDSPVGEILTRSKYNQN SKYINYRDLYIGEKFIIRRKSNSQSINDDI
VRKEDYIYLDFFNLNQEWRVYTYKYFKKEE MKLFLAPIYDSDEFYNTIQIKEYDEQPTYS
CQLLFKKDEESTDEIGLIGIHRFYESGIVE EEYKDYFCISKWYLKEVKRKPYNLKLGCNW
QFIPKDEGWTEHHHHHHHHHH
SEQ ID NO: 64-Polypeptide Sequence of BoNT/XB(0)
MGSMKLEINKFNYNDPIDGINVITMRPPRH SDKINKGKGPFKAFQVIKNIWIVPERYNFT
NNTNDLNIPSEPIMEADAIYNPNYLNTPSE KDEFLQGVIKVLERIKSKPEGEKLLELISS
SIPLPLVSNGALTLSDNETIAYQENNNIVS NLQANLVIYGPGPDIANNATYGLYSTPISN
GEGTLSEVSFSPFYLKPFDESYGNYRSLVN IVNKFVKREFAPDPASTLMHQLVYVTHNLY
GISNRNFYYNFDTGKIETSRQQNSLIFEEL LTFGGIDSKAISSLIIKKIIETAKNNYTTL
ISERLNTVTVENDLLKYIKNKIPVQGRLGN FKLDTAEFEKKLNTILFVLNESNLAQRFSI
LVRKHYLKERPIDPIYVNILDDNSYSTLEG FNISSQGSNDFQGQLLESSYFEKIESNALR
AFIKICHKAIDGRSLYNKTLDCIEVENKDL FLISNKDSLNDINLSEEKIKPETTVFFKDK
LPPQDITLSNYDFTEANSIPSISQQNILER NEELYEPIRNSLFEIKTIYVDKLTTFHFLE
AQNIDESIDSSKIRVELTDSVDEALSNPNK VYSPFKNMSNTINSIETGITSTYIFYQWLR
SIVKDFSDETGKIDVIDKSSDTLAIVPYIG PLLNIGNDIRHGDFVGAIELAGITALLEYV
PEFTIPILVGLEVIGGELAREQVEAIVNNA LDKRDQKWAEVYNITKAQWWGTIHLQINTR
LAHTYKALSRQANAIKMNMEFQLANYKGNI DDKAKIKNAISETEILLNKSVEQAMKNTEK
FMIKLSNSYLTKEMIPKVQDNLKNEDLETK KTLDKFIKEKEDILGTNLSSSLRRKVSIRL
NKNIAFDINDIPFSEFDDLINQYKNEIEDY EVLNLGAEDGKIKDLSGTTSDINIGSDIEI
LNNIILNLRYKDNNLIDLSGYGAKVEVYDG VELNDKNQFKLTSSANSKIRVTQNQNIIFN
SVFLDFSVSFWIRIPKYKNDGIQNYIHNEY TIINCMKNNSGWKISIRGNRIIWTLIDING
KTKSVFFEYNIREDISEYINRWFFVTITNN LNNAKIYINGKLESNTDIKDIREVIANGEI
IFKLDGDIDRTQFIWMKYFSIFNTELSQSN IEERYKIQSYSEYLKDFWGNPLMYNKEYYM
FNAGNKNSYIKLKKDSPVGEILTRSKYNQN SKYINYRDLYIGEKFIIRRKSNSQSINDDI
VRKEDYIYLDFFNLNQEWRVYTYKYFKKEE MKLFLAPIYDSDEFYNTIQIKEYDEQPTYS
CQLLFKKDEESTDEIGLIGIHRFYESGIVE EEYKDYFCISKWYLKEVKRKPYNLKLGCNW
QFIPKDEGWTE
SEQ ID NO: 65-Polypeptide Sequence of BoNT/XB(0) Variant (His-tagged)
MGSMKLEINKFNYNDPIDGINVITMRPPRH SDKINKGKGPFKAFQVIKNIWIVPERYNFT
NNTNDLNIPSEPIMEADAIYNPNYLNTPSE KDEFLQGVIKVLERIKSKPEGEKLLELISS
SIPLPLVSNGALTLSDNETIAYQENNNIVS NLQANLVIYGPGPDIANNATYGLYSTPISN
GEGTLSEVSFSPFYLKPFDESYGNYRSLVN IVNKFVKREFAPDPASTLMHQLVYVTHNLY
GISNRNFYYNFDTGKIETSRQQNSLIFEEL LTFGGIDSKAISSLIIKKIIETAKNNYTTL
ISERLNTVTVENDLLKYIKNKIPVQGRLGN FKLDTAEFEKKLNTILFVLNESNLAQRESI
LVRKHYLKERPIDPIYVNILDDNSYSTLEG FNISSQGSNDFQGQLLESSYFEKIESNALR
AFIKICHKAIDGRSLYNKTLDCIEVENKDL FLISNKDSLNDINLSEEKIKPETTVFFKDK
LPPQDITLSNYDFTEANSIPSISQQNILER NEELYEPIRNSLFEIKTIYVDKLTTFHFLE
AQNIDESIDSSKIRVELTDSVDEALSNPNK VYSPFKNMSNTINSIETGITSTYIFYQWLR
SIVKDFSDETGKIDVIDKSSDTLAIVPYIG PLLNIGNDIRHGDFVGAIELAGITALLEYV
PEFTIPILVGLEVIGGELAREQVEAIVNNA LDKRDQKWAEVYNITKAQWWGTIHLQINTR
LAHTYKALSRQANAIKMNMEFQLANYKGNI DDKAKIKNAISETEILLNKSVEQAMKNTEK
FMIKLSNSYLTKEMIPKVQDNLKNEDLETK KTLDKFIKEKEDILGTNLSSSLRRKVSIRL
NKNIAFDINDIPFSEFDDLINQYKNEILNN IILNLRYKDNNLIDLSGYGAKVEVYDGVEL
NDKNQFKLTSSANSKIRVTQNQNIIFNSVF LDFSVSFWIRIPKYKNDGIQNYIHNEYTII
NCMKNNSGWKISIRGNRIIWTLIDINGKTK SVFFEYNIREDISEYINRWFFVTITNNLNN
AKIYINGKLESNTDIKDIREVIANGEIIFK LDGDIDRTQFIWMKYFSIFNTELSQSNIEE
RYKIQSYSEYLKDFWGNPLMYNKEYYMENA GNKNSYIKLKKDSPVGEILTRSKYNQNSKY
INYRDLYIGEKFIIRRKSNSQSINDDIVRK EDYIYLDFFNLNQEWRVYTYKYFKKEEMKL
FLAPIYDSDEFYNTIQIKEYDEQPTYSCQL LFKKDEESTDEIGLIGIHRFYESGIVFEEY
KDYFCISKWYLKEVKRKPYNLKLGCNWQFI PKDEGWTEHHHHHHHHHH
SEQ ID NO: 66-Polypeptide Sequence of BoNT/XB(0) Variant
MGSMKLEINKFNYNDPIDGINVITMRPPRH SDKINKGKGPFKAFQVIKNIWIVPERYNFT
NNTNDLNIPSEPIMEADAIYNPNYLNTPSE KDEFLQGVIKVLERIKSKPEGEKLLELISS
SIPLPLVSNGALTLSDNETIAYQENNNIVS NLQANLVIYGPGPDIANNATYGLYSTPISN
GEGTLSEVSFSPFYLKPFDESYGNYRSLVN IVNKFVKREFAPDPASTLMHQLVYVTHNLY
GISNRNFYYNFDTGKIETSRQQNSLIFEEL LTFGGIDSKAISSLIIKKIIETAKNNYTTL
ISERLNTVTVENDLLKYIKNKIPVQGRLGN FKLDTAEFEKKLNTILFVLNESNLAQRESI
LVRKHYLKERPIDPIYVNILDDNSYSTLEG FNISSQGSNDFQGQLLESSYFEKIESNALR
AFIKICHKAIDGRSLYNKTLDCIEVENKDL FLISNKDSLNDINLSEEKIKPETTVFFKDK
LPPQDITLSNYDFTEANSIPSISQQNILER NEELYEPIRNSLFEIKTIYVDKLTTFHFLE
AQNIDESIDSSKIRVELTDSVDEALSNPNK VYSPFKNMSNTINSIETGITSTYIFYQWLR
SIVKDFSDETGKIDVIDKSSDTLAIVPYIG PLLNIGNDIRHGDFVGAIELAGITALLEYV
PEFTIPILVGLEVIGGELAREQVEAIVNNA LDKRDQKWAEVYNITKAQWWGTIHLQINTR
LAHTYKALSRQANAIKMNMEFQLANYKGNI DDKAKIKNAISETEILLNKSVEQAMKNTEK
FMIKLSNSYLTKEMIPKVQDNLKNEDLETK KTLDKFIKEKEDILGTNLSSSLRRKVSIRL
NKNIAFDINDIPFSEFDDLINQYKNEILNN IILNLRYKDNNLIDLSGYGAKVEVYDGVEL
NDKNQFKLTSSANSKIRVTQNQNIIFNSVF LDFSVSFWIRIPKYKNDGIQNYIHNEYTII
NCMKNNSGWKISIRGNRIIWTLIDINGKTK SVFFEYNIREDISEYINRWFFVTITNNLNN
AKIYINGKLESNTDIKDIREVIANGEIIFK LDGDIDRTQFIWMKYFSIFNTELSQSNIEE
RYKIQSYSEYLKDFWGNPLMYNKEYYMFNA GNKNSYIKLKKDSPVGEILTRSKYNQNSKY
INYRDLYIGEKFIIRRKSNSQSINDDIVRK EDYIYLDFFNLNQEWRVYTYKYFKKEEMKL
FLAPIYDSDEFYNTIQIKEYDEQPTYSCQL LFKKDEESTDEIGLIGIHRFYESGIVFEEY
KDYFCISKWYLKEVKRKPYNLKLGCNWQFI PKDEGWTE
SEQ ID NO: 67-Polypeptide Sequence of BoNT/XA(0)
MGSMKLEINKFNYNDPIDGINVITMRPPRH SDKINKGKGPFKAFQVIKNIWIVPERYNFT
NNTNDLNIPSEPIMEADAIYNPNYLNTPSE KDEFLQGVIKVLERIKSKPEGEKLLELISS
SIPLPLVSNGALTLSDNETIAYQENNNIVS NLQANLVIYGPGPDIANNATYGLYSTPISN
GEGTLSEVSFSPFYLKPFDESYGNYRSLVN IVNKFVKREFAPDPASTLMHQLVYVTHNLY
GISNRNFYYNFDTGKIETSRQQNSLIFEEL LTFGGIDSKAISSLIIKKIIETAKNNYTTL
ISERLNTVTVENDLLKYIKNKIPVQGRLGN FKLDTAEFEKKLNTILFVLNESNLAQRESI
LVRKHYLKERPIDPIYVNILDDNSYSTLEG FNISSQGSNDFQGQLLESSYFEKIESNALR
AFIKICHKAIDGRSLEVLFQGPLYNKTLDC IEVENKDLFLISNKDSLNDINLSEEKIKPE
TTVFFKDKLPPQDITLSNYDFTEANSIPSI SQQNILERNEELYEPIRNSLFEIKTIYVDK
LTTFHFLEAQNIDESIDSSKIRVELTDSVD EALSNPNKVYSPFKNMSNTINSIETGITST
YIFYQWLRSIVKDFSDETGKIDVIDKSSDT LAIVPYIGPLLNIGNDIRHGDFVGAIELAG
ITALLEYVPEFTIPILVGLEVIGGELAREQ VEAIVNNALDKRDQKWAEVYNITKAQWWGT
IHLQINTRLAHTYKALSRQANAIKMNMEFQ LANYKGNIDDKAKIKNAISETEILLNKSVE
QAMKNTEKFMIKLSNSYLTKEMIPKVQDNL KNFDLETKKTLDKFIKEKEDILGTNLSSSL
RRKVSIRLNKNIAFDINDIPFSEFDDLINQ YKNEIEDYEVLNLGAEDGKIKDLSGTTSDI
NIGSDIEIINTSILNLRYESNHLIDLSRYA SKINIGSKVNFDPIDKNQIQLENLESSKIE
VILKNAIVYNSMYENFSTSFWIRIPKYENS ISLNNEYTIINCMENNSGWKVSLNYGEIIW
TLQDTQEIKQRVVFKYSQMINISDYINRWI FVTITNNRLNNSKIYINGRLIDQKPISNLG
NIHASNNIMFKLDGCRDTHRYIWIKYFNLF DKELNEKEIKDLYDNQSNSGILKDFWGDYL
QYDKPYYMLNLYDPNKYVDVNNVGIRGYMY LKGPRGSVMTTNIYLNSSLYRGTKFIIKKY
ASGNKDNIVRNNDRVYINVVVKNKEYRLAT NASQAGVEKILSALEIPDVGNLSQVVVMKS
KNDQGITNKCKMNLQDNNGNDIGFIGFHQF NNIAKLVASNWYNRQIERSSRTLGCSWEFI
PVDDGWGERPL
SEQ ID NO: 68-Polypeptide Sequence of BoNT/XA(0) Variant
MGSMKLEINKFNYNDPIDGINVITMRPPRH SDKINKGKGPFKAFQVIKNIWIVPERYNFT
NNTNDLNIPSEPIMEADAIYNPNYLNTPSE KDEFLQGVIKVLERIKSKPEGEKLLELISS
SIPLPLVSNGALTLSDNETIAYQENNNIVS NLQANLVIYGPGPDIANNATYGLYSTPISN
GEGTLSEVSFSPFYLKPFDESYGNYRSLVN IVNKFVKREFAPDPASTLMHQLVYVTHNLY
GISNRNFYYNFDTGKIETSRQQNSLIFEEL LTFGGIDSKAISSLIIKKIIETAKNNYTTL
ISERLNTVTVENDLLKYIKNKIPVQGRLGN FKLDTAEFEKKLNTILFVLNESNLAQRFSI
LVRKHYLKERPIDPIYVNILDDNSYSTLEG FNISSQGSNDFQGQLLESSYFEKIESNALR
AFIKICHKAIDGRSLEVLFQGPLYNKTLDC IEVENKDLFLISNKDSLNDINLSEEKIKPE
TTVFFKDKLPPQDITLSNYDFTEANSIPSI SQQNILERNEELYEPIRNSLFEIKTIYVDK
LTTFHFLEAQNIDESIDSSKIRVELTDSVD EALSNPNKVYSPFKNMSNTINSIETGITST
YIFYQWLRSIVKDFSDETGKIDVIDKSSDT LAIVPYIGPLLNIGNDIRHGDFVGAIELAG
ITALLEYVPEFTIPILVGLEVIGGELAREQ VEAIVNNALDKRDQKWAEVYNITKAQWWGT
IHLQINTRLAHTYKALSRQANAIKMNMEFQ LANYKGNIDDKAKIKNAISETEILINKSVE
QAMKNTEKFMIKLSNSYLTKEMIPKVQDNL KNFDLETKKTLDKFIKEKEDILGTNLSSSL
RRKVSIRLNKNIAFDINDIPESEFDDLINQ YKNEIINTSILNLRYESNHLIDLSRYASKI
NIGSKVNFDPIDKNQIQLENLESSKIEVIL KNAIVYNSMYENFSTSFWIRIPKYENSISL
NNEYTIINCMENNSGWKVSLNYGEIIWTLQ DTQEIKQRVVFKYSQMINISDYINRWIFVT
ITNNRLNNSKIYINGRLIDQKPISNLGNIH ASNNIMFKLDGCRDTHRYIWIKYENLEDKE
LNEKEIKDLYDNQSNSGILKDFWGDYLQYD KPYYMLNLYDPNKYVDVNNVGIRGYMYLKG
PRGSVMTTNIYLNSSLYRGTKFIIKKYASG NKDNIVRNNDRVYINVVVKNKEYRLATNAS
QAGVEKILSALEIPDVGNLSQVVVMKSKND QGITNKCKMNLQDNNGNDIGFIGFHQFNNI
AKLVASNWYNRQIERSSRTLGCSWEFIPVD DGWGERPL
SEQ ID NO: 69-Polypeptide Sequence of BoNT/XD(0)
MGSMKLEINKFNYNDPIDGINVITMRPPRH SDKINKGKGPFKAFQVIKNIWIVPERYNFT
NNTNDLNIPSEPIMEADAIYNPNYLNTPSE KDEFLQGVIKVLERIKSKPEGEKLLELISS
SIPLPLVSNGALTLSDNETIAYQENNNIVS NLQANLVIYGPGPDIANNATYGLYSTPISN
GEGTLSEVSFSPFYLKPFDESYGNYRSLVN IVNKFVKREFAPDPASTLMHQLVYVTHNLY
GISNRNFYYNFDTGKIETSRQQNSLIFEEL LTFGGIDSKAISSLIIKKIIETAKNNYTTL
ISERLNTVTVENDLLKYIKNKIPVQGRLGN FKLDTAEFEKKLNTILFVLNESNLAQRFSI
LVRKHYLKERPIDPIYVNILDDNSYSTLEG FNISSQGSNDFQGQLLESSYFEKIESNALR
AFIKICHKAIDGRSLYNKTLDCIEVENKDL FLISNKDSLNDINLSEEKIKPETTVFFKDK
LPPQDITLSNYDFTEANSIPSISQQNILER NEELYEPIRNSLFEIKTIYVDKLTTFHFLE
AQNIDESIDSSKIRVELTDSVDEALSNPNK VYSPFKNMSNTINSIETGITSTYIFYQWLR
SIVKDFSDETGKIDVIDKSSDTLAIVPYIG PLLNIGNDIRHGDFVGAIELAGITALLEYV
PEFTIPILVGLEVIGGELAREQVEAIVNNA LDKRDQKWAEVYNITKAQWWGTIHLQINTR
LAHTYKALSRQANAIKMNMEFQLANYKGNI DDKAKIKNAISETEILLNKSVEQAMKNTEK
FMIKLSNSYLTKEMIPKVQDNLKNEDLETK KTLDKFIKEKEDILGTNLSSSLRRKVSIRL
NKNIAFDINDIPFSEFDDLINQYKNEIEDY EVLNLGAEDGKIKDLSGTTSDINIGSDIEN
DSKILSLQNKKNALVDTSGYNAEVRVGDNV QLNTIYTNDFKLSSSGDKIIVNLNNNILYS
AIYENSSVSFWIKISKDLTNSHNEYTIINS IEQNSGWKLCIRNGNIEWILQDVNRKYKSL
IFDYSESLSHTGYTNKWFFVTITNNIMGYM KLYINGELKQSQKIEDLDEVKLDKTIVFGI
DENIDENQMLWIRDFNIFSKELSNEDINIV YEGQILRNVIKDYWGNPLKEDTEYYIINDN
YIDRYIAPESNVLVLVQYPDRSKLYTGNPI TIKSVSDKNPYSRILNGDNIILHMLYNSRK
YMIIRDTDTIYATQGGECSQNCVYALKLQS NLGNYGIGIFSIKNIVSKNKYCSQIFSSFR
ENTMLLADIYKPWRFSFKNAYTPVAVTNYE TKLLSTSSFWKFISRDPGWVE
SEQ ID NO: 70-Polypeptide Sequence of BoNT/XF(0)
MGSMKLEINKFNYNDPIDGINVITMRPPRH SDKINKGKGPFKAFQVIKNIWIVPERYNFT
NNTNDLNIPSEPIMEADAIYNPNYLNTPSE KDEFLQGVIKVLERIKSKPEGEKLLELISS
SIPLPLVSNGALTLSDNETIAYQENNNIVS NLQANLVIYGPGPDIANNATYGLYSTPISN
GEGTLSEVSFSPFYLKPFDESYGNYRSLVN IVNKFVKREFAPDPASTLMHQLVYVTHNLY
GISNRNFYYNFDTGKIETSRQQNSLIFEEL LTFGGIDSKAISSLIIKKIIETAKNNYTTL
ISERLNTVTVENDLLKYIKNKIPVQGRLGN FKLDTAEFEKKLNTILFVLNESNLAQRFSI
LVRKHYLKERPIDPIYVNILDDNSYSTLEG FNISSQGSNDFQGQLLESSYFEKIESNALR
AFIKICHKAIDGRSLYNKTLDCIEVENKDL FLISNKDSLNDINLSEEKIKPETTVFFKDK
LPPQDITLSNYDFTEANSIPSISQQNILER NEELYEPIRNSLFEIKTIYVDKLTTFHFLE
AQNIDESIDSSKIRVELTDSVDEALSNPNK VYSPFKNMSNTINSIETGITSTYIFYQWLR
SIVKDESDETGKIDVIDKSSDTLAIVPYIG PLLNIGNDIRHGDFVGAIELAGITALLEYV
PEFTIPILVGLEVIGGELAREQVEAIVNNA LDKRDQKWAEVYNITKAQWWGTIHLQINTR
LAHTYKALSRQANAIKMNMEFQLANYKGNI DDKAKIKNAISETEILLNKSVEQAMKNTEK
FMIKLSNSYLTKEMIPKVQDNLKNEDLETK KTLDKFIKEKEDILGTNLSSSLRRKVSIRL
NKNIAFDINDIPFSEFDDLINQYKNEIEDY EVLNLGAEDGKIKDLSGTTSDINIGSDIEI
KDNSILDMRYENNKFIDISGYGSNISINGD VYIYSTNRNQFGIYSSKPSEVNIAQNNDII
YNGRYQNFSISFWVRIPKYFNKVNLNNEYT IIDCIRNNNSGWKISLNYNKIIWTLQDTAG
NNQKLVFNYTQMISISDYINKWIFVTITNN RLGNSRIYINGNLIDEKSISNLGDIHVSDN
ILFKIVGCNDTRYVGIRYFKVEDTELGKTE IETLYSDEPDPSILKDFWGNYLLYNKRYYL
LNLLRTDKSITQNSNFLNINQQRGVYQKPN IFSNTRLYTGVEVIIRKNGSTDISNTDNFV
RKNDLAYINVVDRDVEYRLYADISIAKPEK IIKLIRTSNSNNSLGQIIVMDSIGNNCTMN
FQNNNGGNIGLLGFKSNNLVASSWYYNNIR KNTSSNGCFWSFISKEHGWQEN
SEQ ID NO: 71-C1 Activation Loop Consensus Sequence
Cys-(Xaa)ₐ-Ile-Asp/Glu-Gly-Arg-(Yaa)_{b}-Cys, wherein a = 1-10 and b = 4-15
SEQ ID NO: 72-C1 Activation Loop
CHKAIDGRSLYNKTLDC
SEQ ID NO: 73-C1 Activation Loop Variant
CHKAIEGRSLYNKTLDC
SEQ ID NO: 74-Polypeptide Sequence of rLC/A(0) (His-tagged)
MPFVNKQFNY KDPVNGVDIA YIKIPNAGQM QPVKAFKIHN KIWVIPERDT FTNPEEGDLN
PPPEAKQVPV SYYDSTYLST DNEKDNYLKG VTKLFERIYS TDLGRMLLTS IVRGIPFWGG
STIDTELKVI DTNCINVIQP DGSYRSEELN LVIIGPSADI IQFECKSFGH EVLNLTRNGY
GSTQYIRFSP DFTFGFEESL EVDTNPLLGA GKFATDPAVT LAHQLIYAGH RLYGIAINPN
RVFKVNTNAY YEMSGLEVSF EELRTFGGHD AKFIDSLQEN EFRLYYYNKF KDIASTLNKA
KSIVGTTASL QYMKNVFKEK YLLSEDTSGK FSVDKLKFDK LYKMLTEIYT EDNFVKFFKV
LNRKTYLNED KAVFKINIVP KVNYTIYDGF NLRNTNLAAN ENGQNTEINN MNFTKLKNFT
GLFEENLYFQ GASHHHHHHH H
SEQ ID NO: 75-Polypeptide Sequence of rLHN/A(0) (His-tagged)
MPFVNKQFNY KDPVNGVDIA YIKIPNAGQM QPVKAFKIHN KIWVIPERDT FTNPEEGDLN
PPPEAKQVPV SYYDSTYLST DNEKDNYLKG VTKLFERIYS TDLGRMLLTS IVRGIPFWGG
STIDTELKVI DTNCINVIQP DGSYRSEELN LVIIGPSADI IQFECKSFGH EVLNLTRNGY
GSTQYIRFSP DFTFGFEESL EVDTNPLLGA GKFATDPAVT LAHQLIYAGH RLYGIAINPN
RVFKVNTNAY YEMSGLEVSF EELRTFGGHD AKFIDSLQEN EFRLYYYNKF KDIASTLNKA
KSIVGTTASL QYMKNVFKEK YLLSEDTSGK FSVDKLKFDK LYKMLTEIYT EDNFVKFFKV
LNRKTYLNFD KAVFKINIVP KVNYTIYDGF NLRNTNLAAN FNGQNTEINN MNFTKLKNFT
GLFEFYKLLC VRGIITSKTK SLDKGYNKAL NDLCIKVNNW DLFFSPSEDN FTNDLNKGEE
ITSDTNIEAA EENISLDLIQ QYYLTFNFDN EPENISIENL SSDIIGQLEL MPNIERFPNG
KKYELDKYTM FHYLRAQEFE HGKSRIALTN SVNEALLNPS RVYTFFSSDY VKKVNKATEA
AMFLGWVEQL VYDFTDETSE VSTTDKIADI TIIIPYIGPA LNIGNMLYKD DFVGALIFSG
AVILLEFIPE IAIPVLGTFA LVSYIANKVL TVQTIDNALS KRNEKWDEVY KYIVTNWLAK
VNTQIDLIRK KMKEALENQA EATKAIINYQ YNQYTEEEKN NINFNIDDLS SKLNESINKA
MININKFLNQ CSVSYLMNSM IPYGVKRLED FDASLKDALL KYIYDNRGTL IGQVDRLKDK
VNNTLSTDIP FQLSKYVDNQ RLLSTENLYF QGASHHHHHH HH
SEQ ID NO: 76-Polypeptide Sequence of rLC/X(0)
MKLEINKENY NDPIDGINVI TMRPPRHSDK INKGKGPFKA FQVIKNIWIV PERYNFTNNT
NDLNIPSEPI MEADAIYNPN YLNTPSEKDE FLQGVIKVLE RIKSKPEGEK LLELISSSIP
LPLVSNGALT LSDNETIAYQ ENNNIVSNLQ ANLVIYGPGP DIANNATYGL YSTPISNGEG
TLSEVSFSPF YLKPFDESYG NYRSLVNIVN KFVKREFAPD PASTLMHQLV YVTHNLYGIS
NRNFYYNEDT GKIETSRQQN SLIFEELLTF GGIDSKAISS LIIKKIIETA KNNYTTLISE
RLNTVTVEND LLKYIKNKIP VQGRLGNFKL DTAEFEKKLN TILFVLNESN LAQRFSILVR
KHYLKERPID PIYVNILDDN SYSTLEGENI SSQGSNDFQG QLLESSYFEK IESNALRAFI
KIAPRNGLLY NAIYRNSK
SEQ ID NO: 77-PreScission Protease Site
LEVLFQGP
SEQ ID NO: 78-C1 Activation Loop Variant 2
CHKAIDGRSLEVLFQGPLYNKTLDC

### EXAMPLES

### Example 1

### BoNT/a(0) is Catalytically Inactive In Vitro and In Vivo

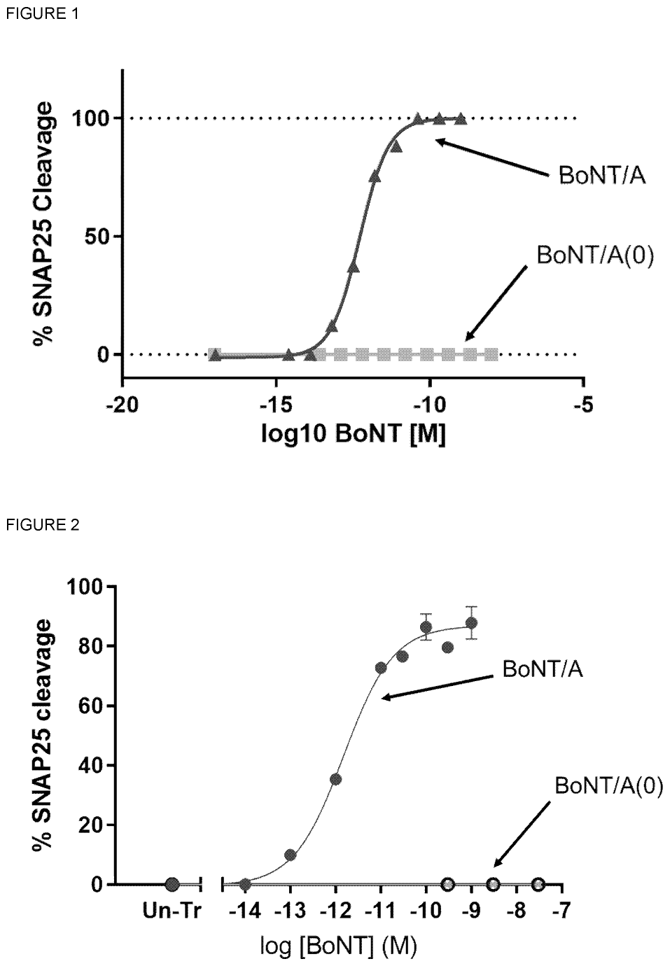

Catalytic activity of BoNT/A(0) (SEQ ID NO: 2) was tested in in vitro cell-based models, which measure cleavage of SNAP25, the BoNT/A target SNARE protein. FIG. 1 shows that, in contrast to wild-type BoNT/A (SEQ ID NO: 60), BoNT/A(0) does not cleave SNAP25 in a human neuronal assay. FIG. 2 confirms this result in a rat neuronal assay.

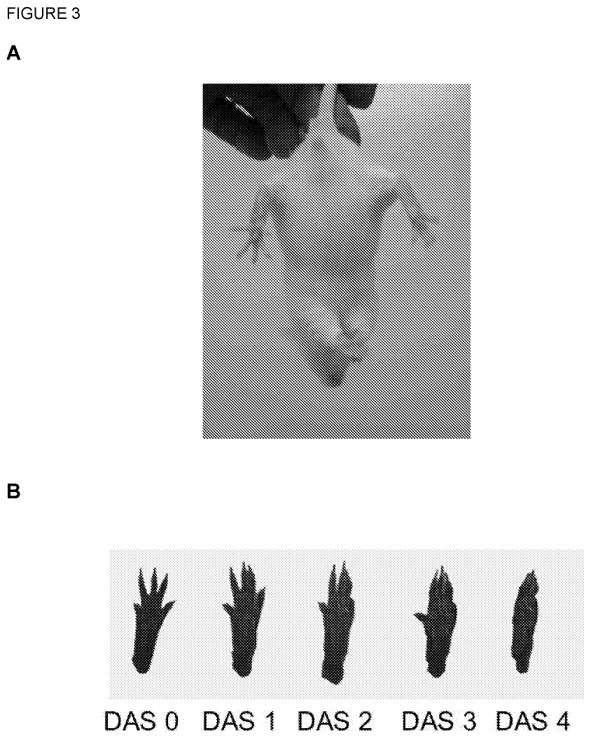

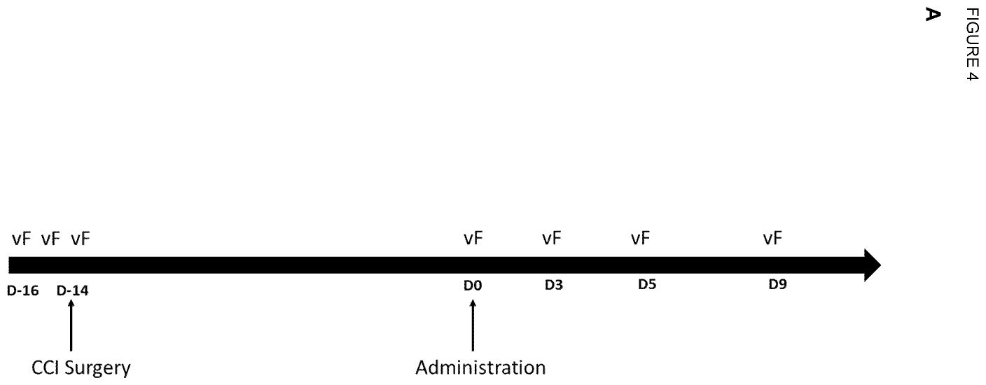

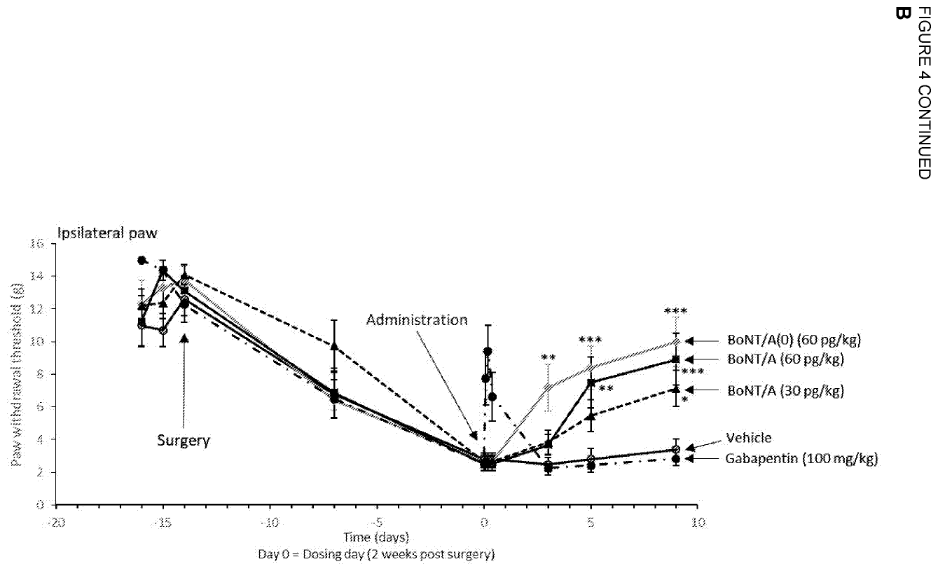

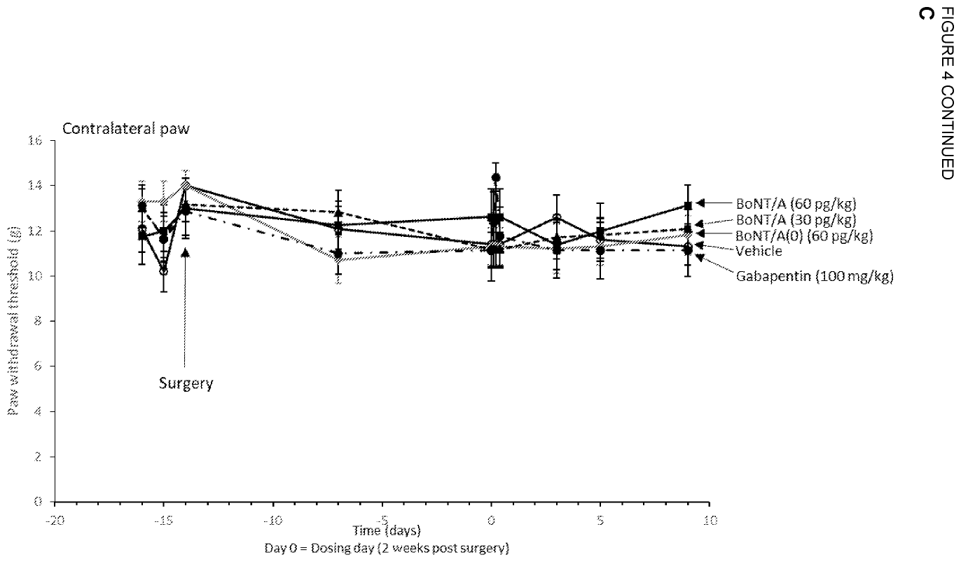

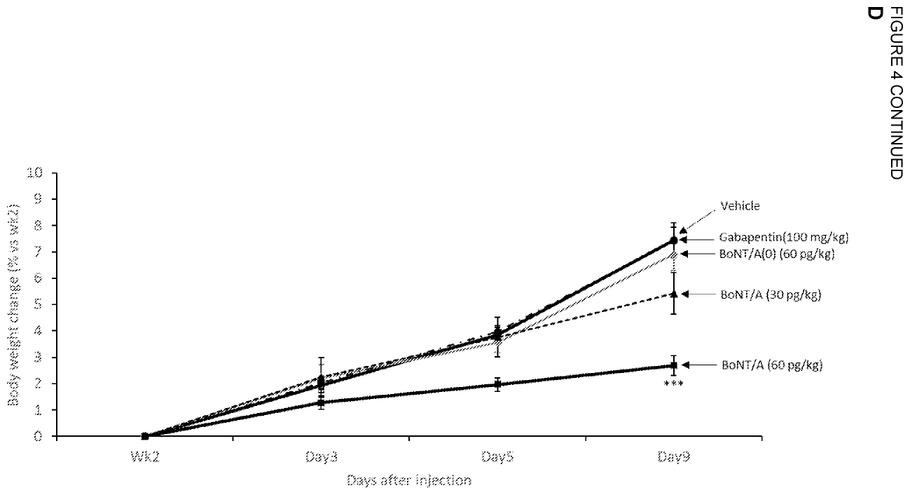

By way of confirmation, an in vivo DAS assay was carried out using BoNT/A and BoNT/A(0). The DAS assay was performed by injection of 20 μl of clostridial toxin, formulated in Gelatine Phosphate Buffer, into the mouse gastrocnemius/soleus complex, followed by assessment of Digital Abduction Score using the method of Aoki (Aoki KR, Toxicon 39: 1815-1820; 2001). In the DAS assay, mice were suspended briefly by the tail in order to elicit a characteristic startle response (FIG. 3A) in which the mouse extends its hind limbs and abducts its hind digits. Following clostridial toxin injection, the varying degrees of digit abduction were scored on a five-point scale (0=normal to 4=maximal reduction in digit abduction—FIG. 3B). This provides a functional measure of the paralysis induced by the neurotoxin's activity in the neuro-muscular junction. In addition, bodyweight change was assessed in the mice within seven days of administration. This provides a measure of toxicity and the undesired effects of toxin diffusion away from the site of administration. Results are presented in Table 1, below.

TABLE 1
DAS score (after 24 hours) and bodyweight change
following administration of BoNT/A(0) or BoNT/A.

| | DAS Score | Bodyweight Change Observed |
|---|---|---|
| BoNT/A | 4 | Yes |
| BoNT/A(0) | 0 | No |

The results confirm that BoNT/A(0) is catalytically inactive in vivo and does not result in any symptoms of toxicity. Thus, BoNT/A(0) is a safe, substantially non-toxic therapeutic.

### Example 2

### Treatment of Chronic Neuropathic Pain (Chronic Constriction Injury (CCI) Rat Model) Using Catalytically Inactive BoNT

### Materials & Methods

The chronic constriction injury (CCI) was performed as previously described by Bennett and Xie (1988), Pain, 33(1):87-107. On day −14, adult, male Sprague-Dawley rats (220-250 g) were anesthetized before a segment of the left sciatic nerve was exposed and four loose ligations of the silk suture were placed on the nerve. On day 0 (DO) the rats were injected with either BoNT/A (30 pg/kg), BoNT/A (60 pg/kg), BoNT/A(0) (60 pg/kg) or vehicle (GPB) administered via intraplantar (i.pl.) route (n=10-11/group), whereas a positive control, gabapentin (100 mg/kg) was administered via oral (p.o.) route (n=8/group). Animals treated with gabapentin were tested 1, 2 and 4 h after treatment. BoNT/A, BoNT/A(0) or vehicle-treated animals were tested on days 3, 5, and 9. Animals were evaluated for mechanical sensitivity in the von Frey test.

### Results

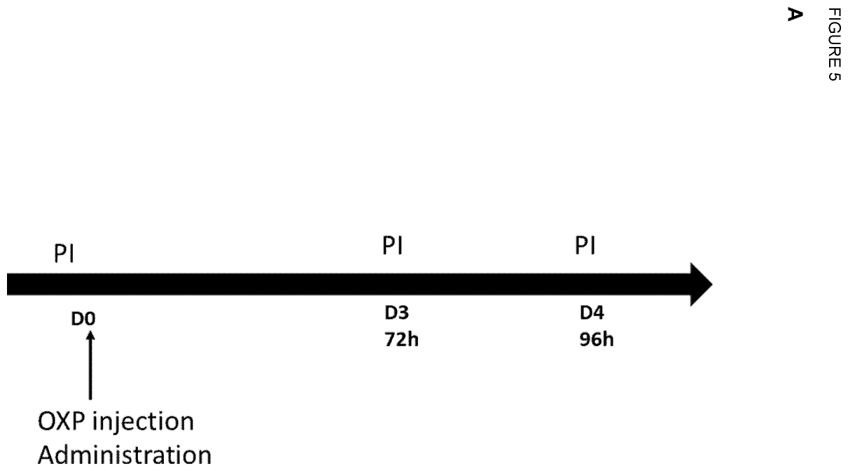

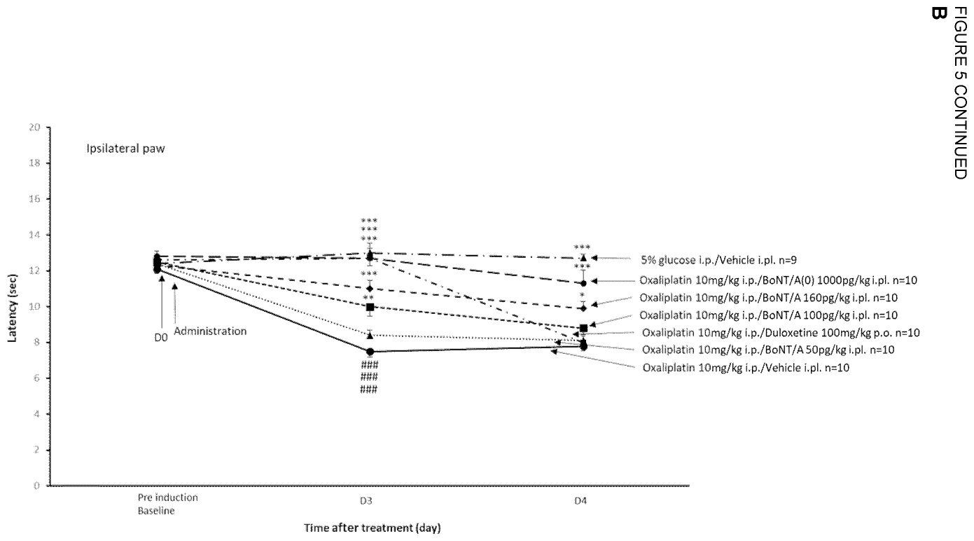

The experiments showed that by administering catalytically inactive BoNT (BoNT/A(0)), mechanical sensitivity in the ipsilateral paw was reduced (FIG. 4B). Moreover, BoNT/A(0) was more effective at reducing mechanical sensitivity when compared to equivalently dosed BoNT/A. At later time points, BoNT/A(0) was also more effective than gabapentin. To confirm that the reduced sensitivity was as a result of BoNT/A(0) administration, mechanical sensitivity of the contralateral paw was also tested. Results showed that differences in mechanical sensitivity of the contralateral paw among conditions were non-significant (FIG. 4C). Furthermore, as confirmation of the substantial non-toxicity of BoNT/A(0), bodyweight change over time was equivalent to that of rats administered vehicle or gabapentin (FIG. 4D). This is in contrast to the change observed in rats administered catalytically active BoNT/A, which was statistically significantly different at day 9.

In conclusion, catalytically inactive clostridial neurotoxins are surprisingly capable of reducing pain (e.g. chronic neuropathic pain), thereby suggesting that such neurotoxins are suitable pain therapeutics.

### Example 3

### Treatment of Acute Neuropathic Pain (Oxaliplatin Rat Model) Using Catalytically Inactive BoNT

### Materials & Methods

The experimental model of oxaliplatin-induced peripheral sensory neuropathy was induced by intraperitoneal injection of oxaliplatin (Ling et al (2007), Pain, 128(3):225-234; Ling et al (2007), Toxicology, 20; 234(3):176-84). On day 0, adult, male Sprague-Dawley rats (100-133 g) received an i.p. injection of a sham-treatment (5% glucose) or oxaliplatin (10 mg/kg). Immediately after, sham-treated animals received an i.pl. injection of vehicle, whereas oxaliplatin-treated animals received an i.pl. injection of BoNT/A(0) (1000 pg/kg), BoNT/A (50 pg/kg), BoNT/A (100 pg/kg), BoNT/A (160 pg/kg) or vehicle (GPB; n=10/group). On D3 a positive control, duloxetine (100 mg/kg) was given via p.o. route. Animals were evaluated for thermal (cold) sensitivity on D3 and D5.

### Results

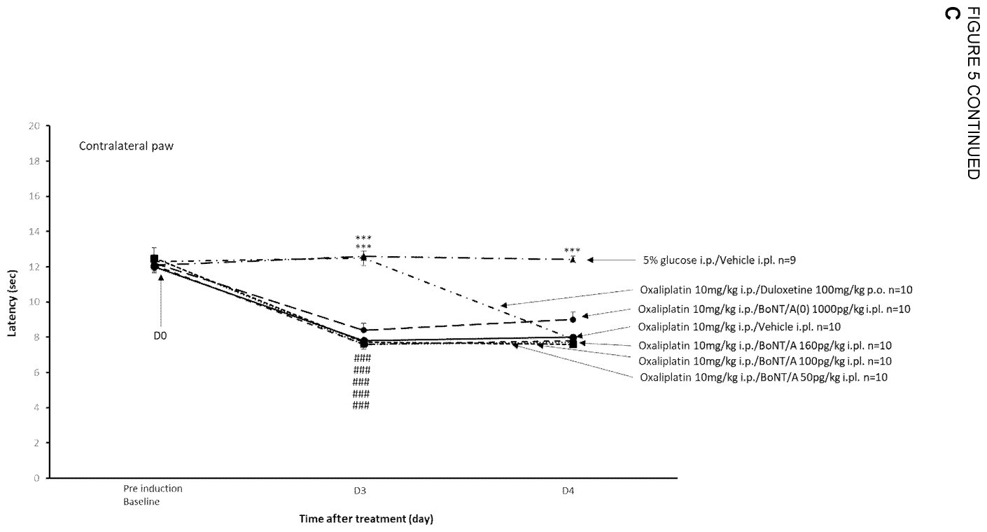

The experiments showed that by administering catalytically inactive BoNT (BoNT/A(0)) cold sensitivity in the ipsilateral paw was reduced (FIG. 5B). There was no difference in thermal sensitivity of the contralateral paw across the groups treated with BoNT/A, BoNT/A(0) or vehicle (FIG. 5C).

In conclusion, catalytically inactive clostridial neurotoxins are surprisingly capable of reducing acute neuropathic pain, thereby suggesting that such neurotoxins have generic application for the treatment of pain.

### Example 4

### Treatment of Chronic Neuropathic Pain (Oxaliplatin Rat Model) Using Catalytically Inactive BoNT

### Materials & Methods

On day −2 (D−2) adult, male Sprague-Dawley rats (180-210 g) received i.p. injection of oxaliplatin (10 mg/kg), before being treated with BoNT/A (100 pg/kg), BoNT/A(0) (100 pg/kg) or vehicle (GPB) via i.pl. route on day 0 (n=11-12/group). A positive control, pregabalin was administered on D3 (n=12). Animals were tested for mechanical sensitivity (von Frey test) and thermal (cold) sensitivity (cold plate test) on days 3, 6 and 9.

### Results

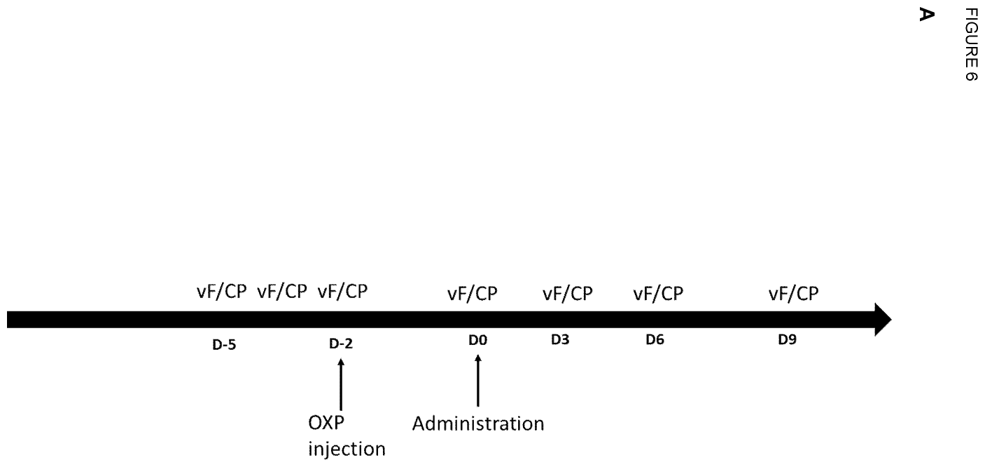

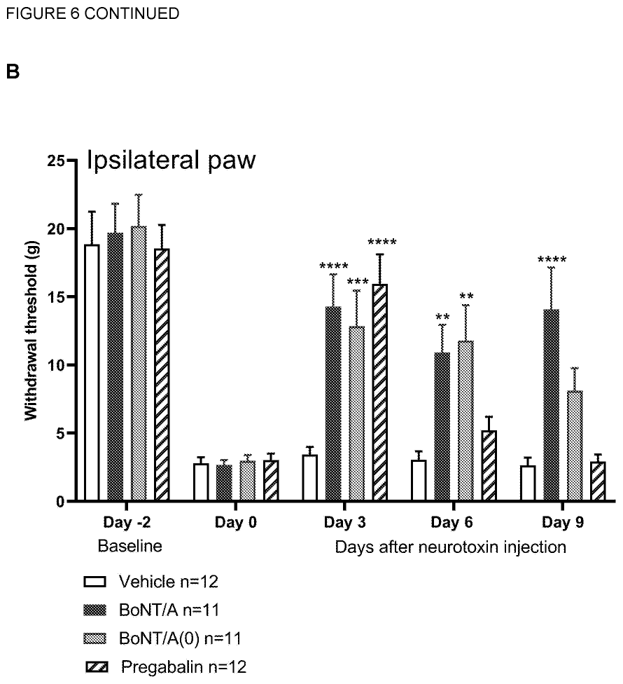

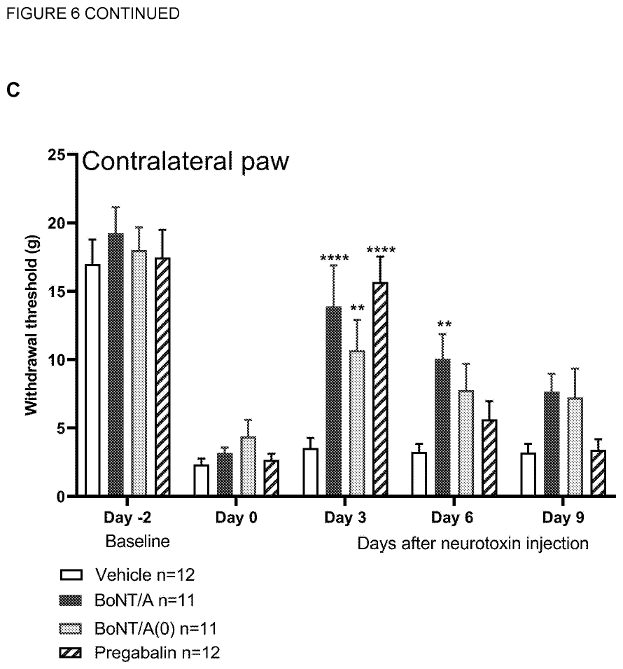

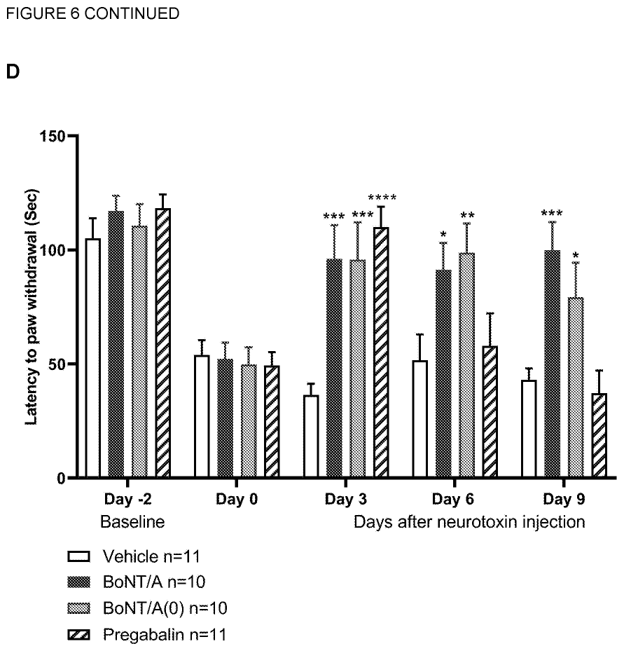

The experiments showed that by administering catalytically inactive BoNT (BoNT/A(0)) mechanical sensitivity in the ipsilateral paw (FIG. 6B) and cold sensitivity (FIG. 6D) was reduced.

In conclusion, catalytically inactive clostridial neurotoxins are surprisingly capable of reducing chronic neuropathic pain in a different chemotherapy-induced pain model.

### Example 5

### Treatment of Inflammatory Pain (UV-B Burn Rat Model) Using Catalytically Inactive BoNT

### Materials & Methods

In humans, as well as in a rodent model ultraviolet (UV)-B radiation results in both mechanical and thermal hyperalgesia. Adult, male Wistar rats (180-210 g) were administered BoNT/A (100 pg/kg), BoNT/A(0) (100 pg/kg) or vehicle (GPB; n=12/group) by i.pl. injection. 24 hours later, the plantar surface of the ipsilateral paw was exposed to ultraviolet-B (UVB) irradiation for approximately 5 min, receiving a dose of 500 mJ/cm². 48 hours after UVB and 72 hours after BoNT/A, BoNT/A(0) or vehicle injection, animals were tested for mechanical sensitivity in the von Frey test. An additional group of UVB-exposed animals were injected with a positive control, indomethacin 48 hours later and tested in the von Frey test 1 hour after the injection (n=12/group).

### Results

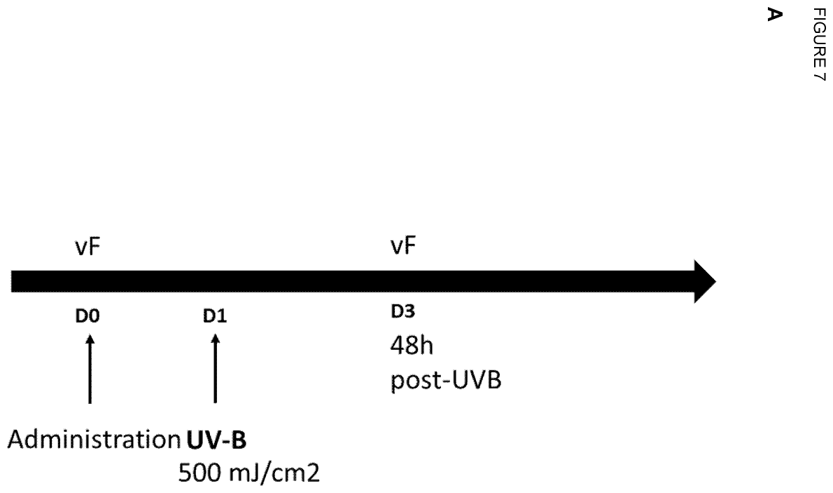

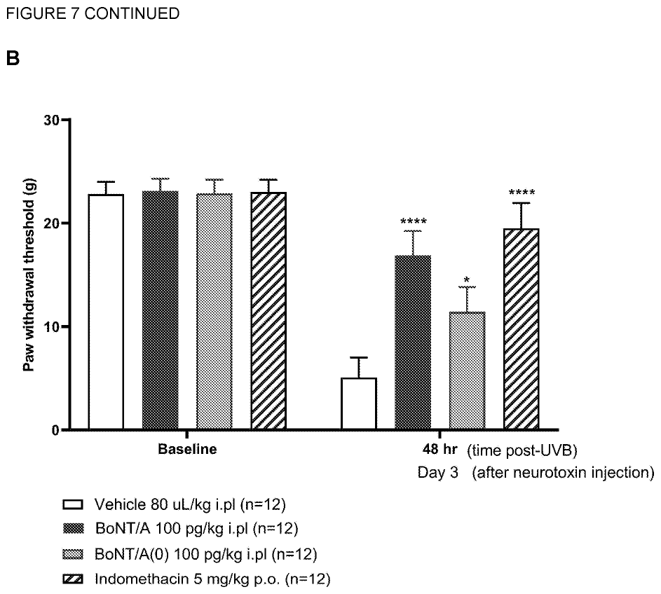

The experiments showed that by administering catalytically inactive BoNT (BoNT/A(0)), mechanical sensitivity was reduced (FIG. 7B). In conclusion, catalytically inactive clostridial neurotoxins are surprisingly capable of reducing inflammatory pain (e.g. acute inflammatory pain), thereby confirming that such neurotoxins have generic application for the treatment of pain.

The surprising finding that a catalytically inactive clostridial neurotoxin reduced inflammatory pain indicated that it finds utility in treating the underlying inflammatory conditions (e.g. including treating at least one symptom of the inflammatory condition, i.e. associated pain). Thus, it was considered credible that catalytically inactive clostridial neurotoxin can be used to treat inflammatory conditions.

### Example 6

### Treatment of Inflammatory Pain (CFA-Induced Inflammatory Pain Model) Using a Catalytically Inactive Chimeric BoNT

### Materials & Methods

Prior to BoNT or vehicle dosing, paw withdrawal threshold (PWT, g) of 70 adult male C57/BL6 mice (22-26 g) was assessed on 3 consecutive days using graduated von Frey filaments of increasing force. The mean of the last 2 days was considered as the baseline. On Day 0, under gas anesthesia, BoNT/XB (0.3 and 30 ng/kg), BoNT/XB(0) (0.3 and 30 ng/kg), BoNT/A (160 pg/kg) or vehicle (840 μl/kg) were injected into the intraplantar footpad of the left hindpaw (n=10/group). On day 2, prior to CFA injection, PWT was reassessed. Then under isoflurane anesthesia, a fixed 20 μL volume of CFA (1.5 mg/mL) was injected into the same hindpaw. On day 3 (day 1 post CFA), 1 hour prior to PWT assessment, animals allocated to the indomethacin group were orally dosed with indomethacin (10 mg/kg, n=9).

### Results

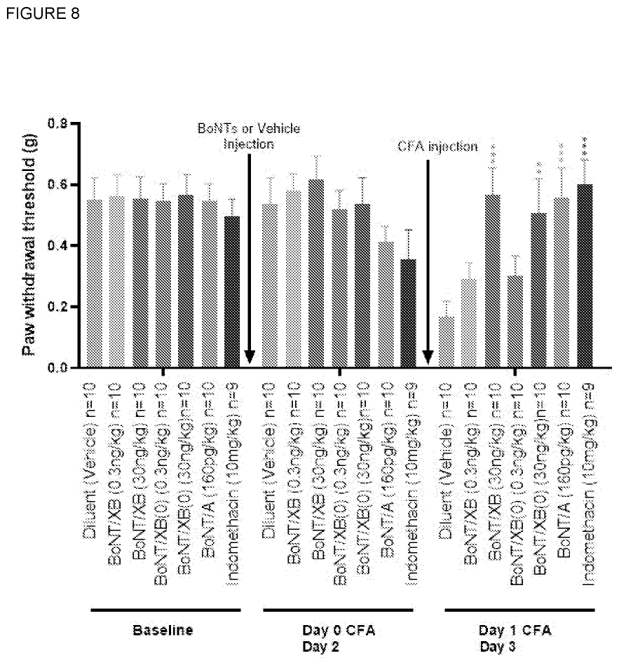

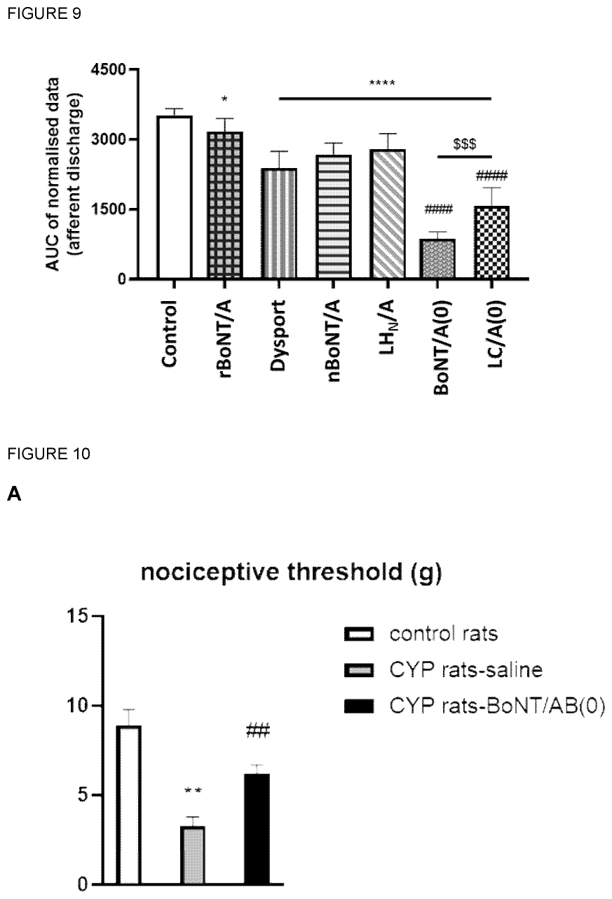

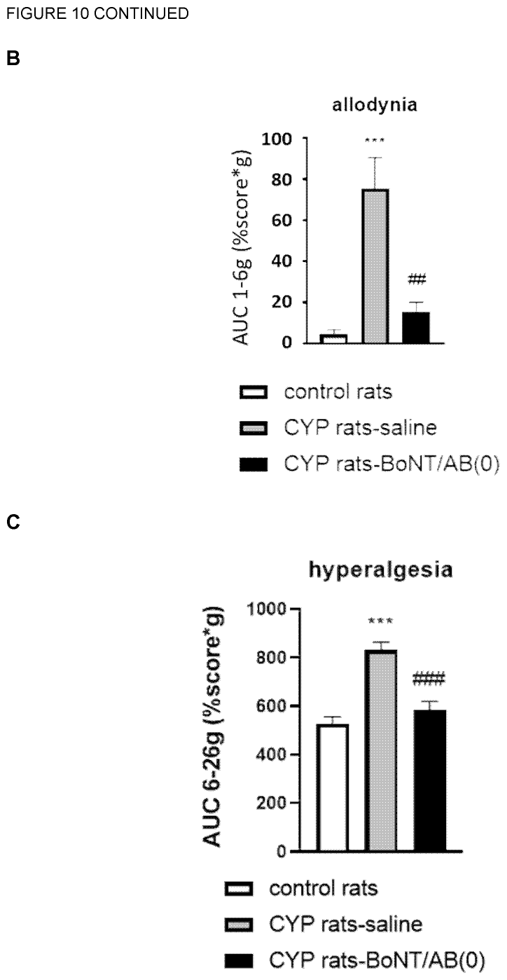

The experiments showed that a catalytically inactive chimeric BoNT (BoNT/XB(0)) comprising a catalytically inactive BoNT/X L-chain and translocation domain (BoNT/X LH_{N}) and a BoNT/B receptor binding domain BoNT/X (H_{C} domain) was effective at treating inflammatory pain. In more detail, FIG. 8 shows that mechanical sensitivity following CFA-induction of inflammatory pain was reduced in mice administered catalytically active BoNT/XB and catalytically inactive BoNT/XB(0) at a dose of 30 ng/kg. The reduction in sensitivity was equivalent to that of BoNT/A or positive control indomethacin.

The surprising finding that BoNT/XB(0) reduced inflammatory pain indicated that it finds utility in treating the underlying inflammatory conditions (e.g. including treating at least one symptom of the inflammatory condition, i.e. associated pain). Thus, it was considered further evidence of the credibility of catalytically inactive clostridial neurotoxin for use in treating inflammatory conditions.

### Example 7

### Treatment of Atopic Dermatitis Using a Catalytically Inactive Chimeric BoNT

Vehicle or BoNT/XB(0) (40 μg/mouse, 100 μg/mouse or 400 μg/mouse) is administered subcutaneously on the medial part of the back of adult C57/BL6 mice one day prior to exposure to Calcipotriol. Mice are then treated with calcipotriol on 5 consecutive days. At the end of the study, animals are euthanized, the skin of the back is collected, fixed and treated for histological analysis. After Hematoxylin and eosin staining, the epidermal thickness is evaluated. Immunolabelling is performed to evidence CD45+ cells.

The experiment shows that a catalytically inactive chimeric BoNT (BoNT/XB(0)) comprising a catalytically inactive BoNT/X L-chain and translocation domain (BoNT/X LH_{N}) and a BoNT/B receptor binding domain (H_{C} domain) is effective at treating atopic dermatitis, a model inflammatory condition. The results show an improvement in dermal thickness following administration of BoNT/XB(0). Dermal thickness is indicative of fibrosis, an inflammatory response to calcipotriol and is shown to be statistically-significant reduced in the BoNT/XB(0) treated animals. Moreover, the anti-inflammatory effect of BoNT/XB(0) is confirmed by a reduction in the number of CD45-positive cells in the BoNT/XB(0) treated animals (CD45 transduces activation signals in inflammatory cells).

Thus, it is concluded that, BoNT/XB(0) has anti-inflammatory properties, thus finding utility in treating inflammatory disorders.

### Example 8

Treatment of Bladder Pain & Lower Urinary Tract Disorders in a Mouse ex vivo Model Materials & Methods (Mouse Bladder Nerve Assay)

After humanely culling mice, the whole pelvic region of the mouse was dissected and placed in an organ bath continually perfused with buffer solution, gassed with carbogen and kept at 35° C. Ureters were tied with suture and the urethra and the dome of the bladder were catheterised and tied with suture. The urethra catheter was attached to an infusion pump to allow filling of the bladder, while the tap attached to the dome catheter was used to allow evacuation of fluid. The pelvic and hypogastric nerve fibres emerging from the bladder were dissected, cut and inserted into a suction glass electrode to allow recording of the nerve activity (afference discharge) in resting and stimulated (bladder filling to induce distension) conditions. Intravesical pressure was slowly increased from 0 to 50 mm of mercury (Hg) and corresponding nerve activity recorded. A pressure-response curve was plotted for control (PBS—phosphate buffered saline) and every polypeptide (recombinant BoNT/A (rBoNT/A); Dysport (holotoxin); non-complexed BoNT/A isolated from clostridia [nBoNT/A], catalytically-active BoNT/A L-chain and translocation domain without the H_{C} domain [LH_{N}/A], catalytically inactive BoNT/A [BoNT/A(0)], or catalytically inactive BoNT/A L-chain [LC/A(0)]) at 3 pM, and area under the curve (AUC) calculated. Four to 9 bladders were tested for each condition. AUCs were statistically compared using a one way ANOVA followed by a Tukey's multiple comparison test.

### Results

Compared to control (PBS), catalytically active BoNT/As (recombinant, natural, complexed) and LH_{N}/A were able to significantly inhibit the activity of the afferent nerves. The catalytically inactive forms of BoNT/A (BoNT/A(0)) and its light-chain (LC/A(0)) showed an even greater effect in inhibiting stretch-induced firing of bladder afferent nerves, compared to the catalytically active molecules tested. Moreover, full-length BoNT/A(0) was significantly more effective than light-chain alone.

The mouse bladder afferent nerve assay allows for the measure of afferent nerve firing and intravesical pressure during bladder distension. The bladder afferent nerve conveys bladder sensations, including pain, and is stimulated by increased pressure within the bladder. A 50 mm of mercury (Hg) employed corresponds to an extremely painful stimulus. These data show that LH_{N}/A, and to a much greater extent, catalytically inactive BoNT/A (BoNT/A(0)) and light-chain alone (LC/A(0)) find utility in treating bladder pain.

The afferent nerve is involved in other lower urinary tract disorders, such as overactive bladder (OAB), bladder pain syndrome, interstitial cystitis, and detrusor overactivity (e.g. neurogenic detrusor overactivity—NDO). The afferent nerve is associated with sensory symptoms of urgency experienced by subject's suffering from said disorders. Without wishing to be bound by theory, said disorders may be associated with afferent hypersensitivity, else an altered central threshold for afferent impulses. For example, the latter may explain the pathophysiology of OAB/NDO and may involve damage to central inhibitory pathways or the sensitization of afferent nerves, which may be particularly likely in NDO. In this case, neurotrophic factors such as nerve growth factor (NGF) or brain-derived neurotrophic factor (BDNF) levels may be increased and affect sensory neurons, which also result in the activation of the voiding reflex. Moreover, in the context of central pathway damage, functional magnetic resonance imaging (MRI) has shown that in OAB patients signalling in the insula, an area linked to the PAG (periacqueductal gray, one of the central structures involved in the control of micturition), may be altered, with incoherent firing thresholds, resulting in urgency. Thus, it is credible that decreasing bladder afferent nerve activity will treat each of these disorders.

### Example 9

Treatment of Bladder Pain & Lower Urinary Tract Disorders in a rat in vivo Model

### Materials & Methods

### Animal Model

A chronic rat model of interstitial cystitis (IC)/bladder pain syndrome (BPS) was developed based on the treatment of rats with cyclophosphamide (CYP). To induce chronic cystitis, at Day 0 (D0), D3 and D6, rats were weighed and an intraperitoneal (i.p.) injection of CYP at a dose of 40 mg/kg in a final volume of 5 mL/kg was performed. CYP was prepared fresh in saline at a final concentration of 8 mg/mL. Control rats received physiological saline under the same experimental conditions as CYP. No severe weight loss occurred. This model shows long-lasting visceral pain, characterized by both allodynia (painful response to a normally innocuous stimulus) and hyperalgesia (increased response to a noxious stimulus).

### Von Frey Assay

Visceral pain was assessed using the Von Frey assay (see Deuis J R, Dvorakova L S, Vetter I. Methods Used to Evaluate Pain Behaviors in Rodents. Front Mol Neurosci. 2017; 10:284. Published 2017 Sep. 6. doi:10.3389/fnmol.2017.00284 and/or Minett M S, Eijkelkamp N, Wood J N. Significant determinants of mouse pain behaviour [published correction appears in PLoS One. 2021 Jan. 15; 16(1):e0245813]. PLoS One. 2014; 9(8):e104458. Published 2014 Aug. 7. doi:10.1371/journal.pone.0104458). Standardized conditions including single-experimenter testing of all animals were applied to minimize variability behaviour-based pain testing. Visceral pain was evaluated in a blinded manner by applying to the lower abdomen, close to the urinary bladder, a set of 8 calibrated Von Frey filaments of increasing forces (1, 2, 4, 6, 8, 10, 15 and 26 g) with an interstimulus interval of 5 seconds. Prior to testing, the abdominal area designed for mechanical stimulation of each animal was shaved. Animals were placed on a raised wire mesh floor under individual transparent Plexiglas boxes and acclimatized for at least 30 minutes before starting the Von Frey test. Filaments were then applied for 1-2 seconds through the mesh floor with enough strength to cause the filament to slightly bend. Each filament was tested 3 times. Care was taken to stimulate different areas within the lower abdominal region in the vicinity of the urinary bladder to avoid desensitization.

Nociceptive behaviours were scored for each animal and each filament as shown below:

| Score | Behaviour |
|---|---|
| 0 | No response |
| 1 | Retraction of the abdomen |
| 2 | Trampling or change of position |
| 3 | Flinching or abdominal curvature or licking of the site stimulated |
| | with von Frey filaments |

### Visceral Pain

Definitions of the nociceptive parameters are provided below:

| Nociceptive parameter | Expression | Description |
|---|---|---|
| Nociceptive threshold | g | Von Frey force for which |
| | | the stimulus is perceived as |
| | | painful (score ≥1 is |
| | | obtained). |
| Nociceptive scores | % | % of the maximal response |
| | | for each filament (on the 3 |
| | | pooled applications*). |
| Area under the curve (AUC) | % scores × | Calculated from the curve |
| 1-6 g - allodynia - | g | of % nociceptive scores |
| | | plotted against painless (1 |
| | | to 6 g) von Frey forces |
| Area under the curve (AUC) | | Calculated from the curve |
| 6-26 g - hyperalgesia - | | of % nociceptive scores |
| | | plotted against painful (6 |
| | | to 26 g) von Frey forces |

*For example, at a given von Frey force, for an animal with a score of 1 at the first application, 1 at the second and 2 at the third, the summation of its scores is 4. The maximal pooled score being 9 (3 + 3 + 3), a pooled score of 4 equals 44% of the maximal response (100 × 4/9).

### Protocol Design

The following protocol was carried out to assess the effect of a catalytically inactive chimeric BoNT (BoNT/AB(0)) comprising a catalytically inactive BoNT/A L-chain and translocation domain (BoNT/A(0) LH_{N}) and a BoNT/B receptor binding domain (H_{C} domain) on a chronic rat model of CYP-induced IC/BPS.
At D−1, rats were acclimatized to the individual Plexiglas box (Von Frey set up) for a minimum of 30 minutes and to the application of the Von Frey filaments, in order to decrease the level of stress due to the new environment.
At D0, before CYP or saline first injection, Von Frey testing was performed in order to obtain basal values for nociceptive behaviour.
At D0, D3 and D6, chronic cystitis was induced to mimic IC/BPS.
At D7 (before beginning treatment), von Frey testing was performed to assess chronic cystitis induction.
At D8, treatment was performed by administration of BoNT/AB(0) or saline (control) to the bladder.
At D10 and D12, von Frey testing was performed to analyse the effects of test (BoNT/AB(0)) or saline on CYP-induced chronic visceral pain.

### Pharmacological Treatments

Prior to the experiments, animals were randomly assigned to treatment groups. Randomization was designed to have at least one animal of each group on each experimental day and to assign a different position in the von Frey chamber for animals of the same group.

### Results

The effects of CYP were characterized by a lower nociceptive threshold and increased nociceptive score associated to allodynia (increased AUC 1-6 g) and hyperalgesia (i.e. increased AUC 6-26 g). Treatment with BoNT/AB(0) (300 pg/rat) significantly reduced visceral pain, as seen in the increased nociceptive thresholds at D10 and D12, as well as the reduced allodynia and hyperalgesia AUCs at D10 and D12 versus saline-treated CYP animals, corresponding to 2 and 4 days after treatment.

These data show that a catalytically inactive chimeric BoNT (BoNT/AB(0)) comprising a catalytically inactive BoNT/A L-chain and translocation domain (BoNT/A(0) LH_{N}) and a BoNT/B receptor binding domain (H_{C} domain) was effective at treating bladder pain in an IC/BPS in vivo model. This is consistent with the role of BoNT/A(0) in reducing afferent nerve firing in the ex vivo mouse model of Example 8. Thus, it is concluded that catalytically inactive clostridial neurotoxins find utility in treating bladder pain, as well as any lower urinary tract disorder, e.g. in which pain and/or the afferent nerve are involved (e.g. overactive bladder (OAB), bladder pain syndrome, interstitial cystitis, and detrusor overactivity).

### Clauses

1. A polypeptide for use in treating pain, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.
2. A method for treating pain, the method comprising administering a polypeptide to a subject, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.
3. Use of a polypeptide in the manufacture of a medicament for treating pain, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.
4. The polypeptide for use according to clause 1, the method according to clause 2 or the use according to clause 3, wherein the polypeptide does not treat pain by any of the following: by promoting neuronal growth, by promoting neuronal repair, or by promoting neuronal growth and repair.
5. A polypeptide for use in treating an inflammatory disorder, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.
6. A method for treating an inflammatory disorder, the method comprising administering a polypeptide to a subject, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.
7. Use of a polypeptide in the manufacture of a medicament for treating an inflammatory disorder, wherein the polypeptide comprises a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.
8. The polypeptide for use according to clause 5, the method according to clause 6 or the use according to clause 7, wherein the polypeptide does not treat the inflammatory condition by any of the following: by promoting neuronal growth, by promoting neuronal repair, or by promoting neuronal growth and repair.
9. The polypeptide for use, the method or the use according to any one of the preceding clauses, wherein the polypeptide does not comprise a further catalytically active domain.
10. The polypeptide for use, method or use according to any one of the preceding clauses, wherein the polypeptide does not comprise a therapeutic or diagnostic agent (e.g. a covalently or non-covalently associated therapeutic or diagnostic agent) additional to the clostridial neurotoxin L-chain, H_{N} domain and/or H_{C} domain.
11. The polypeptide for use, method or use according to any one of the preceding clauses, wherein the polypeptide is not administered (e.g. sequentially or subsequently) with a further therapeutic or diagnostic agent.
12. The polypeptide for use, method or use according to any one of the preceding clauses, wherein the polypeptide comprises a catalytically inactive clostridial neurotoxin L-chain.
13. The polypeptide for use, method or use according to any one of the preceding clauses, wherein the polypeptide comprises a clostridial neurotoxin L-chain, H_{N} domain and an H_{C} domain, wherein the L-chain is catalytically inactive.
14. The polypeptide for use, method or use according to any one of clauses 1-12, wherein the polypeptide consists essentially of a clostridial neurotoxin light-chain (L-chain), a clostridial neurotoxin translocation domain (H_{N} domain) and/or a clostridial neurotoxin receptor binding domain (H_{C} domain), wherein when the polypeptide comprises or consists essentially of a clostridial neurotoxin L-chain, the L-chain is catalytically inactive.
15. The polypeptide for use, method or use according to any one of clauses 1-12 or 14, wherein the polypeptide consists essentially of a clostridial neurotoxin light-chain (L-chain) and a clostridial neurotoxin translocation domain (H_{N} domain), wherein the L-chain is catalytically inactive.
16. The polypeptide for use, method or use according to any one of the preceding clauses, wherein the polypeptide consists essentially of a clostridial neurotoxin light-chain (L-chain), a clostridial neurotoxin translocation domain (H_{N} domain) and a clostridial neurotoxin receptor binding domain (H_{C} domain), wherein the L-chain is catalytically inactive.
17. The polypeptide for use, method or use according to any one of clauses 1-12 or 14, wherein the polypeptide consists of a clostridial neurotoxin light-chain (L-chain), a clostridial neurotoxin translocation domain (H_{N} domain) and/or a clostridial neurotoxin receptor binding domain (H_{C} domain), wherein when the polypeptide comprises or consists of a clostridial neurotoxin L-chain, the L-chain is catalytically inactive.
18. The polypeptide for use, method or use according to any one of clauses 1-12, 15 or 17, wherein the polypeptide consists of a clostridial neurotoxin light-chain (L-chain) and a clostridial neurotoxin translocation domain (H_{N} domain), wherein the L-chain is catalytically inactive.
19. The polypeptide for use, method or use according to any one of clauses 1-13, 14, 16 or 17, wherein the polypeptide consists of a clostridial neurotoxin light-chain (L-chain), a clostridial neurotoxin translocation domain (H_{N} domain) and a clostridial neurotoxin receptor binding domain (H_{C} domain), wherein the L-chain is catalytically inactive.
20. The polypeptide for use, method or use according to any one of clauses 1-12, 14, 15, 17 or 18, wherein the polypeptide does not comprise both a clostridial neurotoxin H_{N} domain and H_{C} domain.
21. The polypeptide for use, method or use according to any one of the preceding clauses, wherein the polypeptide does not further comprise a non-clostridial catalytic domain.
22. The polypeptide for use, method or use according to any one of the preceding clauses, wherein the pain is chronic pain.
23. The polypeptide for use, method or use according to any one of clauses 1-21, wherein the pain is acute pain.
24. The polypeptide for use, method or use according to any one of the preceding clauses, wherein the pain is inflammatory pain.
25. The polypeptide for use, method or use according to clause 24, wherein the inflammatory pain is caused by or associated with sunburn, UV-induced damage, an arthritic disorder, an autoimmune disease, a connective tissue disorder, an injury, an infection, neuritis, joint inflammation or a headache (preferably a muscular/myogenic headache, a vascular headache, a high blood pressure headache, a hormone headache, a rebound headache, a chronic sinusitis headache, an organic headache, or an ictal headache).
26. The polypeptide for use, method or use according to any one of clauses 1-23, wherein the pain is neuropathic pain.
27. The polypeptide for use, method or use according to clause 26, wherein the neuropathic pain is (or is caused by or associated with) neuralgia, deafferentation, a complex regional pain syndrome (CRPS), or a neuropathy (e.g. a central or peripheral neuropathy).
28. The polypeptide for use, method or use according to any one of clauses 1-23, wherein the pain is mixed pain.
29. The polypeptide for use, method or use according to any one of clauses 1-23, wherein the pain is allodynia.
30. The polypeptide for use, method or use according to any one of clauses 1-23, wherein the pain is visceral pain.
31. The polypeptide for use, method or use according to clause 30, wherein the visceral pain is (or is caused by or associated with) functional visceral pain, chronic gastrointestinal inflammation, autoimmune pain, organic visceral pain, or treatment-induced visceral pain.
32. The polypeptide for use, method or use according to any one of clauses 1-23, wherein the pain is headache pain (e.g. a migraine).
33. The polypeptide for use, method or use according to clause 32, wherein the pain is migraine pain.
34. The polypeptide for use, method or use according to clause 32, wherein the headache pain is caused by or associated with a muscular/myogenic headache, a vascular headache, a high blood pressure headache, a hormone headache, a rebound headache, a chronic sinusitis headache, an organic headache, or an ictal headache.
35. The polypeptide for use, method or use according to any one of clauses 1-23, wherein the pain is post-operative pain.
36. The polypeptide for use, method or use according to any one of clauses 1-23, wherein the pain is referred pain.
37. The polypeptide for use, method or use according to any one of clauses 1-23, wherein the pain is somatic pain.
38. The polypeptide for use, method or use according to clause 37, wherein the pain is somatic pain caused by or associated with excessive muscle tension, a repetitive motion disorder, a muscle disorder, myalgia, an infection, or drugs.
39. The polypeptide for use, method or use according to any one of clauses 1-23, wherein the pain is bladder pain syndrome, preferably wherein the bladder pain is caused by or associated with interstitial cystitis.
40. The polypeptide for use, method or use according to any one of clauses 1-23, wherein the pain is phantom limb pain.
41. The polypeptide for use, method or use according to any one of clauses 5-21, wherein the inflammatory disorder is one or more selected from: cystitis, endometriosis, rheumatoid arthritis, complex regional pain syndrome, and neuritis.
42. The polypeptide for use, method or use according to clause 41, wherein the cystitis is interstitial cystitis.
43. The polypeptide for use, method or use according to clause 41, wherein the neuritis is peripheral neuritis.
44. The polypeptide for use, method or use according to any one of the preceding clauses, wherein a single dose of the polypeptide administered is greater than 250 μg.
45. The polypeptide for use, method or use according to any one of the preceding clauses, wherein a single dose of the polypeptide administered is 251 μg to 10 g.
46. The polypeptide for use, method or use according to any one of the preceding clauses, wherein a single dose of the polypeptide administered is 251 μg to 1 g.
47. The polypeptide for use, method or use according to any one of the preceding clauses, wherein a single dose of the polypeptide administered is 251-1000 μg.
48. The polypeptide for use, method or use according to any one of the preceding clauses, wherein the polypeptide is administered iteratively (e.g. as part of a pain treatment regimen).
49. The polypeptide for use, method or use according to any one of the preceding clauses, wherein the polypeptide is administered intradermally.
50. The polypeptide for use, method or use according to any one of the preceding clauses, wherein the polypeptide comprises a polypeptide sequence having at least 70% sequence identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 60, 61, 62, 63, 64, 65, 66, 67, 68, 69, 70, 74, 75 or 76 with the proviso that when the polypeptide comprises a clostridial neurotoxin L-chain, the L-chain is catalytically inactive.
51. The polypeptide for use, method or use according to any one of the preceding clauses, wherein the polypeptide comprises a polypeptide sequence having at least 80% sequence identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 60, 61, 62, 63, 64, 65, 66, 67, 68, 69, 70, 74, 75 or 76 with the proviso that when the polypeptide comprises a clostridial neurotoxin L-chain, the L-chain is catalytically inactive.
52. The polypeptide for use, method or use according to any one of the preceding clauses, wherein the polypeptide comprises a polypeptide sequence having at least 90% sequence identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 60, 61, 62, 63, 64, 65, 66, 67, 68, 69, 70, 74, 75 or 76 with the proviso that when the polypeptide comprises a clostridial neurotoxin L-chain, the L-chain is catalytically inactive.
53. The polypeptide for use, method or use according to any one of the preceding clauses, wherein the polypeptide comprises a polypeptide sequence of any one of SEQ ID NOs: 2, 8, 10, 12, 14, 16, 18, 22, 26, 30, 34, 38, 42, 44, 46, 48, 50, 61, 62, 63, 64, 65, 66, 67, 68, 69, 70, 74, 75 or 76.
54. The polypeptide for use, method or use according to any one of the preceding clauses, wherein the polypeptide is a catalytically inactive BoNT/A.
55. The polypeptide for use, method or use according to any one of the preceding clauses, wherein the polypeptide is a modified clostridial neurotoxin, such as a chimeric clostridial neurotoxin or a hybrid clostridial neurotoxin, preferably wherein the polypeptide does not comprise a native clostridial neurotoxin H-chain.
56. The polypeptide for use, method or use according to any one of clauses 1-53 or 55, wherein the polypeptide lacks a functional H_{CC} domain or H_{C} domain of a clostridial neurotoxin.
57. The polypeptide for use, method or use according to any one of clauses 1-53 or 55-56, wherein the polypeptide is a retargeted clostridial neurotoxin comprising a non-clostridial Targeting Moiety (TM).
58. The polypeptide for use, method or use according to any one of clauses 1-53 or 55-56, wherein the polypeptide lacks a functional H_{C} domain of a clostridial neurotoxin and also lacks any functionally equivalent exogenous ligand Targeting Moiety (TM).
59. The polypeptide for use, method or use according to any one of the preceding clauses, wherein the polypeptide is not expressed in a cell of the subject, e.g. wherein the use or method does not comprise expressing a nucleic acid encoding the polypeptide in a cell of the subject.
60. The polypeptide for use, method or use according to any one of the preceding clauses, wherein the polypeptide further comprises one or more non-clostridial neurotoxin sequences.
61. The polypeptide for use, method or use according to clause 60, wherein the one or more non-clostridial neurotoxin sequence(s) do(es) not bind to a cellular receptor.
62. The polypeptide for use, method or use according to clause 60 or 61, wherein the one or more non-clostridial neurotoxin sequence(s) do(es) not comprise a ligand for a cellular receptor.
63. The polypeptide for use, method or use according to any one of clauses 1-53, 55 or 59-62, wherein the polypeptide is a chimeric botulinum neurotoxin (BoNT) comprising a catalytically inactive BoNT/A light-chain and translocation domain, and a BoNT/B receptor binding domain (H_{C} domain).
64. The polypeptide for use, method or use according to any one of clauses 1-55 or 59-62, wherein the polypeptide comprises a modified BoNT/A H_{C} domain comprising a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.
65. The polypeptide for use, method or use according to any one of clauses 1-53 or 55-62, wherein the polypeptide comprises a catalytically inactive botulinum neurotoxin serotype X (BoNT/X) L-chain, a BoNT/X H_{N} domain, and/or a BoNT/X H_{C} domain.
66. The polypeptide for use, method or use according to any one of clauses 1-53, 59-62 or 65, wherein the polypeptide is a chimeric botulinum neurotoxin (BoNT) comprising a catalytically inactive BoNT/X light-chain and translocation domain, and a receptor binding domain (H_{C} domain) from a different (i.e. non-BoNT/X) clostridial neurotoxin.
67. The polypeptide for use, method or use according to any one of clauses 1-53, 59-62 or 65-66, wherein the polypeptide is a chimeric botulinum neurotoxin (BoNT) comprising a catalytically inactive BoNT/X light-chain and translocation domain, and a BoNT/B receptor binding domain (H_{C} domain).
68. The polypeptide for use, method or use according to any one of clauses 65-67, wherein the pain is inflammatory pain.
69. The polypeptide for use, method or use according to any one of the preceding clauses, wherein the polypeptide comprises Cys-(Xaa)ₐ-Ile-Asp/Glu-Gly-Arg-(Yaa)_{b}-Cys (SEQ ID NO: 71), wherein a=1-10 and b=4-15.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1-93. (canceled)

94. A method for treating a lower urinary tract disorder, the method comprising administering a polypeptide to a subject, wherein the polypeptide comprises:
(a) at least one of the following:
(i) a catalytically-inactive clostridial neurotoxin light-chain (L-chain),
(ii) a clostridial neurotoxin translocation domain (H_{N} domain), and/or
(iii) a clostridial neurotoxin receptor binding domain (H_{C} domain); or
(b) a clostridial neurotoxin L-chain and lacks a functional H_{CC} domain or H_{C} domain of a clostridial neurotoxin.

95. The method of claim 94, wherein the lower urinary tract disorder is: a bladder disorder;
an afferent nerve-associated disorder; bladder pain syndrome; overactive bladder; or detrusor overactivity.

96. A method for treating pain or an inflammatory disorder, the method comprising administering a polypeptide to a subject, wherein the polypeptide comprises:
(a) at least one of the following:
(i) a catalytically inactive clostridial neurotoxin L-chain,
(ii) a clostridial neurotoxin H_{N} domain, and/or
(iii) a clostridial neurotoxin H_{C} domain; or
(b) a clostridial neurotoxin L-chain and lacks a functional H_{CC} domain or H_{C} domain of a clostridial neurotoxin.

97. The method of claim 94, wherein:
(a) the polypeptide comprises a clostridial neurotoxin H_{N} domain;
(b) the polypeptide does not comprise any other catalytically-active domain;
(c) the polypeptide does not comprise an additional therapeutic or diagnostic agent beyond the clostridial neurotoxin L-chain, H_{N} domain, or H_{C} domain;
(d) the polypeptide is not administered with an additional therapeutic or diagnostic agent;
(e) the polypeptide does not comprise a non-clostridial catalytic domain;
(f) the polypeptide is not expressed in a cell of the subject;
(g) the polypeptide further comprises one or more non-clostridial neurotoxin sequences; and/or
(h) the polypeptide comprises the sequence Cys-(Xaa)ₐ-Ile-Asp/Glu-Gly-Arg-(Yaa)_{b}-Cys (SEQ ID NO: 71), wherein a is 1-10 and b is 4-15.

98. The method of claim 94, wherein the polypeptide:
(a) comprises a clostridial neurotoxin L-chain;
(b) comprises a catalytically-inactive clostridial neurotoxin L-chain, a clostridial neurotoxin H_{N} domain, and a clostridial neurotoxin H_{C} domain;
(c) consists essentially of a catalytically-inactive clostridial neurotoxin L-chain, a clostridial neurotoxin H_{N} domain, and/or a clostridial neurotoxin H_{C} domain;
(d) consists essentially of a catalytically-inactive clostridial neurotoxin L-chain and a clostridial neurotoxin H_{N} domain;
(e) consists of a catalytically-inactive clostridial neurotoxin L-chain, a clostridial neurotoxin H_{N} domain, and/or a clostridial neurotoxin H_{C} domain; or
(f) consists of a catalytically-inactive clostridial neurotoxin L-chain and a clostridial neurotoxin H_{N} domain.

99. The method of claim 94, wherein the polypeptide does not comprise both a clostridial neurotoxin H_{N} domain and a clostridial neurotoxin H_{C} domain.

100. The method according to claim 94 wherein:
(a) a greater than 250 μg dose of the polypeptide is administered;
(b) the polypeptide is administered iteratively or intradermally;
(c) the polypeptide comprises a sequence having at least 70% sequence identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 60, 61, 62, 63, 64, 65, 66, 67, 68, 69, 70, 74, 75 or 76 with the proviso that when the polypeptide comprises a clostridial neurotoxin L-chain, the L-chain is catalytically inactive; and/or
(d) the polypeptide is a modified clostridial neurotoxin.

101. The method of claim 94, wherein the polypeptide lacks a functional clostridial neurotoxin H_{C} domain and any functionally equivalent exogenous ligand-targeting moiety.

102. The method according to claim 94, wherein:
(a) the polypeptide is a catalytically inactive botulin neurotoxin serotype A (BoNT/A);
(b) the polypeptide lacks a functional clostridial neurotoxin H_{CC} domain or H_{C} domain;
(c) the polypeptide is a retargeted clostridial neurotoxin comprising a non-clostridial Targeting Moiety (TM);
(d) the polypeptide is a chimeric botulinum neurotoxin (BoNT) comprising a catalytically inactive BoNT/A L-chain and H_{N} domain, and a H_{C} domain from botulinum neurotoxin serotype B (BoNT/B);
(e) the polypeptide comprises a modified BoNT/A H_{C} domain comprising a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
(i) substitution of an acidic surface exposed amino acid residue with a basic amino acid residue;
(ii) substitution of an acidic surface exposed amino acid residue with an uncharged amino acid residue;
(iii) substitution of an uncharged surface exposed amino acid residue with a basic amino acid residue;
(iv) insertion of a basic amino acid residue; and
(v) deletion of an acidic surface exposed amino acid residue;
(f) the polypeptide comprises a catalytically-inactive botulinum neurotoxin serotype X (BoNT/X) L-chain, a BoNT/X H_{N} domain, and/or a BoNT/X H_{C} domain; or
(g) the polypeptide is a chimeric BoNT comprising a catalytically inactive BoNT/X L-chain, a BoNT/X H_{N} domain, and H_{C} domain from a different clostridial neurotoxin.

103. The method according to claim 96, wherein:
(a) the polypeptide comprises a clostridial neurotoxin H_{N} domain;
(b) the polypeptide does not comprise any other catalytically-active domain;
(c) the polypeptide does not comprise an additional therapeutic or diagnostic agent beyond the clostridial neurotoxin L-chain, H_{N} domain, or H_{C} domain;
(d) the polypeptide is not administered with an additional therapeutic or diagnostic agent;
(e) the polypeptide does not comprise a non-clostridial catalytic domain;
(f) the polypeptide is not expressed in a cell of the subject;
(g) the polypeptide further comprises one or more non-clostridial neurotoxin sequences; and/or
(h) the polypeptide comprises the sequence Cys-(Xaa)ₐ-Ile-Asp/Glu-Gly-Arg-(Yaa)_{b}-Cys (SEQ ID NO: 71), wherein a is 1-10 and b is 4-15.

104. The method according to claim 96, wherein the polypeptide:
(a) comprises a clostridial neurotoxin L-chain;
(b) comprises a catalytically-inactive clostridial neurotoxin L-chain, a clostridial neurotoxin H_{N} domain, and a clostridial neurotoxin H_{C} domain;
(c) consists essentially of a catalytically-inactive clostridial neurotoxin L-chain, a clostridial neurotoxin H_{N} domain, and/or a clostridial neurotoxin H_{C} domain;
(d) consists essentially of a catalytically-inactive clostridial neurotoxin L-chain and a clostridial neurotoxin H_{N} domain;
(e) consists of a catalytically-inactive clostridial neurotoxin L-chain, a clostridial neurotoxin H_{N} domain, and/or a clostridial neurotoxin H_{C} domain; or
(f) consists of a catalytically-inactive clostridial neurotoxin L-chain and a clostridial neurotoxin H_{N} domain.

105. The method according to claim 96, wherein the polypeptide does not comprise both a clostridial neurotoxin H_{N} domain and H_{C} domain.

106. The method according to claim 96, wherein the pain is chronic pain or acute pain.

107. The method according to claim 96, wherein the pain is bladder pain, inflammatory pain, neuropathic pain, mixed pain, allodynia, visceral pain, headache pain, post-operative pain, referred pain, somatic pain, or phantom limb pain.

108. The method according to claim 96, wherein the inflammatory disorder is selected from: cystitis, endometriosis, rheumatoid arthritis, complex regional pain syndrome, and neuritis; preferably wherein the cystitis is interstitial cystitis, or the neuritis is peripheral neuritis.

109. The method according to claim 96 wherein:
(a) a greater than 250 μg dose of the polypeptide is administered;
(b) the polypeptide is administered iteratively or intradermally;
(c) the polypeptide comprises a polypeptide sequence having at least 70% sequence identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 60, 61, 62, 63, 64, 65, 66, 67, 68, 69, 70, 74, 75 or 76 with the proviso that when the polypeptide comprises a clostridial neurotoxin L-chain, the L-chain is catalytically inactive; and/or
(d) the polypeptide is a modified clostridial neurotoxin.

110. The method of claim 96, wherein the polypeptide lacks a functional clostridial neurotoxin H_{C} domain and any functionally equivalent exogenous ligand-targeting moiety.

111. The method according to claim 96, wherein:
(a) the polypeptide is a catalytically inactive BoNT/A;
(b) the polypeptide lacks a functional clostridial neurotoxin H_{CC} domain or H_{C} domain;
(c) the polypeptide is a retargeted clostridial neurotoxin comprising a non-clostridial TM;
(d) the polypeptide is a chimeric BoNT comprising a catalytically inactive BoNT/A L-chain and H_{N} domain, and a H_{C} domain from BoNT/B;
(e) the polypeptide comprises a modified BoNT/A H_{C} domain comprising a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
(i) substitution of an acidic surface exposed amino acid residue with a basic amino acid residue;
(ii) substitution of an acidic surface exposed amino acid residue with an uncharged amino acid residue;
(iii) substitution of an uncharged surface exposed amino acid residue with a basic amino acid residue;
(iv) insertion of a basic amino acid residue; and
(v) deletion of an acidic surface exposed amino acid residue;
(f) the polypeptide comprises a catalytically-inactive BoNT/X L-chain, a BoNT/X H_{N} domain, and/or a BoNT/X H_{C} domain; or
(g) the polypeptide is a chimeric BoNT comprising a catalytically inactive BoNT/X L-chain, a BoNT/X H_{N} domain, and H_{C} domain from a different clostridial neurotoxin.
